(19) United States
(12) Patent Application Publication
(10) Pub. No.: US 2026/0021195 A1
(43) Pub. Date: Jan. 22, 2026

(54) RNAI AGENTS FOR INHIBITING EXPRESSION OF HUNTINGTIN (HTT), COMPOSITIONS THEREOF, AND METHODS OF USE

(71) Applicant: ARROWHEAD PHARMACEUTICALS, INC., Pasadena, CA (US)

(72) Inventors: Christine ESAU, San Diego, CA (US); Tao PEI, Middleton, WI (US); Xiaokai LI, San Diego, CA (US); Ivar STEIN, San Diego, CA (US); George NACLERIO, III, Madison, WI (US); Ji Young SUK, San Diego, CA (US)

(21) Appl. No.: 19/244,201

(22) Filed: Jun. 20, 2025

Related U.S. Application Data
(60) Provisional application No. 63/662,268, filed on Jun. 20, 2024.
(60) Provisional application No. 63/703,377, filed on Oct. 4, 2024.
(60) Provisional application No. 63/743,015, filed on Jan. 8, 2025.

(51) Int. Cl. A61K 47/68; C07K 16/18; C12N 15/113

(52) U.S. Cl. CPC A61K 47/6807; A61K 47/6843; C07K 16/18; C12N 15/113; C07K 2317/55; C12N 2310/11; C12N 2310/31; C12N 2310/351

(57) ABSTRACT

Described are RNAi agents, compositions that include RNAi agents, and methods for inhibition of a huntingtin (HTT) gene. The HTT RNAi agents and RNAi agent conjugates disclosed herein inhibit the expression of an HTT gene. The HTT RNAi agents are conjugated to an antigen binding protein that may enable subcutaneous delivery of the RNAi agents by facilitating crossing of the blood brain barrier (BBB). Pharmaceutical compositions that include one or more HTT RNAi agents, optionally with one or more additional therapeutics, are also described. Delivery of the described HTT RNAi agents to central nervous system (CNS) tissue, in vivo, provides for inhibition of HTT gene expression and a reduction in HTT activity, which can provide a therapeutic benefit to subjects, including human subjects, for the treatment of various diseases including Huntington's Disease.

### CROSS REFERENCE TO RELATED APPLICATIONS

This application claims the benefit of priority to U.S. Provisional Patent Application Ser. No. 63/662,268, filed on Jun. 20, 2024, U.S. Provisional Patent Application Ser. No. 63/703,377, filed on Oct. 4, 2024, and U.S. Provisional Patent Application Ser. No. 63/743,015, filed on Jan. 8, 2025, the contents of each of which are incorporated herein by reference in their entirety.

### FIELD OF THE INVENTION

The present disclosure relates to RNA interference (RNAi) agents, e.g., double stranded RNAi agents such as chemically modified small interfering RNAs (siRNAs), for inhibition of huntingtin (“HTT”) gene expression, compositions that include HTT RNAi agents, and methods of use thereof.

### SEQUENCE LISTING

This application contains a Sequence Listing (in compliance with Standard ST26), which has been submitted in xml format and is hereby incorporated by reference in its entirety. The xml sequence listing file is named 4140_1380003_SequenceListing_ST26.xml, created Jun. 20, 2025, and is 5,134,314 bytes in size.

### BACKGROUND

The present disclosure relates to RNA interference (RNAi) agents, e.g., double stranded RNAi agents such as chemically modified small interfering RNAs (siRNAs), for inhibition of huntingtin (“HTT”) gene expression, compositions that include HTT RNAi agents, and methods of use thereof.

Huntington's disease causes nerve cells in the brain to decay over time, affecting a person's movements, thinking ability, and mental health. This disease is inherited in autosomal dominant pattern. 2024 Mayo Foundation for Medical Education and Research (MFMER). Huntington's disease affects approximately 3 to 7 per 100,000 people of European ancestry. MedlinePlus, National Library of Medicine, 2024.

Huntington's disease (HD) is a neurodegenerative disorder caused by an expanded CAG (cytosine, adenine, and guanine) trinucleotide repeat in the huntingtin (HTT) gene. Normal healthy individuals have 10-35 CAG repeats within the HTT gene. Mutant HTT genes contain 36 to more than 120 CAG repeats. The increase in CAG repeats results in the expression of an mutant HTT protein with an abnormally long poly-glutamine tract that leads to the formation of toxic aggregates in neurons. This leads to disruption of normal cellular function. There are treatments that can reduce the severity of some symptoms of Huntington's disease, but currently there are no treatments that can alter the course of the disease. Treatments to reduce the expression of mutant HTT protein are likely to provide long-term management of the disease.

### SUMMARY

There exists a need for novel RNA interference (RNAi) agents (termed RNAi agents, RNAi triggers, or triggers), e.g., double stranded RNAi agents such as siRNAs, that are able to selectively and efficiently inhibit the expression of a mutant HTT gene, including for use as a therapeutic or medicament. Further, there exists a need for compositions of novel HTT-specific RNAi agents for the treatment of diseases or disorders associated mutant HTT gene expression and/or disorders that can be mediated at least in part by a reduction in HTT gene expression.

The nucleotide sequences and chemical modifications of the HTT RNAi agents disclosed herein, as well as their combination with certain specific antigen binding proteins suitable for selectively and efficiently delivering the HTT RNAi agents to relevant CNS cells in vivo, differ from those previously disclosed or known in the art. The HTT RNAi agents disclosed herein provide for highly potent and efficient inhibition of the expression of an HTT gene.

In general, the present disclosure features HTT gene-specific RNAi agents, compositions that include HTT RNAi agents, and methods for inhibiting expression of an HTT gene in vitro and/or in vivo using the HTT RNAi agents and compositions that include HTT RNAi agents described herein. The HTT RNAi agents described herein are able to selectively and efficiently decrease expression of an HTT gene, and thereby reduce the expression of the HTT protein.

The described HTT RNAi agents can be used in methods for therapeutic treatment (including preventative or prophylactic treatment) of symptoms and diseases including, but not limited to, various central nervous system diseases and neurodegenerative diseases (including Huntington's Disease).

In one aspect, the disclosure features RNAi agents for inhibiting expression of an HTT gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing HTT such as endothelial cells, neurons, microglia, and astrocytes, inhibit the expression of one or more HTT gene variants in vivo and/or in vitro.

The HTT RNAi agents disclosed herein target a human HTT gene (see, e.g., SEQ ID NO: 1). In some embodiments, the HTT RNAi agents disclosed herein target a portion of an HTT gene having the sequence of any of the sequences disclosed in Table 1.

In another aspect, the disclosure features compositions, including pharmaceutical compositions, that include one or more of the disclosed HTT RNAi agents that are able to selectively and efficiently decrease expression of an HTT gene. The compositions that include one or more HTT RNAi agents described herein can be administered to a subject, such as a human or animal subject, for the treatment (including prophylactic treatment or inhibition) of symptoms and diseases associated with HTT protein or enzyme levels.

Examples of HTT RNAi agent sense strands and antisense strands that can be used in an HTT RNAi agent are provided in Tables 3, 4, 5, and 6. Examples of HTT RNAi agent duplexes are provided in Tables 7, 8, and 9. Examples of 19-nucleotide core stretch sequences that may consist of or may be included in the sense strands and antisense strands of certain HTT RNAi agents disclosed herein, are provided in Table 2.

In another aspect, the disclosure features methods for delivering HTT RNAi agents to neurons, astrocytes, microglia and endothelial cells in a subject, such as a mammal, in vivo. Also described herein are compositions for use in such methods. In some embodiments, disclosed herein are methods for delivering HTT RNAi agents to central nervous system cells (neurons, astrocytes, microglia and endothelial cells) to a subject in vivo. In some embodiments, the subject is a human subject.

The methods disclosed herein include the administration of one or more HTT RNAi agents to a subject, e.g., a human or animal subject, by any suitable means known in the art. The pharmaceutical compositions disclosed herein that include one or more HTT RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by intrathecal injection, intracerebroventricular injection, or subcutaneous injection.

In some embodiments, it is desired that the HTT RNAi agents described herein inhibit the expression of an HTT gene in central nervous system cells.

The one or more HTT RNAi agents can be delivered to target cells or tissues using any oligonucleotide delivery technology known in the art. In some embodiments, an HTT RNAi agent is delivered to cells or tissues by covalently linking the RNAi agent to a targeting group or an antigen binding protein.

An antigen binding protein can be linked to the 3′ or 5′ end of a sense strand or an antisense strand of an HTT RNAi agent. In some embodiments, an antigen binding protein is linked to the 3′ or 5′ end of the sense strand. In some embodiments, an antigen binding protein is linked to the 5′ end of the sense strand. In some embodiments, an antigen binding protein is linked internally to a nucleotide on the sense strand and/or the antisense strand of the RNAi agent. In some embodiments, an antigen binding protein is linked to the RNAi agent via a linker.

In another aspect, the disclosure features compositions that include one or more HTT RNAi agents that have the duplex structures disclosed in Tables 7, 8, and 9.

The use of HTT RNAi agents provides methods for therapeutic (including prophylactic) treatment of diseases or disorders for which a reduction in HTT protein levels can provide a therapeutic benefit. The HTT RNAi agents disclosed herein can be used to treat various neurodegenerative diseases, including Huntington's Disease. Such methods of treatment include administration of an HTT RNAi agent to a human being or animal having elevated or mutant HTT protein or mutant HTT activity beyond desirable levels.

### BRIEF DESCRIPTION OF THE DRAWINGS

FIG. 1. Shows the chemical structure of AC007867 in free acid form.

FIG. 2. Shows the chemical structure of AC007867 in free base form.

### DETAILED DESCRIPTION

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a chemical composition of matter that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e. HTT mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of an HTT mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. In some embodiments, sequence identity, as applicable to a particular nucleotide or amino acid sequence, is calculated by a pairwise alignment using the Needleman-Wunsch algorithm using a generally available alignment program, e.g., the Needle (EMBOSS) program. The inventions disclosed herein encompass nucleotide sequences substantially identical to those disclosed herein. In particular embodiments, a nucleic acid sequence is 99% identical to a nucleotide sequence disclosed herein. In particular embodiments, a nucleic acid is 95% identical to a nucleotide sequence disclosed herein. In particular embodiments, an amino acid sequence is 99% identical to a polypeptide sequence disclosed herein. In particular embodiments, an amino acid is 95% identical to a polypeptide sequence disclosed herein. As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol

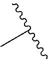

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### RNAi Agents

Described herein are RNAi agents for inhibiting expression of the huntingtin (or HTT) gene (referred to herein as HTT RNAi agents or HTT RNAi triggers). Each HTT RNAi agent disclosed herein comprises a sense strand and an antisense strand. The sense strand can be 15 to 49 nucleotides in length. The antisense strand can be 18 to 30 nucleotides in length. The sense and antisense strands can be either the same length or they can be different lengths. In some embodiments, the sense and antisense strands are each independently 18 to 27 nucleotides in length. In some embodiments, both the sense and antisense strands are each 21-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are each independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. In some embodiments, the RNAi agent antisense strands are each independently 18, 19, 20, 21, 22, 23, 24, 25, 26, 27, 28, 29, or 30 nucleotides in length. In some embodiments, a double-stranded RNAi agent has a duplex length of about 16, 17, 18, 19, 20, 21, 22, 23, or 24 nucleotides.

Examples of nucleotide sequences used in forming HTT RNAi agents are provided in Tables 2, 3, 4, 5, 6, and 9. Examples of RNAi agent duplexes, that include the sense strand and antisense strand sequences in Tables 2, 3, 4, 5, 6, are shown in Tables 7, 8, and 9.

In some embodiments, the region of perfect, substantial, or partial complementarity between the sense strand and the antisense strand is 16-26 (e.g., 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the HTT RNAi agents described herein includes at least 15 consecutive nucleotides that have at least 85% identity to a core stretch sequence (also referred to herein as a “core stretch” or “core sequence”) of the same number of nucleotides in an HTT mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the HTT mRNA target. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length.

An antisense strand of an HTT RNAi agent described herein includes at least 16 consecutive nucleotides that have at least 85% complementarity to a core stretch of the same number of nucleotides in an HTT mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the HTT mRNA target. In some embodiments, this antisense strand core stretch is 16, 17, 18, 19, 20, 21, 22, or 23 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The HTT RNAi agent sense and antisense strands anneal to form a duplex. A sense strand and an antisense strand of an HTT RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 16, at least 17, at least 18, at least 19, at least 20, at least 21, at least 22, or at least 23 nucleotides that is at least 85% or 100% complementary to a corresponding 16, 17, 18, 19, 20, 21, 22, or 23 nucleotide sequence of the antisense strand core stretch sequence (i.e., the sense and antisense core stretch sequences of an HTT RNAi agent have a region of at least 16, at least 17, at least 18, at least 19, at least 20, at least 21, at least 22, or at least 23 nucleotides that is at least 85% base paired or 100% base paired.)

In some embodiments, the antisense strand of an HTT RNAi agent disclosed herein differs by 0, 1, 2, or 3 nucleotides from any of the antisense strand sequences in Table 2 or Table 3. In some embodiments, the sense strand of an HTT RNAi agent disclosed herein differs by 0, 1, 2, or 3 nucleotides from any of the sense strand sequences in Table 2, Table 4, Table 5, Table 6, or Table 9.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the HTT mRNA. The sense strand additional nucleotides, if present, may or may not be identical to the corresponding sequence in the HTT mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or anti sense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, an HTT RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some embodiments, an HTT RNAi agent comprises an antisense strand having a 3′ extension of 1, 2, 3, 4, 5, or 6 nucleotides in length. In other embodiments, an HTT RNAi agent comprises an antisense strand having a 3′ extension of 1, 2, or 3 nucleotides in length. In some embodiments, one or more of the antisense strand extension nucleotides comprise nucleotides that are complementary to the corresponding HTT mRNA sequence. In some embodiments, one or more of the antisense strand extension nucleotides comprise nucleotides that are not complementary to the corresponding HTT mRNA sequence.

In some embodiments, an HTT RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the HTT mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, an HTT RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the HTT mRNA sequence.

Examples of sequences used in forming HTT RNAi agents are provided in Tables 2, 3, 4, 5, 6, and 9. In some embodiments, an HTT RNAi agent antisense strand includes a sequence of any of the sequences in Tables 2, 3, or 9. In certain embodiments, an HTT RNAi agent antisense strand comprises or consists of any one of the modified sequences in Table 3. In some embodiments, an HTT RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2 or 3. In some embodiments, an HTT RNAi agent sense strand includes the sequence of any of the sequences in Tables 2, 4, 5, or 6. In some embodiments, an HTT RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, or 6. In certain embodiments, an HTT RNAi agent sense strand comprises or consists of a modified sequence of any one of the modified sequences in Table 4, 5, 6, or 9.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The HTT RNAi agents disclosed herein may also be comprised of one or more modified nucleotides. In some embodiments, substantially all of the nucleotides of the sense strand and substantially all of the nucleotides of the antisense strand of the HTT RNAi agent are modified nucleotides. The HTT RNAi agents disclosed herein may further be comprised of one or more modified internucleoside linkages, e.g., one or more phosphorothioate and/or phosphorodithioate linkages. In some embodiments, an HTT RNAi agent contains one or more modified nucleotides and one or more modified internucleoside linkages. In some embodiments, a 2′-modified nucleotide is combined with modified internucleoside linkage.

In some embodiments, an HTT RNAi agent is prepared or provided as a salt, mixed salt, or a free-acid. In some embodiments, an HTT RNAi agent is prepared as a pharmaceutically acceptable salt. In some embodiments, an HTT RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, an HTT RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to herein or in the art as 2′-methoxy nucleotides), 2′-fluoro nucleotides (also referred to herein or in the art as 2′-deoxy-2′-fluoro nucleotides), 2′-deoxy nucleotides, 2′-methoxyethyl (2′-O-2-methoxylethyl) nucleotides (also referred herein or in the art as 2′-MOE nucleotides), 2′-amino nucleotides, and 2′-alkyl nucleotides. It is not necessary for all positions in a given compound to be uniformly modified. Conversely, more than one modification can be incorporated in a single HTT RNAi agent or even in a single nucleotide thereof. The HTT RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 10 herein.

### Modified Internucleoside Linkages

In some embodiments, one or more nucleotides of an HTT RNAi agent are linked by non-standard linkages or backbones (i.e., modified internucleoside linkages or modified backbones). Modified internucleoside linkages or backbones include, but are not limited to, phosphorothioate and/or phosphorodithioate groups (represented herein as a lower case “s” for phosphorothioate and “ss” for phosphorodithioate), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S. and CH₂ components.

In some embodiments, a sense strand of an HTT RNAi agent can contain 1, 2, 3, 4, 5, or 6 phosphorothioate and/or phosphorodithioate linkages, an antisense strand of an HTT RNAi agent can contain 1, 2, 3, 4, 5, or 6 phosphorothioate and/or phosphorodithioate linkages, or both the sense strand and the antisense strand independently can contain 1, 2, 3, 4, 5, or 6 phosphorothioate and/or phosphorodithioate linkages. In some embodiments, a sense strand of an HTT RNAi agent can contain 1, 2, 3, or 4 phosphorothioate or phosphorodithioate linkages, an antisense strand of an HTT RNAi agent can contain 1, 2, 3, or 4 phosphorothioate or phosphorodithioate linkages, or both the sense strand and the antisense strand independently can contain 1, 2, 3, or 4 phosphorothioate or phosphorodithioate linkages.

In some embodiments, an HTT RNAi agent sense strand contains at least two phosphorothioate or phosphorodithioate internucleoside linkages. In some embodiments, the phosphorothioate or phosphorodithioate internucleoside linkages are between the nucleotides at positions 1-3 from the 3′ end of the sense strand. In some embodiments, one phosphorothioate or phosphorodithioate internucleoside linkage is at the 5′ end of the sense strand nucleotide sequence, and another phosphorothioate or phosphorodithioate linkage is at the 3′ end of the sense strand nucleotide sequence. In some embodiments, two phosphorothioate or phosphorodithioate internucleoside linkage are located at the 5′ end of the sense strand, and another phosphorothioate or phosphorodithioate linkage is at the 3′ end of the sense strand. In some embodiments, the sense strand does not include any phosphorothioate or phosphorodithioate internucleoside linkages between the nucleotides, but contains one, two, or three phosphorothioate or phosphorodithioate linkages between the terminal nucleotides on both the 5′ and 3′ ends and the optionally present inverted abasic residue terminal caps. In some embodiments, a targeting ligand is linked to the sense strand via a phosphorothioate or phosphorodithioate linkage.

In some embodiments, an HTT RNAi agent antisense strand contains four phosphorothioate or phosphorodithioate internucleoside linkages. In some embodiments, the four phosphorothioate or phosphorodithioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate or phosphorodithioate internucleoside linkages are located between positions 1-4 from the 5′ end of the antisense strand, and a fourth phosphorothioate or phosphorodithioate internucleoside linkage is located between positions 20-21 from the 5′ end of the antisense strand. In some embodiments, an HTT RNAi agent contains at least three or four phosphorothioate or phosphorodithioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 10). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between a targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between a targeting ligand and the nucleotide sequence of the sense strand of the RNAi agent. The inverted abasic residues may be linked via phosphate, phosphorothioate or phosphorodithioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 10 below.

### HTT RNAi Agents

The HTT RNAi agents disclosed herein are designed to target specific positions on an HTT gene (e.g., SEQ ID NO:1 (NM_002111.8)). As defined herein, an antisense strand sequence is designed to target an HTT gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target an HTT gene at position 304 requires that when base pairing to the gene, the 5′ terminal nucleobase of the antisense strand is aligned with position 324 of an HTT gene.

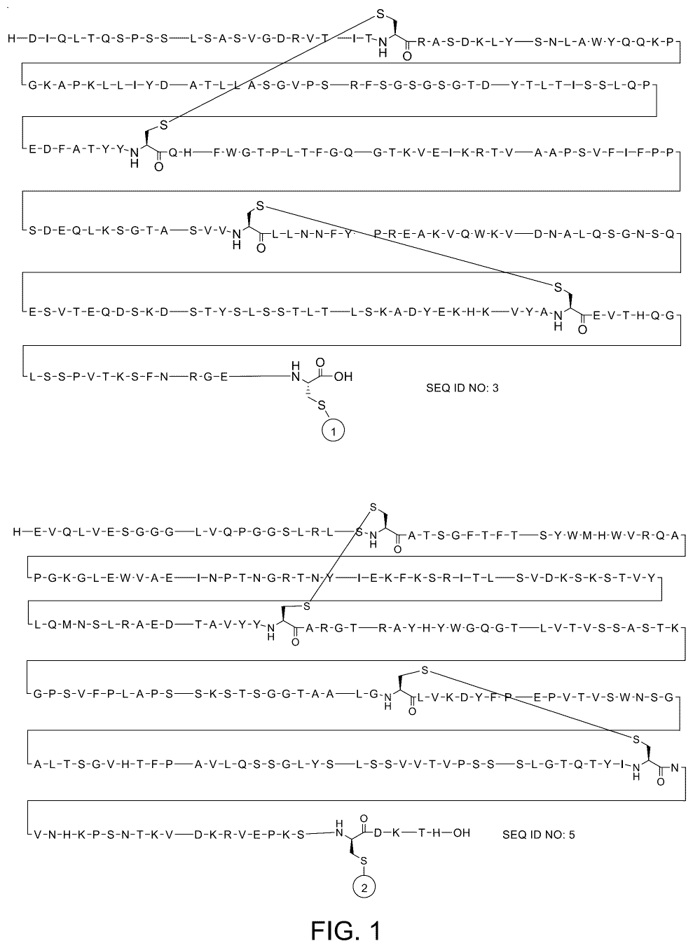

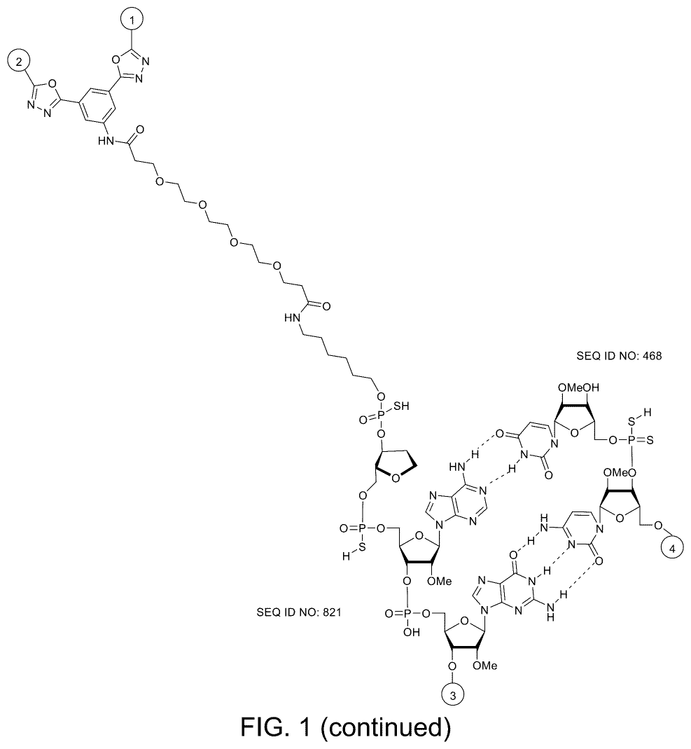

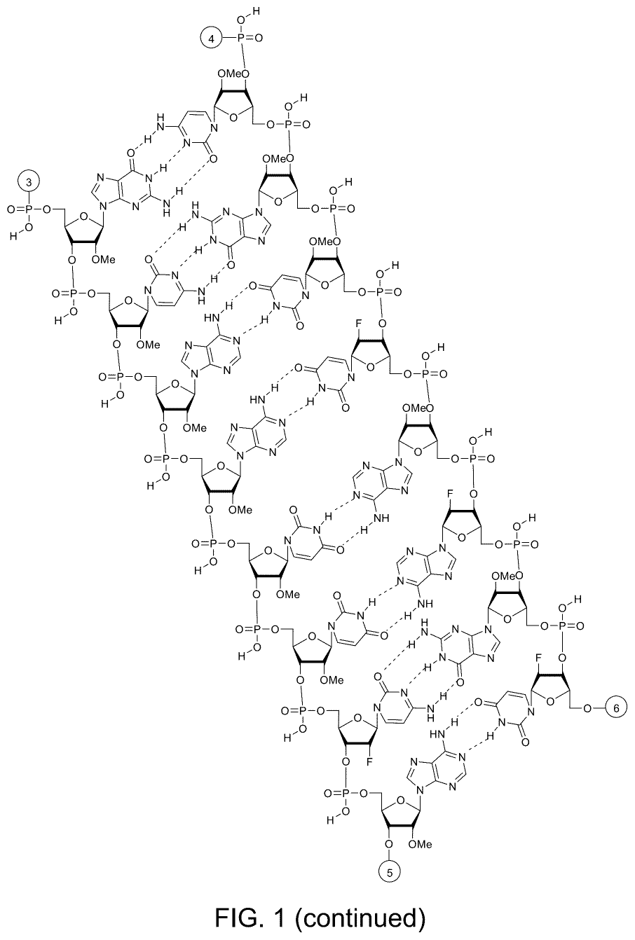

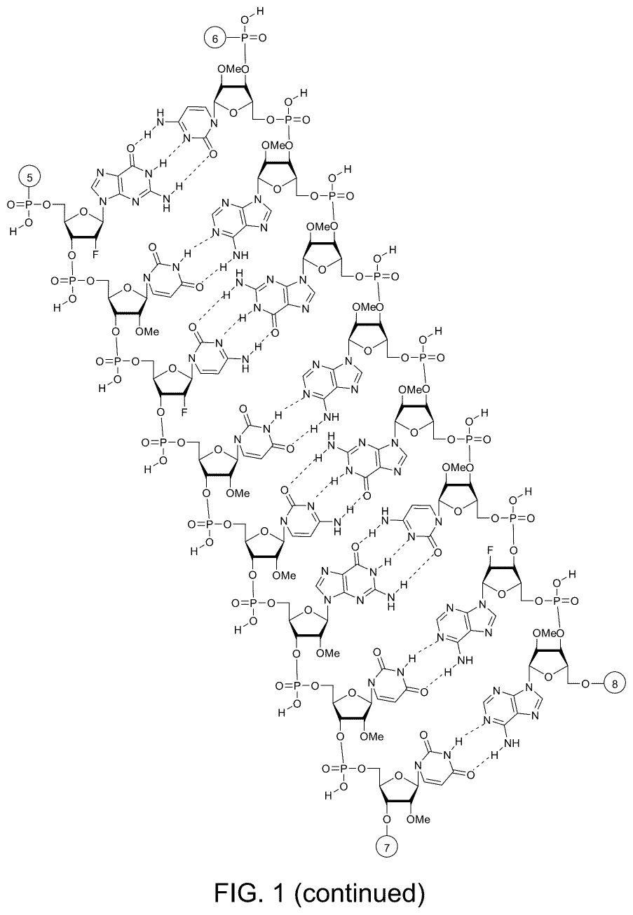

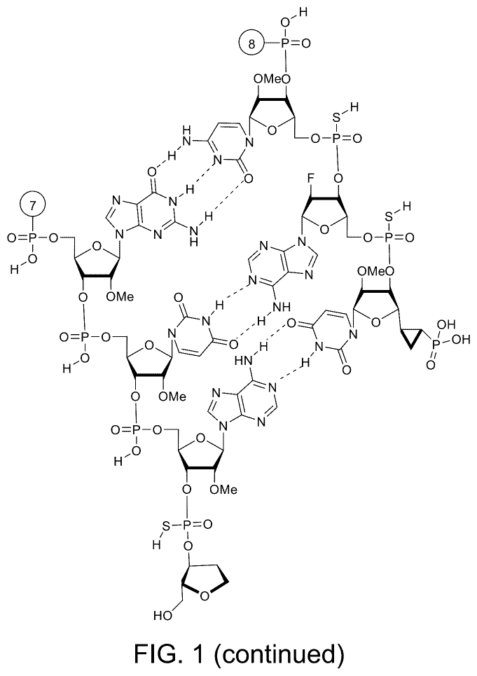

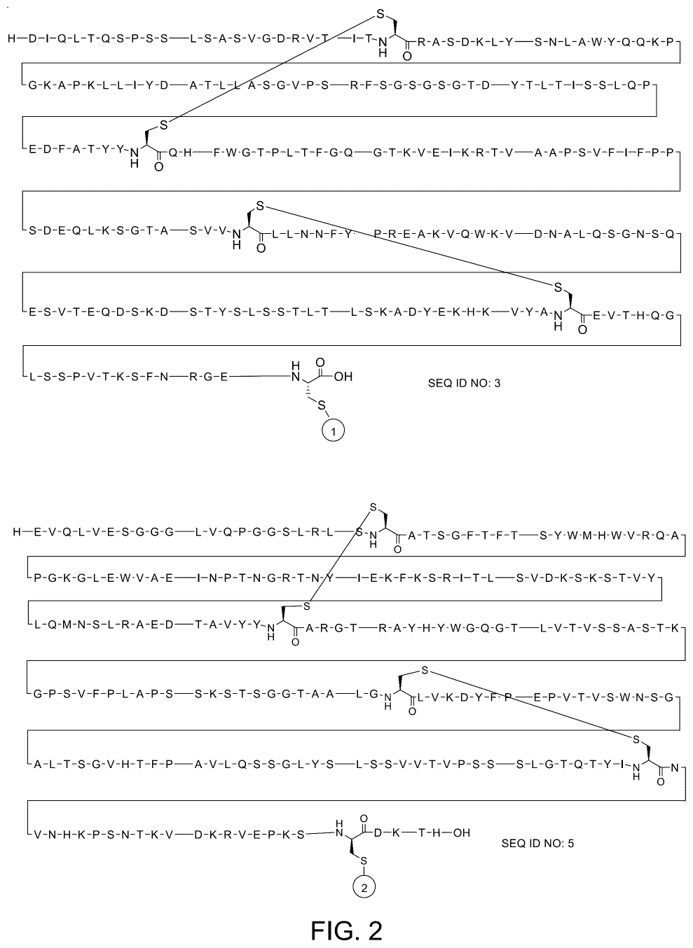

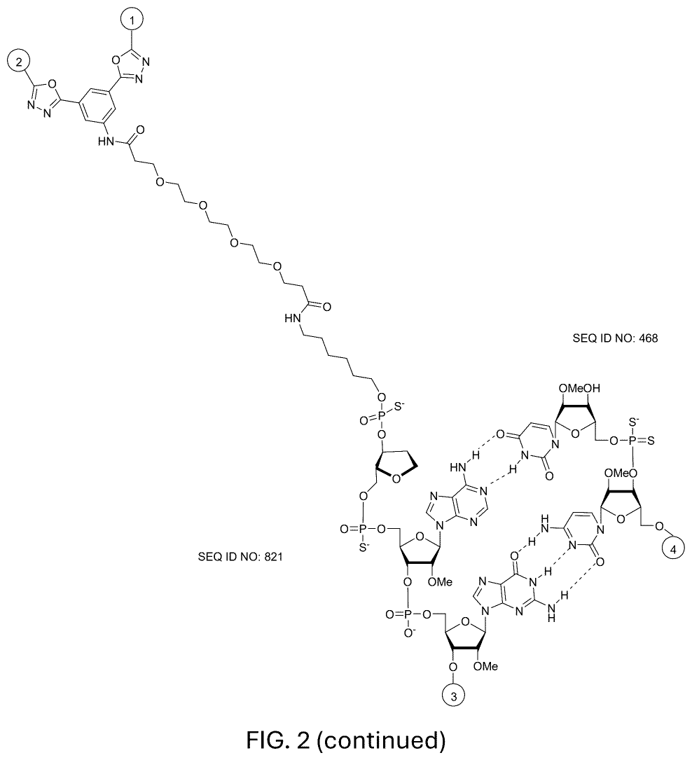

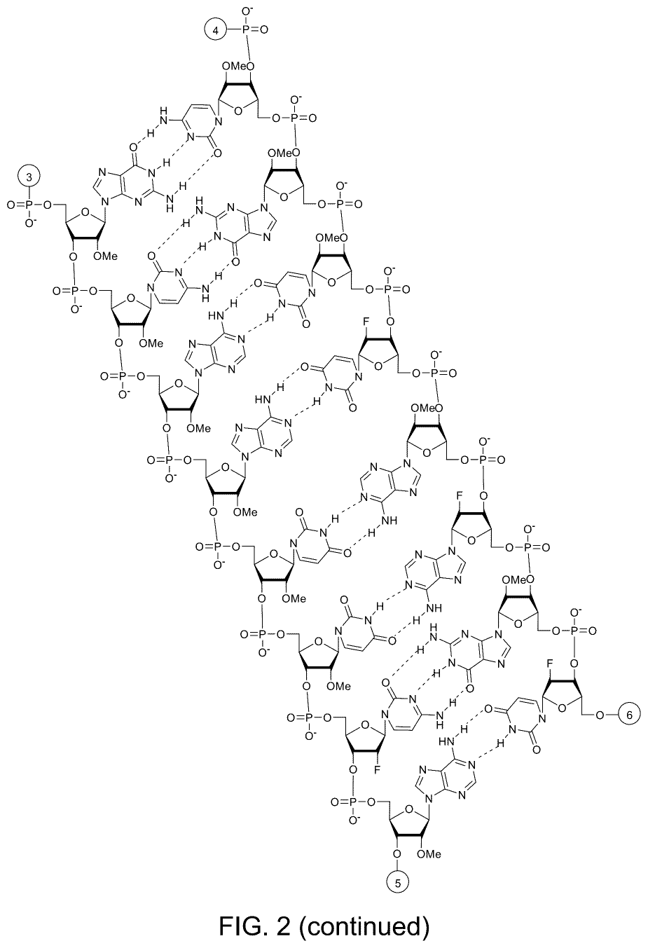

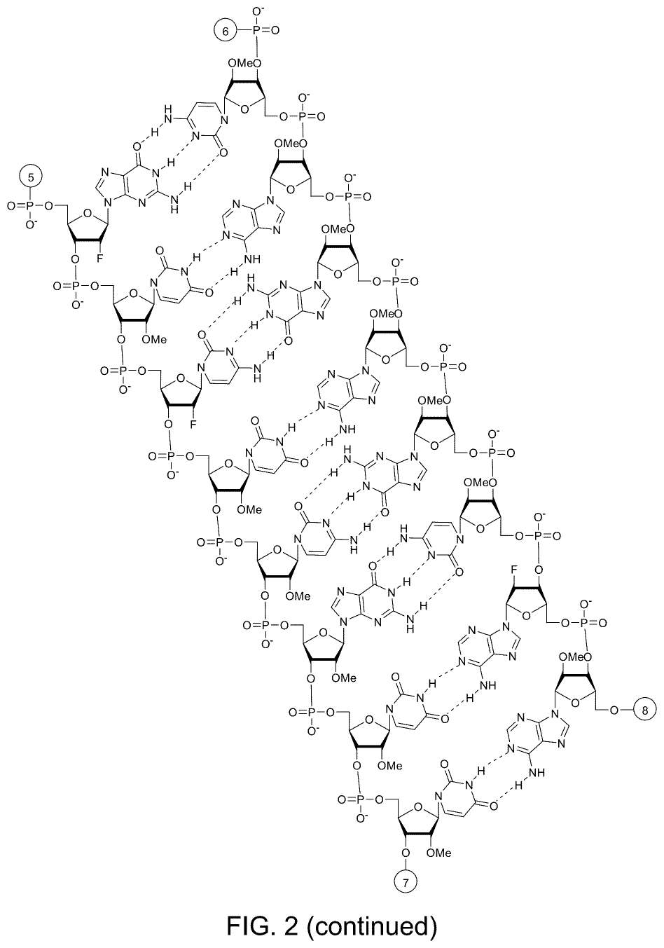

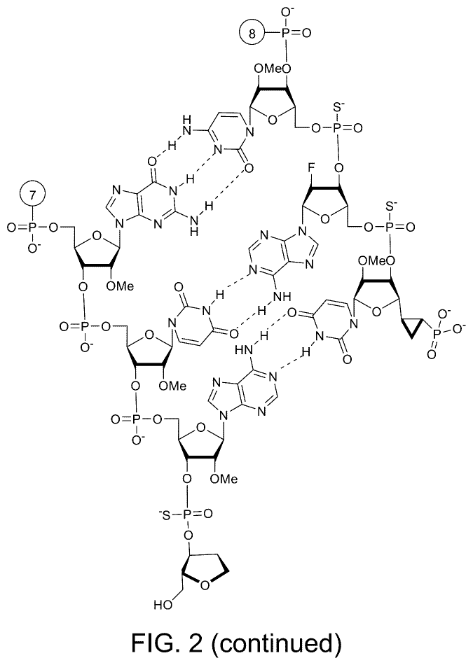

As provided herein, an HTT RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for an HTT RNAi agent disclosed herein that is designed to target position 304 of an HTT gene, the 5′ terminal nucleobase of the antisense strand of the HTT RNAi agent must be aligned with position 324 of the gene; however, the 5′ terminal nucleobase of the antisense strand may be, but is not required to be, complementary to position 324 of an HTT gene, provided that there is at least 85% complementarity (e.g., at least 85, 86, 87, 88, 89, 90, 91, 92, 93, 94, 95, 96, 97, 98, 99, or 100% complementarity) of the antisense strand and the gene transcript across a core stretch sequence of at least 16 consecutive nucleotides. As shown by, among other things, the various examples disclosed herein, the specific site of binding of the gene by the antisense strand of the HTT RNAi agent (e.g., whether the HTT RNAi agent is designed to target an HTT gene at position 304, at position 264, at position 785, or at some other position) is an important factor to the level of inhibition achieved by the HTT RNAi agent. (See, e.g., Kamola et al., The siRNA Non-seed Region and Its Target Sequences are Auxiliary Determinants of Off-Target Effects, PLOS Computational Biology, 11(12), FIG. 1 (2015)).

In some embodiments, the HTT RNAi agents disclosed herein target an HTT gene at or near the positions of the HTT sequence shown in Table 1. In some embodiments, the antisense strand of an HTT RNAi agent disclosed herein includes a core stretch sequence that is fully, substantially, or at least partially complementary to a target HTT 19-mer sequence disclosed in Table 1.

TABLE 1
HTT 19-mer mRNA Target Sequences (taken from homo sapiens huntingtin
(HTT) transcript, mRNA, 13498 bases, GenBank NM_002111.8 (SEQ ID NO:1))

| | HTT 19-mer | Corresponding Positions | Targeted Gene |
|---|---|---|---|
| SEQ ID | Target Sequences | of Sequence | Position (as |
| No. | (5′ → 3′) | on SEQ ID NO: 1 | referred to herein) |
| 46 | CUCAGGUUCUGCUUUUACC | 32-50 | 30 |
| 47 | GACCCUGGAAAAGCUGAUG | 151-169 | 149 |
| 48 | ACCCUGGAAAAGCUGAUGA | 152-170 | 150 |
| 49 | CCCUGGAAAAGCUGAUGAA | 153-171 | 151 |
| 50 | GAAAAGCUGAUGAAGGCCU | 158-176 | 156 |
| 51 | UGAUGAAGGCCUUCGAGUC | 165-183 | 163 |
| 52 | GAUGAAGGCCUUCGAGUCC | 166-184 | 164 |
| 53 | CCUUCGAGUCCCUCAAGUC | 174-192 | 172 |
| 54 | CUUCGAGUCCCUCAAGUCC | 175-193 | 173 |
| 55 | CUGCACCGACCAAAGAAAG | 404-422 | 402 |
| 56 | UGCACCGACCAAAGAAAGA | 405-423 | 403 |
| 57 | GCACCGACCAAAGAAAGAA | 406-424 | 404 |
| 58 | UCGCUAUGGAACUUUUUCU | 531-549 | 529 |
| 59 | UUUGAUGGAUUCUAAUCUU | 622-640 | 620 |
| 60 | UGGAUUCUAAUCUUCCAAG | 627-645 | 625 |
| 61 | GCAAUUUUGCAAAUGACAA | 870-888 | 868 |
| 62 | GACGUUACAUCAUACACAG | 1231-1249 | 1229 |
| 63 | ACCAAGACCACAAUGUUGU | 1251-1269 | 1249 |
| 64 | GUAUUGUGGAACUUAUAGC | 1407-1425 | 1405 |
| 65 | UGAACUACAUCGAUCAUGG | 2412-2430 | 2410 |
| 66 | GAUGCUGUGAAGCUUUGUG | 3222-3240 | 3220 |
| 67 | GGUCCUGUUACAACAAGUA | 3800-3818 | 3798 |
| 68 | ACAAGUAAAUCCUCAUCAC | 3812-3830 | 3810 |
| 69 | AAUGAUGGCAACUGUUUGU | 4066-4084 | 4064 |
| 70 | GGUGUUUAUUGGCUUUGUA | 4558-4576 | 4556 |
| 71 | ACAGAUCAUUGGAAUUCCU | 4687-4705 | 4685 |
| 72 | GCGACUGUCUCGACAGAUA | 4963-4981 | 4961 |
| 73 | CGUGAGCACUGUUCAACUG | 5146-5164 | 5144 |
| 74 | AGCACAAAGUUACUUAGUC | 5744-5762 | 5742 |
| 75 | AGCCAAACUUGGAAUGUGC | 5800-5818 | 5798 |
| 76 | GCAAUAGAGAAAUAGUACG | 5817-5835 | 5815 |
| 77 | UGAUUAUGUCUGUCAGAAC | 5860-5878 | 5858 |
| 78 | GUCAGAACCUCCAUGACUC | 5871-5889 | 5869 |
| 79 | UCAUUGUAAAUCACAUUCA | 5907-5925 | 5905 |
| 80 | GUACAGGACUUCAUCAGUG | 5957-5975 | 5955 |
| 81 | GCAAUUCAGUCUCGUUGUG | 6017-6035 | 6015 |
| 82 | UGCCAAUGGAAGAACUCAA | 6243-6261 | 6241 |
| 83 | AUGCAAGACUCACUUAGUC | 6350-6368 | 6348 |
| 84 | GGAUGAGUGAAAUUUCUGG | 6606-6624 | 6604 |
| 85 | GGAGCAAGUUGAAUGAUCU | 6756-6774 | 6754 |

Homo sapiens huntingtin (HTT), transcript variant 2, mRNA, GenBank NM_002111.8 (SEQ ID NO:1), gene transcript (13498 bases):

```
        1 gctgccggga cgggtccaag atggacggcc gctcaggttc tgcttttacc tgcggcccag
      61 agccccattc attgccccgg tgctgagcgg cgccgcgagt cggcccgagg cctccgggga
    121 ctgccgtgcc gggcgggaga ccgccatggc gaccctggaa aagctgatga aggccttcga
    181 gtccctcaag tccttccagc agcagcagca gcagcagcag cagcagcagc agcagcagca
    241 gcagcagcag cagcagcagc aacagccgcc accgccgccg ccgccgccgc cgcctcctca
    301 gcttcctcag ccgccgccgc aggcacagcc gctgctgcct cagccgcagc cgcccccgcc
    361 gccgcccccg ccgccacccg gcccggctgt ggctgaggag ccgctgcacc gaccaaagaa
    421 agaactttca gctaccaaga aagaccgtgt gaatcattgt ctgacaatat gtgaaaacat
    481 agtggcacag tctgtcagaa attctccaga atttcagaaa cttctgggca tcgctatgga
    541 actttttctg ctgtgcagtg atgacgcaga gtcagatgtc aggatggtgg ctgacgaatg
    601 cctcaacaaa gttatcaaag ctttgatgga ttctaatctt ccaaggttac agctcgagct
    661 ctataaggaa attaaaaaga atggtgcccc tcggagtttg cgtgctgccc tgtggaggtt
    721 tgctgagctg gctcacctgg ttcggcctca gaaatgcagg ccttacctgg tgaaccttct
    781 gccgtgcctg actcgaacaa gcaagagacc cgaagaatca gtccaggaga ccttggctgc
    841 agctgttccc aaaattatgg cttcttttgg caattttgca aatgacaatg aaattaaggt
    901 tttgttaaag gccttcatag cgaacctgaa gtcaagctcc cccaccattc ggcggacagc
    961 ggctggatca gcagtgagca tctgccagca ctcaagaagg acacaatatt tctatagttg
  1021 gctactaaat gtgctcttag gcttactcgt tcctgtcgag gatgaacact ccactctgct
  1081 gattcttggc gtgctgctca ccctgaggta tttggtgccc ttgctgcagc agcaggtcaa
  1141 ggacacaagc ctgaaaggca gcttcggagt gacaaggaaa gaaatggaag tctctccttc
  1201 tgcagagcag cttgtccagg tttatgaact gacgttacat catacacagc accaagacca
  1261 caatgttgtg accggagccc tggagctgtt gcagcagctc ttcagaacgc ctccacccga
  1321 gcttctgcaa accctgaccg cagtcggggg cattgggcag ctcaccgctg ctaaggagga
  1381 gtctggtggc cgaagccgta gtgggagtat tgtggaactt atagctggag ggggttcctc
  1441 atgcagccct gtcctttcaa gaaaacaaaa aggcaaagtg ctcttaggag aagaagaagc
  1501 cttggaggat gactctgaat cgagatcgga tgtcagcagc tctgccttaa cagcctcagt
  1561 gaaggatgag atcagtggag agctggctgc ttcttcaggg gtttccactc cagggtcagc
  1621 aggtcatgac atcatcacag aacagccacg gtcacagcac acactgcagg cggactcagt
  1681 ggatctggcc agctgtgact tgacaagctc tgccactgat ggggatgagg aggatatctt
  1741 gagccacagc tccagccagg tcagcgccgt cccatctgac cctgccatgg acctgaatga
  1801 tgggacccag gcctcgtcgc ccatcagcga cagctcccag accaccaccg aagggcctga
  1861 ttcagctgtt accccttcag acagttctga aattgtgtta gacggtaccg acaaccagta
  1921 tttgggcctg cagattggac agccccagga tgaagatgag gaagccacag gtattcttcc
  1981 tgatgaagcc tcggaggcct tcaggaactc ttccatggcc cttcaacagg cacatttatt
  2041 gaaaaacatg agtcactgca ggcagccttc tgacagcagt gttgataaat ttgtgttgag
  2101 agatgaagct actgaaccgg gtgatcaaga aaacaagcct tgccgcatca aaggtgacat
  2161 tggacagtcc actgatgatg actctgcacc tcttgtccat tgtgtccgcc ttttatctgc
  2221 ttcgtttttg ctaacagggg gaaaaaatgt gctggttccg gacagggatg tgagggtcag
  2281 cgtgaaggcc ctggccctca gctgtgtggg agcagctgtg gccctccacc cggaatcttt
  2341 cttcagcaaa ctctataaag ttcctcttga caccacggaa taccctgagg aacagtatgt
  2401 ctcagacatc ttgaactaca tcgatcatgg agacccacag gttcgaggag ccactgccat
  2461 tctctgtggg accctcatct gctccatcct cagcaggtcc cgcttccacg tgggagattg
  2521 gatgggcacc attagaaccc tcacaggaaa tacattttct ttggcggatt gcattccttt
  2581 gctgcggaaa acactgaagg atgagtcttc tgttacttgc aagttagctt gtacagctgt
  2641 gaggaactgt gtcatgagtc tctgcagcag cagctacagt gagttaggac tgcagctgat
  2701 catcgatgtg ctgactctga ggaacagttc ctattggctg gtgaggacag agcttctgga
  2761 aacccttgca gagattgact tcaggctggt gagctttttg gaggcaaaag cagaaaactt
  2821 acacagaggg gctcatcatt atacagggct tttaaaactg caagaacgag tgctcaataa
  2881 tgttgtcatc catttgcttg gagatgaaga ccccagggtg cgacatgttg ccgcagcatc
  2941 actaattagg cttgtcccaa agctgtttta taaatgtgac caaggacaag ctgatccagt
  3001 agtggccgtg gcaagagatc aaagcagtgt ttacctgaaa cttctcatgc atgagacgca
  3061 gcctccatct catttctccg tcagcacaat aaccagaata tatagaggct ataacctact
  3121 accaagcata acagacgtca ctatggaaaa taacctttca agagttattg cagcagtttc
  3181 tcatgaacta atcacatcaa ccaccagagc actcacattt ggatgctgtg aagctttgtg
  3241 tcttctttcc actgccttcc cagtttgcat ttggagttta ggttggcact gtggagtgcc
  3301 tccactgagt gcctcagatg agtctaggaa gagctgtacc gttgggatgg ccacaatgat
  3361 tctgaccctg ctctcgtcag cttggttccc attggatctc tcagcccatc aagatgcttt
  3421 gattttggcc ggaaacttgc ttgcagccag tgctcccaaa tctctgagaa gttcatgggc
  3481 ctctgaagaa gaagccaacc cagcagccac caagcaagag gaggtctggc cagccctggg
  3541 ggaccgggcc ctggtgccca tggtggagca gctcttctct cacctgctga aggtgattaa
  3601 catttgtgcc cacgtcctgg atgacgtggc tcctggaccc gcaataaagg cagccttgcc
  3661 ttctctaaca aacccccctt ctctaagtcc catccgacga aaggggaagg agaaagaacc
  3721 aggagaacaa gcatctgtac cgttgagtcc caagaaaggc agtgaggcca gtgcagcttc
  3781 tagacaatct gatacctcag gtcctgttac aacaagtaaa tcctcatcac tggggagttt
  3841 ctatcatctt ccttcatacc tcaaactgca tgatgtcctg aaagctacac acgctaacta
  3901 caaggtcacg ctggatcttc agaacagcac ggaaaagttt ggagggtttc tccgctcagc
  3961 cttggatgtt ctttctcaga tactagagct ggccacactg caggacattg ggaagtgtgt
  4021 tgaagagatc ctaggatacc tgaaatcctg ctttagtcga gaaccaatga tggcaactgt
  4081 ttgtgttcaa caattgttga agactctctt tggcacaaac ttggcctccc agtttgatgg
  4141 cttatcttcc aaccccagca agtcacaagg ccgagcacag cgccttggct cctccagtgt
  4201 gaggccaggc ttgtaccact actgcttcat ggccccgtac acccacttca cccaggccct
  4261 cgctgacgcc agcctgagga acatggtgca ggcggagcag gagaacgaca cctcgggatg
  4321 gtttgatgtc ctccagaaag tgtctaccca gttgaagaca aacctcacga gtgtcacaaa
  4381 gaaccgtgca gataagaatg ctattcataa tcacattcgt ttgtttgaac ctcttgttat
  4441 aaaagcttta aaacagtaca cgactacaac atgtgtgcag ttacagaagc aggttttaga
  4501 tttgctggcg cagctggttc agttacgggt taattactgt cttctggatt cagatcaggt
  4561 gtttattggc tttgtattga aacagtttga atacattgaa gtgggccagt tcagggaatc
  4621 agaggcaatc attccaaaca tctttttctt cttggtatta ctatcttatg aacgctatca
  4681 ttcaaaacag atcattggaa ttcctaaaat cattcagctc tgtgatggca tcatggccag
  4741 tggaaggaag gctgtgacac atgccatacc ggctctgcag cccatagtcc acgacctctt
  4801 tgtattaaga ggaacaaata aagctgatgc aggaaaagag cttgaaaccc aaaaagaggt
  4861 ggtggtgtca atgttactga gactcatcca gtaccatcag gtgttggaga tgttcattct
  4921 tgtcctgcag cagtgccaca aggagaatga agacaagtgg aagcgactgt ctcgacagat
  4981 agctgacatc atcctcccaa tgttagccaa acagcagatg cacattgact ctcatgaagc
  5041 ccttggagtg ttaaatacat tatttgagat tttggcccct tcctccctcc gtccggtaga
  5101 catgctttta cggagtatgt tcgtcactcc aaacacaatg gcgtccgtga gcactgttca
  5161 actgtggata tcgggaattc tggccatttt gagggttctg atttcccagt caactgaaga
  5221 tattgttctt tctcgtattc aggagctctc cttctctccg tatttaatct cctgtacagt
  5281 aattaatagg ttaagagatg gggacagtac ttcaacgcta gaagaacaca gtgaagggaa
  5341 acaaataaag aatttgccag aagaaacatt ttcaaggttt ctattacaac tggttggtat
  5401 tcttttagaa gacattgtta caaaacagct gaaggtggaa atgagtgagc agcaacatac
  5461 tttctattgc caggaactag gcacactgct aatgtgtctg atccacatct tcaagtctgg
  5521 aatgttccgg agaatcacag cagctgccac taggctgttc cgcagtgatg gctgtggcgg
  5581 cagtttctac accctggaca gcttgaactt gcgggctcgt tccatgatca ccacccaccc
  5641 ggccctggtg ctgctctggt gtcagatact gctgcttgtc aaccacaccg actaccgctg
  5701 gtgggcagaa gtgcagcaga ccccgaaaag acacagtctg tccagcacaa agttacttag
  5761 tccccagatg tctggagaag aggaggattc tgacttggca gccaaacttg gaatgtgcaa
  5821 tagagaaata gtacgaagag gggctctcat tctcttctgt gattatgtct gtcagaacct
  5881 ccatgactcc gagcacttaa cgtggctcat tgtaaatcac attcaagatc tgatcagcct
  5941 ttcccacgag cctccagtac aggacttcat cagtgccgtt catcggaact ctgctgccag
  6001 cggcctgttc atccaggcaa ttcagtctcg ttgtgaaaac ctttcaactc caaccatgct
  6061 gaagaaaact cttcagtgct tggaggggat ccatctcagc cagtcgggag ctgtgctcac
  6121 gctgtatgtg gacaggcttc tgtgcacccc tttccgtgtg ctggctcgca tggtcgacat
  6181 ccttgcttgt cgccgggtag aaatgcttct ggctgcaaat ttacagagca gcatggccca
  6241 gttgccaatg gaagaactca acagaatcca ggaatacctt cagagcagcg ggctcgctca
  6301 gagacaccaa aggctctatt ccctgctgga caggtttcgt ctctccacca tgcaagactc
  6361 acttagtccc tctcctccag tctcttccca cccgctggac ggggatgggc acgtgtcact
  6421 ggaaacagtg agtccggaca aagactggta cgttcatctt gtcaaatccc agtgttggac
  6481 caggtcagat tctgcactgc tggaaggtgc agagctggtg aatcggattc ctgctgaaga
  6541 tatgaatgcc ttcatgatga actcggagtt caacctaagc ctgctagctc catgcttaag
  6601 cctagggatg agtgaaattt ctggtggcca gaagagtgcc ctttttgaag cagcccgtga
  6661 ggtgactctg gcccgtgtga gcggcaccgt gcagcagctc cctgctgtcc atcatgtctt
  6721 ccagcccgag ctgcctgcag agccggcggc ctactggagc aagttgaatg atctgtttgg
  6781 ggatgctgca ctgtatcagt ccctgcccac tctggcccgg gccctggcac agtacctggt
  6841 ggtggtctcc aaactgccca gtcatttgca ccttcctcct gagaaagaga aggacattgt
  6901 gaaattcgtg gtggcaaccc ttgaggccct gtcctggcat ttgatccatg agcagatccc
  6961 gctgagtctg gatctccagg cagggctgga ctgctgctgc ctggccctgc agctgcctgg
  7021 cctctggagc gtggtctcct ccacagagtt tgtgacccac gcctgctccc tcatctactg
  7081 tgtgcacttc atcctggagg ccgttgcagt gcagcctgga gagcagcttc ttagtccaga
  7141 aagaaggaca aataccccaa aagccatcag cgaggaggag gaggaagtag atccaaacac
  7201 acagaatcct aagtatatca ctgcagcctg tgagatggtg gcagaaatgg tggagtctct
  7261 gcagtcggtg ttggccttgg gtcataaaag gaatagcggc gtgccggcgt ttctcacgcc
  7321 attgctaagg aacatcatca tcagcctggc ccgcctgccc cttgtcaaca gctacacacg
  7381 tgtgccccca ctggtgtgga agcttggatg gtcacccaaa ccgggagggg attttggcac
  7441 agcattccct gagatccccg tggagttcct ccaggaaaag gaagtcttta aggagttcat
  7501 ctaccgcatc aacacactag gctggaccag tcgtactcag tttgaagaaa cttgggccac
  7561 cctccttggt gtcctggtga cgcagcccct cgtgatggag caggaggaga gcccaccaga
  7621 agaagacaca gagaggaccc agatcaacgt cctggccgtg caggccatca cctcactggt
  7681 gctcagtgca atgactgtgc ctgtggccgg caacccagct gtaagctgct tggagcagca
  7741 gccccggaac aagcctctga aagctctcga caccaggttt gggaggaagc tgagcattat
  7801 cagagggatt gtggagcaag agattcaagc aatggtttca aagagagaga atattgccac
  7861 ccatcattta tatcaggcat gggatcctgt cccttctctg tctccggcta ctacaggtgc
  7921 cctcatcagc cacgagaagc tgctgctaca gatcaacccc gagcgggagc tggggagcat
  7981 gagctacaaa ctcggccagg tgtccataca ctccgtgtgg ctggggaaca gcatcacacc
  8041 cctgagggag gaggaatggg acgaggaaga ggaggaggag gccgacgccc ctgcaccttc
  8101 gtcaccaccc acgtctccag tcaactccag gaaacaccgg gctggagttg acatccactc
  8161 ctgttcgcag tttttgcttg agttgtacag ccgctggatc ctgccgtcca gctcagccag
  8221 gaggaccccg gccatcctga tcagtgaggt ggtcagatcc cttctagtgg tctcagactt
  8281 gttcaccgag cgcaaccagt ttgagctgat gtatgtgacg ctgacagaac tgcgaagggt
  8341 gcacccttca gaagacgaga tcctcgctca gtacctggtg cctgccacct gcaaggcagc
  8401 tgccgtcctt gggatggaca aggccgtggc ggagcctgtc agccgcctgc tggagagcac
  8461 gctcaggagc agccacctgc ccagcagggt tggagccctg cacggcgtcc tctatgtgct
  8521 ggagtgcgac ctgctggacg acactgccaa gcagctcatc ccggtcatca gcgactatct
  8581 cctctccaac ctgaaaggga tcgcccactg cgtgaacatt cacagccagc agcacgtact
  8641 ggtcatgtgt gccactgcgt tttacctcat tgagaactat cctctggacg tagggccgga
  8701 attttcagca tcaataatac agatgtgtgg ggtgatgctg tctggaagtg aggagtccac
  8761 cccctccatc atttaccact gtgccctcag aggcctggag cgcctcctgc tctctgagca
  8821 gctctcccgc ctggatgcag aatcgctggt caagctgagt gtggacagag tgaacgtgca
  8881 cagcccgcac cgggccatgg cggctctggg cctgatgctc acctgcatgt acacaggaaa
  8941 ggagaaagtc agtccgggta gaacttcaga ccctaatcct gcagcccccg acagcgagtc
  9001 agtgattgtt gctatggagc gggtatctgt tctttttgat aggatcagga aaggctttcc
  9061 ttgtgaagcc agagtggtgg ccaggatcct gccccagttt ctagacgact tcttcccacc
  9121 ccaggacatc atgaacaaag tcatcggaga gtttctgtcc aaccagcagc cataccccca
  9181 gttcatggcc accgtggtgt ataaggtgtt tcagactctg cacagcaccg ggcagtcgtc
  9241 catggtccgg gactgggtca tgctgtccct ctccaacttc acgcagaggg ccccggtcgc
  9301 catggccacg tggagcctct cctgcttctt tgtcagcgcg tccaccagcc cgtgggtcgc
  9361 ggcgatcctc ccacatgtca tcagcaggat gggcaagctg gagcaggtgg acgtgaacct
  9421 tttctgcctg gtcgccacag acttctacag acaccagata gaggaggagc tcgaccgcag
  9481 ggccttccag tctgtgcttg aggtggttgc agccccagga agcccatatc accggctgct
  9541 gacttgttta cgaaatgtcc acaaggtcac cacctgctga gcgccatggt gggagagact
  9601 gtgaggcggc agctggggcc ggagcctttg gaagtctgcg cccttgtgcc ctgcctccac
  9661 cgagccagct tggtccctat gggcttccgc acatgccgcg ggcggccagg caacgtgcgt
  9721 gtctctgcca tgtggcagaa gtgctctttg tggcagtggc caggcaggga gtgtctgcag
  9781 tcctggtggg gctgagcctg aggccttcca gaaagcagga gcagctgtgc tgcaccccat
  9841 gtgggtgacc aggtcctttc tcctgatagt cacctgctgg ttgttgccag gttgcagctg
  9901 ctcttgcatc tgggccagaa gtcctccctc ctgcaggctg gctgttggcc cctctgctgt
  9961 cctgcagtag aaggtgccgt gagcaggctt tgggaacact ggcctgggtc tccctggtgg
10021 ggtgtgcatg ccacgccccg tgtctggatg cacagatgcc atggcctgtg ctgggccagt
10081 ggctgggggt gctagacacc cggcaccatt ctcccttctc tcttttcttc tcaggattta
10141 aaatttaatt atatcagtaa agagattaat tttaacgtaa ctctttctat gcccgtgtaa
10201 agtatgtgaa tcgcaaggcc tgtgctgcat gcgacagcgt ccggggtggt ggacagggcc
10261 cccggccacg ctccctctcc tgtagccact ggcatagccc tcctgagcac ccgctgacat
10321 ttccgttgta catgttcctg tttatgcatt cacaaggtga ctgggatgta gagaggcgtt
10381 agtgggcagg tggccacagc aggactgagg acaggccccc attatcctag gggtgcgctc
10441 acctgcagcc cctcctcctc gggcacagac gactgtcgtt ctccacccac cagtcaggga
10501 cagcagcctc cctgtcactc agctgagaag gccagccctc cctggctgtg agcagcctcc
10561 actgtgtcca gagacatggg cctcccactc ctgttccttg ctagccctgg ggtggcgtct
10621 gcctaggagc tggctggcag gtgttgggac ctgctgctcc atggatgcat gccctaagag
10681 tgtcactgag ctgtgttttg tctgagcctc tctcggtcaa cagcaaagct tggtgtcttg
10741 gcactgttag tgacagagcc cagcatccct tctgcccccg ttccagctga catcttgcac
10801 ggtgacccct tttagtcagg agagtgcaga tctgtgctca tcggagactg ccccacggcc
10861 ctgtcagagc cgccactcct atccccaggc caggtccctg gaccagcctc ctgtttgcag
10921 gcccagagga gccaagtcat taaaatggaa gtggattctg gatggccggg ctgctgctga
10981 tgtaggagct ggatttggga gctctgcttg ccgactggct gtgagacgag gcaggggctc
11041 tgcttcctca gccctagagg cgagccaggc aaggttggcg actgtcatgt ggcttggttt
11101 ggtcatgccc gtcgatgttt tgggtattga atgtggtaag tggaggaaat gttggaactc
11161 tgtgcaggtg ctgccttgag acccccaagc ttccacctgt ccctctccta tgtggcagct
11221 ggggagcagc tgagatgtgg acttgtatgc tgcccacata cgtgaggggg agctgaaagg
11281 gagcccctcc tctgagcagc ctctgccagg cctgtatgag gcttttccca ccagctccca
11341 acagaggcct cccccagcca ggaccacctc gtcctcgtgg cggggcagca ggagcggtag
11401 aaaggggtcc gatgtttgag gaggccctta agggaagcta ctgaattata acacgtaaga
11461 aaatcaccat tccgtattgg ttgggggctc ctgtttctca tcctagcttt ttcctggaaa
11521 gcccgctaga aggtttggga acgaggggaa agttctcaga actgttggct gctccccacc
11581 cgcctcccgc ctcccccgca ggttatgtca gcagctctga gacagcagta tcacaggcca
11641 gatgttgttc ctggctagat gtttacattt gtaagaaata acactgtgaa tgtaaaacag
11701 agccattccc ttggaatgca tatcgctggg ctcaacatag agtttgtctt cctcttgttt
11761 acgacgtgat ctaaaccagt ccttagcaag gggctcagaa caccccgctc tggcagtagg
11821 tgtcccccac ccccaaagac ctgcctgtgt gctccggaga tgaatatgag ctcattagta
11881 aaaatgactt cacccacgca tatacataaa gtatccatgc atgtgcatat agacacatct
11941 ataattttac acacacacct ctcaagacgg agatgcatgg cctctaagag tgcccgtgtc
12001 ggttcttcct ggaagttgac tttccttaga cccgccaggt caagttagcc gcgtgacgga
12061 catccaggcg tgggacgtgg tcagggcagg gctcattcat tgcccactag gatcccactg
12121 gcgaagatgg tctccatatc agctctctgc agaagggagg aagactttat catgttccta
12181 aaaatctgtg gcaagcaccc atcgtattat ccaaattttg ttgcaaatgt gattaatttg
12241 gttgtcaagt tttgggggtg ggctgtgggg agattgcttt tgttttcctg ctggtaatat
12301 cgggaaagat tttaatgaaa ccagggtaga attgtttggc aatgcactga agcgtgtttc
12361 tttcccaaaa tgtgcctccc ttccgctgcg ggcccagctg agtctatgta ggtgatgttt
12421 ccagctgcca agtgctcttt gttactgtcc accctcattt ctgccagcgc atgtgtcctt
12481 tcaaggggaa aatgtgaagc tgaaccccct ccagacaccc agaatgtagc atctgagaag
12541 gccctgtgcc ctaaaggaca cccctcgccc ccatcttcat ggagggggtc atttcagagc
12601 cctcggagcc aatgaacagc tcctcctctt ggagctgaga tgagccccac gtggagctcg
12661 ggacggatag tagacagcaa taactcggtg tgtggccgcc tggcaggtgg aacttcctcc
12721 cgttgcgggg tggagtgagg ttagttctgt gtgtctggtg ggtggagtca ggcttctctt
12781 gctacctgtg agcatccttc ccagcagaca tcctcatcgg gctttgtccc tcccccgctt
12841 cctccctctg cggggaggac ccgggaccac agctgctggc cagggtagac ttggagctgt
12901 cctccagagg ggtcacgtgt aggagtgaga agaaggaaga tcttgagagc tgctgaggga
12961 ccttggagag ctcaggatgg ctcagacgag gacactcgct tgccgggcct gggcctcctg
13021 ggaaggaggg agctgctcag aatgccgcat gacaactgaa ggcaacctgg aaggttcagg
13081 ggccgctctt cccccatgtg cctgtcacgc tctggtgcag tcaaaggaac gccttcccct
13141 cagttgtttc taagagcaga gtctcccgct gcaatctggg tggtaactgc cagccttgga
13201 ggatcgtggc caacgtggac ctgcctacgg agggtgggct ctgacccaag tggggcctcc
13261 ttgtccaggt ctcactgctt tgcaccgtgg tcagagggac tgtcagctga gcttgagctc
13321 ccctggagcc agcagggctg tgatgggcga gtcccggagc cccacccaga cctgaatgct
13381 tctgagagca aagggaagga ctgacgagag atgtatattt aattttttaa ctgctgcaaa
13441 cattgtacat ccaaattaaa ggaaaaaaat ggaaaccatc aaaaaaaaaa aaaaaaaa
```

In some embodiments, an HTT RNAi agent includes an antisense strand wherein position 19 of the antisense strand (5′→3′) is capable of forming a base pair with position 1 of a 19-mer target sequence disclosed in Table 1. In some embodiments, an HTT agent includes an antisense strand wherein position 1 of the antisense strand (5′→3′) is capable of forming a base pair with position 19 of a 19-mer target sequence disclosed in Table 1.

In some embodiments, an HTT agent includes an antisense strand wherein position 2 of the antisense strand (5′→3′) is capable of forming a base pair with position 18 of a 19-mer target sequence disclosed in Table 1. In some embodiments, an HTT agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to an HTT gene, or can be non-complementary to an HTT gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, an HTT RNAi agent antisense strand comprises the sequence of nucleotides (from 5′ end→3′ end) 2-18 or 2-19 of any of the antisense strand sequences in Table 2 or Table 3. In some embodiments, an HTT RNAi sense strand comprises the sequence of nucleotides (from 5′ end→3′ end) 1-17, 1-18, or 2-18 of any of the sense strand sequences in Table 2, Table 4, Table 5, or Table 6.

In some embodiments, an HTT RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2, Table 4, Table 5, or Table 6.

In some embodiments, the HTT RNAi agents include core 19-mer nucleotide sequences shown in the following Table 2.

TABLE 2
HTT RNAi Agent Antisense Strand and Sense Strand Core Stretch Base
Sequences(N = any nucleobase; I = inosine (hypoxanthine nucleobase)

| | Antisense Strand | | Sense Strand | | |
|---|---|---|---|---|---|
| | Base Sequence | | Base Sequence | Corresponding | |
| | (5′ → 3′) | | (5′ → 3′) | Positions of | |
| SEQ | (Shown as an | SEQ | (Shown as an | Identified | Targeted |
| ID | Unmodified | ID | Unmodified | Sequence on | Gene |
| NO:. | Nucleotide Sequence) | NO:. | Nucleotide Sequence) | SEQ ID NO: 1 | Position |
| 86 | GGUAAAAGCAGAACCUGAG | 269 | CUCAGGUUCUGCUUUUACC | 32-50 | 30 |
| 87 | UGUAAAAGCAGAACCUGAG | 270 | CUCAGGUUCUGCUUUUACA | 32-50 | 30 |
| 88 | AGUAAAAGCAGAACCUGAG | 271 | CUCAGGUUCUGCUUUUACU | 32-50 | 30 |
| 89 | NGUAAAAGCAGAACCUGAG | 272 | CUCAGGUUCUGCUUUUACN | 32-50 | 30 |
| 90 | NGUAAAAGCAGAACCUGAN | 273 | NUCAGGUUCUGCUUUUACN | 32-50 | 30 |
| 91 | CAUCAGCUUUUCCAGGGUC | 274 | GACCCUGGAAAAGCUGAUG | 151-169 | 149 |
| 92 | UAUCAGCUUUUCCAGGGUC | 275 | GACCCUGGAAAAGCUGAUA | 151-169 | 149 |
| 93 | AAUCAGCUUUUCCAGGGUC | 276 | GACCCUGGAAAAGCUGAUU | 151-169 | 149 |
| 94 | NAUCAGCUUUUCCAGGGUC | 277 | GACCCUGGAAAAGCUGAUN | 151-169 | 149 |
| 95 | NAUCAGCUUUUCCAGGGUN | 278 | NACCCUGGAAAAGCUGAUN | 151-169 | 149 |
| 96 | UCAUCAGCUUUUCCAGGGU | 279 | ACCCUGGAAAAGCUGAUGA | 152-170 | 150 |
| 97 | ACAUCAGCUUUUCCAGGGU | 280 | ACCCUGGAAAAGCUGAUGU | 152-170 | 150 |
| 98 | NCAUCAGCUUUUCCAGGGU | 281 | ACCCUGGAAAAGCUGAUGN | 152-170 | 150 |
| 99 | NCAUCAGCUUUUCCAGGGN | 282 | NCCCUGGAAAAGCUGAUGN | 152-170 | 150 |
| 100 | UUCAUCAGCUUUUCCAGGG | 283 | CCCUGGAAAAGCUGAUGAA | 153-171 | 151 |
| 101 | AUCAUCAGCUUUUCCAGGG | 284 | CCCUGGAAAAGCUGAUGAU | 153-171 | 151 |
| 102 | NUCAUCAGCUUUUCCAGGG | 285 | CCCUGGAAAAGCUGAUGAN | 153-171 | 151 |
| 103 | NUCAUCAGCUUUUCCAGGN | 286 | NCCUGGAAAAGCUGAUGAN | 153-171 | 151 |
| 104 | AGGCCUUCAUCAGCUUUUC | 287 | GAAAAGCUGAUGAAGGCCU | 158-176 | 156 |
| 105 | UGGCCUUCAUCAGCUUUUC | 288 | GAAAAGCUGAUGAAGGCCA | 158-176 | 156 |
| 106 | NGGCCUUCAUCAGCUUUUC | 289 | GAAAAGCUGAUGAAGGCCN | 158-176 | 156 |
| 107 | NGGCCUUCAUCAGCUUUUN | 290 | NAAAAGCUGAUGAAGGCCN | 158-176 | 156 |
| 108 | GACUCGAAGGCCUUCAUCA | 291 | UGAUGAAGGCCUUCGAGUC | 165-183 | 163 |
| 109 | UACUCGAAGGCCUUCAUCA | 292 | UGAUGAAGGCCUUCGAGUA | 165-183 | 163 |
| 110 | AACUCGAAGGCCUUCAUCA | 293 | UGAUGAAGGCCUUCGAGUU | 165-183 | 163 |
| 111 | NACUCGAAGGCCUUCAUCA | 294 | UGAUGAAGGCCUUCGAGUN | 165-183 | 163 |
| 112 | NACUCGAAGGCCUUCAUCN | 295 | NGAUGAAGGCCUUCGAGUN | 165-183 | 163 |
| 113 | GGACUCGAAAGCCUUCAUC | 296 | GAUGAAGGCCUUCGAGUCC | 166-184 | 164 |
| 114 | UGACUCGAAAGCCUUCAUC | 297 | GAUGAAGGCCUUCGAGUCA | 166-184 | 164 |
| 115 | AGACUCGAAAGCCUUCAUC | 298 | GAUGAAGGCCUUCGAGUCU | 166-184 | 164 |
| 116 | NGACUCGAAAGCCUUCAUC | 299 | GAUGAAGGCCUUCGAGUCN | 166-184 | 164 |
| 117 | NGACUCGAAAGCCUUCAUN | 300 | NAUGAAGGCCUUCGAGUCN | 166-184 | 164 |
| 118 | GACUUGAGGGACUCGAAGG | 301 | CCUUCGAGUCCCUCAAGUC | 174-192 | 172 |
| 119 | UACUUGAGGGACUCGAAGG | 302 | CCUUCGAGUCCCUCAAGUA | 174-192 | 172 |
| 120 | AACUUGAGGGACUCGAAGG | 303 | CCUUCGAGUCCCUCAAGUU | 174-192 | 172 |
| 121 | NACUUGAGGGACUCGAAGG | 304 | CCUUCGAGUCCCUCAAGUN | 174-192 | 172 |
| 122 | NACUUGAGGGACUCGAAGN | 305 | NCUUCGAGUCCCUCAAGUN | 174-192 | 172 |
| 123 | GGACUUGAGGGACUCGAAG | 306 | CUUCGAGUCCCUCAAGUCC | 175-193 | 173 |
| 124 | UGACUUGAGGGACUCGAAG | 307 | CUUCGAGUCCCUCAAGUCA | 175-193 | 173 |
| 125 | AGACUUGAGGGACUCGAAG | 308 | CUUCGAGUCCCUCAAGUCU | 175-193 | 173 |
| 126 | NGACUUGAGGGACUCGAAG | 309 | CUUCGAGUCCCUCAAGUCN | 175-193 | 173 |
| 127 | NGACUUGAGGGACUCGAAN | 310 | NUUCGAGUCCCUCAAGUCN | 175-193 | 173 |
| 128 | CUUUCUUUGGUCGGUGCAG | 311 | CUGCACCGACCAAAGAAAG | 404-422 | 402 |
| 129 | UUUUCUUUGGUCGGUGCAG | 312 | CUGCACCGACCAAAGAAAA | 404-422 | 402 |
| 130 | AUUUCUUUGGUCGGUGCAG | 313 | CUGCACCGACCAAAGAAAU | 404-422 | 402 |
| 131 | NUUUCUUUGGUCGGUGCAG | 314 | CUGCACCGACCAAAGAAAN | 404-422 | 402 |
| 132 | NUUUCUUUGGUCGGUGCAN | 315 | NUGCACCGACCAAAGAAAN | 404-422 | 402 |
| 133 | UCUUUCUUUGGUCGGUGCA | 316 | UGCACCGACCAAAGAAAGA | 405-423 | 403 |
| 134 | ACUUUCUUUGGUCGGUGCA | 317 | UGCACCGACCAAAGAAAGU | 405-423 | 403 |
| 135 | NCUUUCUUUGGUCGGUGCA | 318 | UGCACCGACCAAAGAAAGN | 405-423 | 403 |
| 136 | NCUUUCUUUGGUCGGUGCN | 319 | NGCACCGACCAAAGAAAGN | 405-423 | 403 |
| 137 | UUCUUUCUUUGGUCGGUGC | 320 | GCACCGACCAAAGAAAGAA | 406-424 | 404 |
| 138 | AUCUUUCUUUGGUCGGUGC | 321 | GCACCGACCAAAGAAAGAU | 406-424 | 404 |
| 139 | NUCUUUCUUUGGUCGGUGC | 322 | GCACCGACCAAAGAAAGAN | 406-424 | 404 |
| 140 | NUCUUUCUUUGGUCGGUGN | 323 | NCACCGACCAAAGAAAGAN | 406-424 | 404 |
| 141 | AGAAAAAGUUCCAUAGCGA | 324 | UCGCUAUGGAACUUUUUCU | 531-549 | 529 |
| 142 | UGAAAAAGUUCCAUAGCGA | 325 | UCGCUAUGGAACUUUUUCA | 531-549 | 529 |
| 143 | NGAAAAAGUUCCAUAGCGA | 326 | UCGCUAUGGAACUUUUUCN | 531-549 | 529 |
| 144 | NGAAAAAGUUCCAUAGCGN | 327 | NCGCUAUGGAACUUUUUCN | 531-549 | 529 |
| 145 | AAGAUUAGAAUCCAUCAAA | 328 | UUUGAUGGAUUCUAAUCUU | 622-640 | 620 |
| 146 | UAGAUUAGAAUCCAUCAAA | 329 | UUUGAUGGAUUCUAAUCUA | 622-640 | 620 |
| 147 | NAGAUUAGAAUCCAUCAAA | 330 | UUUGAUGGAUUCUAAUCUN | 622-640 | 620 |
| 148 | NAGAUUAGAAUCCAUCAAN | 331 | NUUGAUGGAUUCUAAUCUN | 622-640 | 620 |
| 149 | CUUGGAAGAUUAGAAUCCA | 332 | UGGAUUCUAAUCUUCCAAG | 627-645 | 625 |
| 150 | UUUGGAAGAUUAGAAUCCA | 333 | UGGAUUCUAAUCUUCCAAA | 627-645 | 625 |
| 151 | AUUGGAAGAUUAGAAUCCA | 334 | UGGAUUCUAAUCUUCCAAU | 627-645 | 625 |
| 152 | NUUGGAAGAUUAGAAUCCA | 335 | UGGAUUCUAAUCUUCCAAN | 627-645 | 625 |
| 153 | NUUGGAAGAUUAGAAUCCN | 336 | NGGAUUCUAAUCUUCCAAN | 627-645 | 625 |
| 154 | UUGUCAUUUGCAAAAUUGC | 337 | GCAAUUUUGCAAAUGACAA | 870-888 | 868 |
| 155 | AUGUCAUUUGCAAAAUUGC | 338 | GCAAUUUUGCAAAUGACAU | 870-888 | 868 |
| 156 | NUGUCAUUUGCAAAAUUGC | 339 | GCAAUUUUGCAAAUGACAN | 870-888 | 868 |
| 157 | NUGUCAUUUGCAAAAUUGN | 340 | NCAAUUUUGCAAAUGACAN | 870-888 | 868 |
| 158 | CUGUGUAUGAUGUAACGUC | 341 | GACGUUACAUCAUACACAG | 1231-1249 | 1229 |
| 159 | UUGUGUAUGAUGUAACGUC | 342 | GACGUUACAUCAUACACAA | 1231-1249 | 1229 |
| 160 | AUGUGUAUGAUGUAACGUC | 343 | GACGUUACAUCAUACACAU | 1231-1249 | 1229 |
| 161 | NUGUGUAUGAUGUAACGUC | 344 | GACGUUACAUCAUACACAN | 1231-1249 | 1229 |
| 162 | NUGUGUAUGAUGUAACGUN | 345 | NACGUUACAUCAUACACAN | 1231-1249 | 1229 |
| 163 | ACAACAUUGUGGUCUUGGU | 346 | ACCAAGACCACAAUGUUGU | 1251-1269 | 1249 |
| 164 | UCAACAUUGUGGUCUUGGU | 347 | ACCAAGACCACAAUGUUGA | 1251-1269 | 1249 |
| 165 | NCAACAUUGUGGUCUUGGU | 348 | ACCAAGACCACAAUGUUGN | 1251-1269 | 1249 |
| 166 | NCAACAUUGUGGUCUUGGN | 349 | NCCAAGACCACAAUGUUGN | 1251-1269 | 1249 |
| 167 | GCUAUAAGUUCCACAAUAC | 350 | GUAUUGUGGAACUUAUAGC | 1407-1425 | 1405 |
| 168 | UCUAUAAGUUCCACAAUAC | 351 | GUAUUGUGGAACUUAUAGA | 1407-1425 | 1405 |
| 169 | ACUAUAAGUUCCACAAUAC | 352 | GUAUUGUGGAACUUAUAGU | 1407-1425 | 1405 |
| 170 | NCUAUAAGUUCCACAAUAC | 353 | GUAUUGUGGAACUUAUAGN | 1407-1425 | 1405 |
| 171 | NCUAUAAGUUCCACAAUAN | 354 | NUAUUGUGGAACUUAUAGN | 1407-1425 | 1405 |
| 172 | CCAUGAUCGAUGUAGUUCA | 355 | UGAACUACAUCGAUCAUGG | 2412-2430 | 2410 |
| 173 | UCAUGAUCGAUGUAGUUCA | 356 | UGAACUACAUCGAUCAUGA | 2412-2430 | 2410 |
| 174 | ACAUGAUCGAUGUAGUUCA | 357 | UGAACUACAUCGAUCAUGU | 2412-2430 | 2410 |
| 175 | NCAUGAUCGAUGUAGUUCA | 358 | UGAACUACAUCGAUCAUGN | 2412-2430 | 2410 |
| 176 | NCAUGAUCGAUGUAGUUCN | 359 | NGAACUACAUCGAUCAUGN | 2412-2430 | 2410 |
| 177 | CACAAAGCUUCACAGCAUC | 360 | GAUGCUGUGAAGCUUUGUG | 3222-3240 | 3220 |
| 178 | UACAAAGCUUCACAGCAUC | 361 | GAUGCUGUGAAGCUUUGUA | 3222-3240 | 3220 |
| 179 | AACAAAGCUUCACAGCAUC | 362 | GAUGCUGUGAAGCUUUGUU | 3222-3240 | 3220 |
| 180 | NACAAAGCUUCACAGCAUC | 363 | GAUGCUGUGAAGCUUUGUN | 3222-3240 | 3220 |
| 181 | NACAAAGCUUCACAGCAUN | 364 | NAUGCUGUGAAGCUUUGUN | 3222-3240 | 3220 |
| 182 | UACUUGUUGUAACAGGACC | 365 | GGUCCUGUUACAACAAGUA | 3800-3818 | 3798 |
| 183 | AACUUGUUGUAACAGGACC | 366 | GGUCCUGUUACAACAAGUU | 3800-3818 | 3798 |
| 184 | NACUUGUUGUAACAGGACC | 367 | GGUCCUGUUACAACAAGUN | 3800-3818 | 3798 |
| 185 | NACUUGUUGUAACAGGACN | 368 | NGUCCUGUUACAACAAGUN | 3800-3818 | 3798 |
| 186 | GUGAUGAGGAUUUACUUGU | 369 | ACAAGUAAAUCCUCAUCAC | 3812-3830 | 3810 |
| 187 | UUGAUGAGGAUUUACUUGU | 370 | ACAAGUAAAUCCUCAUCAA | 3812-3830 | 3810 |
| 188 | AUGAUGAGGAUUUACUUGU | 371 | ACAAGUAAAUCCUCAUCAU | 3812-3830 | 3810 |
| 189 | NUGAUGAGGAUUUACUUGU | 372 | ACAAGUAAAUCCUCAUCAN | 3812-3830 | 3810 |
| 190 | NUGAUGAGGAUUUACUUGN | 373 | NCAAGUAAAUCCUCAUCAN | 3812-3830 | 3810 |
| 191 | ACAAACAGUUGCCAUCAUU | 374 | AAUGAUGGCAACUGUUUGU | 4066-4084 | 4064 |
| 192 | UCAAACAGUUGCCAUCAUU | 375 | AAUGAUGGCAACUGUUUGA | 4066-4084 | 4064 |
| 193 | NCAAACAGUUGCCAUCAUU | 376 | AAUGAUGGCAACUGUUUGN | 4066-4084 | 4064 |
| 194 | NCAAACAGUUGCCAUCAUN | 377 | NAUGAUGGCAACUGUUUGN | 4066-4084 | 4064 |
| 195 | UACAAAGCCAAUAAACACC | 378 | GGUGUUUAUUGGCUUUGUA | 4558-4576 | 4556 |
| 196 | AACAAAGCCAAUAAACACC | 379 | GGUGUUUAUUGGCUUUGUU | 4558-4576 | 4556 |
| 197 | NACAAAGCCAAUAAACACC | 380 | GGUGUUUAUUGGCUUUGUN | 4558-4576 | 4556 |
| 198 | NACAAAGCCAAUAAACACN | 381 | NGUGUUUAUUGGCUUUGUN | 4558-4576 | 4556 |
| 199 | AGGAAUUCCAAUGAUCUGU | 382 | ACAGAUCAUUGGAAUUCCU | 4687-4705 | 4685 |
| 200 | UGGAAUUCCAAUGAUCUGU | 383 | ACAGAUCAUUGGAAUUCCA | 4687-4705 | 4685 |
| 201 | NGGAAUUCCAAUGAUCUGU | 384 | ACAGAUCAUUGGAAUUCCN | 4687-4705 | 4685 |
| 202 | NGGAAUUCCAAUGAUCUGN | 385 | NCAGAUCAUUGGAAUUCCN | 4687-4705 | 4685 |
| 203 | UAUCUGUCGAGACAGUCGC | 386 | GCGACUGUCUCGACAGAUA | 4963-4981 | 4961 |
| 204 | AAUCUGUCGAGACAGUCGC | 387 | GCGACUGUCUCGACAGAUU | 4963-4981 | 4961 |
| 205 | NAUCUGUCGAGACAGUCGC | 388 | GCGACUGUCUCGACAGAUN | 4963-4981 | 4961 |
| 206 | NAUCUGUCGAGACAGUCGN | 389 | NCGACUGUCUCGACAGAUN | 4963-4981 | 4961 |
| 207 | CAGUUGAACAGUGCUCACG | 390 | CGUGAGCACUGUUCAACUG | 5146-5164 | 5144 |
| 208 | UAGUUGAACAGUGCUCACG | 391 | CGUGAGCACUGUUCAACUA | 5146-5164 | 5144 |
| 209 | AAGUUGAACAGUGCUCACG | 392 | CGUGAGCACUGUUCAACUU | 5146-5164 | 5144 |
| 210 | NAGUUGAACAGUGCUCACG | 393 | CGUGAGCACUGUUCAACUN | 5146-5164 | 5144 |
| 211 | NAGUUGAACAGUGCUCACN | 394 | NGUGAGCACUGUUCAACUN | 5146-5164 | 5144 |
| 212 | GACUAAGUAACUUUGUGCU | 395 | AGCACAAAGUUACUUAGUC | 5744-5762 | 5742 |
| 213 | UACUAAGUAACUUUGUGCU | 396 | AGCACAAAGUUACUUAGUA | 5744-5762 | 5742 |
| 214 | AACUAAGUAACUUUGUGCU | 397 | AGCACAAAGUUACUUAGUU | 5744-5762 | 5742 |
| 215 | NACUAAGUAACUUUGUGCU | 398 | AGCACAAAGUUACUUAGUN | 5744-5762 | 5742 |
| 216 | NACUAAGUAACUUUGUGCN | 399 | NGCACAAAGUUACUUAGUN | 5744-5762 | 5742 |
| 217 | GCACAUUCCAAGUUUGGCU | 400 | AGCCAAACUUGGAAUGUGC | 5800-5818 | 5798 |
| 218 | UCACAUUCCAAGUUUGGCU | 401 | AGCCAAACUUGGAAUGUGA | 5800-5818 | 5798 |
| 219 | ACACAUUCCAAGUUUGGCU | 402 | AGCCAAACUUGGAAUGUGU | 5800-5818 | 5798 |
| 220 | NCACAUUCCAAGUUUGGCU | 403 | AGCCAAACUUGGAAUGUGN | 5800-5818 | 5798 |
| 221 | NCACAUUCCAAGUUUGGCN | 404 | NGCCAAACUUGGAAUGUGN | 5800-5818 | 5798 |
| 222 | CGUACUAUUUCUCUAUUGC | 405 | GCAAUAGAGAAAUAGUACG | 5817-5835 | 5815 |
| 223 | UGUACUAUUUCUCUAUUGC | 406 | GCAAUAGAGAAAUAGUACA | 5817-5835 | 5815 |
| 224 | AGUACUAUUUCUCUAUUGC | 407 | GCAAUAGAGAAAUAGUACU | 5817-5835 | 5815 |
| 225 | NGUACUAUUUCUCUAUUGC | 408 | GCAAUAGAGAAAUAGUACN | 5817-5835 | 5815 |
| 226 | NGUACUAUUUCUCUAUUGN | 409 | NCAAUAGAGAAAUAGUACN | 5817-5835 | 5815 |
| 227 | GUUCUGACAGACAUAAUCA | 410 | UGAUUAUGUCUGUCAGAAC | 5860-5878 | 5858 |
| 228 | UUUCUGACAGACAUAAUCA | 411 | UGAUUAUGUCUGUCAGAAA | 5860-5878 | 5858 |
| 229 | AUUCUGACAGACAUAAUCA | 412 | UGAUUAUGUCUGUCAGAAU | 5860-5878 | 5858 |
| 230 | NUUCUGACAGACAUAAUCA | 413 | UGAUUAUGUCUGUCAGAAN | 5860-5878 | 5858 |
| 231 | NUUCUGACAGACAUAAUCN | 414 | NGAUUAUGUCUGUCAGAAN | 5860-5878 | 5858 |
| 232 | GAGUCAUGGAGGUUCUGAC | 415 | GUCAGAACCUCCAUGACUC | 5871-5889 | 5869 |
| 233 | UAGUCAUGGAGGUUCUGAC | 416 | GUCAGAACCUCCAUGACUA | 5871-5889 | 5869 |
| 234 | AAGUCAUGGAGGUUCUGAC | 417 | GUCAGAACCUCCAUGACUU | 5871-5889 | 5869 |
| 235 | NAGUCAUGGAGGUUCUGAC | 418 | GUCAGAACCUCCAUGACUN | 5871-5889 | 5869 |
| 236 | NAGUCAUGGAGGUUCUGAN | 419 | NUCAGAACCUCCAUGACUN | 5871-5889 | 5869 |
| 237 | UGAAUGUGAUUUACAAUGA | 420 | UCAUUGUAAAUCACAUUCA | 5907-5925 | 5905 |
| 238 | AGAAUGUGAUUUACAAUGA | 421 | UCAUUGUAAAUCACAUUCU | 5907-5925 | 5905 |
| 239 | NGAAUGUGAUUUACAAUGA | 422 | UCAUUGUAAAUCACAUUCN | 5907-5925 | 5905 |
| 240 | NGAAUGUGAUUUACAAUGN | 423 | NCAUUGUAAAUCACAUUCN | 5907-5925 | 5905 |
| 241 | CACUGAUGAAGUCCUGUAC | 424 | GUACAGGACUUCAUCAGUG | 5957-5975 | 5955 |
| 242 | UACUGAUGAAGUCCUGUAC | 425 | GUACAGGACUUCAUCAGUA | 5957-5975 | 5955 |
| 243 | AACUGAUGAAGUCCUGUAC | 426 | GUACAGGACUUCAUCAGUU | 5957-5975 | 5955 |
| 244 | NACUGAUGAAGUCCUGUAC | 427 | GUACAGGACUUCAUCAGUN | 5957-5975 | 5955 |
| 245 | NACUGAUGAAGUCCUGUAN | 428 | NUACAGGACUUCAUCAGUN | 5957-5975 | 5955 |
| 246 | CACAACGAGACUGAAUUGC | 429 | GCAAUUCAGUCUCGUUGUG | 6017-6035 | 6015 |
| 247 | UACAACGAGACUGAAUUGC | 430 | GCAAUUCAGUCUCGUUGUA | 6017-6035 | 6015 |
| 248 | AACAACGAGACUGAAUUGC | 431 | GCAAUUCAGUCUCGUUGUU | 6017-6035 | 6015 |
| 249 | NACAACGAGACUGAAUUGC | 432 | GCAAUUCAGUCUCGUUGUN | 6017-6035 | 6015 |
| 250 | NACAACGAGACUGAAUUGN | 433 | NCAAUUCAGUCUCGUUGUN | 6017-6035 | 6015 |
| 251 | UUGAGUUCUUCCAUUGGCA | 434 | UGCCAAUGGAAGAACUCAA | 6243-6261 | 6241 |
| 252 | AUGAGUUCUUCCAUUGGCA | 435 | UGCCAAUGGAAGAACUCAU | 6243-6261 | 6241 |
| 253 | NUGAGUUCUUCCAUUGGCA | 436 | UGCCAAUGGAAGAACUCAN | 6243-6261 | 6241 |
| 254 | NUGAGUUCUUCCAUUGGCN | 437 | NGCCAAUGGAAGAACUCAN | 6243-6261 | 6241 |
| 255 | GACUAAGUGAGUCUUGCAU | 438 | AUGCAAGACUCACUUAGUC | 6350-6368 | 6348 |
| 256 | UACUAAGUGAGUCUUGCAU | 439 | AUGCAAGACUCACUUAGUA | 6350-6368 | 6348 |
| 257 | AACUAAGUGAGUCUUGCAU | 440 | AUGCAAGACUCACUUAGUU | 6350-6368 | 6348 |
| 258 | NACUAAGUGAGUCUUGCAU | 441 | AUGCAAGACUCACUUAGUN | 6350-6368 | 6348 |
| 259 | NACUAAGUGAGUCUUGCAN | 442 | NUGCAAGACUCACUUAGUN | 6350-6368 | 6348 |
| 260 | CCAGAAAUUUCACUCAUCC | 443 | GGAUGAGUGAAAUUUCUGG | 6606-6624 | 6604 |
| 261 | UCAGAAAUUUCACUCAUCC | 444 | GGAUGAGUGAAAUUUCUGA | 6606-6624 | 6604 |
| 262 | ACAGAAAUUUCACUCAUCC | 445 | GGAUGAGUGAAAUUUCUGU | 6606-6624 | 6604 |
| 263 | NCAGAAAUUUCACUCAUCC | 446 | GGAUGAGUGAAAUUUCUGN | 6606-6624 | 6604 |
| 264 | NCAGAAAUUUCACUCAUCN | 447 | NGAUGAGUGAAAUUUCUGN | 6606-6624 | 6604 |
| 265 | AGAUCAUUCAAUUUGCUCC | 448 | GGAGCAAGUUGAAUGAUCU | 6756-6774 | 6754 |
| 266 | UGAUCAUUCAAUUUGCUCC | 449 | GGAGCAAGUUGAAUGAUCA | 6756-6774 | 6754 |
| 267 | NGAUCAUUCAAUUUGCUCC | 450 | GGAGCAAGUUGAAUGAUCN | 6756-6774 | 6754 |
| 268 | NGAUCAUUCAAUUUGCUCN | 451 | NGAGCAAGUUGAAUGAUCN | 6756-6774 | 6754 |

The HTT RNAi agent sense strands and antisense strands that comprise or consist of the nucleotide sequences in Table 2 can be modified nucleotides or unmodified nucleotides. In some embodiments, the HTT RNAi agents having the sense and antisense strand sequences that comprise or consist of any of the nucleotide sequences in Table 2 are all or substantially all modified nucleotides.

In some embodiments, the antisense strand of an HTT RNAi agent disclosed herein differs by 0, 1, 2, or 3 nucleotides from any of the antisense strand sequences in Table 2. In some embodiments, the sense strand of an HTT RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified HTT RNAi agent sense and antisense strands are provided in Table 3, Table 4, Table 5, Table 6, and Table 9. Certain modified HTT RNAi agent antisense strands, as well as their underlying unmodified nucleobase sequences, are provided in Table 3. Certain modified HTT RNAi agent sense strands, as well as their underlying unmodified nucleobase sequences, are provided in Tables 4, 5, and 6. In forming HTT RNAi agents, each of the nucleotides in each of the underlying base sequences listed in Tables 3, 4, 5, and 6, as well as in Table 2, above, can be a modified nucleotide.

The HTT RNAi agents described herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

In some embodiments, an HTT RNAi agent antisense strand comprises a nucleotide sequence of any of the sequences in Table 2 or Table 3.

In some embodiments, an HTT RNAi agent comprises or consists of a duplex having the nucleobase sequences of the sense strand and the antisense strand of any of the sequences in Table 2, Table 3, Table 4, Table 5, Table 6, or Table 9.

Examples of antisense strands containing modified nucleotides are provided in Table 3. Examples of sense strands containing modified nucleotides are provided in Tables 4, 5 and 6.

As used in Tables 3, 4, 5, 6, and 9, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is=2′-O-methylinosine-3′-phosphorothioate
t=2′-O-methyl-5-methyluridine-3′-phosphate
is=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNAS}=2′,3′-seco-adenosine-3′-phosphorothioate
C_{UNA}=2′,3′-seco-cytidine-3′-phosphate
C_{UNAS}=2′,3′-seco-cytidine-3′-phosphorothioate
G_{UNA}=2′,3′-seco-guanosine-3′-phosphate
G_{UNAS}=2′,3′-seco-guanosine-3′-phosphorothioate
U_{UNA}=2′,3′-seco-uridine-3′-phosphate
U_{UNAS}=2′,3′-seco-uridine-3′-phosphorothioate
a_2N=see Table 10
a_2Ns=see Table 10
(invAb)=inverted abasic deoxyribonucleotide-5′-phosphate, see Table 10
(invAb)s=inverted abasic deoxyribonucleotide-5′-phosphorothioate, see Table 10
s=phosphorothioate linkage
ss=phosphorodithioate linkage
p=terminal phosphate (as synthesized)
vpu=vinyl phosphonate 2′-O-methyluridine-3′-phosphate
cPrpa=5′-cyclopropyl phosphonate-2′-O-methyladenosine-3′-phosphate (see Table 10)
cPrpas=5′-cyclopropyl phosphonate-2′-O-methyladenosine-3′-phosphorothioate (see Table 10)
cPrpu=5′-cyclopropyl phosphonate-2′-O-methyluridine-3′-phosphate (see Table 10)
cPrpus=5′-cyclopropyl phosphonate-2′-O-methyluridine-3′-phosphorothioate (see Table 10)
(Alk-SS-C6)=see Table 10
(C6-SS-Alk)=see Table 10
(C6-SS-C6)=see Table 10
(6-SS-6)=see Table 10
(C6-SS-Alk-Me)=see Table 10
(NH2-C6)=see Table 10
(NH-C6)=see Table 10
(NH-C6)s=see Table 10
L20=see Table 10
L1026=see Table 10
[CP-1113]=Fabs were capped according to the procedure in Example 1E; (see also Table 10 for structure)
LP293=see Table 10
LP183=see Table 10
Fab0016=see Antigen Binding Proteins, infra
Fab0070=see Antigen Binding Proteins, infra

As the person of ordinary skill in the art would readily understand, unless otherwise indicated by the sequence (such as, for example, by a phosphorothioate linkage “s” or by a phosphorodithioate linkage “ss”), when present in an oligonucleotide, the nucleotide monomers are mutually linked by 5′-3′-phosphodiester bonds. As the person of ordinary skill in the art would clearly understand, the inclusion of a phosphorothioate or phosphorodithioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 10). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the HTT RNAi agents and compositions of HTT RNAi agents disclosed herein.

Certain examples of antigen binding proteins and linking groups used with the HTT RNAi agents disclosed herein are included in the chemical structures provided below in Table 10. Each sense strand and/or antisense strand can have any antigen binding protein or linking group listed herein, as well as other targeting groups, antigen binding proteins, linking groups, conjugated to the 5′ and/or 3′ end of the sequence.

TABLE 3
HTT RNAi Agent Antisense Strand Sequences

| | | | Underlying Base Sequence | |
|---|---|---|---|---|
| AS | | SEQ | (5′ → 3′) | SEQ |
| Strand | Modified Antisense Strand | ID | (Shown as an Unmodified | ID |
| ID | (5′ → 3′) | NO. | Nucleotide Sequence) | NO. |
| CA005452 | cPrpusAfscaacGfagacUfgAfaUfugccsu | 452 | UACAACGAGACUGAAUUGCCU | 879 |
| CA005655 | cPrpasGfsaucaUfucaaCfuUfgCfuccasg | 453 | AGAUCAUUCAACUUGCUCCAG | 880 |
| CA005656 | cPrpusAfscuaaGfugagUfcUfuGfcaugsg | 454 | UACUAAGUGAGUCUUGCAUGG | 881 |
| CA005667 | cPrpasGfsaucaUfucaaCfuUfgCfuccassg | 455 | AGAUCAUUCAACUUGCUCCAG | 880 |
| CA005668 | cPrpusAfscuaaGfugagUfcUfuGfcaugssg | 456 | UACUAAGUGAGUCUUGCAUGG | 881 |
| CA005669 | cPrpusAfscaacGfagacUfgAfaUfugccssu | 457 | UACAACGAGACUGAAUUGCCU | 879 |
| CA006561 | cPrpusGfsasCfuCfgaaagCfcUfuCfaUfcasg | 460 | UGACUCGAAAGCCUUCAUCAG | 883 |
| CA006699 | cPrpasGfsauCfauucaaCfuUfgCfuccassg | 461 | AGAUCAUUCAACUUGCUCCAG | 880 |
| CA006701 | cPrpusGfsaucaUfucaaCfuUfgCfuccassg | 462 | UGAUCAUUCAACUUGCUCCAG | 884 |
| CA006702 | cPrpisGfsaucaUfucaaCfuUfgCfuccassg | 463 | IGAUCAUUCAACUUGCUCCAG | 885 |
| CA006706 | cPrpasGfsaucaU_{UNA}ucaaCfuUfgCfuccassg | 464 | AGAUCAUUCAACUUGCUCCAG | 880 |
| CA006707 | cPrpasGfsaucA_{UNA}UfucaaCfuUfgCfuccassg | 465 | AGAUCAUUCAACUUGCUCCAG | 880 |
| CA006709 | cPrpasGfsaucaUfucaaUfuUfgCfuccassg | 466 | AGAUCAUUCAAUUUGCUCCAG | 886 |
| CA006710 | cPrpasGfsaucaUfucaaCfuUfgUfuccassg | 467 | AGAUCAUUCAACUUGUUCCAG | 887 |
| CA006711 | cPrpusAfscaAfcgagacUfgAfaUfugccssu | 468 | UACAACGAGACUGAAUUGCCU | 879 |
| CA006715 | cPrpusAfscaacG_{UNA}agacUfgAfaUfugccssu | 469 | UACAACGAGACUGAAUUGCCU | 879 |
| CA006716 | cPrpusAfscaaC_{UNA}GfagacUfgAfaUfugccssu | 470 | UACAACGAGACUGAAUUGCCU | 879 |
| CA006718 | cPrpusAfscaauGfagacUfgAfaUfugccssu | 471 | UACAAUGAGACUGAAUUGCCU | 888 |
| CA006721 | cPrpussAfcaacGfagacUfgAfaUfugccssu | 472 | UACAACGAGACUGAAUUGCCU | 879 |
| CA006722 | cPrpusAfscuAfagugagUfcUfuGfcaugssg | 473 | UACUAAGUGAGUCUUGCAUGG | 881 |
| CA006726 | cPrpusAfscuaaG_{UNA}ugagUfcUfuGfcaugssg | 474 | UACUAAGUGAGUCUUGCAUGG | 881 |
| CA006727 | cPrpusAfscuaA_{UNA}GfugagUfcUfuGfcaugssg | 475 | UACUAAGUGAGUCUUGCAUGG | 881 |
| CA006729 | cPrpusAfscuaaGfugagUfuUfuGfcaugssg | 476 | UACUAAGUGAGUUUUGCAUGG | 889 |
| CA006731 | cPrpusAfscuaaGfugagUfcUfuGfuaugssg | 477 | UACUAAGUGAGUCUUGUAUGG | 890 |
| CA006733 | cPrpussAfcuaaGfugagUfcUfuGfcaugssg | 478 | UACUAAGUGAGUCUUGCAUGG | 881 |
| CA006734 | cPrpasGfsauuaUfucaaCfuUfgCfuccassg | 479 | AGAUUAUUCAACUUGCUCCAG | 891 |
| CA008019 | cPrpisGfsauCfauucaaCfuUfgCfuccassg | 480 | IGAUCAUUCAACUUGCUCCAG | 885 |
| CA008021 | cPrpusGfsauCfauucaaCfuUfgCfuccassg | 481 | UGAUCAUUCAACUUGCUCCAG | 884 |
| CA911803 | cPrpasGfsgsAfaUfuccaaUfgAfuCfuGfuusu | 482 | AGGAAUUCCAAUGAUCUGUUU | 892 |
| CA911821 | cPrpusUfsusCfuGfacagaCfaUfaAfuCfacsa | 483 | UUUCUGACAGACAUAAUCACA | 893 |
| CA913562 | cPrpasGfsgsAfauuccaaUfgAfuCfuguusu | 484 | AGGAAUUCCAAUGAUCUGUUU | 892 |
| CA913563 | cPrpasGfsgsaAfuuccaaUfgAfuCfuguusu | 485 | AGGAAUUCCAAUGAUCUGUUU | 892 |
| CA913564 | cPrpasGfsgsaauUfccaaUfgAfuCfuguusu | 486 | AGGAAUUCCAAUGAUCUGUUU | 892 |
| CA913565 | cPrpasGfsgsaauucCfaaUfgAfuCfuguusu | 487 | AGGAAUUCCAAUGAUCUGUUU | 892 |
| CA913566 | cPrpasGfsgsAfaUfuccaaugAfuCfuguusu | 488 | AGGAAUUCCAAUGAUCUGUUU | 892 |
| CA913567 | cPrpasGfsgsaauuccaaugAfuCfuguusu | 489 | AGGAAUUCCAAUGAUCUGUUU | 892 |
| CA913568 | cPrpasGfsgsaauuccaaUfgAfucuguusu | 490 | AGGAAUUCCAAUGAUCUGUUU | 892 |
| CA913569 | cPrpasGfsgsaAfuuccaaugAfucuguusu | 491 | AGGAAUUCCAAUGAUCUGUUU | 892 |
| CA913570 | cPrpasGfsgsAfauU_{UNA}ccaaUfgAfuCfuguusu | 492 | AGGAAUUCCAAUGAUCUGUUU | 892 |
| CA914577 | cPrpusGfsusAfaAfagcagAfaCfcUfgAfgcsg | 493 | UGUAAAAGCAGAACCUGAGCG | 894 |
| CA914579 | cPrpusUfscsAfuCfagcuuUfuCfcAfgGfgusc | 494 | UUCAUCAGCUUUUCCAGGGUC | 895 |
| CA914581 | cPrpasGfsgsCfcUfucaucAfgCfuUfuUfccsa | 495 | AGGCCUUCAUCAGCUUUUCCA | 896 |
| CA914583 | cPrpusGfsasCfuCfgaaggCfcUfuCfaUfcasg | 496 | UGACUCGAAGGCCUUCAUCAG | 897 |
| CA914585 | cPrpusAfscsUfuGfagggaCfuCfgAfaGfgcsc | 497 | UACUUGAGGGACUCGAAGGCC | 898 |
| CA914587 | cPrpusGfsasCfuUfgagggAfcUfcGfaAfggsc | 498 | UGACUUGAGGGACUCGAAGGC | 899 |
| CA914589 | cPrpasGfsasAfaAfaguucCfaUfaGfcGfausg | 499 | AGAAAAAGUUCCAUAGCGAUG | 900 |
| CA914591 | cPrpasAfsgsAfuUfagaauCfcAfuCfaAfagsc | 500 | AAGAUUAGAAUCCAUCAAAGC | 901 |
| CA914593 | cPrpusUfsusGfgAfagauuAfgAfaUfcCfausc | 501 | UUUGGAAGAUUAGAAUCCAUC | 902 |
| CA914595 | cPrpusUfsgsUfcAfuuugcAfaAfaUfuGfccsa | 502 | UUGUCAUUUGCAAAAUUGCCA | 903 |
| CA914597 | cPrpusUfsgsUfgUfaugauGfuAfaCfgUfcasg | 503 | UUGUGUAUGAUGUAACGUCAG | 904 |
| CA914599 | cPrpasCfsasAfcAfuugugGfuCfuUfgGfugsc | 504 | ACAACAUUGUGGUCUUGGUGC | 905 |
| CA914601 | cPrpusCfsusAfuAfaguucCfaCfaAfuAfcusc | 505 | UCUAUAAGUUCCACAAUACUC | 906 |
| CA914603 | cPrpusCfsasUfgAfucgauGfuAfgUfuCfaasg | 506 | UCAUGAUCGAUGUAGUUCAAG | 907 |
| CA914605 | cPrpusAfscsAfaAfgcuucAfcAfgCfaUfccsa | 507 | UACAAAGCUUCACAGCAUCCA | 908 |
| CA914607 | cPrpusAfscsUfuGfuuguaAfcAfgGfaCfcusg | 508 | UACUUGUUGUAACAGGACCUG | 909 |
| CA914609 | cPrpusUfsgsAfuGfaggauUfuAfcUfuGfuusg | 509 | UUGAUGAGGAUUUACUUGUUG | 910 |
| CA914611 | cPrpasCfsasAfaCfaguugCfcAfuCfaUfugsg | 510 | ACAAACAGUUGCCAUCAUUGG | 911 |
| CA914613 | cPrpusAfscsAfaAfgccaaUfaAfaCfaCfcusg | 511 | UACAAAGCCAAUAAACACCUG | 912 |
| CA914615 | cPrpusAfsusCfuGfucgagAfcAfgUfcGfcusu | 512 | UAUCUGUCGAGACAGUCGCUU | 913 |
| CA914617 | cPrpusAfsgsUfuGfaacagUfgCfuCfaCfggsa | 513 | UAGUUGAACAGUGCUCACGGA | 914 |
| CA914619 | cPrpusAfscsUfaAfguaacUfuUfgUfgCfugsg | 514 | UACUAAGUAACUUUGUGCUGG | 915 |
| CA914621 | cPrpusCfsasCfaUfuccaaGfuUfuGfgCfugsc | 515 | UCACAUUCCAAGUUUGGCUGC | 916 |
| CA914623 | cPrpusGfsusAfcUfauuucUfcUfaUfuGfcasc | 516 | UGUACUAUUUCUCUAUUGCAC | 917 |
| CA914625 | cPrpusAfsgsUfcAfuggagGfuUfcUfgAfcasg | 517 | UAGUCAUGGAGGUUCUGACAG | 918 |
| CA914627 | cPrpusGfsasAfuGfugauuUfaCfaAfuGfagsc | 518 | UGAAUGUGAUUUACAAUGAGC | 919 |
| CA914629 | cPrpusAfscsUfgAfugaagUfcCfuGfuAfcusg | 519 | UACUGAUGAAGUCCUGUACUG | 920 |
| CA914631 | cPrpusAfscsAfaCfgagacUfgAfaUfuGfccsu | 520 | UACAACGAGACUGAAUUGCCU | 879 |
| CA914633 | cPrpusUfsgsAfgUfucuucCfaUfuGfgCfaasc | 521 | UUGAGUUCUUCCAUUGGCAAC | 921 |
| CA914635 | cPrpusAfscsUfaAfgugagUfcUfuGfcAfugsg | 522 | UACUAAGUGAGUCUUGCAUGG | 881 |
| CA914637 | cPrpusCfsasGfaAfauuucAfcUfcAfuCfccsu | 523 | UCAGAAAUUUCACUCAUCCCU | 922 |
| CA914639 | cPrpasGfsasUfcAfuucaaCfuUfgCfuCfcasg | 524 | AGAUCAUUCAACUUGCUCCAG | 880 |
| CA914972 | cPrpusCfsasUfcAfgcuuuUfcCfaGfgGfucsg | 525 | UCAUCAGCUUUUCCAGGGUCG | 923 |
| CA914974 | cPrpusAfscsUfcGfaaggcCfuUfcAfuCfagsc | 526 | UACUCGAAGGCCUUCAUCAGC | 924 |
| CA914976 | cPrpusUfsusUfcUfuugguCfgGfuGfcAfgcsg | 527 | UUUUCUUUGGUCGGUGCAGCG | 925 |
| CA914978 | cPrpusCfsusUfuCfuuuggUfcGfgUfgCfagsc | 528 | UCUUUCUUUGGUCGGUGCAGC | 926 |
| CA914980 | cPrpusUfscsUfuUfcuuugGfuCfgGfuGfcasg | 529 | UUCUUUCUUUGGUCGGUGCAG | 927 |
| CA915153 | cPrpusGfsascUfcgaaggCfcUfuCfaucasg | 530 | UGACUCGAAGGCCUUCAUCAG | 897 |
| CA915154 | cPrpusGfsascucGfaaggCfcUfuCfaucasg | 531 | UGACUCGAAGGCCUUCAUCAG | 897 |
| CA915155 | cPrpusGfsascucgaAfggCfcUfuCfaucasg | 532 | UGACUCGAAGGCCUUCAUCAG | 897 |
| CA915156 | cPrpusGfsacucGfaaggCfcUfuCfaucasg | 533 | UGACUCGAAGGCCUUCAUCAG | 897 |
| CA915160 | cPrpusGfsascucGfaaggccUfucaucasg | 534 | UGACUCGAAGGCCUUCAUCAG | 897 |
| CA915161 | cPrpusGfsascucgaAfggccUfucaucasg | 535 | UGACUCGAAGGCCUUCAUCAG | 897 |
| CA915162 | cPrpusGfsascucGfaaggccUfucaucAfsg | 536 | UGACUCGAAGGCCUUCAUCAG | 897 |
| CA915860 | cPrpusCfsasuGfaucgauGfuAfgUfucaasg | 537 | UCAUGAUCGAUGUAGUUCAAG | 907 |
| CA915861 | cPrpusCfsasugaUfcgauGfuAfgUfucaasg | 538 | UCAUGAUCGAUGUAGUUCAAG | 907 |
| CA915862 | cPrpusCfsaugaUfcgauGfuAfgUfucaasg | 539 | UCAUGAUCGAUGUAGUUCAAG | 907 |
| CA915863 | cPrpusCfsaugaUfcgauGfuAfgUfucaassg | 540 | UCAUGAUCGAUGUAGUUCAAG | 907 |
| CA915864 | cPrpussCfsaugaUfcgauGfuAfgUfucaassg | 541 | UCAUGAUCGAUGUAGUUCAAG | 907 |
| CA915868 | cPrpusCfsaugaUfcgauguAfguucaAfsg | 542 | UCAUGAUCGAUGUAGUUCAAG | 907 |
| CA915869 | cPrpusdCsaugadTcgaudGuAfgdUucaasg | 543 | UCAUGATCGAUGUAGUUCAAG | 928 |
| CA915870 | cPrpusCfsaugadTcgaudGuAfgdUucaasg | 544 | UCAUGATCGAUGUAGUUCAAG | 928 |
| CA915871 | cPrpusCfsaugadTcgauGfuAfgdUucaasg | 545 | UCAUGATCGAUGUAGUUCAAG | 928 |
| CA915872 | cPrpusUfsgsuguAfugauGfuAfaCfgucasg | 546 | UUGUGUAUGAUGUAACGUCAG | 904 |
| CA915873 | cPrpusUfsgsuGfuaugauGfuAfaCfgucasg | 547 | UUGUGUAUGAUGUAACGUCAG | 904 |
| CA915874 | cPrpusUfsguguAfugauGfuAfaCfgucasg | 548 | UUGUGUAUGAUGUAACGUCAG | 904 |
| CA915875 | cPrpusUfsguguAfugauGfuAfaCfgucassg | 549 | UUGUGUAUGAUGUAACGUCAG | 904 |
| CA915876 | cPrpussUfsguguAfugauGfuAfaCfgucassg | 550 | UUGUGUAUGAUGUAACGUCAG | 904 |
| CA915880 | cPrpusUfsguguAfugauguAfacgucAfsg | 551 | UUGUGUAUGAUGUAACGUCAG | 904 |
| CA915881 | cPrpusdTsgugudAugaudGuAfadCgucasg | 552 | UTGUGUAUGAUGUAACGUCAG | 929 |
| CA915882 | cPrpusUfsgugudAugaudGuAfadCgucasg | 553 | UUGUGUAUGAUGUAACGUCAG | 904 |
| CA915883 | cPrpusUfsgugudAugauGfuAfadCgucasg | 554 | UUGUGUAUGAUGUAACGUCAG | 904 |
| CA915884 | cPrpusAfscsaAfagccaaUfaAfaCfaccusg | 555 | UACAAAGCCAAUAAACACCUG | 912 |
| CA915885 | cPrpusAfscsaaaGfccaaUfaAfaCfaccusg | 556 | UACAAAGCCAAUAAACACCUG | 912 |
| CA915886 | cPrpusAfscaaaGfccaaUfaAfaCfaccusg | 557 | UACAAAGCCAAUAAACACCUG | 912 |
| CA915887 | cPrpusAfscaaaGfccaaUfaAfaCfaccussg | 558 | UACAAAGCCAAUAAACACCUG | 912 |
| CA915888 | cPrpussAfscaaaGfccaaUfaAfaCfaccussg | 559 | UACAAAGCCAAUAAACACCUG | 912 |
| CA915892 | cPrpusAfscaaaGfccaauaAfacaccUfsg | 560 | UACAAAGCCAAUAAACACCUG | 912 |
| CA915893 | cPrpusdAscaaadGccaadTaAfadCaccusg | 561 | UACAAAGCCAATAAACACCUG | 930 |
| CA915894 | cPrpusAfscaaadGccaadTaAfadCaccusg | 562 | UACAAAGCCAATAAACACCUG | 930 |
| CA915895 | cPrpusAfscaaadGccaaUfaAfadCaccusg | 563 | UACAAAGCCAAUAAACACCUG | 912 |

I = hypoxanthine (inosine) nucleotide

TABLE 4
HTT Agent Sense Strand Sequences (Shown Without Linkers,
Conjugates, or Capping Moieties)

| | | | Underlying Base Sequence | |
|---|---|---|---|---|
| | | SEQ | (5′ → 3′) | SEQ |
| | Modified Sense Strand | ID | (Shown as an Unmodified | ID |
| Strand ID | (5′ → 3′) | NO. | Nucleotide Sequence) | NO. |
| CS005389-NL | cugacguuAfCfAfucauacacaa | 564 | CUGACGUUACAUCAUACACAA | 931 |
| CS005390-NL | cuugaacuAfCfAfucgaucauga | 565 | CUUGAACUACAUCGAUCAUGA | 932 |
| CS005391-NL | cagguguuUfAfUfuggcuuugua | 566 | CAGGUGUUUAUUGGCUUUGUA | 933 |
| CS005392-NL | cugacguuAfCfAfucauacacaa | 567 | CUGACGUUACAUCAUACACAA | 931 |
| CS005393-NL | cuugaacuAfCfAfucgaucauga | 568 | CUUGAACUACAUCGAUCAUGA | 932 |
| CS005394-NL | cagguguuUfAfUfuggcuuugua | 569 | CAGGUGUUUAUUGGCUUUGUA | 933 |
| CS005534-NL | aggcaauuCfAfGfucucguugua | 570 | AGGCAAUUCAGUCUCGUUGUA | 934 |
| CS005535-NL | aggcaauuCfAfGfucucguugua | 571 | AGGCAAUUCAGUCUCGUUGUA | 934 |
| CS005558-NL | cugacguuAfCfAfucauacacaa | 572 | CUGACGUUACAUCAUACACAA | 931 |
| CS005559-NL | cuugaacuAfCfAfucgaucauga | 573 | CUUGAACUACAUCGAUCAUGA | 932 |
| CS005560-NL | cagguguuUfAfUfuggcuuugua | 574 | CAGGUGUUUAUUGGCUUUGUA | 933 |
| CS005561-NL | aggcaauuCfAfGfucucguugua | 575 | AGGCAAUUCAGUCUCGUUGUA | 934 |
| CS006560-NL | cugaugaaGfGfCfuuucgaguca | 577 | CUGAUGAAGGCUUUCGAGUCA | 936 |
| CS006700-NL | cuggagcaAfGfUfugaaugauca | 578 | CUGGAGCAAGUUGAAUGAUCA | 937 |
| CS006703-NL | cuggagcaAfgUfugaaugaucu | 579 | CUGGAGCAAGUUGAAUGAUCU | 938 |
| CS006704-NL | cuggagcaAfgUfUfgaaugaucu | 580 | CUGGAGCAAGUUGAAUGAUCU | 938 |
| CS006705-NL | cuggagcaAfgUfuGfaaugaucu | 581 | CUGGAGCAAGUUGAAUGAUCU | 938 |
| CS006708-NL | cuggagcaAfGfUfugaaugauuu | 582 | CUGGAGCAAGUUGAAUGAUUU | 939 |
| CS006712-NL | aggcaauuCfaGfucucguugua | 583 | AGGCAAUUCAGUCUCGUUGUA | 934 |
| CS006713-NL | aggcaauuCfaGfUfcucguugua | 584 | AGGCAAUUCAGUCUCGUUGUA | 934 |
| CS006714-NL | aggcaauuCfaGfuCfucguugua | 585 | AGGCAAUUCAGUCUCGUUGUA | 934 |
| CS006717-NL | aggcaauuCfAfGfucuuguugua | 586 | AGGCAAUUCAGUCUUGUUGUA | 940 |
| CS006719-NL | agguaauuCfAfGfucucguugua | 587 | AGGUAAUUCAGUCUCGUUGUA | 941 |
| CS006720-NL | aggcaauuCfAfGfuuucguugua | 588 | AGGCAAUUCAGUUUCGUUGUA | 942 |
| CS006723-NL | ccaugcaaGfaCfucacuuagua | 589 | CCAUGCAAGACUCACUUAGUA | 943 |
| CS006724-NL | ccaugcaaGfaCfUfcacuuagua | 590 | CCAUGCAAGACUCACUUAGUA | 943 |
| CS006725-NL | ccaugcaaGfaCfuCfacuuagua | 591 | CCAUGCAAGACUCACUUAGUA | 943 |
| CS006728-NL | ccauguaaGfAfCfucacuuagua | 592 | CCAUGUAAGACUCACUUAGUA | 944 |
| CS006730-NL | ccaugcaaGfAfCfucauuuagua | 593 | CCAUGCAAGACUCAUUUAGUA | 945 |
| CS006732-NL | ccaugcaaGfAfCfuuacuuagua | 594 | CCAUGCAAGACUUACUUAGUA | 946 |
| CS007251-NL | cugaugaaGfGfCfcuucgaguca | 595 | CUGAUGAAGGCCUUCGAGUCA | 947 |
| CS007252-NL | ccagcacaAfAfGfuuacuuagua | 596 | CCAGCACAAAGUUACUUAGUA | 948 |
| CS007253-NL | caguacagGfAfCfuucaucagua | 597 | CAGUACAGGACUUCAUCAGUA | 949 |
| CS007254-NL | ccaugcaaGfAfCfucacuuagua | 598 | CCAUGCAAGACUCACUUAGUA | 943 |
| CS007255-NL | cuggagcaAfGfUfugaaugaucu | 599 | CUGGAGCAAGUUGAAUGAUCU | 938 |
| CS007256-NL | cugaugaaGfGfCfcuucgaguca | 600 | CUGAUGAAGGCCUUCGAGUCA | 947 |
| CS007257-NL | ccagcacaAfAfGfuuacuuagua | 601 | CCAGCACAAAGUUACUUAGUA | 948 |
| CS007258-NL | caguacagGfAfCfuucaucagua | 602 | CAGUACAGGACUUCAUCAGUA | 949 |
| CS007259-NL | ccaugcaaGfAfCfucacuuagua | 603 | CCAUGCAAGACUCACUUAGUA | 943 |
| CS007260-NL | cuggagcaAfGfUfugaaugaucu | 604 | CUGGAGCAAGUUGAAUGAUCU | 938 |
| CS008020-NL | cuggagcaAfgUfuGfaaugauca | 605 | CUGGAGCAAGUUGAAUGAUCA | 937 |
| CS008022-NL | cuggagcaAfgUfuGfaaugauua | 606 | CUGGAGCAAGUUGAAUGAUUA | 950 |
| CS008023-NL | agguaauuCfaGfuCfucguugua | 607 | AGGUAAUUCAGUCUCGUUGUA | 941 |
| CS008137-NL | ccaugcaaGfaCfUfuacuuagua | 608 | CCAUGCAAGACUUACUUAGUA | 946 |
| CS009038-NL | aggcaauuCfaGfuCfucguugua | 609 | AGGCAAUUCAGUCUCGUUGUA | 934 |
| CS009039-NL | cuggagcaAfgUfuGfaaugauca | 610 | CUGGAGCAAGUUGAAUGAUCA | 937 |
| CS009183-NL | ccaugcaaGfaCfUfcacuuagua | 611 | CCAUGCAAGACUCACUUAGUA | 943 |
| CS009303-NL | aggcaauuCfAfGfucucguugua | 612 | AGGCAAUUCAGUCUCGUUGUA | 934 |
| CS009304-NL | cuggagcaAfGfUfugaaugaucu | 613 | CUGGAGCAAGUUGAAUGAUCU | 938 |
| CS009305-NL | aggcaauuCfaGfuCfucguugua | 614 | AGGCAAUUCAGUCUCGUUGUA | 934 |
| CS009306-NL | cuggagcaAfgUfuGfaaugauca | 615 | CUGGAGCAAGUUGAAUGAUCA | 937 |
| CS009307-NL | aggcaauuCfaGfuCfucguugua | 616 | AGGCAAUUCAGUCUCGUUGUA | 934 |
| CS009417-NL | ccaugcaaGfaCfUfcacuuagua | 617 | CCAUGCAAGACUCACUUAGUA | 943 |
| CS009418-NL | cuggagcaAfgUfuGfaaugauca | 618 | CUGGAGCAAGUUGAAUGAUCA | 937 |
| CS009419-NL | aggcaauuCfAfGfucucguugua | 619 | AGGCAAUUCAGUCUCGUUGUA | 934 |
| CS009420-NL | cuggagcaAfGfUfugaaugaucu | 620 | CUGGAGCAAGUUGAAUGAUCU | 938 |
| CS011230-NL | aggcaauuCfAfGfucucguugua | 621 | AGGCAAUUCAGUCUCGUUGUA | 934 |
| CS913031-NL | aaacagauCfAfUfuggaauuccu | 622 | AAACAGAUCAUUGGAAUUCCU | 951 |
| CS913032-NL | ugugauuaUfGfUfcugucagaaa | 623 | UGUGAUUAUGUCUGUCAGAAA | 952 |
| CS914492-NL | ugugauuaUfGfUfcugucagaaa | 624 | UGUGAUUAUGUCUGUCAGAAA | 952 |
| CS914493-NL | aaacagauCfAfUfuggaauuccu | 625 | AAACAGAUCAUUGGAAUUCCU | 951 |
| CS914576-NL | cgcucaggUfUfCfugcuuuuaca | 626 | CGCUCAGGUUCUGCUUUUACA | 953 |
| CS914578-NL | gacccuggAfAfAfagcugaugaa | 627 | GACCCUGGAAAAGCUGAUGAA | 954 |
| CS914580-NL | uggaaaagCfUfGfaugaaggccu | 628 | UGGAAAAGCUGAUGAAGGCCU | 955 |
| CS914582-NL | cugaugaaGfGfCfcuucgaguca | 629 | CUGAUGAAGGCCUUCGAGUCA | 947 |
| CS914584-NL | ggccuucgAfGfUfcccucaagua | 630 | GGCCUUCGAGUCCCUCAAGUA | 956 |
| CS914586-NL | gccuucgaGfUfCfccucaaguca | 631 | GCCUUCGAGUCCCUCAAGUCA | 957 |
| CS914588-NL | caucgcuaUfGfGfaacuuuuucu | 632 | CAUCGCUAUGGAACUUUUUCU | 958 |
| CS914590-NL | gcuuugauGfGfAfuucuaaucuu | 633 | GCUUUGAUGGAUUCUAAUCUU | 959 |
| CS914592-NL | gauggauuCfUfAfaucuuccaaa | 634 | GAUGGAUUCUAAUCUUCCAAA | 960 |
| CS914594-NL | uggcaauuUfUfGfcaaaugacaa | 635 | UGGCAAUUUUGCAAAUGACAA | 961 |
| CS914596-NL | cugacguuAfCfAfucauacacaa | 636 | CUGACGUUACAUCAUACACAA | 931 |
| CS914598-NL | gcaccaagAfCfCfacaauguugu | 637 | GCACCAAGACCACAAUGUUGU | 962 |
| CS914600-NL | gaguauugUfGfGfaacuuauaga | 638 | GAGUAUUGUGGAACUUAUAGA | 963 |
| CS914602-NL | cuugaacuAfCfAfucgaucauga | 639 | CUUGAACUACAUCGAUCAUGA | 932 |
| CS914604-NL | uggaugcuGfUfGfaagcuuugua | 640 | UGGAUGCUGUGAAGCUUUGUA | 964 |
| CS914606-NL | cagguccuGfUfUfacaacaagua | 641 | CAGGUCCUGUUACAACAAGUA | 965 |
| CS914608-NL | caacaaguAfAfAfuccucaucaa | 642 | CAACAAGUAAAUCCUCAUCAA | 966 |
| CS914610-NL | ccaaugauGfGfCfaacuguuugu | 643 | CCAAUGAUGGCAACUGUUUGU | 967 |
| CS914612-NL | cagguguuUfAfUfuggcuuugua | 644 | CAGGUGUUUAUUGGCUUUGUA | 933 |
| CS914614-NL | aagcgacuGfUfCfucgacagaua | 645 | AAGCGACUGUCUCGACAGAUA | 968 |
| CS914616-NL | uccgugagCfAfCfuguucaacua | 646 | UCCGUGAGCACUGUUCAACUA | 969 |
| CS914618-NL | ccagcacaAfAfGfuuacuuagua | 647 | CCAGCACAAAGUUACUUAGUA | 948 |
| CS914620-NL | gcagccaaAfCfUfuggaauguga | 648 | GCAGCCAAACUUGGAAUGUGA | 970 |
| CS914622-NL | gugcaauaGfAfGfaaauaguaca | 649 | GUGCAAUAGAGAAAUAGUACA | 971 |
| CS914624-NL | cugucagaAfCfCfuccaugacua | 650 | CUGUCAGAACCUCCAUGACUA | 972 |
| CS914626-NL | gcucauugUfAfAfaucacauuca | 651 | GCUCAUUGUAAAUCACAUUCA | 973 |
| CS914628-NL | caguacagGfAfCfuucaucagua | 652 | CAGUACAGGACUUCAUCAGUA | 949 |
| CS914630-NL | aggcaauuCfAfGfucucguugua | 653 | AGGCAAUUCAGUCUCGUUGUA | 934 |
| CS914632-NL | guugccaaUfGfGfaagaacucaa | 654 | GUUGCCAAUGGAAGAACUCAA | 974 |
| CS914634-NL | ccaugcaaGfAfCfucacuuagua | 655 | CCAUGCAAGACUCACUUAGUA | 943 |
| CS914636-NL | agggaugaGfUfGfaaauuucuga | 656 | AGGGAUGAGUGAAAUUUCUGA | 975 |
| CS914638-NL | cuggagcaAfGfUfugaaugaucu | 657 | CUGGAGCAAGUUGAAUGAUCU | 938 |
| CS914971-NL | cgacccugGfAfAfaagcuiauga | 658 | CGACCCUGGAAAAGCUIAUGA | 976 |
| CS914973-NL | gcugaugaAfGfGfccuuciagua | 659 | GCUGAUGAAGGCCUUCIAGUA | 977 |
| CS914975-NL | cgcugcacCfGfAfccaaagaaaa | 660 | CGCUGCACCGACCAAAGAAAA | 978 |
| CS914977-NL | gcugcaccGfAfCfcaaagaaaga | 661 | GCUGCACCGACCAAAGAAAGA | 979 |
| CS914979-NL | cugcaccgAfCfCfaaagaaagaa | 662 | CUGCACCGACCAAAGAAAGAA | 980 |
| CS915157-NL | cugaugaaGfgCfcuucgaguca | 663 | CUGAUGAAGGCCUUCGAGUCA | 947 |
| CS915158-NL | cugaugAfaGfgCfcuucgaguca | 664 | CUGAUGAAGGCCUUCGAGUCA | 947 |
| CS915159-NL | cugaugaaGfgCfCfuucgaguca | 665 | CUGAUGAAGGCCUUCGAGUCA | 947 |
| CS915865-NL | cuugaacuAfcAfucgaucauga | 666 | CUUGAACUACAUCGAUCAUGA | 932 |
| CS915866-NL | cuugaaCfuAfcAfucgaucauga | 667 | CUUGAACUACAUCGAUCAUGA | 932 |
| CS915867-NL | cuugaacuAfcAfUfcgaucauga | 668 | CUUGAACUACAUCGAUCAUGA | 932 |
| CS915877-NL | cugacguuAfcAfucauacacaa | 669 | CUGACGUUACAUCAUACACAA | 931 |
| CS915878-NL | cugacgUfuAfcAfucauacacaa | 670 | CUGACGUUACAUCAUACACAA | 931 |
| CS915879-NL | cugacguuAfcAfUfcauacacaa | 671 | CUGACGUUACAUCAUACACAA | 931 |
| CS915889-NL | cagguguuUfaUfuggcuuugua | 672 | CAGGUGUUUAUUGGCUUUGUA | 933 |
| CS915890-NL | caggugUfuUfaUfuggcuuugua | 673 | CAGGUGUUUAUUGGCUUUGUA | 933 |
| CS915891-NL | cagguguuUfaUfUfggcuuugua | 674 | CAGGUGUUUAUUGGCUUUGUA | 933 |

I = hypoxanthine (inosine) nucleotide

TABLE 5
HTT Agent Sense Strand Sequences (Shown without antigen binding protein
conjugate and with terminal caps (see Table 10 for structure information.))

| | | | Underlying Base Sequence | |
|---|---|---|---|---|
| | | SEQ | (5′ → 3′) | SEQ |
| | | ID | (Shown as an Unmodified | ID |
| Strand ID | Modified Sense Strand (5′ → 3′) | NO. | Nucleotide Sequence) | NO. |
| CS005389-C | NH2-C6s(invAb)scugacguuAfCfAfucauacacaas | 675 | CUGACGUUACAUCAUACACAA | 931 |
| | (invAb) | | | |
| CS005390-C | NH2-C6s(invAb)scuugaacuAfCfAfucgaucaugas | 676 | CUUGAACUACAUCGAUCAUGA | 932 |
| | (invAb) | | | |
| CS005391-C | NH2-C6s(invAb)scagguguuUfAfUfuggcuuuguas | 677 | CAGGUGUUUAUUGGCUUUGUA | 933 |
| | (invAb) | | | |
| CS005392-C | NH2-C6s(invAb)scugacguuAfCfAfucauacacaas | 678 | CUGACGUUACAUCAUACACAA | 931 |
| | (invAb) | | | |
| CS005393-C | NH2-C6s(invAb)scuugaacuAfCfAfucgaucaugas | 679 | CUUGAACUACAUCGAUCAUGA | 932 |
| | (invAb) | | | |
| CS005394-C | NH2-C6s(invAb)scagguguuUfAfUfuggcuuuguas | 680 | CAGGUGUUUAUUGGCUUUGUA | 933 |
| | (invAb) | | | |
| CS005534-C | NH2-C6s(invAb)saggcaauuCfAfGfucucguuguas | 681 | AGGCAAUUCAGUCUCGUUGUA | 934 |
| | (invAb) | | | |
| CS005535-C | NH2-C6s(invAb)saggcaauuCfAfGfucucguuguas | 682 | AGGCAAUUCAGUCUCGUUGUA | 934 |
| | (invAb) | | | |
| CS005558-C | (invAb)scugacguuAfCfAfucauacacaas(invAb) | 683 | CUGACGUUACAUCAUACACAA | 931 |
| CS005559-C | (invAb)scuugaacuAfCfAfucgaucaugas(invAb) | 684 | CUUGAACUACAUCGAUCAUGA | 932 |
| CS005560-C | (invAb)scagguguuUfAfUfuggcuuuguas(invAb) | 685 | CAGGUGUUUAUUGGCUUUGUA | 933 |
| CS005561-C | (invAb)saggcaauuCfAfGfucucguuguas(invAb) | 686 | AGGCAAUUCAGUCUCGUUGUA | 934 |
| CS006560-C | (invAb)scugaugaaGfGfCfuuucgagucas(invAb) | 688 | CUGAUGAAGGCUUUCGAGUCA | 936 |
| CS006700-C | (invAb)scuggagcaAfGfUfugaaugaucas(invAb) | 689 | CUGGAGCAAGUUGAAUGAUCA | 937 |
| CS006703-C | (invAb)scuggagcaAfgUfugaaugaucus(invAb) | 690 | CUGGAGCAAGUUGAAUGAUCU | 938 |
| CS006704-C | (invAb)scuggagcaAfgUfUfgaaugaucus(invAb) | 691 | CUGGAGCAAGUUGAAUGAUCU | 938 |
| CS006705-C | (invAb)scuggagcaAfgUfuGfaaugaucus(invAb) | 692 | CUGGAGCAAGUUGAAUGAUCU | 938 |
| CS006708-C | (invAb)scuggagcaAfGfUfugaaugauuus(invAb) | 693 | CUGGAGCAAGUUGAAUGAUUU | 939 |
| CS006712-C | (invAb)saggcaauuCfaGfucucguuguas(invAb) | 694 | AGGCAAUUCAGUCUCGUUGUA | 934 |
| CS006713-C | (invAb)saggcaauuCfaGfUfcucguuguas(invAb) | 695 | AGGCAAUUCAGUCUCGUUGUA | 934 |
| CS006714-C | (invAb)saggcaauuCfaGfuCfucguuguas(invAb) | 696 | AGGCAAUUCAGUCUCGUUGUA | 934 |
| CS006717-C | (invAb)saggcaauuCfAfGfucuuguuguas(invAb) | 697 | AGGCAAUUCAGUCUUGUUGUA | 940 |
| CS006719-C | (invAb)sagguaauuCfAfGfucucguuguas(invAb) | 698 | AGGUAAUUCAGUCUCGUUGUA | 941 |
| CS006720-C | (invAb)saggcaauuCfAfGfuuucguuguas(invAb) | 699 | AGGCAAUUCAGUUUCGUUGUA | 942 |
| CS006723-C | (invAb)sccaugcaaGfaCfucacuuaguas(invAb) | 700 | CCAUGCAAGACUCACUUAGUA | 943 |
| CS006724-C | (invAb)sccaugcaaGfaCfUfcacuuaguas(invAb) | 701 | CCAUGCAAGACUCACUUAGUA | 943 |
| CS006725-C | (invAb)sccaugcaaGfaCfuCfacuuaguas(invAb) | 702 | CCAUGCAAGACUCACUUAGUA | 943 |
| CS006728-C | (invAb)sccauguaaGfAfCfucacuuaguas(invAb) | 703 | CCAUGUAAGACUCACUUAGUA | 944 |
| CS006730-C | (invAb)sccaugcaaGfAfCfucauuuaguas(invAb) | 704 | CCAUGCAAGACUCAUUUAGUA | 945 |
| CS006732-C | (invAb)sccaugcaaGfAfCfuuacuuaguas(invAb) | 705 | CCAUGCAAGACUUACUUAGUA | 946 |
| CS007251-C | NH2-C6s(invAb)scugaugaaGfGfCfcuucgagucas | 706 | CUGAUGAAGGCCUUCGAGUCA | 947 |
| | (invAb) | | | |
| CS007252-C | NH2-C6s(invAb)sccagcacaAfAfGfuuacuuaguas | 707 | CCAGCACAAAGUUACUUAGUA | 948 |
| | (invAb) | | | |
| CS007253-C | NH2-C6s(invAb)scaguacagGfAfCfuucaucaguas | 708 | CAGUACAGGACUUCAUCAGUA | 949 |
| | (invAb) | | | |
| CS007254-C | NH2-C6s(invAb)sccaugcaaGfAfCfucacuuaguas | 709 | CCAUGCAAGACUCACUUAGUA | 943 |
| | (invAb) | | | |
| CS007255-C | NH2-C6s(invAb)scuggagcaAfGfUfugaaugaucus | 710 | CUGGAGCAAGUUGAAUGAUCU | 938 |
| | (invAb) | | | |
| CS007256-C | (invAb)scugaugaaGfGfCfcuucgagucas(invAb) | 711 | CUGAUGAAGGCCUUCGAGUCA | 947 |
| CS007257-C | (invAb)sccagcacaAfAfGfuuacuuaguas(invAb) | 712 | CCAGCACAAAGUUACUUAGUA | 948 |
| CS007258-C | (invAb)scaguacagGfAfCfuucaucaguas(invAb) | 713 | CAGUACAGGACUUCAUCAGUA | 949 |
| CS007259-C | (invAb)sccaugcaaGfAfCfucacuuaguas(invAb) | 714 | CCAUGCAAGACUCACUUAGUA | 943 |
| CS007260-C | (invAb)scuggagcaAfGfUfugaaugaucus(invAb) | 715 | CUGGAGCAAGUUGAAUGAUCU | 938 |
| CS008020-C | (invAb)scuggagcaAfgUfuGfaaugaucas(invAb) | 716 | CUGGAGCAAGUUGAAUGAUCA | 937 |
| CS008022-C | (invAb)scuggagcaAfgUfuGfaaugauuas(invAb) | 717 | CUGGAGCAAGUUGAAUGAUUA | 950 |
| CS008023-C | (invAb)sagguaauuCfaGfuCfucguuguas(invAb) | 718 | AGGUAAUUCAGUCUCGUUGUA | 941 |
| CS008137-C | (invAb)sccaugcaaGfaCfUfuacuuaguas(invAb) | 719 | CCAUGCAAGACUUACUUAGUA | 946 |
| CS009038-C | NH2-C6s(invAb)saggcaauuCfaGfuCfucguuguas | 720 | AGGCAAUUCAGUCUCGUUGUA | 934 |
| | (invAb) | | | |
| CS009039-C | NH2-C6s(invAb)scuggagcaAfgUfuGfaaugaucas | 721 | CUGGAGCAAGUUGAAUGAUCA | 937 |
| | (invAb) | | | |
| CS009183-C | NH2-C6s(invAb)sccaugcaaGfaCfUfcacuuaguas | 722 | CCAUGCAAGACUCACUUAGUA | 943 |
| | (invAb) | | | |
| CS009303-C | (invAb)saggcaauuCfAfGfucucguuguas(invAb) | 723 | AGGCAAUUCAGUCUCGUUGUA | 934 |
| CS009304-C | (invAb)scuggagcaAfGfUfugaaugaucus(invAb) | 724 | CUGGAGCAAGUUGAAUGAUCU | 938 |
| CS009305-C | (invAb)saggcaauuCfaGfuCfucguuguas(invAb) | 725 | AGGCAAUUCAGUCUCGUUGUA | 934 |
| CS009306-C | (invAb)scuggagcaAfgUfuGfaaugaucas(invAb) | 726 | CUGGAGCAAGUUGAAUGAUCA | 937 |
| CS009307-C | (invAb)saggcaauuCfaGfuCfucguuguas(invAb) | 727 | AGGCAAUUCAGUCUCGUUGUA | 934 |
| CS009417-C | (invAb)sccaugcaaGfaCfUfcacuuaguas(invAb) | 728 | CCAUGCAAGACUCACUUAGUA | 943 |
| CS009418-C | (invAb)scuggagcaAfgUfuGfaaugaucas(invAb) | 729 | CUGGAGCAAGUUGAAUGAUCA | 937 |
| CS009419-C | (invAb)saggcaauuCfAfGfucucguuguas(invAb) | 730 | AGGCAAUUCAGUCUCGUUGUA | 934 |
| CS009420-C | (invAb)scuggagcaAfGfUfugaaugaucus(invAb) | 731 | CUGGAGCAAGUUGAAUGAUCU | 938 |
| CS913031-C | (invAb)saaacagauCfAfUfuggaauuccus(invAb) | 733 | AAACAGAUCAUUGGAAUUCCU | 951 |
| CS913032-C | (invAb)sugugauuaUfGfUfcugucagaaas(invAb) | 734 | UGUGAUUAUGUCUGUCAGAAA | 952 |
| CS914492-C | (invAb)sugugauuaUfGfUfcugucagaaas(invAb) | 735 | UGUGAUUAUGUCUGUCAGAAA | 952 |
| CS914493-C | (invAb)saaacagauCfAfUfuggaauuccus(invAb) | 736 | AAACAGAUCAUUGGAAUUCCU | 951 |
| CS914576-C | (invAb)scgcucaggUfUfCfugcuuuuacas(invAb) | 737 | CGCUCAGGUUCUGCUUUUACA | 953 |
| CS914578-C | (invAb)sgacccuggAfAfAfagcugaugaas(invAb) | 738 | GACCCUGGAAAAGCUGAUGAA | 954 |
| CS914580-C | (invAb)suggaaaagCfUfGfaugaaggccus(invAb) | 739 | UGGAAAAGCUGAUGAAGGCCU | 955 |
| CS914582-C | (invAb)scugaugaaGfGfCfcuucgagucas(invAb) | 740 | CUGAUGAAGGCCUUCGAGUCA | 947 |
| CS914584-C | (invAb)sggccuucgAfGfUfcccucaaguas(invAb) | 741 | GGCCUUCGAGUCCCUCAAGUA | 956 |
| CS914586-C | (invAb)sgccuucgaGfUfCfccucaagucas(invAb) | 742 | GCCUUCGAGUCCCUCAAGUCA | 957 |
| CS914588-C | (invAb)scaucgcuaUfGfGfaacuuuuucus(invAb) | 743 | CAUCGCUAUGGAACUUUUUCU | 958 |
| CS914590-C | (invAb)sgcuuugauGfGfAfuucuaaucuus(invAb) | 744 | GCUUUGAUGGAUUCUAAUCUU | 959 |
| CS914592-C | (invAb)sgauggauuCfUfAfaucuuccaaas(invAb) | 745 | GAUGGAUUCUAAUCUUCCAAA | 960 |
| CS914594-C | (invAb)suggcaauuUfUfGfcaaaugacaas(invAb) | 746 | UGGCAAUUUUGCAAAUGACAA | 961 |
| CS914596-C | (invAb)scugacguuAfCfAfucauacacaas(invAb) | 747 | CUGACGUUACAUCAUACACAA | 931 |
| CS914598-C | (invAb)sgcaccaagAfCfCfacaauguugus(invAb) | 748 | GCACCAAGACCACAAUGUUGU | 962 |
| CS914600-C | (invAb)sgaguauugUfGfGfaacuuauagas(invAb) | 749 | GAGUAUUGUGGAACUUAUAGA | 963 |
| CS914602-C | (invAb)scuugaacuAfCfAfucgaucaugas(invAb) | 750 | CUUGAACUACAUCGAUCAUGA | 932 |
| CS914604-C | (invAb)suggaugcuGfUfGfaagcuuuguas(invAb) | 751 | UGGAUGCUGUGAAGCUUUGUA | 964 |
| CS914606-C | (invAb)scagguccuGfUfUfacaacaaguas(invAb) | 752 | CAGGUCCUGUUACAACAAGUA | 965 |
| CS914608-C | (invAb)scaacaaguAfAfAfuccucaucaas(invAb) | 753 | CAACAAGUAAAUCCUCAUCAA | 966 |
| CS914610-C | (invAb)sccaaugauGfGfCfaacuguuugus(invAb) | 754 | CCAAUGAUGGCAACUGUUUGU | 967 |
| CS914612-C | (invAb)scagguguuUfAfUfuggcuuuguas(invAb) | 755 | CAGGUGUUUAUUGGCUUUGUA | 933 |
| CS914614-C | (invAb)saagcgacuGfUfCfucgacagauas(invAb) | 756 | AAGCGACUGUCUCGACAGAUA | 968 |
| CS914616-C | (invAb)succgugagCfAfCfuguucaacuas(invAb) | 757 | UCCGUGAGCACUGUUCAACUA | 969 |
| CS914618-C | (invAb)sccagcacaAfAfGfuuacuuaguas(invAb) | 758 | CCAGCACAAAGUUACUUAGUA | 948 |
| CS914620-C | (invAb)sgcagccaaAfCfUfuggaaugugas(invAb) | 759 | GCAGCCAAACUUGGAAUGUGA | 970 |
| CS914622-C | (invAb)sgugcaauaGfAfGfaaauaguacas(invAb) | 760 | GUGCAAUAGAGAAAUAGUACA | 971 |
| CS914624-C | (invAb)scugucagaAfCfCfuccaugacuas(invAb) | 761 | CUGUCAGAACCUCCAUGACUA | 972 |
| CS914626-C | (invAb)sgcucauugUfAfAfaucacauucas(invAb) | 762 | GCUCAUUGUAAAUCACAUUCA | 973 |
| CS914628-C | (invAb)scaguacagGfAfCfuucaucaguas(invAb) | 763 | CAGUACAGGACUUCAUCAGUA | 949 |
| CS914630-C | (invAb)saggcaauuCfAfGfucucguuguas(invAb) | 764 | AGGCAAUUCAGUCUCGUUGUA | 934 |
| CS914632-C | (invAb)sguugccaaUfGfGfaagaacucaas(invAb) | 765 | GUUGCCAAUGGAAGAACUCAA | 974 |
| CS914634-C | (invAb)sccaugcaaGfAfCfucacuuaguas(invAb) | 766 | CCAUGCAAGACUCACUUAGUA | 943 |
| CS914636-C | (invAb)sagggaugaGfUfGfaaauuucugas(invAb) | 767 | AGGGAUGAGUGAAAUUUCUGA | 975 |
| CS914638-C | (invAb)scuggagcaAfGfUfugaaugaucus(invAb) | 768 | CUGGAGCAAGUUGAAUGAUCU | 938 |
| CS914971-C | (invAb)scgacccugGfAfAfaagcuiaugas(invAb) | 769 | CGACCCUGGAAAAGCUIAUGA | 976 |
| CS914973-C | (invAb)sgcugaugaAfGfGfccuuciaguas(invAb) | 770 | GCUGAUGAAGGCCUUCIAGUA | 977 |
| CS914975-C | (invAb)scgcugcacCfGfAfccaaagaaaas(invAb) | 771 | CGCUGCACCGACCAAAGAAAA | 978 |
| CS914977-C | (invAb)sgcugcaccGfAfCfcaaagaaagas(invAb) | 772 | GCUGCACCGACCAAAGAAAGA | 979 |
| CS914979-C | (invAb)scugcaccgAfCfCfaaagaaagaas(invAb) | 773 | CUGCACCGACCAAAGAAAGAA | 980 |
| CS915157-C | (invAb)scugaugaaGfgCfcuucgagucas(invAb) | 774 | CUGAUGAAGGCCUUCGAGUCA | 947 |
| CS915158-C | (invAb)scugaugAfaGfgCfcuucgagucas(invAb) | 775 | CUGAUGAAGGCCUUCGAGUCA | 947 |
| CS915159-C | (invAb)scugaugaaGfgCfCfuucgagucas(invAb) | 776 | CUGAUGAAGGCCUUCGAGUCA | 947 |
| CS915865-C | (invAb)scuugaacuAfcAfucgaucaugas(invAb) | 777 | CUUGAACUACAUCGAUCAUGA | 932 |
| CS915866-C | (invAb)scuugaaCfuAfcAfucgaucaugas(invAb) | 778 | CUUGAACUACAUCGAUCAUGA | 932 |
| CS915867-C | (invAb)scuugaacuAfcAfUfcgaucaugas(invAb) | 779 | CUUGAACUACAUCGAUCAUGA | 932 |
| CS915877-C | (invAb)scugacguuAfcAfucauacacaas(invAb) | 780 | CUGACGUUACAUCAUACACAA | 931 |
| CS915878-C | (invAb)scugacgUfuAfcAfucauacacaas(invAb) | 781 | CUGACGUUACAUCAUACACAA | 931 |
| CS915879-C | (invAb)scugacguuAfcAfUfcauacacaas(invAb) | 782 | CUGACGUUACAUCAUACACAA | 931 |
| CS915889-C | (invAb)scagguguuUfaUfuggcuuuguas(invAb) | 783 | CAGGUGUUUAUUGGCUUUGUA | 933 |
| CS915890-C | (invAb)scaggugUfuUfaUfuggcuuuguas(invAb) | 784 | CAGGUGUUUAUUGGCUUUGUA | 933 |
| CS915891-C | (invAb)scagguguuUfaUfUfggcuuuguas9invAb) | 785 | CAGGUGUUUAUUGGCUUUGUA | 933 |

I = hypoxanthine (inosine) nucleotide

TABLE 6
HTT Agent Sense Strand Sequences (shown with lipid binding moiety or antigen binding moiety).
The structure of the lipid binding moieties are shown in Table 10,
and antigen binding moieties are shown in Tables A and B.

| | | | Corresponding |
|---|---|---|---|
| | | | Sense Strand |
| | | | AM Number |
| | | | Without Linker |
| | | | or Conjugate |
| Strand ID | Modified Sense Strand (5′ -> 3′) | SEQ ID NO. | (See Table 4) |
| CS005558 | Fab0016-L20-(NH- | 786 | CS005558-NL |
| | C6)s(invAb)scugacguuAfCfAfucauacacaas(invAb) | | |
| CS005559 | Fab0016-L20-(NH- | 787 | CS005559-NL |
| | C6)s(invAb)scuugaacuAfCfAfucgaucaugas(invAb) | | |
| CS005560 | Fab0016-L20-(NH- | 788 | CS005560-NL |
| | C6)s(invAb)scagguguuUfAfUfuggcuuuguas(invAb) | | |
| CS005561 | Fab0016-L20-(NH- | 789 | CS005561-NL |
| | C6)s(invAb)saggcaauuCfAfGfucucguuguas(invAb) | | |
| CS006560 | LP293-(NH-C6)s(invAb)scugaugaaGfGfCfuuucgagucas(invAb) | 790 | CS006560-NL |
| CS006700 | LP293-(NH-C6)s(invAb)scuggagcaAfGfUfugaaugaucas(invAb) | 791 | CS006700-NL |
| CS006703 | LP293-(NH-C6)s(invAb)scuggagcaAfgUfugaaugaucus(invAb) | 792 | CS006703-NL |
| CS006704 | LP293-(NH-C6)s(invAb)scuggagcaAfgUfUfgaaugaucus(invAb) | 793 | CS006704-NL |
| CS006705 | LP293-(NH-C6)s(invAb)scuggagcaAfgUfuGfaaugaucus(invAb) | 794 | CS006705-NL |
| CS006708 | LP293-(NH-C6)s(invAb)scuggagcaAfGfUfugaaugauuus(invAb) | 795 | CS006708-NL |
| CS006712 | LP293-(NH-C6)s(invAb)saggcaauuCfaGfucucguuguas(invAb) | 796 | CS006712-NL |
| CS006713 | LP293-(NH-C6)s(invAb)saggcaauuCfaGfUfcucguuguas(invAb) | 797 | CS006713-NL |
| CS006714 | LP293-(NH-C6)s(invAb)saggcaauuCfaGfuCfucguuguas(invAb) | 798 | CS006714-NL |
| CS006717 | LP293-(NH-C6)s(invAb)saggcaauuCfAfGfucuuguuguas(invAb) | 799 | CS006717-NL |
| CS006719 | LP293-(NH-C6)s(invAb)sagguaauuCfAfGfucucguuguas(invAb) | 800 | CS006719-NL |
| CS006720 | LP293-(NH-C6)s(invAb)saggcaauuCfAfGfuuucguuguas(invAb) | 801 | CS006720-NL |
| CS006723 | LP293-(NH-C6)s(invAb)sccaugcaaGfaCfucacuuaguas(invAb) | 802 | CS006723-NL |
| CS006724 | LP293-(NH-C6)s(invAb)sccaugcaaGfaCfUfcacuuaguas(invAb) | 803 | CS006724-NL |
| CS006725 | LP293-(NH-C6)s(invAb)sccaugcaaGfaCfuCfacuuaguas(invAb) | 804 | CS006725-NL |
| CS006728 | LP293-(NH-C6)s(invAb)sccauguaaGfAfCfucacuuaguas(invAb) | 805 | CS006728-NL |
| CS006730 | LP293-(NH-C6)s(invAb)sccaugcaaGfAfCfucauuuaguas(invAb) | 806 | CS006730-NL |
| CS006732 | LP293-(NH-C6)s(invAb)sccaugcaaGfAfCfuuacuuaguas(invAb) | 807 | CS006732-NL |
| CS007256 | Fab0016-L20-(NH- | 808 | CS007256-NL |
| | C6)s(invAb)scugaugaaGfGfCfcuucgagucas(invAb) | | |
| CS007257 | Fab0016-L20-(NH- | 809 | CS007257-NL |
| | C6)s(invAb)sccagcacaAfAfGfuuacuuaguas(invAb) | | |
| CS007258 | Fab0016-L20-(NH- | 810 | CS007258-NL |
| | C6)s(invAb)scaguacagGfAfCfuucaucaguas(invAb) | | |
| CS007259 | Fab0016-L20-(NH- | 811 | CS007259-NL |
| | C6)s(invAb)sccaugcaaGfAfCfucacuuaguas(invAb) | | |
| CS007260 | Fab0016-L20-(NH- | 812 | CS007260-NL |
| | C6)s(invAb)scuggagcaAfGfUfugaaugaucus(invAb) | | |
| CS008020 | LP293-(NH-C6)s(invAb)scuggagcaAfgUfuGfaaugaucas(invAb) | 813 | CS008020-NL |
| CS008022 | LP293-(NH-C6)s(invAb)scuggagcaAfgUfuGfaaugauuas(invAb) | 814 | CS008022-NL |
| CS008023 | LP293-(NH-C6)s(invAb)sagguaauuCfaGfuCfucguuguas(invAb) | 815 | CS008023-NL |
| CS008137 | LP293-(NH-C6)s(invAb)sccaugcaaGfaCfUfuacuuaguas(invAb) | 816 | CS008137-NL |
| CS009303 | Fab0070-L20-(NH- | 817 | CS009303-NL |
| | C6)s(invAb)saggcaauuCfAfGfucucguuguas(invAb) | | |
| CS009304 | Fab0070-L20-(NH- | 818 | CS009304-NL |
| | C6)s(invAb)scuggagcaAfGfUfugaaugaucus(invAb) | | |
| CS009305 | Fab0070-L20-(NH- | 819 | CS009305-NL |
| | C6)s(invAb)saggcaauuCfaGfuCfucguuguas(invAb) | | |
| CS009306 | Fab0070-L20-(NH- | 820 | CS009306-NL |
| | C6)s(invAb)scuggagcaAfgUfuGfaaugaucas(invAb) | | |
| CS009307 | Fab0070-L1026-C6s(invAb)saggcaauuCfaGfuCfucguuguas(invAb) | 821 | CS009307-NL |
| CS009417 | Fab0070-L1026-C6s(invAb)sccaugcaaGfaCfUfcacuuaguas(invAb) | 822 | CS009417-NL |
| CS009418 | Fab0070-L1026-C6s(invAb)scuggagcaAfgUfuGfaaugaucas(invAb) | 823 | CS009418-NL |
| CS009419 | Fab0070-L1026-C6s(invAb)saggcaauuCfAfGfucucguuguas(invAb) | 824 | CS009419-NL |
| CS009420 | Fab0070-L1026-C6s(invAb)scuggagcaAfGfUfugaaugaucus(invAb) | 825 | CS009420-NL |
| CS913031 | LP183s(invAb)saaacagauCfAfUfuggaauuccus(invAb) | 826 | CS913031-NL |
| CS913032 | LP183s(invAb)sugugauuaUfGfUfcugucagaaas(invAb) | 827 | CS913032-NL |
| CS914492 | LP293-(NH-C6)s(invAb)sugugauuaUfGfUfcugucagaaas(invAb) | 828 | CS914492-NL |
| CS914493 | LP293-(NH-C6)s(invAb)saaacagauCfAfUfuggaauuccus(invAb) | 829 | CS914493-NL |
| CS914576 | LP293-(NH-C6)s(invAb)scgcucaggUfUfCfugcuuuuacas(invAb) | 830 | CS914576-NL |
| CS914578 | LP293-(NH-C6)s(invAb)sgacccuggAfAfAfagcugaugaas(invAb) | 831 | CS914578-NL |
| CS914580 | LP293-(NH-C6)s(invAb)suggaaaagCfUfGfaugaaggccus(invAb) | 832 | CS914580-NL |
| CS914582 | LP293-(NH-C6)s(invAb)scugaugaaGfGfCfcuucgagucas(invAb) | 833 | CS914582-NL |
| CS914584 | LP293-(NH-C6)s(invAb)sggccuucgAfGfUfcccucaaguas(invAb) | 834 | CS914584-NL |
| CS914586 | LP293-(NH-C6)s(invAb)sgccuucgaGfUfCfccucaagucas(invAb) | 835 | CS914586-NL |
| CS914588 | LP293-(NH-C6)s(invAb)scaucgcuaUfGfGfaacuuuuucus(invAb) | 836 | CS914588-NL |
| CS914590 | LP293-(NH-C6)s(invAb)sgcuuugauGfGfAfuucuaaucuus(invAb) | 837 | CS914590-NL |
| CS914592 | LP293-(NH-C6)s(invAb)sgauggauuCfUfAfaucuuccaaas(invAb) | 838 | CS914592-NL |
| CS914594 | LP293-(NH-C6)s(invAb)suggcaauuUfUfGfcaaaugacaas(invAb) | 839 | CS914594-NL |
| CS914596 | LP293-(NH-C6)s(invAb)scugacguuAfCfAfucauacacaas(invAb) | 840 | CS914596-NL |
| CS914598 | LP293-(NH-C6)s(invAb)sgcaccaagAfCfCfacaauguugus(invAb) | 841 | CS914598-NL |
| CS914600 | LP293-(NH-C6)s(invAb)sgaguauugUfGfGfaacuuauagas(invAb) | 842 | CS914600-NL |
| CS914602 | LP293-(NH-C6)s(invAb)scuugaacuAfCfAfucgaucaugas(invAb) | 843 | CS914602-NL |
| CS914604 | LP293-(NH-C6)s(invAb)suggaugcuGfUfGfaagcuuuguas(invAb) | 844 | CS914604-NL |
| CS914606 | LP293-(NH-C6)s(invAb)scagguccuGfUfUfacaacaaguas(invAb) | 845 | CS914606-NL |
| CS914608 | LP293-(NH-C6)s(invAb)scaacaaguAfAfAfuccucaucaas(invAb) | 846 | CS914608-NL |
| CS914610 | LP293-(NH-C6)s(invAb)sccaaugauGfGfCfaacuguuugus(invAb) | 847 | CS914610-NL |
| CS914612 | LP293-(NH-C6)s(invAb)scagguguuUfAfUfuggcuuuguas(invAb) | 848 | CS914612-NL |
| CS914614 | LP293-(NH-C6)s(invAb)saagcgacuGfUfCfucgacagauas(invAb) | 849 | CS914614-NL |
| CS914616 | LP293-(NH-C6)s(invAb)succgugagCfAfCfuguucaacuas(invAb) | 850 | CS914616-NL |
| CS914618 | LP293-(NH-C6)s(invAb)sccagcacaAfAfGfuuacuuaguas(invAb) | 851 | CS914618-NL |
| CS914620 | LP293-(NH-C6)s(invAb)sgcagccaaAfCfUfuggaaugugas(invAb) | 852 | CS914620-NL |
| CS914622 | LP293-(NH-C6)s(invAb)sgugcaauaGfAfGfaaauaguacas(invAb) | 853 | CS914622-NL |
| CS914624 | LP293-(NH-C6)s(invAb)scugucagaAfCfCfuccaugacuas(invAb) | 854 | CS914624-NL |
| CS914626 | LP293-(NH-C6)s(invAb)sgcucauugUfAfAfaucacauucas(invAb) | 855 | CS914626-NL |
| CS914628 | LP293-(NH-C6)s(invAb)scaguacagGfAfCfuucaucaguas(invAb) | 856 | CS914628-NL |
| CS914630 | LP293-(NH-C6)s(invAb)saggcaauuCfAfGfucucguuguas(invAb) | 857 | CS914630-NL |
| CS914632 | LP293-(NH-C6)s(invAb)sguugccaaUfGfGfaagaacucaas(invAb) | 858 | CS914632-NL |
| CS914634 | LP293-(NH-C6)s(invAb)sccaugcaaGfAfCfucacuuaguas(invAb) | 859 | CS914634-NL |
| CS914636 | LP293-(NH-C6)s(invAb)sagggaugaGfUfGfaaauuucugas(invAb) | 860 | CS914636-NL |
| CS914638 | LP293-(NH-C6)s(invAb)scuggagcaAfGfUfugaaugaucus(invAb) | 861 | CS914638-NL |
| CS914971 | LP293-(NH-C6)s(invAb)scgacccugGfAfAfaagcuiaugas(invAb) | 862 | CS914971-NL |
| CS914973 | LP293-(NH-C6)s(invAb)sgcugaugaAfGfGfccuuciaguas(invAb) | 863 | CS914973-NL |
| CS914975 | LP293-(NH-C6)s(invAb)scgcugcacCfGfAfccaaagaaaas(invAb) | 864 | CS914975-NL |
| CS914977 | LP293-(NH-C6)s(invAb)sgcugcaccGfAfCfcaaagaaagas(invAb) | 865 | CS914977-NL |
| CS914979 | LP293-(NH-C6)s(invAb)scugcaccgAfCfCfaaagaaagaas(invAb) | 866 | CS914979-NL |
| CS915157 | LP293-(NH-C6)s(invAb)scugaugaaGfgCfcuucgagucas(invAb) | 867 | CS915157-NL |
| CS915158 | LP293-(NH-C6)s(invAb)scugaugAfaGfgCfcuucgagucas(invAb) | 868 | CS915158-NL |
| CS915159 | LP293-(NH-C6)s(invAb)scugaugaaGfgCfCfuucgagucas(invAb) | 869 | CS915159-NL |
| CS915865 | LP293-(NH-C6)s(invAb)scuugaacuAfcAfucgaucaugas(invAb) | 870 | CS915865-NL |
| CS915866 | LP293-(NH-C6)s(invAb)scuugaaCfuAfcAfucgaucaugas(invAb) | 871 | CS915866-NL |
| CS915867 | LP293-(NH-C6)s(invAb)scuugaacuAfcAfUfcgaucaugas(invAb) | 872 | CS915867-NL |
| CS915877 | LP293-(NH-C6)s(invAb)scugacguuAfcAfucauacacaas(invAb) | 873 | CS915877-NL |
| CS915878 | LP293-(NH-C6)s(invAb)scugacgUfuAfcAfucauacacaas(invAb) | 874 | CS915878-NL |
| CS915879 | LP293-(NH-C6)s(invAb)scugacguuAfcAfUfcauacacaas(invAb) | 875 | CS915879-NL |
| CS915889 | LP293-(NH-C6)s(invAb)scagguguuUfaUfuggcuuuguas(invAb) | 876 | CS915889-NL |
| CS915890 | LP293-(NH-C6)s(invAb)scaggugUfuUfaUfuggcuuuguas(invAb) | 877 | CS915890-NL |
| CS915891 | LP293-(NH-C6)s(invAb)scagguguuUfaUfUfggcuuuguas(invAb) | 878 | CS915891-NL |

The HTT RNAi agents disclosed herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

As shown in Table 5 above, certain of the example HTT RNAi agent nucleotide sequences are shown to further include reactive linking groups at one or both of the 5′ terminal end and the 3′ terminal end of the sense strand. For example, many of the HTT RNAi agent sense strand sequences shown in Table 5 above have a (NH2-C6) linking group at the 5′ end of the nucleotide sequence. Other linking groups, such as a (6-SS-6) linking group or a (C6-SS-C6) linking group, may be present as well or alternatively in certain embodiments. Such reactive linking groups are positioned to facilitate the linking of targeting ligands, targeting groups, and/or antigen binding proteins to the HTT RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse demand Diels-Alder cycloaddition reaction, oxime ligation, and Copper (I)—catalyzed or strain-promoted azide-alkyne cycloaddition reaction.

In some embodiments, targeting ligands, can be synthesized as activated esters, such as tetrafluorophenyl (TFP) esters, which can be displaced by a reactive amino group (e.g., NH₂-C₆) to attach the targeting ligand to the HTT RNAi agents disclosed herein. In some embodiments, targeting ligands are synthesized as azides, which can be conjugated to a propargyl or DBCO group, for example, via Copper (I)—catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, an antigen binding protein or one or more targeting ligands. As described herein, the disulfide bond of C6-SS-C6 is first reduced, removing the dT from the molecule, which can then facilitate the conjugation of the desired component. The terminal dT nucleotide therefore is not a part of the fully conjugated construct.

In some embodiments, the antisense strand of an HTT RNAi agent disclosed herein differs by 0, 1, 2, or 3 nucleotides from any of the antisense strand sequences in Table 3 or Table 9. In some embodiments, the sense strand of an HTT RNAi agent disclosed herein differs by 0, 1, 2, or 3 nucleotides from any of the sense strand sequences in Table 4, Table 5, Table 6, or Table 9.

In some embodiments, an HTT RNAi agent antisense strand comprises a nucleotide sequence of any of the sequences in Table 2 or Table 3. In some embodiments, an HTT RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 9. In certain embodiments, an HTT RNAi agent antisense strand comprises or consists of a modified sequence of any one of the modified sequences in Table 3 or Table 9.

In some embodiments, an HTT RNAi agent sense strand comprises the nucleotide sequence of any of the sequences in Table 2 or Table 4. In some embodiments, an HTT RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, or Table 9. In certain embodiments, an HTT RNAi agent sense strand comprises or consists of a modified sequence of any one of the modified sequences in Table 6 or Table 9.

For the RNAi agents disclosed herein, the nucleotide at position 1 of the antisense strand (from 5′ end→3′ end) can be perfectly complementary to an HTT gene, or can be non-complementary to an HTT gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, an HTT RNAi agent antisense strand comprises the sequence of nucleotides (from 5′ end→3′ end) 2-18 or 2-19 of any of the antisense strand sequences in Table 2, Table 3, or Table 9. In some embodiments, an HTT RNAi sense strand comprises the sequence of nucleotides (from 5′ end→3′ end) 1-17 or 1-18 of any of the sense strand sequences in Table 2, Table 4, Table 5, Table 6, or Table 9.

In some embodiments, an HTT RNAi agent includes (i) an antisense strand comprising the sequence of nucleotides (from 5′ end→3′ end) 2-18 or 2-19 of any of the antisense strand sequences in Table 2, Table 3, or Table 9, and (ii) a sense strand comprising the sequence of nucleotides (from 5′ end→3′ end) 1-17 or 1-18 of any of the sense strand sequences in Table 2, Table 4, Table 5, Table 6, or Table 9.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the HTT RNAi agent has a sense strand consisting of the modified sequence of any of the modified sequences in Table 4, Table 5, Table 6, or Table 9, and an antisense strand consisting of the modified sequence of any of the modified sequences in Table 3 or Table 9. Certain representative sequence pairings are exemplified by the Duplex ID Nos. shown in Tables 7, 8, and 9.

In some embodiments, an HTT RNAi agent comprises, consists of, or consists essentially of a duplex represented by any one of the Duplex ID Nos. presented herein. In some embodiments, an HTT RNAi agent consists of any of the Duplex ID Nos. presented herein. In some embodiments, an HTT RNAi agent comprises the sense strand and antisense strand nucleotide sequences of any of the Duplex ID Nos. presented herein. In some embodiments, an HTT RNAi agent comprises the sense strand and antisense strand nucleotide sequences of any of the Duplex ID Nos. presented herein and a targeting group, linking group, antigen binding protein and/or other non-nucleotide group wherein the targeting group, linking group, antigen binding protein and/or other non-nucleotide group is covalently linked (i.e., conjugated) to the sense strand or the antisense strand. In some embodiments, an HTT RNAi agent includes the sense strand and antisense strand modified nucleotide sequences of any of the Duplex ID Nos. presented herein. In some embodiments, an HTT RNAi agent comprises the sense strand and antisense strand modified nucleotide sequences of any of the Duplex ID Nos. presented herein and a targeting group, linking group, and/or other non-nucleotide group, wherein the targeting group, linking group, antigen binding protein and/or other non-nucleotide group is covalently linked to the sense strand or the antisense strand.

In some embodiments, an HTT RNAi agent comprises an antisense strand and a sense strand having the nucleotide sequences of any of the antisense strand/sense strand duplexes of Tables 2, 7, 8, or 9, and comprises an antigen binding protein. In some embodiments, an HTT RNAi agent comprises an antisense strand and a sense strand having the nucleotide sequences of any of the antisense strand/sense strand duplexes of Tables 2, 7, 8, or 9, and comprises one or more antigen binding proteins.

In some embodiments, an HTT RNAi agent comprises an antisense strand and a sense strand having the nucleotide sequences of any of the antisense strand/sense strand duplexes of Tables 2, 7, 8, or 9, and comprises an antigen binding protein. In some embodiments, an HTT RNAi agent comprises an antisense strand and a sense strand having the nucleotide sequences of any of the antisense strand/sense strand duplexes of Tables 2, 7, 8, or 9, and comprises one or more antigen binding protein.

In some embodiments, an HTT RNAi agent comprises an antisense strand and a sense strand having the modified nucleotide sequences of any of the antisense strand/sense strand duplexes of Tables 7, 8, and 9.

In some embodiments, an HTT RNAi agent comprises an antisense strand and a sense strand having the modified nucleotide sequences of any of the antisense strand/sense strand duplexes of Tables 7, 8, and 9, and comprises an antigen binding protein.

In some embodiments, an HTT RNAi agent comprises, consists of, or consists essentially of any of the duplexes of Tables 7, 8, and 9.

TABLE 7
HTT RNAi Agent Duplexes with Corresponding Sense and Antisense Strand ID Numbers
and Sequence ID numbers for the modified and unmodified nucleotide sequences.

| | | AS modified | AS unmodified | | SS modified | SS unmodified |
|---|---|---|---|---|---|---|
| Duplex | AS ID | SEQ ID NO: | SEQ ID NO: | SS ID | SEQ ID NO: | SEQ ID NO: |
| AC004413 | CA005452 | 452 | 879 | CS914630 | 857 | 934 |
| AC004496 | CA914597 | 503 | 904 | CS005558 | 786 | 931 |
| AC004497 | CA914603 | 506 | 907 | CS005559 | 787 | 932 |
| AC004498 | CA914613 | 511 | 912 | CS005560 | 788 | 933 |
| AC004499 | CA914631 | 520 | 879 | CS005561 | 789 | 934 |
| AC004563 | CA005655 | 453 | 880 | CS914638 | 861 | 938 |
| AC004564 | CA005656 | 454 | 881 | CS914634 | 859 | 943 |
| AC004575 | CA005667 | 455 | 880 | CS914638 | 861 | 938 |
| AC004576 | CA005668 | 456 | 881 | CS914634 | 859 | 943 |
| AC004577 | CA005669 | 457 | 879 | CS914630 | 857 | 934 |
| AC005385 | CA006561 | 460 | 883 | CS006560 | 790 | 936 |
| AC005500 | CA006699 | 461 | 880 | CS914638 | 861 | 938 |
| AC005501 | CA006701 | 462 | 884 | CS006700 | 791 | 937 |
| AC005502 | CA006702 | 463 | 885 | CS914638 | 861 | 938 |
| AC005503 | CA005667 | 455 | 880 | CS006703 | 792 | 938 |
| AC005504 | CA005667 | 455 | 880 | CS006704 | 793 | 938 |
| AC005505 | CA006706 | 464 | 880 | CS914638 | 861 | 938 |
| AC005506 | CA006707 | 465 | 880 | CS914638 | 861 | 938 |
| AC005507 | CA005667 | 455 | 880 | CS006708 | 795 | 939 |
| AC005508 | CA006709 | 466 | 886 | CS914638 | 861 | 938 |
| AC005509 | CA006710 | 467 | 887 | CS914638 | 861 | 938 |
| AC005510 | CA005667 | 455 | 880 | CS006705 | 794 | 938 |
| AC005511 | CA006711 | 468 | 879 | CS914630 | 857 | 934 |
| AC005512 | CA005669 | 457 | 879 | CS006712 | 796 | 934 |
| AC005513 | CA005669 | 457 | 879 | CS006713 | 797 | 934 |
| AC005514 | CA005669 | 457 | 879 | CS006714 | 798 | 934 |
| AC005515 | CA006715 | 469 | 879 | CS914630 | 857 | 934 |
| AC005516 | CA006716 | 470 | 879 | CS914630 | 857 | 934 |
| AC005517 | CA005669 | 457 | 879 | CS006717 | 799 | 940 |
| AC005518 | CA006718 | 471 | 888 | CS914630 | 857 | 934 |
| AC005519 | CA005669 | 457 | 879 | CS006719 | 800 | 941 |
| AC005520 | CA005669 | 457 | 879 | CS006720 | 801 | 942 |
| AC005521 | CA006721 | 472 | 879 | CS914630 | 857 | 934 |
| AC005522 | CA006722 | 473 | 881 | CS914634 | 859 | 943 |
| AC005523 | CA005668 | 456 | 881 | CS006723 | 802 | 943 |
| AC005524 | CA005668 | 456 | 881 | CS006724 | 803 | 943 |
| AC005525 | CA005668 | 456 | 881 | CS006725 | 804 | 943 |
| AC005526 | CA006726 | 474 | 881 | CS914634 | 859 | 943 |
| AC005527 | CA006727 | 475 | 881 | CS914634 | 859 | 943 |
| AC005528 | CA006729 | 476 | 889 | CS006728 | 805 | 944 |
| AC005529 | CA005668 | 456 | 881 | CS006730 | 806 | 945 |
| AC005530 | CA006731 | 477 | 890 | CS914634 | 859 | 943 |
| AC005531 | CA005668 | 456 | 881 | CS006732 | 807 | 946 |
| AC005532 | CA006733 | 478 | 881 | CS914634 | 859 | 943 |
| AC005533 | CA006734 | 479 | 891 | CS914638 | 861 | 938 |
| AC005956 | CA914597 | 503 | 904 | CS005558 | 786 | 931 |
| AC005957 | CA914603 | 506 | 907 | CS005559 | 787 | 932 |
| AC005958 | CA914613 | 511 | 912 | CS005560 | 788 | 933 |
| AC005959 | CA914631 | 520 | 879 | CS005561 | 789 | 934 |
| AC005998 | CA914619 | 514 | 915 | CS007257 | 809 | 948 |
| AC005999 | CA914629 | 519 | 920 | CS007258 | 810 | 949 |
| AC006000 | CA914635 | 522 | 881 | CS007259 | 811 | 943 |
| AC006001 | CA914639 | 524 | 880 | CS007260 | 812 | 938 |
| AC006002 | CA914583 | 496 | 897 | CS007256 | 808 | 947 |
| AC006704 | CA008019 | 480 | 885 | CS006705 | 794 | 938 |
| AC006705 | CA008021 | 481 | 884 | CS008020 | 813 | 937 |
| AC006706 | CA008021 | 481 | 884 | CS008022 | 814 | 950 |
| AC006707 | CA006711 | 468 | 879 | CS006714 | 798 | 934 |
| AC006708 | CA006711 | 468 | 879 | CS006719 | 800 | 941 |
| AC006709 | CA006711 | 468 | 879 | CS008023 | 815 | 941 |
| AC006805 | CA006722 | 473 | 881 | CS006724 | 803 | 943 |
| AC006806 | CA005668 | 456 | 881 | CS008137 | 816 | 946 |
| AC006807 | CA006722 | 473 | 881 | CS008137 | 816 | 946 |
| AC007863 | CA914631 | 520 | 879 | CS009303 | 817 | 934 |
| AC007864 | CA914639 | 524 | 880 | CS009304 | 818 | 938 |
| AC007865 | CA006711 | 468 | 879 | CS009305 | 819 | 934 |
| AC007866 | CA008021 | 481 | 884 | CS009306 | 820 | 937 |
| AC007867 | CA006711 | 468 | 879 | CS009307 | 821 | 934 |
| AC007952 | CA005668 | 456 | 881 | CS009417 | 822 | 943 |
| AC007953 | CA008021 | 481 | 884 | CS009418 | 823 | 937 |
| AC007968 | CA914631 | 520 | 879 | CS009419 | 824 | 934 |
| AC007969 | CA914639 | 524 | 880 | CS009420 | 825 | 938 |
| AC910139 | CA911803 | 482 | 892 | CS913031 | 826 | 951 |
| AC910140 | CA911821 | 483 | 893 | CS913032 | 827 | 952 |
| AC910577 | CA913562 | 484 | 892 | CS913031 | 826 | 951 |
| AC910578 | CA913563 | 485 | 892 | CS913031 | 826 | 951 |
| AC910579 | CA913564 | 486 | 892 | CS913031 | 826 | 951 |
| AC910580 | CA913565 | 487 | 892 | CS913031 | 826 | 951 |
| AC910581 | CA913566 | 488 | 892 | CS913031 | 826 | 951 |
| AC910582 | CA913567 | 489 | 892 | CS913031 | 826 | 951 |
| AC910583 | CA913568 | 490 | 892 | CS913031 | 826 | 951 |
| AC910584 | CA913569 | 491 | 892 | CS913031 | 826 | 951 |
| AC910585 | CA913570 | 492 | 892 | CS913031 | 826 | 951 |
| AC911226 | CA911821 | 483 | 893 | CS914492 | 828 | 952 |
| AC911227 | CA911803 | 482 | 892 | CS914493 | 829 | 951 |
| AC911290 | CA914577 | 493 | 894 | CS914576 | 830 | 953 |
| AC911291 | CA914579 | 494 | 895 | CS914578 | 831 | 954 |
| AC911292 | CA914581 | 495 | 896 | CS914580 | 832 | 955 |
| AC911293 | CA914583 | 496 | 897 | CS914582 | 833 | 947 |
| AC911294 | CA914585 | 497 | 898 | CS914584 | 834 | 956 |
| AC911295 | CA914587 | 498 | 899 | CS914586 | 835 | 957 |
| AC911296 | CA914589 | 499 | 900 | CS914588 | 836 | 968 |
| AC911297 | CA914591 | 500 | 901 | CS914590 | 837 | 959 |
| AC911298 | CA914593 | 501 | 902 | CS914592 | 838 | 960 |
| AC911299 | CA914595 | 502 | 903 | CS914594 | 839 | 961 |
| AC911300 | CA914597 | 503 | 904 | CS914596 | 840 | 931 |
| AC911301 | CA914599 | 504 | 905 | CS914598 | 841 | 962 |
| AC911302 | CA914601 | 505 | 906 | CS914600 | 842 | 963 |
| AC911303 | CA914603 | 506 | 907 | CS914602 | 843 | 932 |
| AC911304 | CA914605 | 507 | 908 | CS914604 | 844 | 964 |
| AC911305 | CA914607 | 508 | 909 | CS914606 | 845 | 965 |
| AC911306 | CA914609 | 509 | 910 | CS914608 | 846 | 966 |
| AC911307 | CA914611 | 510 | 911 | CS914610 | 847 | 967 |
| AC911308 | CA914613 | 511 | 912 | CS914612 | 848 | 933 |
| AC911309 | CA914615 | 512 | 913 | CS914614 | 849 | 968 |
| AC911310 | CA914617 | 513 | 914 | CS914616 | 850 | 969 |
| AC911311 | CA914619 | 514 | 915 | CS914618 | 851 | 948 |
| AC911312 | CA914621 | 515 | 916 | CS914620 | 852 | 970 |
| AC911313 | CA914623 | 516 | 917 | CS914622 | 853 | 971 |
| AC911314 | CA914625 | 517 | 918 | CS914624 | 854 | 972 |
| AC911315 | CA914627 | 518 | 919 | CS914626 | 855 | 973 |
| AC911316 | CA914629 | 519 | 920 | CS914628 | 856 | 949 |
| AC911317 | CA914631 | 520 | 879 | CS914630 | 857 | 934 |
| AC911318 | CA914633 | 521 | 921 | CS914632 | 858 | 974 |
| AC911319 | CA914635 | 522 | 881 | CS914634 | 859 | 943 |
| AC911320 | CA914637 | 523 | 922 | CS914636 | 860 | 975 |
| AC911321 | CA914639 | 524 | 880 | CS914638 | 861 | 938 |
| AC911599 | CA914972 | 525 | 923 | CS914971 | 862 | 976 |
| AC911600 | CA914974 | 526 | 924 | CS914973 | 863 | 977 |
| AC911601 | CA914976 | 527 | 925 | CS914975 | 864 | 978 |
| AC911602 | CA914978 | 528 | 926 | CS914977 | 865 | 979 |
| AC911603 | CA914980 | 529 | 927 | CS914979 | 866 | 980 |
| AC911758 | CA915153 | 530 | 897 | CS914582 | 833 | 947 |
| AC911759 | CA915154 | 531 | 897 | CS914582 | 833 | 947 |
| AC911760 | CA915155 | 532 | 897 | CS914582 | 833 | 947 |
| AC911761 | CA915156 | 533 | 89 | CS914582 | 833 | 947 |
| AC911762 | CA915154 | 531 | 897 | CS915157 | 867 | 947 |
| AC911763 | CA915154 | 531 | 897 | CS915158 | 868 | 947 |
| AC911764 | CA915154 | 531 | 897 | CS915159 | 869 | 947 |
| AC911765 | CA915160 | 534 | 897 | CS914582 | 833 | 947 |
| AC911766 | CA915161 | 535 | 897 | CS914582 | 833 | 947 |
| AC911767 | CA915162 | 536 | 897 | CS914582 | 833 | 947 |
| AC912419 | CA915860 | 537 | 907 | CS914602 | 843 | 932 |
| AC912420 | CA915861 | 538 | 907 | CS914602 | 843 | 932 |
| AC912421 | CA915862 | 539 | 907 | CS914602 | 843 | 932 |
| AC912422 | CA915863 | 540 | 907 | CS914602 | 843 | 932 |
| AC912423 | CA915864 | 541 | 907 | CS914602 | 843 | 932 |
| AC912424 | CA915862 | 539 | 907 | CS915865 | 870 | 932 |
| AC912425 | CA915862 | 539 | 907 | CS915866 | 871 | 932 |
| AC912426 | CA915862 | 539 | 907 | CS915867 | 872 | 932 |
| AC912427 | CA915868 | 542 | 907 | CS914602 | 843 | 932 |
| AC912428 | CA915869 | 543 | 928 | CS914602 | 843 | 932 |
| AC912429 | CA915870 | 544 | 928 | CS914602 | 843 | 932 |
| AC912430 | CA915871 | 545 | 928 | CS914602 | 843 | 932 |
| AC912431 | CA915872 | 546 | 904 | CS914596 | 840 | 931 |
| AC912432 | CA915873 | 547 | 904 | CS914596 | 840 | 931 |
| AC912433 | CA915874 | 548 | 904 | CS914596 | 840 | 931 |
| AC912434 | CA915875 | 549 | 904 | CS914596 | 840 | 931 |
| AC912435 | CA915876 | 550 | 904 | CS914596 | 840 | 931 |
| AC912436 | CA915874 | 548 | 904 | CS915877 | 873 | 931 |
| AC912437 | CA915874 | 548 | 904 | CS915878 | 874 | 931 |
| AC912438 | CA915874 | 548 | 904 | CS915879 | 875 | 931 |
| AC912439 | CA915880 | 551 | 904 | CS914596 | 840 | 931 |
| AC912440 | CA915881 | 552 | 929 | CS914596 | 840 | 931 |
| AC912441 | CA915882 | 553 | 904 | CS914596 | 840 | 931 |
| AC912442 | CA915883 | 554 | 904 | CS914596 | 840 | 931 |
| AC912443 | CA915884 | 555 | 912 | CS914612 | 848 | 933 |
| AC912444 | CA915885 | 556 | 912 | CS914612 | 848 | 933 |
| AC912445 | CA915886 | 557 | 912 | CS914612 | 848 | 933 |
| AC912446 | CA915887 | 558 | 912 | CS914612 | 848 | 933 |
| AC912447 | CA915888 | 559 | 912 | CS914612 | 848 | 933 |
| AC912448 | CA915886 | 557 | 912 | CS915889 | 876 | 933 |
| AC912449 | CA915886 | 557 | 912 | CS915890 | 877 | 933 |
| AC912450 | CA915886 | 557 | 911 | CS915891 | 878 | 933 |
| AC912451 | CA915892 | 560 | 912 | CS914612 | 848 | 933 |
| AC912452 | CA915893 | 561 | 930 | CS914612 | 848 | 933 |
| AC912453 | CA915894 | 562 | 930 | CS914612 | 848 | 933 |
| AC912454 | CA915895 | 563 | 912 | CS914612 | 848 | 933 |

TABLE 8
Conjugate Duplex ID Numbers Referencing Position
Targeted On Huntingtin (HTT) Gene

| | | | Targeted HTT |
|---|---|---|---|
| | | | Gene Position |
| Duplex | AS ID | SS ID | (Of SEQ ID NO: 1) |
| AC004413 | CA005452 | CS914630 | 6015 |
| AC004496 | CA914597 | CS005558 | 1229 |
| AC004497 | CA914603 | CS005559 | 2410 |
| AC004498 | CA914613 | CS005560 | 4556 |
| AC004499 | CA914631 | CS005561 | 6015 |
| AC004563 | CA005655 | CS914638 | 6754 |
| AC004564 | CA005656 | CS914634 | 6348 |
| AC004575 | CA005667 | CS914638 | 6754 |
| AC004576 | CA005668 | CS914634 | 6348 |
| AC004577 | CA005669 | CS914630 | 6015 |
| AC005385 | CA006561 | CS006560 | 164 |
| AC005500 | CA006699 | CS914638 | 6754 |
| AC005501 | CA006701 | CS006700 | 6754 |
| AC005502 | CA006702 | CS914638 | 6754 |
| AC005503 | CA005667 | CS006703 | 6754 |
| AC005504 | CA005667 | CS006704 | 6754 |
| AC005505 | CA006706 | CS914638 | 6754 |
| AC005506 | CA006707 | CS914638 | 6754 |
| AC005507 | CA005667 | CS006708 | 6754 |
| AC005508 | CA006709 | CS914638 | 6754 |
| AC005509 | CA006710 | CS914638 | 6754 |
| AC005510 | CA005667 | CS006705 | 6754 |
| AC005511 | CA006711 | CS914630 | 6015 |
| AC005512 | CA005669 | CS006712 | 6015 |
| AC005513 | CA005669 | CS006713 | 6015 |
| AC005514 | CA005669 | CS006714 | 6015 |
| AC005515 | CA006715 | CS914630 | 6015 |
| AC005516 | CA006716 | CS914630 | 6015 |
| AC005517 | CA005669 | CS006717 | 6015 |
| AC005518 | CA006718 | CS914630 | 6015 |
| AC005519 | CA005669 | CS006719 | 6015 |
| AC005520 | CA005669 | CS006720 | 6015 |
| AC005521 | CA006721 | CS914630 | 6015 |
| AC005522 | CA006722 | CS914634 | 6348 |
| AC005523 | CA005668 | CS006723 | 6348 |
| AC005524 | CA005668 | CS006724 | 6348 |
| AC005525 | CA005668 | CS006725 | 6348 |
| AC005526 | CA006726 | CS914634 | 6348 |
| AC005527 | CA006727 | CS914634 | 6348 |
| AC005528 | CA006729 | CS006728 | 6348 |
| AC005529 | CA005668 | CS006730 | 6348 |
| AC005530 | CA006731 | CS914634 | 6348 |
| AC005531 | CA005668 | CS006732 | 6348 |
| AC005532 | CA006733 | CS914634 | 6348 |
| AC005533 | CA006734 | CS914638 | 6754 |
| AC005956 | CA914597 | CS005558 | 1229 |
| AC005957 | CA914603 | CS005559 | 2410 |
| AC005958 | CA914613 | CS005560 | 4556 |
| AC005959 | CA914631 | CS005561 | 6015 |
| AC005998 | CA914619 | CS007257 | 5742 |
| AC005999 | CA914629 | CS007258 | 5955 |
| AC006000 | CA914635 | CS007259 | 6348 |
| AC006001 | CA914639 | CS007260 | 6754 |
| AC006002 | CA914583 | CS007256 | 164 |
| AC006704 | CA008019 | CS006705 | 6754 |
| AC006705 | CA008021 | CS008020 | 6754 |
| AC006706 | CA008021 | CS008022 | 6754 |
| AC006707 | CA006711 | CS006714 | 6015 |
| AC006708 | CA006711 | CS006719 | 6015 |
| AC006709 | CA006711 | CS008023 | 6015 |
| AC006805 | CA006722 | CS006724 | 6348 |
| AC006806 | CA005668 | CS008137 | 6348 |
| AC006807 | CA006722 | CS008137 | 6348 |
| AC007863 | CA914631 | CS009303 | 6015 |
| AC007864 | CA914639 | CS009304 | 6754 |
| AC007865 | CA006711 | CS009305 | 6015 |
| AC007866 | CA008021 | CS009306 | 6754 |
| AC007867 | CA006711 | CS009307 | 6015 |
| AC007952 | CA005668 | CS009417 | 6348 |
| AC007953 | CA008021 | CS009418 | 6754 |
| AC007968 | CA914631 | CS009419 | 6015 |
| AC007969 | CA914639 | CS009420 | 6754 |
| AC910139 | CA911803 | CS913031 | 4685 |
| AC910140 | CA911821 | CS913032 | 5858 |
| AC910577 | CA913562 | CS913031 | 4685 |
| AC910578 | CA913563 | CS913031 | 4685 |
| AC910579 | CA913564 | CS913031 | 4685 |
| AC910580 | CA913565 | CS913031 | 4685 |
| AC910581 | CA913566 | CS913031 | 4685 |
| AC910582 | CA913567 | CS913031 | 4685 |
| AC910583 | CA913568 | CS913031 | 4685 |
| AC910584 | CA913569 | CS913031 | 4685 |
| AC910585 | CA913570 | CS913031 | 4685 |
| AC911226 | CA911821 | CS914492 | 5858 |
| AC911227 | CA911803 | CS914493 | 4685 |
| AC911290 | CA914577 | CS914576 | 30 |
| AC911291 | CA914579 | CS914578 | 151 |
| AC911292 | CA914581 | CS914580 | 156 |
| AC911293 | CA914583 | CS914582 | 164 |
| AC911294 | CA914585 | CS914584 | 172 |
| AC911295 | CA914587 | CS914586 | 173 |
| AC911296 | CA914589 | CS914588 | 529 |
| AC911297 | CA914591 | CS914590 | 620 |
| AC911298 | CA914593 | CS914592 | 625 |
| AC911299 | CA914595 | CS914594 | 868 |
| AC911300 | CA914597 | CS914596 | 1229 |
| AC911301 | CA914599 | CS914598 | 1249 |
| AC911302 | CA914601 | CS914600 | 1405 |
| AC911303 | CA914603 | CS914602 | 2410 |
| AC911304 | CA914605 | CS914604 | 3220 |
| AC911305 | CA914607 | CS914606 | 3798 |
| AC911306 | CA914609 | CS914608 | 3810 |
| AC911307 | CA914611 | CS914610 | 4064 |
| AC911308 | CA914613 | CS914612 | 4556 |
| AC911309 | CA914615 | CS914614 | 4961 |
| AC911310 | CA914617 | CS914616 | 5144 |
| AC911311 | CA914619 | CS914618 | 5742 |
| AC911312 | CA914621 | CS914620 | 5798 |
| AC911313 | CA914623 | CS914622 | 5815 |
| AC911314 | CA914625 | CS914624 | 5869 |
| AC911315 | CA914627 | CS914626 | 5905 |
| AC911316 | CA914629 | CS914628 | 5955 |
| AC911317 | CA914631 | CS914630 | 6015 |
| AC911318 | CA914633 | CS914632 | 6241 |
| AC911319 | CA914635 | CS914634 | 6348 |
| AC911320 | CA914637 | CS914636 | 6604 |
| AC911321 | CA914639 | CS914638 | 6754 |
| AC911599 | CA914972 | CS914971 | 150 |
| AC911600 | CA914974 | CS914973 | 163 |
| AC911601 | CA914976 | CS914975 | 402 |
| AC911602 | CA914978 | CS914977 | 403 |
| AC911603 | CA914980 | CS914979 | 404 |
| AC911758 | CA915153 | CS914582 | 164 |
| AC911759 | CA915154 | CS914582 | 164 |
| AC911760 | CA915155 | CS914582 | 164 |
| AC911761 | CA915156 | CS914582 | 164 |
| AC911762 | CA915154 | CS915157 | 164 |
| AC911763 | CA915154 | CS915158 | 164 |
| AC911764 | CA915154 | CS915159 | 164 |
| AC911765 | CA915160 | CS914582 | 164 |
| AC911766 | CA915161 | CS914582 | 164 |
| AC911767 | CA915162 | CS914582 | 164 |
| AC912419 | CA915860 | CS914602 | 2410 |
| AC912420 | CA915861 | CS914602 | 2410 |
| AC912421 | CA915862 | CS914602 | 2410 |
| AC912422 | CA915863 | CS914602 | 2410 |
| AC912423 | CA915864 | CS914602 | 2410 |
| AC912424 | CA915862 | CS915865 | 2410 |
| AC912425 | CA915862 | CS915866 | 2410 |
| AC912426 | CA915862 | CS915867 | 2410 |
| AC912427 | CA915868 | CS914602 | 2410 |
| AC912428 | CA915869 | CS914602 | 2410 |
| AC912429 | CA915870 | CS914602 | 2410 |
| AC912430 | CA915871 | CS914602 | 2410 |
| AC912431 | CA915872 | CS914596 | 1229 |
| AC912432 | CA915873 | CS914596 | 1229 |
| AC912433 | CA915874 | CS914596 | 1229 |
| AC912434 | CA915875 | CS914596 | 1229 |
| AC912435 | CA915876 | CS914596 | 1229 |
| AC912436 | CA915874 | CS915877 | 1229 |
| AC912437 | CA915874 | CS915878 | 1229 |
| AC912438 | CA915874 | CS915879 | 1229 |
| AC912439 | CA915880 | CS914596 | 1229 |
| AC912440 | CA915881 | CS914596 | 1229 |
| AC912441 | CA915882 | CS914596 | 1229 |
| AC912442 | CA915883 | CS914596 | 1229 |
| AC912443 | CA915884 | CS914612 | 4556 |
| AC912444 | CA915885 | CS914612 | 4556 |
| AC912445 | CA915886 | CS914612 | 4556 |
| AC912446 | CA915887 | CS914612 | 4556 |
| AC912447 | CA915888 | CS914612 | 4556 |
| AC912448 | CA915886 | CS915889 | 4556 |
| AC912449 | CA915886 | CS915890 | 4556 |
| AC912450 | CA915886 | CS915891 | 4556 |
| AC912451 | CA915892 | CS914612 | 4556 |
| AC912452 | CA915893 | CS914612 | 4556 |
| AC912453 | CA915894 | CS914612 | 4556 |
| AC912454 | CA915895 | CS914612 | 4556 |

TABLE 9
Conjugate ID Numbers With Chemically Modified Antisense and Sense Strands
(including Linkers and Conjugates)

| | | SEQ | | SEQ |
|---|---|---|---|---|
| ACID | Sense Strand (Fully Modified with Conjugated | ID | | ID |
| Number | antigen binding protein) (5′ -> 3′) | NO: | Antisense Strand (5′ -> 3′) | NO: |
| AC004413 | LP293-(NH- | 857 | cPrpusAfscaacGfagacUfgAfaUfugccsu | 452 |
| | C6)s(invAb)saggcaauuCfAfGfucucguuguas(invAb) | | | |
| AC004496 | Fab0016-L20-(NH- | 786 | cPrpusUfsgsUfgUfaugauGfuAfaCfgUfcasg | 503 |
| | C6)s(invAb)scugacguuAfCfAfucauacacaas(invAb) | | | |
| AC004497 | Fab0016-L20-(NH- | 787 | cPrpusCfsasUfgAfucgauGfuAfgUfuCfaasg | 506 |
| | C6)s(invAb)scuugaacuAfCfAfucgaucaugas(invAb) | | | |
| AC004498 | Fab0016-L20-(NH- | 788 | cPrpusAfscsAfaAfgccaaUfaAfaCfaCfcusg | 511 |
| | C6)s(invAb)scagguguuUfAfUfuggcuuuguas(invAb) | | | |
| AC004499 | Fab0016-L20-(NH- | 789 | cPrpusAfscsAfaCfgagacUfgAfaUfuGfccsu | 520 |
| | C6)s(invAb)saggcaauuCfAfGfucucguuguas(invAb) | | | |
| AC004563 | LP293-(NH- | 861 | cPrpasGfsaucaUfucaaCfuUfgCfuccasg | 453 |
| | C6)s(invAb)scuggagcaAfGfUfugaaugaucus(invAb) | | | |
| AC004564 | LP293-(NH- | 859 | cPrpusAfscuaaGfugagUfcUfuGfcaugsg | 454 |
| | C6)s(invAb)sccaugcaaGfAfCfucacuuaguas(invAb) | | | |
| AC004575 | LP293-(NH- | 861 | cPrpasGfsaucaUfucaaCfuUfgCfuccassg | 455 |
| | C6)s(invAb)scuggagcaAfGfUfugaaugaucus(invAb) | | | |
| AC004576 | LP293-(NH- | 859 | cPrpusAfscuaaGfugagUfcUfuGfcaugssg | 456 |
| | C6)s(invAb)sccaugcaaGfAfCfucacuuaguas(invAb) | | | |
| AC004577 | LP293-(NH- | 857 | cPrpusAfscaacGfagacUfgAfaUfugccssu | 457 |
| | C6)s(invAb)saggcaauuCfAfGfucucguuguas(invAb) | | | |
| AC005385 | LP293-(NH- | 790 | cPrpusGfsasCfuCfgaaagCfcUfuCfaUfcasg | 460 |
| | C6)s(invAb)scugaugaaGfGfCfuuucgagucas(invAb) | | | |
| AC005500 | LP293-(NH- | 861 | cPrpasGfsauCfauucaaCfuUfgCfuccassg | 461 |
| | C6)s(invAb)scuggagcaAfGfUfugaaugaucus(invAb) | | | |
| AC005501 | LP293-(NH- | 791 | cPrpusGfsaucaUfucaaCfuUfgCfuccassg | 462 |
| | C6)s(invAb)scuggagcaAfGfUfugaaugaucas(invAb) | | | |
| AC005502 | LP293-(NH- | 861 | cPrpisGfsaucaUfucaaCfuUfgCfuccassg | 463 |
| | C6)s(invAb)scuggagcaAfGfUfugaaugaucus(invAb) | | | |
| AC005503 | LP293-(NH- | 792 | cPrpasGfsaucaUfucaaCfuUfgCfuccassg | 455 |
| | C6)s(invAb)scuggagcaAfgUfugaaugaucus(invAb) | | | |
| AC005504 | LP293-(NH- | 793 | cPrpasGfsaucaUfucaaCfuUfgCfuccassg | 455 |
| | C6)s(invAb)scuggagcaAfgUfUfgaaugaucus(invAb) | | | |
| AC005505 | LP293-(NH- | 861 | cPrpasGfsaucaUUNAucaaCfuUfgCfuccassg | 464 |
| | C6)s(invAb)scuggagcaAfGfUfugaaugaucus(invAb) | | | |
| AC005506 | LP293-(NH- | 861 | cPrpasGfsaucAUNAUfucaaCfuUfgCfuccassg | 465 |
| | C6)s(invAb)scuggagcaAfGfUfugaaugaucus(invAb) | | | |
| AC005507 | LP293-(NH- | 795 | cPrpasGfsaucaUfucaaCfuUfgCfuccassg | 455 |
| | C6)s(invAb)scuggagcaAfGfUfugaaugauuus(invAb) | | | |
| AC005508 | LP293-(NH- | 86 | cPrpasGfsaucaUfucaaUfuUfgCfuccassg | 466 |
| | C6)s(invAb)scuggagcaAfGfUfugaaugaucus(invAb) | | | |
| AC005509 | LP293-(NH- | 86 | cPrpasGfsaucaUfucaaCfuUfgUfuccassg | 467 |
| | C6)s(invAb)scuggagcaAfGfUfugaaugaucus(invAb) | | | |
| AC005510 | LP293-(NH- | 794 | cPrpasGfsaucaUfucaaCfuUfgCfuccassg | 455 |
| | C6)s(invAb)scuggagcaAfgUfuGfaaugaucus(invAb) | | | |
| AC005511 | LP293-(NH- | 857 | cPrpusAfscaAfcgagacUfgAfaUfugccssu | 468 |
| | C6)s(invAb)saggcaauuCfAfGfucucguuguas(invAb) | | | |
| AC005512 | LP293-(NH- | 796 | cPrpusAfscaacGfagacUfgAfaUfugccssu | 457 |
| | C6)s(invAb)saggcaauuCfaGfucucguuguas(invAb) | | | |
| AC005513 | LP293-(NH- | 797 | cPrpusAfscaacGfagacUfgAfaUfugccssu | 457 |
| | C6)s(invAb)saggcaauuCfaGfUfcucguuguas(invAb) | | | |
| AC005514 | LP293-(NH- | 798 | cPrpusAfscaacGfagacUfgAfaUfugccssu | 457 |
| | C6)s(invAb)saggcaauuCfaGfuCfucguuguas(invAb) | | | |
| AC005515 | LP293-(NH- | 857 | cPrpusAfscaacGUNAagacUfgAfaUfugccssu | 469 |
| | C6)s(invAb)saggcaauuCfAfGfucucguuguas(invAb) | | | |
| AC005516 | LP293-(NH- | 857 | cPrpusAfscaaCUNAGfagacUfgAfaUfugccssu | 470 |
| | C6)s(invAb)saggcaauuCfAfGfucucguuguas(invAb) | | | |
| AC005517 | LP293-(NH- | 799 | cPrpusAfscaacGfagacUfgAfaUfugccssu | 457 |
| | C6)s(invAb)saggcaauuCfAfGfucuuguuguas(invAb) | | | |
| AC005518 | LP293-(NH- | 857 | cPrpusAfscaauGfagacUfgAfaUfugccssu | 471 |
| | C6)s(invAb)saggcaauuCfAfGfucucguuguas(invAb) | | | |
| AC005519 | LP293-(NH- | 800 | cPrpusAfscaacGfagacUfgAfaUfugccssu | 457 |
| | C6)s(invAb)sagguaauuCfAfGfucucguuguas(invAb) | | | |
| AC005520 | LP293-(NH- | 801 | cPrpusAfscaacGfagacUfgAfaUfugccssu | 457 |
| | C6)s(invAb)saggcaauuCfAfGfuuucguuguas(invAb) | | | |
| AC005521 | LP293-(NH- | 857 | cPrpussAfcaacGfagacUfgAfaUfugccssu | 472 |
| | C6)s(invAb)saggcaauuCfAfGfucucguuguas(invAb) | | | |
| AC005522 | LP293-(NH- | 859 | cPrpusAfscuAfagugagUfcUfuGfcaugssg | 473 |
| | C6)s(invAb)sccaugcaaGfAfCfucacuuaguas(invAb) | | | |
| AC005523 | LP293-(NH- | 802 | cPrpusAfscuaaGfugagUfcUfuGfcaugssg | 456 |
| | C6)s(invAb)sccaugcaaGfaCfucacuuaguas(invAb) | | | |
| AC005524 | LP293-(NH- | 803 | cPrpusAfscuaaGfugagUfcUfuGfcaugssg | 456 |
| | C6)s(invAb)sccaugcaaGfaCfUfcacuuaguas(invAb) | | | |
| AC005525 | LP293-(NH- | 804 | cPrpusAfscuaaGfugagUfcUfuGfcaugssg | 456 |
| | C6)s(invAb)sccaugcaaGfaCfuCfacuuaguas(invAb) | | | |
| AC005526 | LP293-(NH- | 859 | cPrpusAfscuaaGUNAugagUfcUfuGfcaugssg | 474 |
| | C6)s(invAb)sccaugcaaGfAfCfucacuuaguas(invAb) | | | |
| AC005527 | LP293-(NH- | 859 | cPrpusAfscuaAUNAGfugagUfcUfuGfcaugssg | 475 |
| | C6)s(invAb)sccaugcaaGfAfCfucacuuaguas(invAb) | | | |
| AC005528 | LP293-(NH- | 805 | cPrpusAfscuaaGfugagUfuUfuGfcaugssg | 476 |
| | C6)s(invAb)sccauguaaGfAfCfucacuuaguas(invAb) | | | |
| AC005529 | LP293-(NH- | 806 | cPrpusAfscuaaGfugagUfcUfuGfcaugssg | 456 |
| | C6)s(invAb)sccaugcaaGfAfCfucauuuaguas(invAb) | | | |
| AC005530 | LP293-(NH- | 859 | cPrpusAfscuaaGfugagUfcUfuGfuaugssg | 477 |
| | C6)s(invAb)sccaugcaaGfAfCfucacuuaguas(invAb) | | | |
| AC005531 | LP293-(NH- | 807 | cPrpusAfscuaaGfugagUfcUfuGfcaugssg | 456 |
| | C6)s(invAb)sccaugcaaGfAfCfuuacuuaguas(invAb) | | | |
| AC005532 | LP293-(NH- | 859 | cPrpussAfcuaaGfugagUfcUfuGfcaugssg | 478 |
| | C6)s(invAb)sccaugcaaGfAfCfucacuuaguas(invAb) | | | |
| AC005533 | LP293-(NH- | 861 | cPrpasGfsauuaUfucaaCfuUfgCfuccassg | 479 |
| | C6)s(invAb)scuggagcaAfGfUfugaaugaucus(invAb) | | | |
| AC005956 | Fab0016-L20-(NH- | 786 | cPrpusUfsgsUfgUfaugauGfuAfaCfgUfcasg | 503 |
| | C6)s(invAb)scugacguuAfCfAfucauacacaas(invAb) | | | |
| AC005957 | Fab0016-L20-(NH- | 787 | cPrpusCfsasUfgAfucgauGfuAfgUfuCfaasg | 506 |
| | C6)s(invAb)scuugaacuAfCfAfucgaucaugas(invAb) | | | |
| AC005958 | Fab0016-L20-(NH- | 788 | cPrpusAfscsAfaAfgccaaUfaAfaCfaCfcusg | 511 |
| | C6)s(invAb)scagguguuUfAfUfuggcuuuguas(invAb) | | | |
| AC005959 | Fab0016-L20-(NH- | 789 | cPrpusAfscsAfaCfgagacUfgAfaUfuGfccsu | 520 |
| | C6)s(invAb)saggcaauuCfAfGfucucguuguas(invAb) | | | |
| AC005998 | Fab0016-L20-(NH- | 809 | cPrpusAfscsUfaAfguaacUfuUfgUfgCfugsg | 514 |
| | C6)s(invAb)sccagcacaAfAfGfuuacuuaguas(invAb) | | | |
| AC005999 | Fab0016-L20-(NH- | 810 | cPrpusAfscsUfgAfugaagUfcCfuGfuAfcusg | 519 |
| | C6)s(invAb)scaguacagGfAfCfuucaucaguas(invAb) | | | |
| AC006000 | Fab0016-L20-(NH- | 811 | cPrpusAfscsUfaAfgugagUfcUfuGfcAfugsg | 522 |
| | C6)s(invAb)sccaugcaaGfAfCfucacuuaguas(invAb) | | | |
| AC006001 | Fab0016-L20-(NH- | 812 | cPrpasGfsasUfcAfuucaaCfuUfgCfuCfcasg | 524 |
| | C6)s(invAb)scuggagcaAfGfUfugaaugaucus(invAb) | | | |
| AC006002 | Fab0016-L20-(NH- | 808 | cPrpusGfsasCfuCfgaaggCfcUfuCfaUfcasg | 496 |
| | C6)s(invAb)scugaugaaGfGfCfcuucgagucas(invAb) | | | |
| AC006704 | LP293-(NH- | 794 | cPrpisGfsauCfauucaaCfuUfgCfuccassg | 480 |
| | C6)s(invAb)scuggagcaAfgUfuGfaaugaucus(invAb) | | | |
| AC006705 | LP293-(NH- | 813 | cPrpusGfsauCfauucaaCfuUfgCfuccassg | 481 |
| | C6)s(invAb)scuggagcaAfgUfuGfaaugaucas(invAb) | | | |
| AC006706 | LP293-(NH- | 814 | cPrpusGfsauCfauucaaCfuUfgCfuccassg | 481 |
| | C6)s(invAb)scuggagcaAfgUfuGfaaugauuas(invAb) | | | |
| AC006707 | LP293-(NH- | 798 | cPrpusAfscaAfcgagacUfgAfaUfugccssu | 468 |
| | C6)s(invAb)saggcaauuCfaGfuCfucguuguas(invAb) | | | |
| AC006708 | LP293-(NH- | 800 | cPrpusAfscaAfcgagacUfgAfaUfugccssu | 468 |
| | C6)s(invAb)sagguaauuCfAfGfucucguuguas(invAb) | | | |
| AC006709 | LP293-(NH- | 815 | cPrpusAfscaAfcgagacUfgAfaUfugccssu | 468 |
| | C6)s(invAb)sagguaauuCfaGfuCfucguuguas(invAb) | | | |
| AC006805 | LP293-(NH- | 803 | cPrpusAfscuAfagugagUfcUfuGfcaugssg | 473 |
| | C6)s(invAb)sccaugcaaGfaCfUfcacuuaguas(invAb) | | | |
| AC006806 | LP293-(NH- | 816 | cPrpusAfscuaaGfugagUfcUfuGfcaugssg | 456 |
| | C6)s(invAb)sccaugcaaGfaCfUfuacuuaguas(invAb) | | | |
| AC006807 | LP293-(NH- | 816 | cPrpusAfscuAfagugagUfcUfuGfcaugssg | 473 |
| | C6)s(invAb)sccaugcaaGfaCfUfuacuuaguas(invAb) | | | |
| AC007863 | Fab0070-L20-(NH- | 817 | cPrpusAfscsAfaCfgagacUfgAfaUfuGfccsu | 520 |
| | C6)s(invAb)saggcaauuCfAfGfucucguuguas(invAb) | | | |
| AC007864 | Fab0070-L20-(NH- | 818 | cPrpasGfsasUfcAfuucaaCfuUfgCfuCfcasg | 524 |
| | C6)s(invAb)scuggagcaAfGfUfugaaugaucus(invAb) | | | |
| AC007865 | Fab0070-L20-(NH- | 819 | cPrpusAfscaAfcgagacUfgAfaUfugccssu | 468 |
| | C6)s(invAb)saggcaauuCfaGfuCfucguuguas(invAb) | | | |
| AC007866 | Fab0070-L20-(NH- | 820 | cPrpusGfsauCfauucaaCfuUfgCfuccassg | 481 |
| | C6)s(invAb)scuggagcaAfgUfuGfaaugaucas(invAb) | | | |
| AC007867 | Fab0070-L1026- | 821 | cPrpusAfscaAfcgagacUfgAfaUfugccssu | 468 |
| | C6s(invAb)saggcaauuCfaGfuCfucguuguas(invAb) | | | |
| AC007952 | Fab0070-L1026- | 822 | cPrpusAfscuaaGfugagUfcUfuGfcaugssg | 456 |
| | C6s(invAb)sccaugcaaGfaCfUfcacuuaguas(invAb) | | | |
| AC007953 | Fab0070-L1026- | 823 | cPrpusGfsauCfauucaaCfuUfgCfuccassg | 481 |
| | C6s(invAb)scuggagcaAfgUfuGfaaugaucas(invAb) | | | |
| AC007968 | Fab0070-L1026- | 824 | cPrpusAfscsAfaCfgagacUfgAfaUfuGfccsu | 520 |
| | C6s(invAb)saggcaauuCfAfGfucucguuguas(invAb) | | | |
| AC007969 | Fab0070-L1026- | 825 | cPrpasGfsasUfcAfuucaaCfuUfgCfuCfcasg | 524 |
| | C6s(invAb)scuggagcaAfGfUfugaaugaucus(invAb) | | | |
| AC910139 | LP183s(invAb)saaacagauCfAfUfuggaauuccus(invAb) | 826 | cPrpasGfsgsAfaUfuccaaUfgAfuCfuGfuusu | 482 |
| AC910140 | LP183s(invAb)sugugauuaUfGfUfcugucagaaas(invAb) | 827 | cPrpusUfsusCfuGfacagaCfaUfaAfuCfacsa | 483 |
| AC910577 | LP183s(invAb)saaacagauCfAfUfuggaauuccus(invAb) | 826 | cPrpasGfsgsAfauuccaaUfgAfuCfuguusu | 484 |
| AC910578 | LP183s(invAb)saaacagauCfAfUfuggaauuccus(invAb) | 826 | cPrpasGfsgsaAfuuccaaUfgAfuCfuguusu | 485 |
| AC910579 | LP183s(invAb)saaacagauCfAfUfuggaauuccus(invAb) | 826 | cPrpasGfsgsaauUfccaaUfgAfuCfuguusu | 486 |
| AC910580 | LP183s(invAb)saaacagauCfAfUfuggaauuccus(invAb) | 826 | cPrpasGfsgsaauucCfaaUfgAfuCfuguusu | 487 |
| AC910581 | LP183s(invAb)saaacagauCfAfUfuggaauuccus(invAb) | 826 | cPrpasGfsgsAfaUfuccaaugAfuCfuguusu | 488 |
| AC910582 | LP183s(invAb)saaacagauCfAfUfuggaauuccus(invAb) | 826 | cPrpasGfsgsaauuccaaugAfuCfuguusu | 489 |
| AC910583 | LP183s(invAb)saaacagauCfAfUfuggaauuccus(invAb) | 826 | cPrpasGfsgsaauuccaaUfgAfucuguusu | 490 |
| AC910584 | LP183s(invAb)saaacagauCfAfUfuggaauuccus(invAb) | 826 | cPrpasGfsgsaAfuuccaaugAfucuguusu | 491 |
| AC910585 | LP183s(invAb)saaacagauCfAfUfuggaauuccus(invAb) | 826 | cPrpasGfsgsAfauUUNAccaaUfgAfuCfuguusu | 492 |
| AC911226 | LP293-(NH- | 828 | cPrpusUfsusCfuGfacagaCfaUfaAfuCfacsa | 483 |
| | C6)s(invAb)sugugauuaUfGfUfcugucagaaas(invAb) | | | |
| AC911227 | LP293-(NH- | 829 | cPrpasGfsgsAfaUfuccaaUfgAfuCfuGfuusu | 482 |
| | C6)s(invAb)saaacagauCfAfUfuggaauuccus(invAb) | | | |
| AC911290 | LP293-(NH- | 830 | cPrpusGfsusAfaAfagcagAfaCfcUfgAfgcsg | 493 |
| | C6)s(invAb)scgcucaggUfUfCfugcuuuuacas(invAb) | | | |
| AC911291 | LP293-(NH- | 831 | cPrpusUfscsAfuCfagcuuUfuCfcAfgGfgusc | 494 |
| | C6)s(invAb)sgacccuggAfAfAfagcugaugaas(invAb) | | | |
| AC911292 | LP293-(NH- | 832 | cPrpasGfsgsCfcUfucaucAfgCfuUfuUfccsa | 495 |
| | C6)s(invAb)suggaaaagCfUfGfaugaaggccus(invAb) | | | |
| AC911293 | LP293-(NH- | 833 | cPrpusGfsasCfuCfgaaggCfcUfuCfaUfcasg | 496 |
| | C6)s(invAb)scugaugaaGfGfCfcuucgagucas(invAb) | | | |
| AC911294 | LP293-(NH- | 834 | cPrpusAfscsUfuGfagggaCfuCfgAfaGfgcsc | 497 |
| | C6)s(invAb)sggccuucgAfGfUfcccucaaguas(invAb) | | | |
| AC911295 | LP293-(NH- | 835 | cPrpusGfsasCfuUfgagggAfcUfcGfaAfggsc | 498 |
| | C6)s(invAb)sgccuucgaGfUfCfccucaagucas(invAb) | | | |
| AC911296 | LP293-(NH- | 836 | cPrpasGfsasAfaAfaguucCfaUfaGfcGfausg | 499 |
| | C6)s(invAb)scaucgcuaUfGfGfaacuuuuucus(invAb) | | | |
| AC911297 | LP293-(NH- | 837 | cPrpasAfsgsAfuUfagaauCfcAfuCfaAfagsc | 500 |
| | C6)s(invAb)sgcuuugauGfGfAfuucuaaucuus(invAb) | | | |
| AC911298 | LP293-(NH- | 838 | cPrpusUfsusGfgAfagauuAfgAfaUfcCfausc | 501 |
| | C6)s(invAb)sgauggauuCfUfAfaucuuccaaas(invAb) | | | |
| AC911299 | LP293-(NH- | 839 | cPrpusUfsgsUfcAfuuugcAfaAfaUfuGfccsa | 502 |
| | C6)s(invAb)suggcaauuUfUfGfcaaaugacaas(invAb) | | | |
| AC911300 | LP293-(NH- | 840 | cPrpusUfsgsUfgUfaugauGfuAfaCfgUfcasg | 503 |
| | C6)s(invAb)scugacguuAfCfAfucauacacaas(invAb) | | | |
| AC911301 | LP293-(NH- | 841 | cPrpasCfsasAfcAfuugugGfuCfuUfgGfugsc | 504 |
| | C6)s(invAb)sgcaccaagAfCfCfacaauguugus(invAb) | | | |
| AC911302 | LP293-(NH- | 842 | cPrpusCfsusAfuAfaguucCfaCfaAfuAfcusc | 505 |
| | C6)s(invAb)sgaguauugUfGfGfaacuuauagas(invAb) | | | |
| AC911303 | LP293-(NH- | 843 | cPrpusCfsasUfgAfucgauGfuAfgUfuCfaasg | 506 |
| | C6)s(invAb)scuugaacuAfCfAfucgaucaugas(invAb) | | | |
| AC911304 | LP293-(NH- | 844 | cPrpusAfscsAfaAfgcuucAfcAfgCfaUfccsa | 507 |
| | C6)s(invAb)suggaugcuGfUfGfaagcuuuguas(invAb) | | | |
| AC911305 | LP293-(NH- | 845 | cPrpusAfscsUfuGfuuguaAfcAfgGfaCfcusg | 508 |
| | C6)s(invAb)scagguccuGfUfUfacaacaaguas(invAb) | | | |
| AC911306 | LP293-(NH- | 846 | cPrpusUfsgsAfuGfaggauUfuAfcUfuGfuusg | 509 |
| | C6)s(invAb)scaacaaguAfAfAfuccucaucaas(invAb) | | | |
| AC911307 | LP293-(NH- | 847 | cPrpasCfsasAfaCfaguugCfcAfuCfaUfugsg | 510 |
| | C6)s(invAb)sccaaugauGfGfCfaacuguuugus(invAb) | | | |
| AC911308 | LP293-(NH- | 848 | cPrpusAfscsAfaAfgccaaUfaAfaCfaCfcusg | 511 |
| | C6)s(invAb)scagguguuUfAfUfuggcuuuguas(invAb) | | | |
| AC911309 | LP293-(NH- | 849 | cPrpusAfsusCfuGfucgagAfcAfgUfcGfcusu | 512 |
| | C6)s(invAb)saagcgacuGfUfCfucgacagauas(invAb) | | | |
| AC911310 | LP293-(NH- | 850 | cPrpusAfsgsUfuGfaacagUfgCfuCfaCfggsa | 513 |
| | C6)s(invAb)succgugagCfAfCfuguucaacuas(invAb) | | | |
| AC911311 | LP293-(NH- | 851 | cPrpusAfscsUfaAfguaacUfuUfgUfgCfugsg | 514 |
| | C6)s(invAb)sccagcacaAfAfGfuuacuuaguas(invAb) | | | |
| AC911312 | LP293-(NH- | 852 | cPrpusCfsasCfaUfuccaaGfuUfuGfgCfugsc | 515 |
| | C6)s(invAb)sgcagccaaAfCfUfuggaaugugas(invAb) | | | |
| AC911313 | LP293-(NH- | 853 | cPrpusGfsusAfcUfauuucUfcUfaUfuGfcasc | 516 |
| | C6)s(invAb)sgugcaauaGfAfGfaaauaguacas(invAb) | | | |
| AC911314 | LP293-(NH- | 854 | cPrpusAfsgsUfcAfuggagGfuUfcUfgAfcasg | 517 |
| | C6)s(invAb)scugucagaAfCfCfuccaugacuas(invAb) | | | |
| AC911315 | LP293-(NH- | 855 | cPrpusGfsasAfuGfugauuUfaCfaAfuGfagsc | 518 |
| | C6)s(invAb)sgcucauugUfAfAfaucacauucas(invAb) | | | |
| AC911316 | LP293-(NH- | 856 | cPrpusAfscsUfgAfugaagUfcCfuGfuAfcusg | 519 |
| | C6)s(invAb)scaguacagGfAfCfuucaucaguas(invAb) | | | |
| AC911317 | LP293-(NH- | 857 | cPrpusAfscsAfaCfgagacUfgAfaUfuGfccsu | 520 |
| | C6)s(invAb)saggcaauuCfAfGfucucguuguas(invAb) | | | |
| AC911318 | LP293-(NH- | 858 | cPrpusUfsgsAfgUfucuucCfaUfuGfgCfaasc | 521 |
| | C6)s(invAb)sguugccaaUfGfGfaagaacucaas(invAb) | | | |
| AC911319 | LP293-(NH- | 859 | cPrpusAfscsUfaAfgugagUfcUfuGfcAfugsg | 522 |
| | C6)s(invAb)sccaugcaaGfAfCfucacuuaguas(invAb) | | | |
| AC911320 | LP293-(NH- | 860 | cPrpusCfsasGfaAfauuucAfcUfcAfuCfccsu | 523 |
| | C6)s(invAb)sagggaugaGfUfGfaaauuucugas(invAb) | | | |
| AC911321 | LP293-(NH- | 861 | cPrpasGfsasUfcAfuucaaCfuUfgCfuCfcasg | 524 |
| | C6)s(invAb)scuggagcaAfGfUfugaaugaucus(invAb) | | | |
| AC911599 | LP293-(NH- | 862 | cPrpusCfsasUfcAfgcuuuUfcCfaGfgGfucsg | 525 |
| | C6)s(invAb)scgacccugGfAfAfaagcuiaugas(invAb) | | | |
| AC911600 | LP293-(NH- | 863 | cPrpusAfscsUfcGfaaggcCfuUfcAfuCfagsc | 526 |
| | C6)s(invAb)sgcugaugaAfGfGfccuuciaguas(invAb) | | | |
| AC911601 | LP293-(NH- | 864 | cPrpusUfsusUfcUfuugguCfgGfuGfcAfgcsg | 527 |
| | C6)s(invAb)scgcugcacCfGfAfccaaagaaaas(invAb) | | | |
| AC911602 | LP293-(NH- | 865 | cPrpusCfsusUfuCfuuuggUfcGfgUfgCfagsc | 528 |
| | C6)s(invAb)sgcugcaccGfAfCfcaaagaaagas(invAb) | | | |
| AC911603 | LP293-(NH- | 866 | cPrpusUfscsUfuUfcuuugGfuCfgGfuGfcasg | 529 |
| | C6)s(invAb)scugcaccgAfCfCfaaagaaagaas(invAb) | | | |
| AC911758 | LP293-(NH- | 833 | cPrpusGfsascUfcgaaggCfcUfuCfaucasg | 530 |
| | C6)s(invAb)scugaugaaGfGfCfcuucgagucas(invAb) | | | |
| AC911759 | LP293-(NH- | 833 | cPrpusGfsascucGfaaggCfcUfuCfaucasg | 531 |
| | C6)s(invAb)scugaugaaGfGfCfcuucgagucas(invAb) | | | |
| AC911760 | LP293-(NH- | 833 | cPrpusGfsascucgaAfggCfcUfuCfaucasg | 532 |
| | C6)s(invAb)scugaugaaGfGfCfcuucgagucas(invAb) | | | |
| AC911761 | LP293-(NH- | 833 | cPrpusGfsacucGfaaggCfcUfuCfaucasg | 533 |
| | C6)s(invAb)scugaugaaGfGfCfcuucgagucas(invAb) | | | |
| AC911762 | LP293-(NH- | 867 | cPrpusGfsascucGfaaggCfcUfuCfaucasg | 531 |
| | C6)s(invAb)scugaugaaGfgCfcuucgagucas(invAb) | | | |
| AC911763 | LP293-(NH- | 868 | cPrpusGfsascucGfaaggCfcUfuCfaucasg | 531 |
| | C6)s(invAb)scugaugAfaGfgCfcuucgagucas(invAb) | | | |
| AC911764 | LP293-(NH- | 869 | cPrpusGfsascucGfaaggCfcUfuCfaucasg | 531 |
| | C6)s(invAb)scugaugaaGfgCfCfuucgagucas(invAb) | | | |
| AC911765 | LP293-(NH- | 833 | cPrpusGfsascucGfaaggccUfucaucasg | 534 |
| | C6)s(invAb)scugaugaaGfGfCfcuucgagucas(invAb) | | | |
| AC911766 | LP293-(NH- | 833 | cPrpusGfsascucgaAfggccUfucaucasg | 535 |
| | C6)s(invAb)scugaugaaGfGfCfcuucgagucas(invAb) | | | |
| AC911767 | LP293-(NH- | 833 | cPrpusGfsascucGfaaggccUfucaucAfsg | 536 |
| | C6)s(invAb)scugaugaaGfGfCfcuucgagucas(invAb) | | | |
| AC912419 | LP293-(NH- | 843 | cPrpusCfsasuGfaucgauGfuAfgUfucaasg | 537 |
| | C6)s(invAb)scuugaacuAfCfAfucgaucaugas(invAb) | | | |
| AC912420 | LP293-(NH- | 843 | cPrpusCfsasugaUfcgauGfuAfgUfucaasg | 538 |
| | C6)s(invAb)scuugaacuAfCfAfucgaucaugas(invAb) | | | |
| AC912421 | LP293-(NH- | 843 | cPrpusCfsaugaUfcgauGfuAfgUfucaasg | 539 |
| | C6)s(invAb)scuugaacuAfCfAfucgaucaugas(invAb) | | | |
| AC912422 | LP293-(NH- | 843 | cPrpusCfsaugaUfcgauGfuAfgUfucaassg | 540 |
| | C6)s(invAb)scuugaacuAfCfAfucgaucaugas(invAb) | | | |
| AC912423 | LP293-(NH- | 843 | cPrpussCfsaugaUfcgauGfuAfgUfucaassg | 541 |
| | C6)s(invAb)scuugaacuAfCfAfucgaucaugas(invAb) | | | |
| AC912424 | LP293-(NH- | 870 | cPrpusCfsaugaUfcgauGfuAfgUfucaasg | 539 |
| | C6)s(invAb)scuugaacuAfcAfucgaucaugas(invAb) | | | |
| AC912425 | LP293-(NH- | 871 | cPrpusCfsaugaUfcgauGfuAfgUfucaasg | 539 |
| | C6)s(invAb)scuugaaCfuAfcAfucgaucaugas(invAb) | | | |
| AC912426 | LP293-(NH- | 872 | cPrpusCfsaugaUfcgauGfuAfgUfucaasg | 539 |
| | C6)s(invAb)scuugaacuAfcAfUfcgaucaugas(invAb) | | | |
| AC912427 | LP293-(NH- | 843 | cPrpusCfsaugaUfcgauguAfguucaAfsg | 542 |
| | C6)s(invAb)scuugaacuAfCfAfucgaucaugas(invAb) | | | |
| AC912428 | LP293-(NH- | 843 | cPrpusdCsaugadTcgaudGuAfgdUucaasg | 543 |
| | C6)s(invAb)scuugaacuAfCfAfucgaucaugas(invAb) | | | |
| AC912429 | LP293-(NH- | 843 | cPrpusCfsaugadTcgaudGuAfgdUucaasg | 544 |
| | C6)s(invAb)scuugaacuAfCfAfucgaucaugas(invAb) | | | |
| AC912430 | LP293-(NH- | 843 | cPrpusCfsaugadTcgauGfuAfgdUucaasg | 545 |
| | C6)s(invAb)scuugaacuAfCfAfucgaucaugas(invAb) | | | |
| AC912431 | LP293-(NH- | 840 | cPrpusUfsgsuguAfugauGfuAfaCfgucasg | 546 |
| | C6)s(invAb)scugacguuAfCfAfucauacacaas(invAb) | | | |
| AC912432 | LP293-(NH- | 840 | cPrpusUfsgsuGfuaugauGfuAfaCfgucasg | 547 |
| | C6)s(invAb)scugacguuAfCfAfucauacacaas(invAb) | | | |
| AC912433 | LP293-(NH- | 840 | cPrpusUfsguguAfugauGfuAfaCfgucasg | 548 |
| | C6)s(invAb)scugacguuAfCfAfucauacacaas(invAb) | | | |
| AC912434 | LP293-(NH- | 840 | cPrpusUfsguguAfugauGfuAfaCfgucassg | 549 |
| | C6)s(invAb)scugacguuAfCfAfucauacacaas(invAb) | | | |
| AC912435 | LP293-(NH- | 840 | cPrpussUfsguguAfugauGfuAfaCfgucassg | 550 |
| | C6)s(invAb)scugacguuAfCfAfucauacacaas(invAb) | | | |
| AC912436 | LP293-(NH- | 873 | cPrpusUfsguguAfugauGfuAfaCfgucasg | 548 |
| | C6)s(invAb)scugacguuAfcAfucauacacaas(invAb) | | | |
| AC912437 | LP293-(NH- | 874 | cPrpusUfsguguAfugauGfuAfaCfgucasg | 548 |
| | C6)s(invAb)scugacgUfuAfcAfucauacacaas(invAb) | | | |
| AC912438 | LP293-(NH- | 875 | cPrpusUfsguguAfugauGfuAfaCfgucasg | 548 |
| | C6)s(invAb)scugacguuAfcAfUfcauacacaas(invAb) | | | |
| AC912439 | LP293-(NH- | 840 | cPrpusUfsguguAfugauguAfacgucAfsg | 551 |
| | C6)s(invAb)scugacguuAfCfAfucauacacaas(invAb) | | | |
| AC912440 | LP293-(NH- | 840 | cPrpusdTsgugudAugaudGuAfadCgucasg | 552 |
| | C6)s(invAb)scugacguuAfCfAfucauacacaas(invAb) | | | |
| AC912441 | LP293-(NH- | 840 | cPrpusUfsgugudAugaudGuAfadCgucasg | 553 |
| | C6)s(invAb)scugacguuAfCfAfucauacacaas(invAb) | | | |
| AC912442 | LP293-(NH- | 840 | cPrpusUfsgugudAugauGfuAfadCgucasg | 554 |
| | C6)s(invAb)scugacguuAfCfAfucauacacaas(invAb) | | | |
| AC912443 | LP293-(NH- | 848 | cPrpusAfscsaAfagccaaUfaAfaCfaccusg | 555 |
| | C6)s(invAb)scagguguuUfAfUfuggcuuuguas(invAb) | | | |
| AC912444 | LP293-(NH- | 848 | cPrpusAfscsaaaGfccaaUfaAfaCfaccusg | 556 |
| | C6)s(invAb)scagguguuUfAfUfuggcuuuguas(invAb) | | | |
| AC912445 | LP293-(NH- | 848 | cPrpusAfscaaaGfccaaUfaAfaCfaccusg | 557 |
| | C6)s(invAb)scagguguuUfAfUfuggcuuuguas(invAb) | | | |
| AC912446 | LP293-(NH- | 848 | cPrpusAfscaaaGfccaaUfaAfaCfaccussg | 558 |
| | C6)s(invAb)scagguguuUfAfUfuggcuuuguas(invAb) | | | |
| AC912447 | LP293-(NH- | 848 | cPrpussAfscaaaGfccaaUfaAfaCfaccussg | 559 |
| | C6)s(invAb)scagguguuUfAfUfuggcuuuguas(invAb) | | | |
| AC912448 | LP293-(NH- | 876 | cPrpusAfscaaaGfccaaUfaAfaCfaccusg | 557 |
| | C6)s(invAb)scagguguuUfaUfuggcuuuguas(invAb) | | | |
| AC912449 | LP293-(NH- | 877 | cPrpusAfscaaaGfccaaUfaAfaCfaccusg | 557 |
| | C6)s(invAb)scaggugUfuUfaUfuggcuuuguas(invAb) | | | |
| AC912450 | LP293-(NH- | 878 | cPrpusAfscaaaGfccaaUfaAfaCfaccusg | 557 |
| | C6)s(invAb)scagguguuUfaUfUfggcuuuguas(invAb) | | | |
| AC912451 | LP293-(NH- | 848 | cPrpusAfscaaaGfccaauaAfacaccUfsg | 560 |
| | C6)s(invAb)scagguguuUfAfUfuggcuuuguas(invAb) | | | |
| AC912452 | LP293-(NH- | 848 | cPrpusdAscaaadGccaadTaAfadCaccusg | 561 |
| | C6)s(invAb)scagguguuUfAfUfuggcuuuguas(invAb) | | | |
| AC912453 | LP293-(NH- | 848 | cPrpusAfscaaadGccaadTaAfadCaccusg | 562 |
| | C6)s(invAb)scagguguuUfAfUfuggcuuuguas(invAb) | | | |
| AC912454 | LP293-(NH- | 848 | cPrpusAfscaaadGccaaUfaAfadCaccusg | 563 |
| | C6)s(invAb)scagguguuUfAfUfuggcuuuguas(invAb) | | | |

In some embodiments, an HTT RNAi agent is prepared or provided as a salt, mixed salt, or a free-acid. In some embodiments, an HTT RNAi agent is prepared or provided as a pharmaceutically acceptable salt. In some embodiments, an HTT RNAi agent is prepared or provided as a pharmaceutically acceptable sodium or potassium salt. The RNAi agents described herein, upon delivery to a cell expressing an HTT gene, inhibit or knockdown expression of one or more HTT genes in vivo and/or in vitro.

### Targeting Groups, Linking Groups, PK/PD Modulators and Delivery Vehicles

In some embodiments, an HTT RNAi agent contains or is conjugated to one or more non-nucleotide groups including, but not limited to, a targeting group, a linking group, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, an HTT RNAi agent contains a non-nucleotide group linked to the 3′ and/or 5′ end of the sense strand. In some embodiments, a non-nucleotide group is linked to the 5′ end of an HTT RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, and 9. A linker, with or without a targeting group, can be attached to the 5′ or 3′ end of any of the sense and/or antisense strands disclosed in Tables 2, 3, 4, 5, 6, and 9.

The HTT RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the HTT RNAi agents disclosed herein are synthesized having an NH₂-C₆ group at the 5′-terminus of the sense strand of the RNAi agent. The terminal amino group subsequently can be reacted to form a conjugate with, for example, a group that includes an antigen binding protein. In some embodiments, the HTT RNAi agents disclosed herein are synthesized having one or more alkyne groups at the 5′-terminus of the sense strand of the RNAi agent.

In some embodiments, targeting groups are linked to the HTT RNAi agents without the use of an additional linker. In some embodiments, the targeting group is designed having a linker readily present to facilitate the linkage to an HTT RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 10.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not to be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, an HTT RNAi agent is conjugated to a polyethylene glycol (PEG) moiety, or to a hydrophobic group having 12 or more carbon atoms, such as a cholesterol or palmitoyl group.

In some embodiments, an HTT RNAi agent is linked to one or more antigen binding proteins. Antigen binding proteins may enhance the bioavailability of the RNAi agent, the delivery of the RNAi agent to a cell of interest, or the facilitation of shuttling the RNAi agent across the blood brain barrier. In some embodiments, the antigen binding protein may be conjugated to a linker at the 3′ or 5′ end of a sense strand or an antisense strand of an RNAi agent described herein. In some embodiments, an antigen binding protein may be linked at both the 3′ or 5′ end of either the sense strand or the antisense strand of an RNAi agent described herein.

In some embodiments, an antigen binding protein may be conjugated to an HTT RNAi agent by reacting an HTT RNAi agent comprising an amine-comprising linker, for example, (NH2-C6) (see Table 10). In some embodiments, the amine-comprising linker may be located on the 5′ end of the sense strand or the antisense strand of an HTT RNAi agent. In some embodiments, the amine-comprising linker may be located on the 3′ end of the sense strand or the antisense strand of an RNAi agent.

Any of the HTT RNAi agent nucleotide sequences listed in Tables 2, 3, 4, 5, 6, and 9, whether modified or unmodified, can contain 3′ and/or 5′ targeting group(s), linking group(s), and/or antigen binding fragments. Any of the HTT RNAi agent duplexes listed in Tables 7, 8, and 9, whether modified or unmodified, can further comprise a targeting group or linking group, but not limited to, those depicted in Table 10, and the targeting group or linking group can be attached to the 3′ or 5′ terminus of either the sense strand or the antisense strand of the HTT RNAi agent duplex.

In some embodiments, an HTT RNAi agent is linked to one or more lipid PK/PD moieties (referred to herein as “lipid moieties” or “PK/PD modulators”.) Lipid PK/PD moieties may enhance the pharmacodynamic or pharmacokinetic properties of the RNAi agent. In some embodiments, the lipid moiety may be conjugated to a linker at the 3′ or 5′ end of a sense strand or an antisense strand of an RNAi agent described herein. In some embodiments, a lipid moiety may be linked at both the 3′ or 5′ end of either the sense strand or the antisense strand of an RNAi agent described herein.

In some embodiments, a lipid moiety may be conjugated to an HTT RNAi agent by reacting an HTT RNAi agent comprising an amine-comprising linker, for example, (NH2-C6) (see Table 10). In some embodiments, the amine-comprising linker may be located on the 5′ end of the sense strand or the antisense strand of an HTT RNAi agent. In some embodiments, the amine-comprising linker may be located on the 3′ end of the sense strand or the antisense strand of an RNAi agent.

In some embodiments, an RNAi agent comprising an amine-comprising linker, such as (NH2-C6) or (NH2-C6)s, may be reacted with a lipid comprising an activated ester moiety. Example lipids with activated ester moieties include LP-183-p and LP293-p as shown in Table 10 below.

In some embodiments, an HTT RNAi agent may be conjugated to a lipid moiety using phosphoramidite synthesis. Synthesizing oligonucleotides using phosphoramidites is well-known in the art. In some embodiments, a lipid moiety may be conjugated to the 5′ end of the sense strand or the antisense strand of an HTT RNAi agent using a phosphoramidite. In some embodiments, a lipid moiety may be conjugated to the 3′ end of the sense strand or the antisense strand of an HTT RNAi agent using a phosphoramidite. In some embodiments, a phosphoramidite such as LP-183 phosphoramidite, shown in Table 10 below, may be used to conjugate a lipid moiety to an HTT RNAi agent.

In some embodiments, HTT RNAi agents may comprise a lipid moiety on an internal nucleotide (i.e., not on the 3′ or 5′ terminal nucleotides.) In some embodiments, an internal nucleotide may be linked to the 2′ position of ribose.

Examples of certain modified nucleotides, capping moieties, PK/PD modulators and linking groups are provided in Table 10.

TABLE 10

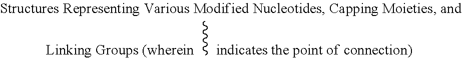

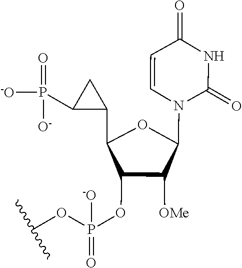

cPrpu

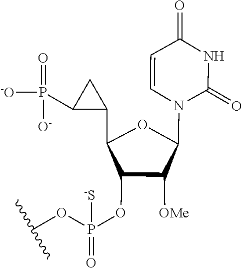

cPrpus

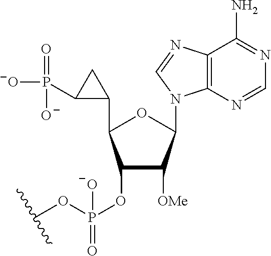

cPrpa

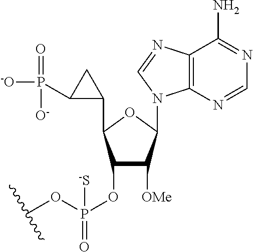

cPrpas

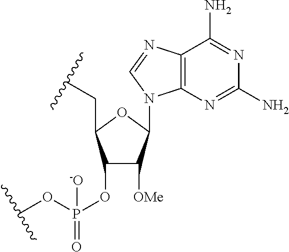

a_2N

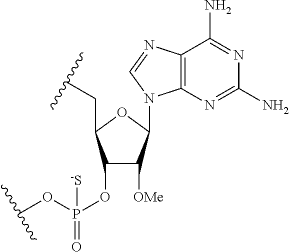

a_2Ns
When positioned internally:

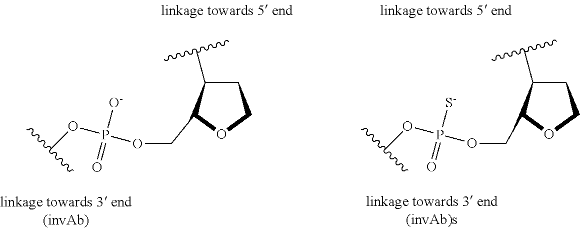

When positioned at the 3′ terminal end:

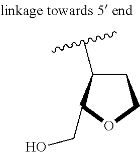

(invAb)
When position at the 3′ terminal end:

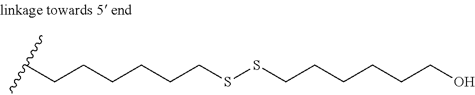 [SMILES: *CCCCCCSSCCCCCCO]

(C6-SS-C6)
When positioned internally:

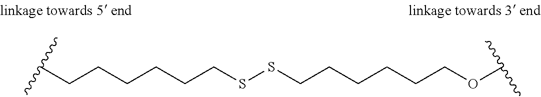 [SMILES: *CCCCCCSSCCCCCCO*]

(C6-SS-C6)
When position at the 3′ terminal end:

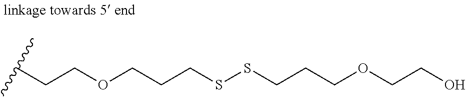 [SMILES: *CCOCCCSSCCCOCCO]

(6-SS-6)
When positioned internally:

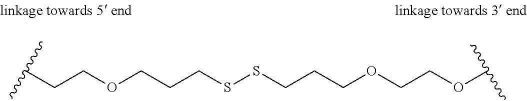 [SMILES: *CCOCCCSSCCCOCCO*]

(6-SS-6)

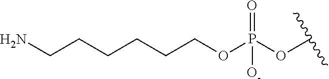

(NH2-C6)

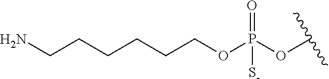

(NH2-C6)s

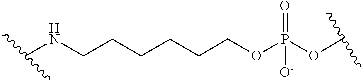 [SMILES: *NCCCCCCOP(=O)([O-])O*]

(NH-C6)

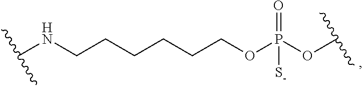

(NH-C6)s

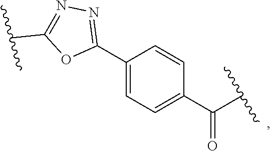 [SMILES: *C(=O)c1ccc(-c2nnc(*)o2)cc1]

L20

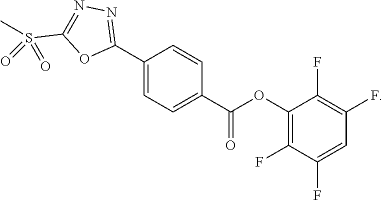 [SMILES: CS(=O)(=O)c1nnc(-c2ccc(C(=O)Oc3c(F)c(F)cc(F)c3F)cc2)o1]

L20-p

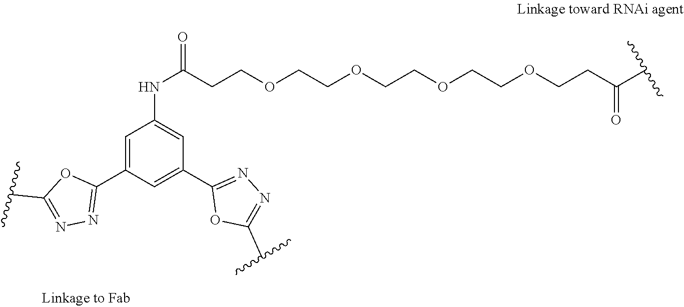 [SMILES: *C(=O)CCOCCOCCOCCOCCC(=O)Nc1cc(-c2nnc(*)o2)cc(-c2nnc(*)o2)c1]

(L1026)

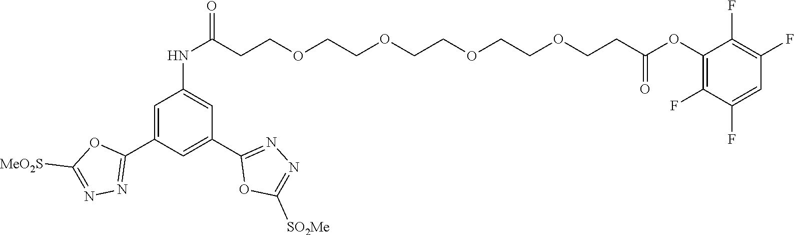 [SMILES: CS(=O)(=O)c1nnc(-c2cc(NC(=O)CCOCCOCCOCCOCCC(=O)Oc3c(F)c(F)cc(F)c3F)cc(-c3nnc(S(C)(=O)=O)o3)c2)o1]

L1026-p

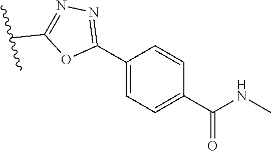 [SMILES: *c1nnc(-c2ccc(C(=O)NC)cc2)o1]

[CP-1113]

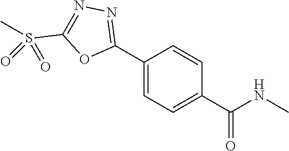

CP-1113-p

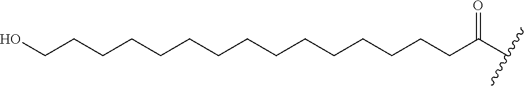 [SMILES: *C(=O)CCCCCCCCCCCCCCCO]

LP293

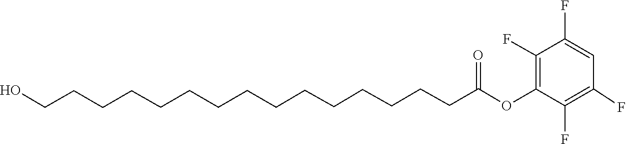 [SMILES: O=C(CCCCCCCCCCCCCCCO)Oc1c(F)c(F)cc(F)c1F]

LP293-p

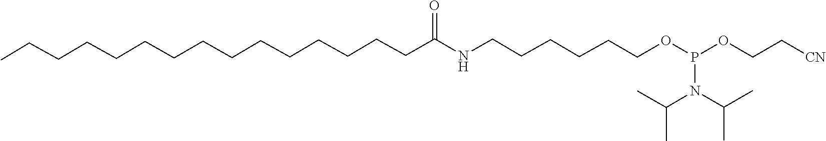 [SMILES: CCCCCCCCCCCCCCCC(=O)NCCCCCCOP(OCCC#N)N(C(C)C)C(C)C]

LP183 phosphoramidite

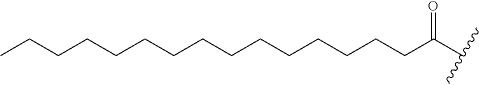 [SMILES: *C(=O)CCCCCCCCCCCCCCC]

LP183

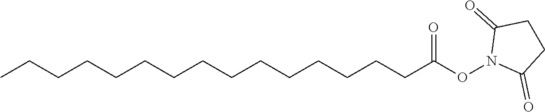 [SMILES: CCCCCCCCCCCCCCCC(=O)ON1C(=O)CCC1=O]

LP-183-p (commerically available)

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, an HTT RNAi agent is delivered without being conjugated to an antigen binding protein or other targeting group (referred to as being “naked” or a “naked RNAi agent”).

In some embodiments, an HTT RNAi agent is conjugated to a targeting group, a linking group, a PK modulator, and/or another non-nucleotide group to facilitate delivery of the HTT RNAi agent to the cell or tissue of choice, for example, to a CNS cell in vivo. In some embodiments, an HTT RNAi agent is conjugated to an antigen binding protein.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for CNS cells. In some embodiments, the RNAi agents can be linked to targeting ligands that have affinity for CNS cells or receptors present on CNS cells.

### Antigen Binding Proteins

In one aspect, HTT RNAi agents are conjugates to antigen binding proteins. In some embodiments, the antigen binding protein may be selected from the group consisting of: an antibody, an antibody fragment (e.g., an antigen binding fragment, or Fab), scFv, or other functional component or derivative of an antibody encompassing a Fab and/or complementary-determining regions (CDRs) disclosed herein.

In some embodiments, the antigen binding protein may act as a shuttle to facilitate the crossing of the blood brain barrier (BBB) of the RNAi agent, such that the RNAi agent may be administered subcutaneously and reach CNS tissue. In some embodiments, the antigen binding protein is an anti-Transferrin 1 (TfR1) antibody or Fab.

In some embodiments, the antigen binding protein is a Fab. In some embodiments, the Fab comprises (i) 6 complementary determining regions (CDRs), (ii) 3 CDRs on the variable light chain (VL), or (iii) 3 CDRs on the variable heavy chain (VH).

In some embodiments, the Fab comprises a light chain and a heavy chain. In some embodiments, the light chain comprises a variable light chain (VL) and a light constant chain 1 (CL). In some embodiments, the VL comprises three CDRs. In some embodiments, the VL comprises a VL CDR1, a VL CDR2, and a VL CDR3. In some embodiments, the heavy chain comprises a variable heavy chain (VH) and a heavy constant chain 1 (CH). In some embodiments, the VH comprises three CDRs. In some embodiments, the VH comprises a VH CDR1, a VH CDR2, and a VH CDR3.

In some embodiments, the light constant chain 1 (CL) comprises or consists of the sequence: RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 2). In some embodiments, the light chain comprises or consists of the sequence:

(SEQ ID NO: 3)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC.

In some embodiments, the heavy constant chain 1 (CH) comprises or consists of the sequence:

(SEQ ID NO: 5)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKRVEPKSCDKTH.

In some embodiments, the antigen binding protein may have a VL CDR1 sequence selected from the group consisting of: RASDGLYSNLA (SEQ ID NO: 6), RASDNLYRNLA (SEQ ID NO: 7), and RASDKLYSNLA (SEQ ID NO: 8).

In some embodiments, the antigen binding protein may have a VL CDR2 sequence selected from the group consisting of: DATLLAS (SEQ ID NO: 9), DARNLAS (SEQ ID NO: 10), DAFNLAS (SEQ ID NO: 11), DATRLAS (SEQ ID NO: 12), DATKLAS (SEQ ID NO: 13), and DAKNLAS (SEQ ID NO: 14).

In some embodiments, the antigen binding protein may have a VL CDR 3 sequence of QHFWGTPLT (SEQ ID NO: 15).

In some embodiments, the antigen binding protein may have a VH CDR1 sequence selected from the group consisting of: GYTFNSYWMH (SEQ ID NO: 16), GYTFKSYWMH (SEQ ID NO: 17), GFTFTSYWMH (SEQ ID NO: 18), GYTFTSYWVH (SEQ ID NO: 19), and GYTFTSYWMH (SEQ ID NO: 20).

In some embodiments, the antigen binding protein may have a VH CDR2 sequence selected from the group consisting of: EINPTNGRVNYIEKFKS (SEQ ID NO: 21), EINPTNGRFNYIEKFKS (SEQ ID NO: 22), EINPTNGRTNYIEKFKS (SEQ ID NO: 23), and EINPTNGRSNYIEKFKS (SEQ ID NO: 24).

In some embodiments, the antigen binding protein may have a VH CDR3 sequence of: GTRAYHY (SEQ ID NO: 25).

In some embodiments, the antigen binding protein may have a VL CDR1 sequence of RASDKLYSNLA (SEQ ID NO: 8), a VL CDR2 sequence of DATLLAS (SEQ ID NO: 9), and a VL CDR 3 sequence of QHFWGTPLT (SEQ ID NO: 15).

In some embodiments, the antigen binding protein may have a VH CDR1 sequence of GFTFTSYWMH (SEQ ID NO: 18), a VH CDR2 sequence of EINPTNGRTNYIEKFKS (SEQ ID NO: 23), and a VH CDR 3 sequence of GTRAYHY (SEQ ID NO: 25).

In some embodiments, the antigen binding protein may have a VL CDR1 sequence of RASDKLYSNLA (SEQ ID NO: 8), a VL CDR2 sequence of DATLLAS (SEQ ID NO: 9), a VL CDR 3 sequence of QHFWGTPLT (SEQ ID NO: 15), a VH CDR1 sequence of GFTFTSYWMH (SEQ ID NO: 18), a VH CDR2 sequence of EINPTNGRTNYIEKFKS (SEQ ID NO: 23), and a VH CDR 3 sequence of GTRAYHY (SEQ ID NO: 25).

In some embodiments, the VL comprises a sequence of any one of the sequences listed in Table A. Each of the Fabs described in Table A may have a light chain constant region that comprises or consists of the sequence:

(SEQ ID NO: 2)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the antigen binding protein may have a VL sequence of:

(SEQ ID NO: 32)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIK.

In some embodiments, the antigen binding protein may have a VH sequence of:

(SEQ ID NO: 40)
EVQLVESGGGLVQPGGSLRLSCATSGFTFTSYWMHWVRQAPGKGLEWVAE
INPTNGRTNYIEKFKSRITLSVDKSKSTVYLQMNSLRAEDTAVYYCARGT
RAYHYWGQGTLVTVSS.

In some embodiments, the antigen binding protein may have a VL sequence of: DIQLTQSPSSLSASVGDRVTITCRASDKLYSNLAWYQQKPGKAPKLLIYDATLLASG VPSRFSGSGSGTDYTLTISSLQPEDFATYYCQHFWGTPLTFGQGTKVEIK (SEQ ID NO: 32) and a VH sequence of:

(SEQ ID NO: 40)
EVQLVESGGGLVQPGGSLRLSCATSGFTFTSYWMHWVRQAPGKGLEWVAE
INPTNGRTNYIEKFKSRITLSVDKSKSTVYLQMNSLRAEDTAVYYCARGT
RAYHYWGQGTLVTVSS.

TABLE A
VL chains with CDR mutation combinations in bold.

| SEQ | | |
|---|---|---|
| ID | | |
| NO. | Fab | VL SEQUENCE |
| 26 | Fab0002 | DIQMTQSPSSLSASVGDRVTITCRASDNLYSNLAWYQQKPGKSP |
| | | KLLVYDATNLADGVPSRFSGSGSGTDYTLTISSLQPEDFATYYC |
| | | QHFWGTPLTFGQGTKVEIK |
| 27 | Fab0060 | DIQLTQSPSSLSASVGDRVTITCRASDGLYSNLAWYQQKPGKAP |
| | | KLLIYDATKLASGVPSRFSGSGSGTDYTLTISSLQPEDFATYYCQ |
| | | HFWGTPLTFGQGTKVEIK |
| 27 | Fab0061 | DIQLTQSPSSLSASVGDRVTITCRASDGLYSNLAWYQQKPGKAP |
| | | KLLIYDATKLASGVPSRFSGSGSGTDYTLTISSLQPEDFATYYCQ |
| | | HFWGTPLTFGQGTKVEIK |
| 28 | Fab0062 | DIQLTQSPSSLSASVGDRVTITCRASDGLYSNLAWYQQKPGKAP |
| | | KLLIYDAFNLASGVPSRFSGSGSGTDYTLTISSLQPEDFATYYCQ |
| | | HFWGTPLTFGQGTKVEIK |
| 29 | Fab0063 | DIQLTQSPSSLSASVGDRVTITCRASDGLYSNLAWYQQKPGKAP |
| | | KLLIYDATRLASGVPSRFSGSGSGTDYTLTISSLQPEDFATYYCQ |
| | | HFWGTPLTFGQGTKVEIK |
| 30 | Fab0064 | DIQLTQSPSSLSASVGDRVTITCRASDGLYSNLAWYQQKPGKAP |
| | | KLLIYDAKNLASGVPSRFSGSGSGTDYTLTISSLQPEDFATYYCQ |
| | | HFWGTPLTFGQGTKVEIK |
| 31 | Fab0065 | DIQLTQSPSSLSASVGDRVTITCRASDNLYRNLAWYQQKPGKAP |
| | | KLLIYDATKLASGVPSRFSGSGSGTDYTLTISSLQPEDFATYYCQ |
| | | HFWGTPLTFGQGTKVEIK |
| 28 | Fab0066 | DIQLTQSPSSLSASVGDRVTITCRASDGLYSNLAWYQQKPGKAP |
| | | KLLIYDAFNLASGVPSRFSGSGSGTDYTLTISSLQPEDFATYYCQ |
| | | HFWGTPLTFGQGTKVEIK |
| 31 | Fab0067 | DIQLTQSPSSLSASVGDRVTITCRASDNLYRNLAWYQQKPGKAP |
| | | KLLIYDATKLASGVPSRFSGSGSGTDYTLTISSLQPEDFATYYCQ |
| | | HFWGTPLTFGQGTKVEIK |
| 27 | Fab0068 | DIQLTQSPSSLSASVGDRVTITCRASDGLYSNLAWYQQKPGKAP |
| | | KLLIYDATKLASGVPSRFSGSGSGTDYTLTISSLQPEDFATYYCQ |
| | | HFWGTPLTFGQGTKVEIK |
| 27 | Fab0069 | DIQLTQSPSSLSASVGDRVTITCRASDGLYSNLAWYQQKPGKAP |
| | | KLLIYDATKLASGVPSRFSGSGSGTDYTLTISSLQPEDFATYYCQ |
| | | HFWGTPLTFGQGTKVEIK |
| 32 | Fab0070 | DIQLTQSPSSLSASVGDRVTITCRASDKLYSNLAWYQQKPGKAP |
| | | KLLIYDATLLASGVPSRFSGSGSGTDYTLTISSLQPEDFATYYCQ |
| | | HFWGTPLTFGQGTKVEIK |
| 33 | Fab0071 | DIQLTQSPSSLSASVGDRVTITCRASDGLYSNLAWYQQKPGKAP |
| | | KLLIYDARNLASGVPSRFSGSGSGTDYTLTISSLQPEDFATYYCQ |
| | | HFWGTPLTFGQGTKVEIK |
| 34 | Fab0072 | DIQLTQSPSSLSASVGDRVTITCRASDNLYRNLAWYQQKPGKAP |
| | | KLLIYDARNLASGVPSRFSGSGSGTDYTLTISSLQPEDFATYYCQ |
| | | HFWGTPLTFGQGTKVEIK |
| 31 | Fab0073 | DIQLTQSPSSLSASVGDRVTITCRASDNLYRNLAWYQQKPGKAP |
| | | KLLIYDATKLASGVPSRFSGSGSGTDYTLTISSLQPEDFATYYCQ |
| | | HFWGTPLTFGQGTKVEIK |
| 30 | Fab0074 | DIQLTQSPSSLSASVGDRVTITCRASDGLYSNLAWYQQKPGKAP |
| | | KLLIYDAKNLASGVPSRFSGSGSGTDYTLTISSLQPEDFATYYCQ |
| | | HFWGTPLTFGQGTKVEIK |

In some embodiments, the VL comprises a sequence of any one of the sequences listed in Table B. Each of the Fabs described in Table B may have a heavy chain constant region that comprises or consists of the sequence:

(SEQ ID NO: 4)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTH.

TABLE B
VH chains with CDR mutation combinations in bold.

| SEQ | | |
|---|---|---|
| ID | | |
| NO. | Fab | VH SEQUENCE |
| 35 | Fab0002 | EVQLVQSGAEVKKPGASVKVSCKASGYTFTSYWMHWVRQAP |
| | | GQRLEWIGEINPTNGRTNYIEKFKSRATLTVDKSASTAYMELSS |
| | | LRSEDTAVYYCARGTRAYHYWGQGTMVTVSS |
| 36 | Fab0060 | EVQLVESGGGLVQPGGSLRLSCATSGYTFTSYWVHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |
| 37 | Fab0061 | EVQLVESGGGLVQPGGSLRLSCATSGYTFTSYWMHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 37 | Fab0062 | EVQLVESGGGLVQPGGSLRLSCATSGYTFTSYWMHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 37 | Fab0063 | EVQLVESGGGLVQPGGSLRLSCATSGYTFTSYWMHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 37 | Fab0064 | EVQLVESGGGLVQPGGSLRLSCATSGYTFTSYWMHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 38 | Fab0065 | EVQLVESGGGLVQPGGSLRLSCATSGYTFTSYWVHWVRQAP |
| | | GKGLEWVAEINPTNGRSNYIEKFKSRITLSVDKSKSTVYLQM |
| | | NSLRAEDTAVYYCARGTRAYHYWGQGTLVTVSS |
| 39 | Fab0066 | EVQLVESGGGLVQPGGSLRLSCATSGYTFTSYWVHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 36 | Fab0067 | EVQLVESGGGLVQPGGSLRLSCATSGYTFTSYWVHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |
| 39 | Fab0068 | EVQLVESGGGLVQPGGSLRLSCATSGYTFTSYWVHWVRQAP |
| | | GKGLEWVAEINPTNGRVNYIEKFKSRITLSVDKSKSTVYLQM |
| | | NSLRAEDTAVYYCARGTRAYHYWGQGTLVTVSS |
| 38 | Fab0069 | EVQLVESGGGLVQPGGSLRLSCATSGYTFTSYWVHWVRQAP |
| | | GKGLEWVAEINPTNGRSNYIEKFKSRITLSVDKSKSTVYLQM |
| | | NSLRAEDTAVYYCARGTRAYHYWGQGTLVTVSS |
| 40 | Fab0070 | EVQLVESGGGLVQPGGSLRLSCATSGFTFTSYWMHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |
| 41 | Fab0071 | EVQLVESGGGLVQPGGSLRLSCATSGYTFNSYWMHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |
| 42 | Fab0072 | EVQLVESGGGLVQPGGSLRLSCATSGYTFNSYWMHWVRQAP |
| | | GKGLEWVAEINPTNGRFNYIEKFKSRITLSVDKSKSTVYLQM |
| | | NSLRAEDTAVYYCARGTRAYHYWGQGTLVTVSS |
| 43 | Fab0073 | EVQLVESGGGLVQPGGSLRLSCATSGYTFKSYWMHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |
| 43 | Fab0074 | EVQLVESGGGLVQPGGSLRLSCATSGYTFKSYWMHWVRQAP |
| | | GKGLEWVAEINPTNGRTNYIEKFKSRITLSVDKSKSTVYLQM |
| | | NSLRAEDTAVYYCARGTRAYHYWGQGTLVTVSS |

Tables C-H show the CDR1, CDR2, and CDR3 variants from VL and VH with the combined beneficial mutations.

TABLE C
VL CDR1 variant

| SEQ ID NO: | Fab | CDR1 |
|---|---|---|
| 6 | Fab0060 | RASDGLYSNLA |
| 6 | Fab0061 | RASDGLYSNLA |
| 6 | Fab0062 | RASDGLYSNLA |
| 6 | Fab0063 | RASDGLYSNLA |
| 6 | Fab0064 | RASDGLYSNLA |
| 7 | Fab0065 | RASDNLYRNLA |
| 6 | Fab0066 | RASDGLYSNLA |
| 7 | Fab0067 | RASDNLYRNLA |
| 6 | Fab0068 | RASDGLYSNLA |
| 6 | Fab0069 | RASDGLYSNLA |
| 8 | Fab0070 | RASDKLYSNLA |
| 6 | Fab0071 | RASDGLYSNLA |
| 7 | Fab0072 | RASDNLYRNLA |
| 7 | Fab0073 | RASDNLYRNLA |
| 6 | Fab0074 | RASDGLYSNLA |

TABLE D
VL CDR2 variants

| SEQ ID NO: | Fab | CDR2 |
|---|---|---|
| 13 | Fab0060 | DATKLAS |
| 13 | Fab0061 | DATKLAS |
| 11 | Fab0062 | DAFNLAS |
| 12 | Fab0063 | DATRLAS |
| 14 | Fab0064 | DAKNLAS |
| 13 | Fab0065 | DATKLAS |
| 11 | Fab0066 | DAFNLAS |
| 13 | Fab0067 | DATKLAS |
| 13 | Fab0068 | DATKLAS |
| 13 | Fab0069 | DATKLAS |
| 9 | Fab0070 | DATLLAS |
| 10 | Fab0071 | DARNLAS |
| 10 | Fab0072 | DARNLAS |
| 13 | Fab0073 | DATKLAS |
| 14 | Fab0074 | DAKNLAS |

TABLE E
VL CDR3 variant

| SEQ ID NO: | Fab | CDR3 |
|---|---|---|
| 15 | Fab0060 | QHFWGTPLT |
| 15 | Fab0061 | QHFWGTPLT |
| 15 | Fab0062 | QHFWGTPLT |
| 15 | Fab0063 | QHFWGTPLT |
| 15 | Fab0064 | QHFWGTPLT |
| 15 | Fab0065 | QHFWGTPLT |
| 15 | Fab0066 | QHFWGTPLT |
| 15 | Fab0067 | QHFWGTPLT |
| 15 | Fab0068 | QHFWGTPLT |
| 15 | Fab0069 | QHFWGTPLT |
| 15 | Fab0070 | QHFWGTPLT |
| 15 | Fab0071 | QHFWGTPLT |
| 15 | Fab0072 | QHFWGTPLT |
| 15 | Fab0073 | QHFWGTPLT |
| 15 | Fab0074 | QHFWGTPLT |

TABLE F
VH CDR1 variants

| SEQ ID NO: | Fab | CDR1 |
|---|---|---|
| 19 | Fab0060 | GYTFTSYWVH |
| 20 | Fab0061 | GYTFTSYWMH |
| 20 | Fab0062 | GYTFTSYWMH |
| 20 | Fab0063 | GYTFTSYWMH |
| 20 | Fab0064 | GYTFTSYWMH |
| 19 | Fab0065 | GYTFTSYWVH |
| 19 | Fab0066 | GYTFTSYWVH |
| 19 | Fab0067 | GYTFTSYWVH |
| 19 | Fab0068 | GYTFTSYWVH |
| 19 | Fab0069 | GYTFTSYWVH |
| 18 | Fab0070 | GFTFTSYWMH |
| 16 | Fab0071 | GYTFNSYWMH |
| 16 | Fab0072 | GYTFNSYWMH |
| 17 | Fab0073 | GYTFKSYWMH |
| 17 | Fab0074 | GYTFKSYWMH |

TABLE G
VH CDR2 variants

| SEQ ID NO: | Fab | CDR2 |
|---|---|---|
| 23 | Fab0060 | EINPTNGRTNYIEKFKS |
| 21 | Fab0061 | EINPTNGRVNYIEKFKS |
| 21 | Fab0062 | EINPTNGRVNYIEKFKS |
| 21 | Fab0063 | EINPTNGRVNYIEKFKS |
| 21 | Fab0064 | EINPTNGRVNYIEKFKS |
| 24 | Fab0065 | EINPTNGRSNYIEKFKS |
| 21 | Fab0066 | EINPTNGRVNYIEKFKS |
| 23 | Fab0067 | EINPTNGRTNYIEKFKS |
| 21 | Fab0068 | EINPTNGRVNYIEKFKS |
| 24 | Fab0069 | EINPTNGRSNYIEKFKS |
| 23 | Fab0070 | EINPTNGRTNYIEKFKS |
| 23 | Fab0071 | EINPTNGRTNYIEKFKS |
| 22 | Fab0072 | EINPTNGRFNYIEKFKS |
| 23 | Fab0073 | EINPTNGRTNYIEKFKS |
| 23 | Fab0074 | EINPTNGRTNYIEKFKS |

TABLE H
VH CDR3 variants

| SEQ ID NO: | Fab | CDR3 |
|---|---|---|
| 25 | Fab0060 | GTRAYHY |
| 25 | Fab0061 | GTRAYHY |
| 25 | Fab0062 | GTRAYHY |
| 25 | Fab0063 | GTRAYHY |
| 25 | Fab0064 | GTRAYHY |
| 25 | Fab0065 | GTRAYHY |
| 25 | Fab0066 | GTRAYHY |
| 25 | Fab0067 | GTRAYHY |
| 25 | Fab0068 | GTRAYHY |
| 25 | Fab0069 | GTRAYHY |
| 25 | Fab0070 | GTRAYHY |
| 25 | Fab0071 | GTRAYHY |
| 25 | Fab0072 | GTRAYHY |
| 25 | Fab0073 | GTRAYHY |
| 25 | Fab0074 | GTRAYHY |

In some embodiments, RNAi agents may be conjugated to an antigen binding protein specific to a non-human mammal to carry out studies. RNAi agents may be conjugated to a mouse-specific anti-transferrin antibody such as Fab0016. Fab0016 as used herein refers to an antibody fragment having a light chain sequence of: DIQMTQSPASLSASLEEIVTITCQASQDIGNWLAWYQQKPGKSPQLLIYGATSLADGV PSRFSGSRSGTQFSLKISRVQVEDIGIYYCLQAYNTPWTFGGGTKLELKRADAAPTVS IFPPSTEQLATGGASWCLMNNFYPRDISVKWKIDGTERRDGVLDSVTDQDSKDSTY SMSSTLSLTKADYESHNLYTCEVVHKTSSSPVVKSFNRNEC (SEQ ID NO: 44), and a heavy chain sequence of:

(SEQ ID NO: 45)
EVQLVESGGGLVQPGNSLTLSCVASGFTFSNYGMHWIRQAPKKGLEWIAM
IYYDSSKMNYADTVKGRFTISRDNSKNTLYLEMNSLRSEDTAMYYCAVPT
SHYVVDVWGQGVSVTVSSAETTAPSVYPLAPGTALKSNSMVTLGCLVKGY
FPEPVTVTWNSGALSSGVHTFPAVLQSGLYTLTSSVTVPSSTWSSQAVTC
NVAHPASSTKVDKKIVPREC.

In some embodiments, the Fab binds TfR1. In some embodiments, the Fab binds TfR1 with an affinity of at least about 1, 2, 3, 4, 5, 6, 7, 8, 9, 10, 20, 30, 40, 50, 60, 70, 80, 90, 100 nM. In some embodiments, the Fab binds TfR1 with an affinity of about 1, 2, 3, 4, 5, 6, 7, 8, 9, 10, 20, 30, 40, 50, 60, 70, 80, 90, 100 nM. In some embodiments, the Fab binds TfR1 with an affinity of at least about 1 nM. In some embodiments, the Fab binds TfR1 with a KD value of at least about 1, 2, 3, 4, 5, 6, 7, 8, 9, 10, 20, 30, 40, 50, 60, 70, 80, 90, 100 nM. In some embodiments, the Fab binds TfR1 with a KD value of about 1, 2, 3, 4, 5, 6, 7, 8, 9, 10, 20, 30, 40, 50, 60, 70, 80, 90, 100 nM. In some embodiments, the Fab binds TfR1 with a KD value of at least about 1 nM.

In some embodiments, the Fab is conjugated to an RNAi agent disclosed herein. In some embodiments, the RNAi agent is conjugated to the Fab using a covalent or non-covalent bond, ionic bond, hydrogen bond, hydrophobic interaction, peptide, polymer, or a nucleic acid binding protein. In some embodiments, the RNAi agent is conjugated to the Fab using a covalent bond. In some embodiments, the RNAi agent is conjugated to the Fab via a lysine residue or a cysteine residue. In some embodiments, the RNAi agent is conjugated to the Fab via a lysine residue. In some embodiments, the RNAi agent is conjugated to the Fab via a cysteine residue. In some embodiments, the RNAi agent is conjugated to the Fab in a site-specific manner. In some embodiments, the RNAi agent is conjugated to the Fab in a non-site-specific manner.

In some embodiments, the RNAi agent is conjugated to the Fab at the 5′ terminus or the 3′ terminus of the RNAi agent. In some embodiments, the RNAi agent is conjugated to the Fab at the 5′ terminus of the RNAi agent. In some embodiments, the RNAi agent is conjugated to the Fab at the 3′ terminus of the RNAi agent. In some embodiments, the RNAi agent is conjugated to the Fab at the 5′ terminus or the 3′ terminus of the sense strand of the RNAi agent. In some embodiments, the RNAi agent is conjugated to the Fab at the 5′ terminus of the sense strand of the RNAi agent. In some embodiments, the RNAi agent is conjugated to the Fab at the 3′ terminus of the sense strand of the RNAi agent.

### Pharmaceutical Compositions and Formulations

The HTT RNAi agents disclosed herein can be prepared as pharmaceutical compositions or formulations (also referred to herein as “medicaments”). In some embodiments, pharmaceutical compositions include at least one HTT RNAi agent. These pharmaceutical compositions are particularly useful in the inhibition of the expression of HTT mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering an HTT RNAi agent linked to an antigen binding protein as described herein, to a subject to be treated. In some embodiments, one or more pharmaceutically acceptable excipients (including vehicles, carriers, diluents, and/or delivery polymers) are added to the pharmaceutical compositions that include an HTT RNAi agent, thereby forming a pharmaceutical formulation or medicament suitable for in vivo delivery to a subject, including a human.

The pharmaceutical compositions that include an HTT RNAi agent and methods disclosed herein decrease the level of the target mRNA in a cell, group of cells, tissue, organ, or subject, including by administering to the subject a therapeutically effective amount of a herein described HTT RNAi agent, thereby inhibiting the expression of HTT mRNA in the subject. In some embodiments, the subject has been previously identified or diagnosed as having a disease or disorder that can be mediated at least in part by a reduction in HTT gene expression. In some embodiments, the subject has been previously diagnosed with having one or more neurodegenerative diseases such as Huntington's Disease. In some embodiments the neurodegenerative disease is Huntington's Disease.

In some embodiments the subject has been previously diagnosed with having neurodegenerative disease.

Embodiments of the present disclosure include pharmaceutical compositions for delivering an HTT RNAi agent to a CNS cell in vivo. Such pharmaceutical compositions can include, for example, an HTT RNAi agent conjugated to an antigen binding protein.

In some embodiments, the described pharmaceutical compositions including an HTT RNAi agent are used for treating or managing clinical presentations in a subject that would benefit from the inhibition of expression of HTT. In some embodiments, a therapeutically or prophylactically effective amount of one or more of pharmaceutical compositions is administered to a subject in need of such treatment. In some embodiments, administration of any of the disclosed HTT RNAi agents can be used to decrease the number, severity, and/or frequency of symptoms of a disease in a subject.

In some embodiments, the described HTT RNAi agents are optionally combined with one or more additional (i.e., second, third, etc.) therapeutics. A second therapeutic can be another HTT RNAi agent (e.g., an HTT RNAi agent that targets a different sequence within an HTT gene). In some embodiments, a second therapeutic can be an RNAi agent that targets the HTT gene. An additional therapeutic can also be a small molecule drug, antibody, antibody fragment, and/or aptamer. The HTT RNAi agents, with or without the one or more additional therapeutics, can be combined with one or more excipients to form pharmaceutical compositions.

The described pharmaceutical compositions that include an HTT RNAi agent can be used to treat at least one symptom in a subject having a disease or disorder that would benefit from reduction or inhibition in expression of HTT mRNA. In some embodiments, the subject is administered a therapeutically effective amount of one or more pharmaceutical compositions that include an HTT RNAi agent thereby treating the symptom. In other embodiments, the subject is administered a prophylactically effective amount of one or more HTT RNAi agents, thereby preventing or inhibiting the at least one symptom.

In some embodiments, one or more of the described HTT RNAi agents are administered to a mammal in a pharmaceutically acceptable carrier or diluent. In some embodiments, the mammal is a human.

The route of administration is the path by which an HTT RNAi agent is brought into contact with the body. In general, methods of administering drugs, oligonucleotides, and nucleic acids including the CNS, for treatment of a mammal are well known in the art and can be applied to administration of the compositions described herein. The HTT RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intracerebroventricularly, intraarticularly, intraocularly, or intraperitoneally, or topically.

The pharmaceutical compositions including an HTT RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intracerebroventricular, intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration. For example, in some embodiments, it is desired that the HTT RNAi agents described herein inhibit the expression of an HTT gene in the CNS.

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., HTT RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The HTT RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, HTT RNAi agent pharmaceutical compositions may contain salts such as sodium chloride, calcium chloride, magnesium chloride, potassium chloride, sodium phosphate dibasic, sodium phosphate monobasic, or combinations thereof.

In some embodiments, the methods disclosed herein further comprise the step of administering a second therapeutic or treatment in addition to administering an RNAi agent disclosed herein. In some embodiments, the second therapeutic is another HTT RNAi agent (e.g., an HTT RNAi agent that targets a different sequence within the HTT target). In other embodiments, the second therapeutic can be a small molecule drug, an antibody, an antibody fragment, and/or an aptamer.

In some embodiments, described herein are compositions that include a combination or cocktail of at least two HTT RNAi agents having different sequences. In some embodiments, the two or more HTT RNAi agents are each separately and independently linked to antigen binding proteins.

Described herein are compositions for delivery of HTT RNAi agents to central nervous system (CNS) cells. Furthermore, compositions for delivery of HTT RNAi agents to cells, including neurons, astrocytes, microglia and endothelial cells, in vivo, are generally described herein.

Generally, an effective amount of an HTT RNAi agent disclosed herein will be in the range of from about 0.0001 to about 20 mg/kg of body weight dose, e.g., from about 0.001 to about 5 mg/kg of body weight dose. In some embodiments, an effective amount of an HTT RNAi agent will be in the range of from about 0.01 mg/kg to about 3.0 mg/kg of body weight per dose. In some embodiments, an effective amount of an HTT RNAi agent will be in the range of from about 0.03 mg/kg to about 2.0 mg/kg of body weight per dose. In some embodiments, an effective amount of an HTT RNAi agent will be in the range of from about 0.01 to about 1.0 mg/kg. In some embodiments, an effective amount of an HTT RNAi agent will be in the range of from about 0.50 to about 1.0 mg/kg. In some embodiments, a fixed dose of HTT RNAi agent is administered to the subject. In some embodiments the dose administered to the human subject is between about 1.0 mg and about 750 mg. In some embodiments, the dose of HTT RNAi agent administered to the human subject is between about 10 mg and about 450 mg. In some embodiments, the dose of HTT RNAi agent administered to the human subject is between about 25 mg and about 450 mg. In some embodiments, the dose of HTT RNAi agent administered to the human subject is about 50 mg, about 75 mg, about 100 mg, about 150 mg, about 200 mg, about 250 mg, about 300 mg, about 350 mg, about 400 mg, or about 450 mg. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including an HTT RNAi agent can be combined with an excipient or with a second therapeutic agent or treatment including, but not limited to: a second or other RNAi agent, a small molecule drug, an antibody, an antibody fragment, peptide, and/or an aptamer.

The described HTT RNAi agents, when added to pharmaceutically acceptable excipients or adjuvants, can be packaged into kits, containers, packs, or dispensers.

### Methods of Treatment and Inhibition of HTT Gene Expression

The HTT RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of HTT mRNA and/or a reduction in HTT protein levels.

In some embodiments, the RNAi agents disclosed herein can be used to treat a subject (e.g., a human) having a disease or disorder for which the subject would benefit from reduction in HTT protein, including but not limited to, Huntington's Disease. Treatment of a subject can include therapeutic and/or prophylactic treatment. The subject is administered a therapeutically effective amount of any one or more HTT RNAi agents described herein. The subject can be a human, patient, or human patient. The subject may be an adult, adolescent, child, or infant. Administration of a pharmaceutical composition described herein can be to a human being or animal.

Mutant HTT activity is known to promote neurodegenerative disorders. In some embodiments, the described HTT RNAi agents are used to treat at least one symptom mediated at least in part by a reduction in mutant HTT protein levels, in a subject. The subject is administered a therapeutically effective amount of any one or more of the described HTT RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by HTT gene expression, in a patient in need thereof, wherein the methods include administering to the patient any of the HTT RNAi agents described herein.

In some embodiments, the HTT RNAi agents are used to treat or manage a clinical presentation or pathological state in a subject, wherein the clinical presentation or pathological state is mediated at least in part by a reduction in HTT gene expression. The subject is administered a therapeutically effective amount of one or more of the HTT RNAi agents or HTT RNAi agent-containing compositions described herein. In some embodiments, the method comprises administering a composition comprising an HTT RNAi agent described herein to a subject to be treated.

In a further aspect, the disclosure features methods of treatment (including prophylactic or preventative treatment) of diseases or symptoms that may be addressed by a reduction in HTT protein levels, the methods comprising administering to a subject in need thereof an HTT RNAi agent that includes an antisense strand comprising the sequence of any of the sequences in Table 2, Table 3, or Table 9. Also described herein are compositions for use in such methods.

The described HTT RNAi agents and/or compositions that include HTT RNAi agents can be used in methods for therapeutic treatment of disease or conditions caused by enhanced or elevated HTT protein levels. Such methods include administration of an HTT RNAi agent as described herein to a subject, e.g., a human or animal subject.

In another aspect, the disclosure provides methods for the treatment (including prophylactic treatment) of a pathological state (such as a condition or disease) mediated at least in part by HTT gene expression, wherein the methods include administering to a subject a therapeutically effective amount of an RNAi agent that includes an antisense strand comprising the sequence of any of the sequences in Table 2, Table 3, or Table 9.

In some embodiments, methods for inhibiting expression of an HTT gene are disclosed herein, wherein the methods include administering to a cell an RNAi agent that includes an antisense strand comprising the sequence of any of the sequences in Table 2, Table 3, or Table 9.

In some embodiments, methods for the treatment (including prophylactic treatment) of a pathological state mediated at least in part by HTT gene expression are disclosed herein, wherein the methods include administering to a subject a therapeutically effective amount of an RNAi agent that includes a sense strand comprising the sequence of any of the sequences in Table 2, Table 4, Table 5, Table 6, or Table 9.

In some embodiments, methods for inhibiting expression of an HTT gene are disclosed herein, wherein the methods comprise administering to a cell an RNAi agent that includes a sense strand comprising the sequence of any of the sequences in Table 2, Table 4, Table 5, Table 6, or Table 9.

In some embodiments, methods for the treatment (including prophylactic treatment) of a pathological state mediated at least in part by HTT gene expression are disclosed herein, wherein the methods include administering to a subject a therapeutically effective amount of an RNAi agent that includes a sense strand comprising the sequence of any of the sequences in Table 4, Table 5, Table 6, or Table 9, and an antisense strand comprising the sequence of any of the sequences in Table 3 or Table 9.

In some embodiments, methods for inhibiting expression of an HTT gene are disclosed herein, wherein the methods include administering to a cell an RNAi agent that includes a sense strand comprising the sequence of any of the sequences in Table 4, Table 5, Table 6, or Table 9, and an antisense strand comprising the sequence of any of the sequences in Table 3 or Table 9.

In some embodiments, methods of inhibiting expression of an HTT gene are disclosed herein, wherein the methods include administering to a subject an HTT RNAi agent that includes a sense strand consisting of the nucleobase sequence of any of the sequences in Table 4, Table 5, Table 6, or Table 9, and the antisense strand consisting of the nucleobase sequence of any of the sequences in Table 3 or Table 9. In other embodiments, disclosed herein are methods of inhibiting expression of an HTT gene, wherein the methods include administering to a subject an HTT RNAi agent that includes a sense strand consisting of the modified sequence of any of the modified sequences in Table 4, Table 5, Table 6, or Table 9, and the antisense strand consisting of the modified sequence of any of the modified sequences in Table 3 or Table 9.

In some embodiments, methods for inhibiting expression of an HTT gene in a cell are disclosed herein, wherein the methods include administering one or more HTT RNAi agents comprising a duplex structure of one of the duplexes set forth in Tables 7, 8, and 9.

In some embodiments, the gene expression level and/or mRNA level of an HTT gene in certain CNS cells of subject to whom a described HTT RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the HTT RNAi agent or to a subject not receiving the HTT RNAi agent. In some embodiments, the HTT protein levels in certain CNS cells of a subject to whom a described HTT RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the HTT RNAi agent or to a subject not receiving the HTT RNAi agent. The gene expression level, protein level, and/or mRNA level in the subject may be reduced in a cell, group of cells, and/or tissue of the subject. In some embodiments, the HTT mRNA levels in certain CNS cells subject to whom a described HTT RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the HTT RNAi agent or to a subject not receiving the HTT RNAi agent.

A reduction in gene expression, mRNA, and protein levels can be assessed by any methods known in the art. Reduction or decrease in HTT protein and/or enzyme levels are collectively referred to herein as a decrease in, reduction of, or inhibition of HTT expression. The Examples set forth herein illustrate known methods for assessing inhibition of HTT gene expression, including but not limited to determining HTT protein levels.

### Cells, Tissues, Organs, and Non-Human Organisms

Cells, tissues, organs, and non-human organisms that include at least one of the HTT RNAi agents described herein are contemplated. The cell, tissue, organ, or non-human organism is made by delivering the RNAi agent to the cell, tissue, organ, or non-human organism.

### Additional Illustrative Embodiments

Provided here are certain additional illustrative embodiments of the disclosed technology. These embodiments are illustrative only and do not limit the scope of the present disclosure or of the claims attached hereto.

Embodiment 1. An RNAi agent for inhibiting expression of a huntingtin (HTT) gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

Embodiment 2. The RNAi agent of claim 1, wherein the antisense strand comprises nucleotides 2-18 of any one of the sequences provided in Table 2 or Table 3.

Embodiment 3. The RNAi agent of claim 1 or claim 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

Embodiment 4. The RNAi agent of any one of claims 1-3, wherein at least one nucleotide of the HTT RNAi agent is a modified nucleotide or includes a modified internucleoside linkage.

Embodiment 5. The RNAi agent of any one of claims 1-4, wherein all or substantially all of the nucleotides are modified nucleotides.

Embodiment 6. The RNAi agent of any one of claims 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 7. The RNAi agent of claim 5, wherein all or substantially all of the nucleotides are modified with 2′-O-methyl nucleotides, 2′-fluoro nucleotides, or combinations thereof.

Embodiment 8. The RNAi agent of any one of claims 1-7, wherein the antisense strand comprises the nucleotide sequence of any one of the modified sequences provided in Table 3.

Embodiment 9. The RNAi agent of any one of claims 1-8, wherein the sense strand comprises the nucleotide sequence of any one of the modified sequences provided in Table 4.

Embodiment 10. The RNAi agent of claim 1, wherein the antisense strand comprises the nucleotide sequence of any one of the modified sequences provided in Table 3 and the sense strand comprises the nucleotide sequence of any one of the modified sequences provided in Table 4.

Embodiment 11. The RNAi agent of any one of claims 1-10, wherein the sense strand is between 18 and 30 nucleotides in length, and the antisense strand is between 18 and 30 nucleotides in length.

Embodiment 12. The RNAi agent of claim 11, wherein the sense strand and the antisense strand are each between 18 and 27 nucleotides in length.

Embodiment 13. The RNAi agent of claim 12, wherein the sense strand and the antisense strand are each between 18 and 24 nucleotides in length.

Embodiment 14. The RNAi agent of claim 13, wherein the sense strand and the antisense strand are each 21 nucleotides in length.

Embodiment 15. The RNAi agent of claim 14, wherein the RNAi agent has two blunt ends.

Embodiment 16. The RNAi agent of any one of claims 1-15, wherein the sense strand comprises one or two terminal caps.

Embodiment 17. The RNAi agent of any one of claims 1-16, wherein the sense strand comprises one or two inverted abasic residues.

Embodiment 18. The RNAi agent of claim 1, wherein the RNAi agent is comprised of a sense strand and an antisense strand that form a duplex having the structure of any one of the duplexes in Table 7, Table 8, or Table 9.

Embodiment 19. The RNAi agent of claim 18, wherein all or substantially all of the nucleotides are modified nucleotides.

Embodiment 20. The RNAi agent of any one of claims 1-19, wherein the RNAi agent is conjugated to an antigen binding protein.

Embodiment 21. The RNAi agent of claim 20, wherein the antigen binding protein is conjugated to the sense strand.

Embodiment 22. The RNAi agent of any one of claim 20 or 21, wherein the antigen binding protein is an antibody fragment (Fab) that specifically binds to one or more epitopes on a transferrin receptor (TfR1).

Embodiment 23. The RNAi agent of claim 22, wherein the Fab comprises (i) 6 complementary determining regions (CDRs), (ii) 3 CDRs on the variable light chain (VL), and/or (iii) 3 CDRs on the variable heavy chain (VH).

Embodiment 24. The RNAi agent of claim 23, wherein the variable light chain has a VL CDR1 sequence selected from the group consisting of: RASDGLYSNLA (SEQ ID NO: 6), RASDNLYRNLA (SEQ ID NO: 7), and RASDKLYSNLA (SEQ ID NO: 8); a VL CDR2 sequence selected from the group consisting of: DATLLAS (SEQ ID NO: 9), DARNLAS (SEQ ID NO: 10), DAFNLAS (SEQ ID NO: 11), DATRLAS (SEQ ID NO: 12), DATKLAS (SEQ ID NO: 13), and DAKNLAS (SEQ ID NO: 14); and/or a VL CDR 3 sequence of QHFWGTPLT (SEQ ID NO: 15).

Embodiment 25. The RNAi agent of claim 23 or 24, wherein the variable light chain is selected from any one of the VL chains shown in Table A.

Embodiment 26. The RNAi agent of any one of claims 23-25, wherein the variable light chain comprises the sequence:

(SEQ ID NO: 32)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIK

Embodiment 27. The RNAi agent of any one of claims 23-26, wherein the variable heavy chain has a VH CDR1 sequence selected from the group consisting of: GYTFNSYWMH (SEQ ID NO: 16), GYTFKSYWMH (SEQ ID NO: 17), GFTFTSYWMH (SEQ ID NO: 18), GYTFTSYWVH (SEQ ID NO: 19), and GYTFTSYWMH (SEQ ID NO: 20), a VH CDR2 sequence selected from the group consisting of: EINPTNGRVNYIEKFKS (SEQ ID NO: 21), EINPTNGRFNYIEKFKS (SEQ ID NO: 22), EINPTNGRTNYIEKFKS (SEQ ID NO: 23), and EINPTNGRSNYIEKFKS (SEQ ID NO: 24); and/or a VH CDR3 sequence of: GTRAYHY (SEQ ID NO: 25).

Embodiment 28. The RNAi agent of any one of claims 23-27, wherein the variable heavy chain is selected from any one of the VH chains shown in Table B.

Embodiment 29. The RNAi agent of any one of claims 23-28, wherein the variable chain comprises the

(SEQ ID NO: 40)
EVQLVESGGGLVQPGGSLRLSCATSGFTFTSYWMHWVRQAPGKGLEWVAE
INPTNGRTNYIEKFKSRITLSVDKSKSTVYLQMNSLRAEDTAVYYCARGT
RAYHYWGQGTLVTVSS

Embodiment 30. The RNAi agent of any one of claims 22-29, wherein the Fab further comprises a light constant chain 1 (CL).

Embodiment 31. The RNAi agent of claim 30, wherein the light constant chain 1 (CL) sequence is:

(SEQ ID NO: 2)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Embodiment 32. The RNAi agent of any one of claims 22-31, wherein the Fab further comprises a heavy constant chain 1 (CH).

Embodiment 33. The RNAi agent of claim 32, wherein the heavy constant chain 1 (CH) sequence is:

(SEQ ID NO: 4)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTH.

Embodiment 34. The RNAi agent of any one of claims 22-33, wherein the antibody fragment (Fab) binds TfR1 with an affinity of at least 1 nM KD.

Embodiment 35. The RNAi agent of any one of claims 1-19, wherein the RNAi agent is conjugated to a lipid.

Embodiment 36. The RNAi agent of claim 35, wherein the lipid is selected from:

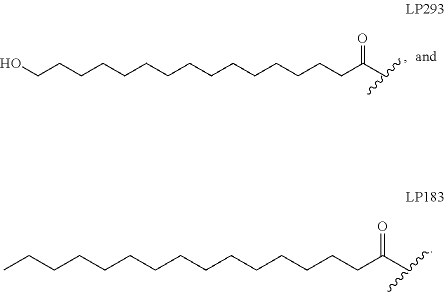 [SMILES: *C(=O)CCCCCCCCCCCCCCCO ; *C(=O)CCCCCCCCCCCCCCC]

Embodiment 37. The RNAi agent of claim 35 or 36, wherein the lipid is conjugated to the sense strand.

Embodiment 38. The RNAi agent of claim 37, wherein the lipid is conjugated to the 5′ end of the sense strand.

Embodiment 39. The RNAi agent of claim 37, wherein the lipid is conjugated to the 3′ end of the sense strand.

Embodiment 40. A conjugate comprising the RNAi agent of any one of claims 1-21 conjugated to an antibody fragment (Fab) that specifically binds to one or more epitopes on a transferrin receptor (TfR1).

Embodiment 41. The conjugate of claim 40, wherein the Fab comprises (i) 6 complementary determining regions (CDRs), (ii) 3 CDRs on the variable light chain (VL), or (iii) 3 CDRs on the variable heavy chain (VH).

Embodiment 42. The conjugate of claim 41, wherein the variable light chain has a VL CDR1 sequence selected from the group consisting of: RASDGLYSNLA (SEQ ID NO: 6), RASDNLYRNLA (SEQ ID NO: 7), and RASDKLYSNLA (SEQ ID NO: 8); a VL CDR2 sequence selected from the group consisting of: DATLLAS (SEQ ID NO: 9), DARNLAS (SEQ ID NO: 10), DAFNLAS (SEQ ID NO: 11), DATRLAS (SEQ ID NO: 12), DATKLAS (SEQ ID NO: 13), and DAKNLAS (SEQ ID NO: 14); and/or a VL CDR 3 sequence of QHFWGTPLT (SEQ ID NO: 15).

Embodiment 43. The conjugate of claim 41 or 42, wherein the variable light chain is selected from any one of the VL chains shown in Table A.

Embodiment 44. The conjugate of any one of claims 41-43, wherein the variable light chain comprises the sequence:

(SEQ ID NO: 32)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIK

Embodiment 45. The conjugate of any one of claims 41-44, wherein the variable heavy chain has a VH CDR1 sequence selected from the group consisting of: GYTFNSYWMH (SEQ ID NO: 16), GYTFKSYWMH (SEQ ID NO: 17), GFTFTSYWMH (SEQ ID NO: 18), GYTFTSYWVH (SEQ ID NO: 19), and GYTFTSYWMH (SEQ ID NO: 20), a VH CDR2 sequence selected from the group consisting of: EINPTNGRVNYIEKFKS (SEQ ID NO: 21), EINPTNGRFNYIEKFKS (SEQ ID NO: 22), EINPTNGRTNYIEKFKS (SEQ ID NO: 23), and EINPTNGRSNYIEKFKS (SEQ ID NO: 24); and/or a VH CDR3 sequence of: GTRAYHY (SEQ ID NO: 25).

Embodiment 46. The conjugate of any one of claims 41-45, wherein the variable heavy chain is selected from any one of the VH chains shown in Table B.

Embodiment 47. The conjugate of any one of claims 41-46, wherein the variable heavy chain comprises the sequence:

(SEQ ID NO: 40)
EVQLVESGGGLVQPGGSLRLSCATSGFTFTSYWMHWVRQAPGKGLEWVAE
INPTNGRTNYIEKFKSRITLSVDKSKSTVYLQMNSLRAEDTAVYYCARGT
RAYHYWGQGTLVTVSS

Embodiment 48. The conjugate of any one of claims 40-47, wherein the Fab further comprises a light constant chain 1 (CL).

Embodiment 49. The conjugate of claim 48, wherein the light constant chain 1 (CL) sequence is:

(SEQ ID NO: 2)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Embodiment 50. The conjugate of any one of claims 40-49, wherein the Fab further comprises a heavy constant chain 1 (CH).

Embodiment 51. The conjugate of claim 50, wherein the heavy constant chain 1 (CH) sequence is:

(SEQ ID NO: 4)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTH.

Embodiment 52. The conjugate of any one of claims 40-51, wherein the antibody fragment (Fab) binds TfR1 with an affinity of at least 1 nM KD.

Embodiment 53. The conjugate of any one of claims 40-52, wherein the RNAi agent is conjugated to the Fab using a covalent or non-covalent bond, ionic bond, hydrogen bond, hydrophobic interaction, peptide, polymer, or a nucleic acid binding protein.

Embodiment 54. The conjugate of any one of claims 40-53, wherein the RNAi agent is conjugated to the Fab through a linker comprising a structure selected from the group consisting of:

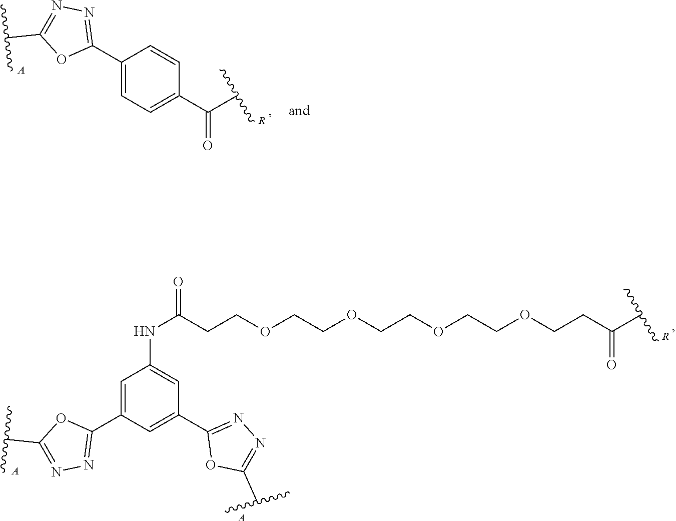

wherein [symbol] _{A} represents a point of attachment to the Fab, and [symbol] _{R} represents a point of attachment to the RNAi agent portion of the conjugate.

Embodiment 55. The conjugate of any one of claims 40-54, wherein the RNAi agent is conjugated to the Fab through a linker comprising the structure:

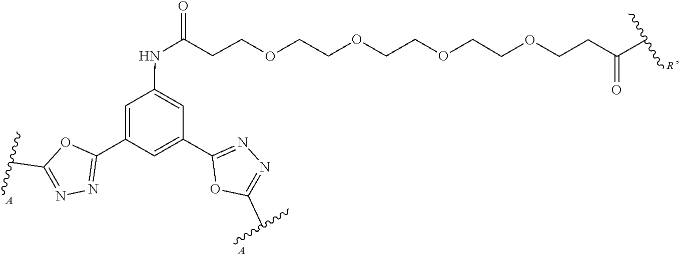

wherein [symbol] _{A} represents a point of attachment to the Fab, and [symbol] _{R} represents a point of attachment to the RNAi agent portion of the conjugate.

Embodiment 56. A composition comprising the RNAi agent of any one of claims 1-39, or the conjugate of any one of claims 40-55, wherein the composition further comprises a pharmaceutically acceptable excipient.

Embodiment 57. The composition of claim 56, further comprising a second RNAi agent capable of inhibiting the expression of HTT gene expression.

Embodiment 58. The composition of any one of claims 56-57, further comprising one or more additional therapeutics.

Embodiment 59. The composition of any one of claims 56-58, wherein the RNAi agent is a sodium salt.

Embodiment 60. The composition of any one of claims 56-59, wherein the pharmaceutically acceptable excipient is water for injection.

Embodiment 61. The composition of any one of claims 56-59, wherein the pharmaceutically acceptable excipient is a buffered saline solution.

Embodiment 62. A method for inhibiting expression of an HTT gene in a cell, the method comprising introducing into a cell an effective amount of an RNAi agent of any one of claims 1-39, the conjugate of any one of claims 40-55, or the composition of any one of claims 56-61.

Embodiment 63. The method of claim 62, wherein the cell is within a subject.

Embodiment 64. The method of claim 63, wherein the subject is a human subject.

Embodiment 65. The method of any one of claims 62-64, wherein following the administration of the RNAi agent the HTT gene expression is inhibited by at least about 30%.

Embodiment 66. A method of treating one or more symptoms or diseases associated with enhanced or elevated mutant HTT activity levels, the method comprising administering to a human subject in need thereof a therapeutically effective amount of the RNAi agent of any one of claims 1-39, the conjugate of any one of claims 40-55, or the composition of any one of claims 56-61.

Embodiment 67. The method of claim 66, wherein the disease is a neurodegenerative disease.

Embodiment 68. The method of claim 67, wherein the neurodegenerative disease is Huntington's Disease.

Embodiment 69. The method of claim 66, wherein the disease is Huntington's Disease.

Embodiment 70. The method of any one of claims 62-69, wherein the RNAi agent is administered at a dose of about 0.01 mg/kg to about 5.0 mg/kg of body weight of the subject.

Embodiment 71. The method of any one of claims 62-69, wherein the RNAi agent is administered at a dose of about 0.03 mg/kg to about 2.0 mg/kg of body weight of the subject.

Embodiment 72. The method of any one of claims 62-71, wherein the RNAi agent is administered in two or more doses.

Embodiment 73. Use of the RNAi agent of any one of claims 1-39, or the conjugate of any one of claims 40-55, for the treatment of a disease, disorder, or symptom that is mediated at least in part by mutant HTT activity and/or HTT gene expression.

Embodiment 74. Use of the composition according to any one of claims 56-61, for the treatment of a disease, disorder, or symptom that is mediated at least in part by HTT activity and/or HTT gene expression.

Embodiment 75. The use of any one of claims 72-74, wherein the disease is a neurodegenerative disease.

Embodiment 76. Use of the composition according to any one of claims 56-61, for the manufacture of a medicament for treatment of a disease, disorder, or symptom that is mediated at least in part by HTT activity and/or HTT gene expression.

Embodiment 77. The use of claim 76, wherein the neurodegenerative disease is Huntington's disease.

Embodiment 78. A method of making an RNAi agent of any one of claims 1-39, comprising annealing a sense strand and an antisense strand to form a double-stranded ribonucleic acid molecule.

### EXAMPLES

### Example 1. Synthesis of HTT RNAi Agents

HTT RNAi agent duplexes disclosed herein were synthesized in accordance with the following:

A. Synthesis. The sense and antisense strands of the HTT RNAi agents were synthesized according to phosphoramidite technology on solid phase used in oligonucleotide synthesis. Depending on the scale, a MerMade96E® (Bioautomation), a MerMadel2® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropylamino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-yanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEGS-NHS from BroadPharm (catalog #BP-20907) and coupled to the NH₂-C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)).

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

B. Cleavage and deprotection of support bound oligomer. After finalization of the solid phase synthesis. the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

C. Purfication. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

D. Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL-cm)) and the dilution factor to determine the duplex concentration.

### E. Synthesis of Antibody-siRNA Linkers

The synthesis of various Fab linkers used throughout the present application are provided below.

Synthesis of 2,3,5,6-tetrafluorophenyl 16-((3,5-bis(5-(methylsulfonyl)-1,3,4-oxadiazol-2-yl)phenyl)amino)-16-oxo-4,7,10,13-tetraoxahexadecanoate (i.e., I-1026-p)

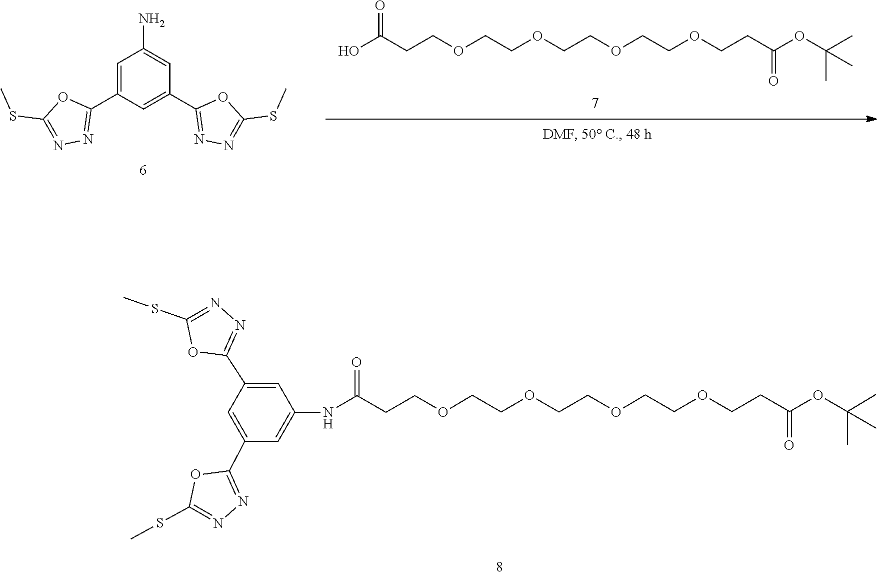

Compound 6 (2.35 g, 7.31 mmol; prepared according to Sarbisheh et al. Bioconjugate Chemistry 2020 31 (12), 2789-2806), EDC-HCl (2.38 g, 12.43 mmol), and K-Oxyma (2.50 g, 13.9 mmol) were combined as solids and slurried in DMF (190 mL) under N2 at ambient temperature. Compound 7 (1.92 g, 5.48 mmol) was added as a solution in DMF (10 mL). After 5 m, triethylamine (4.5 mL, 32.2 mmol) was added dropwise at ambient temperature. The reaction mixture was heated at 50° C. for 2 days. The reaction mixture was concentrated under reduced pressure to a red oil which was slurried in DCM (250 mL) and washed with sat. aq. sodium bicarbonate (200 mL). The layers were separated, and the aqueous layer was further extracted with DCM (100 mL). The combined organic phase was washed with water (200 mL) and brine (200 mL). The organic phase was dried over sodium sulfate, filtered, and concentrated. The residue was purified by normal phase SiO₂ chromatography with a gradient of ethyl acetate in DCM (0-100%). Yield of compound 8: 1.77 g (49%), partially contaminated with compound 6. Calculated mw for compound 8: 653.77 g/mol, found m/z (ESI, positive mode): 654.83.

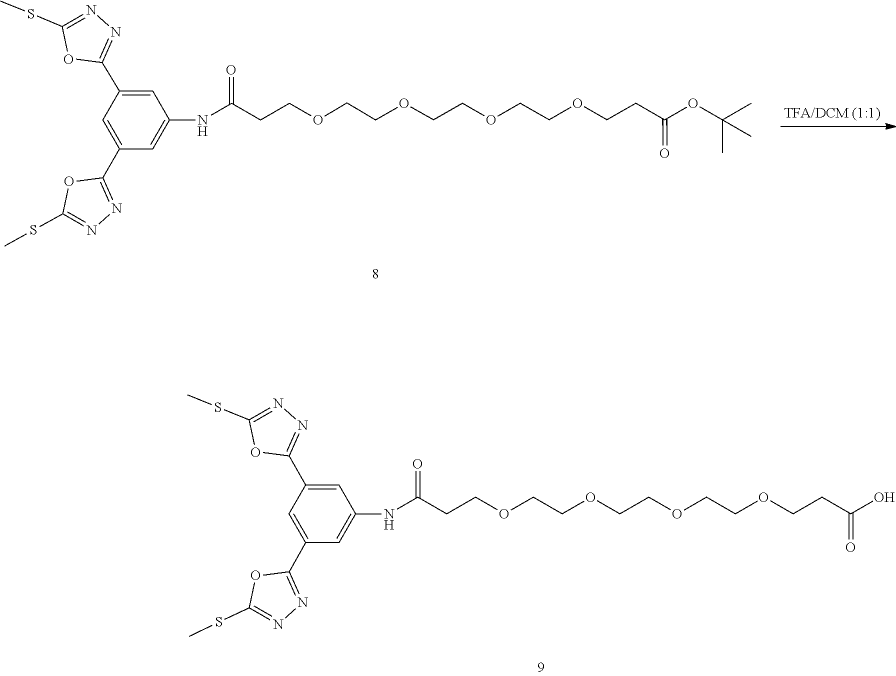

Compound 8 (1.77 g, 2.71 mmol) was dissolved in TFA:DCM [1:1] (18 mL) and stirred at ambient temperature for 1 hour. The reaction mixture was concentrated under reduced pressure then co-evaporated with toluene (3×30 mL). The residue was purified by normal phase SiO₂ chromatography with a gradient of DCM containing 0.1% formic acid and methanol (0-7%). Yield of compound 9: 1.30 g (80%). Calculated mw for compound 9: 597.66 g/mol, found m/z (ESI, positive mode): 598.79.

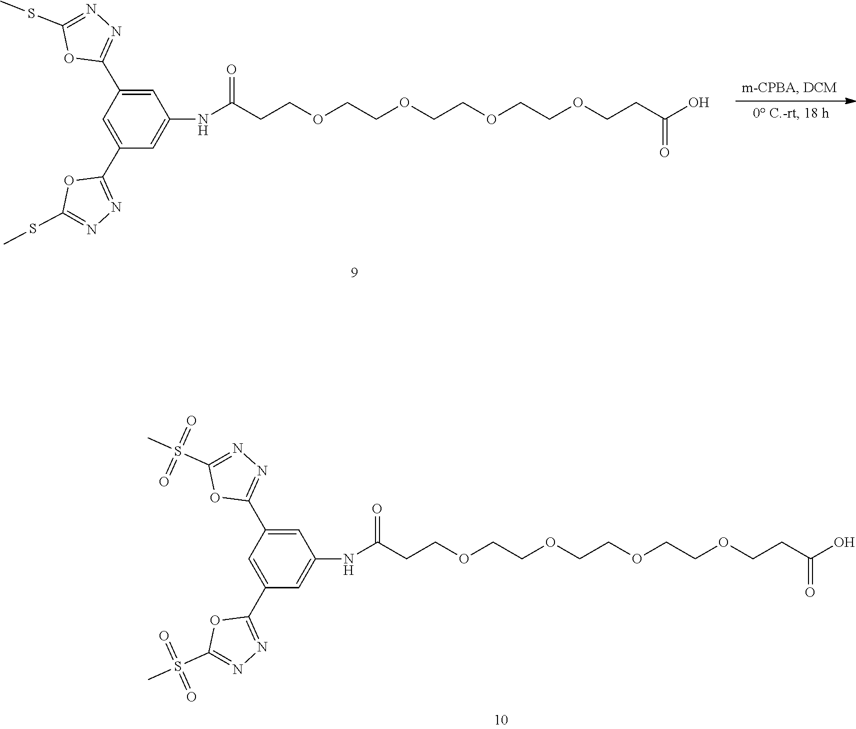 [SMILES: CSc1nnc(-c2cc(NC(=O)CCOCCOCCOCCOCCC(=O)O)cc(-c3nnc(SC)o3)c2)o1 ; CS(=O)(=O)c1nnc(-c2cc(NC(=O)CCOCCOCCOCCOCCC(=O)O)cc(-c3nnc(S(C)(=O)=O)o3)c2)o1]

Compound 9 (1.30 g, 2.18 mmol) was dissolved in DCM (50 mL) and cooled to 0° C. A 100 mg/mL solution of m-CPBA solution was prepared by dissolving 10.38 g m-CPBA (77 wt %) in 80 mL DCM and drying with sodium sulfate until clear. To the solution of compound 9 was added 58 mL m-CPBA (5.85 g, 26.1 mmol) dropwise at 0° C. The reaction mixture was warmed to ambient temperature and allowed to proceed overnight. The reaction mixture was concentrated, slurried in DCM 0.1% formic acid (50 mL), and filtered. The filtrate was purified by normal phase SiO₂ chromatography with a gradient of DCM containing 0.1% formic acid and methanol (0-10%). Yield of compound 10: 1.03 g (72%). Calculated mw for compound 10: 661.65 g/mol, found m/z (ESI, positive mode): 662.65.

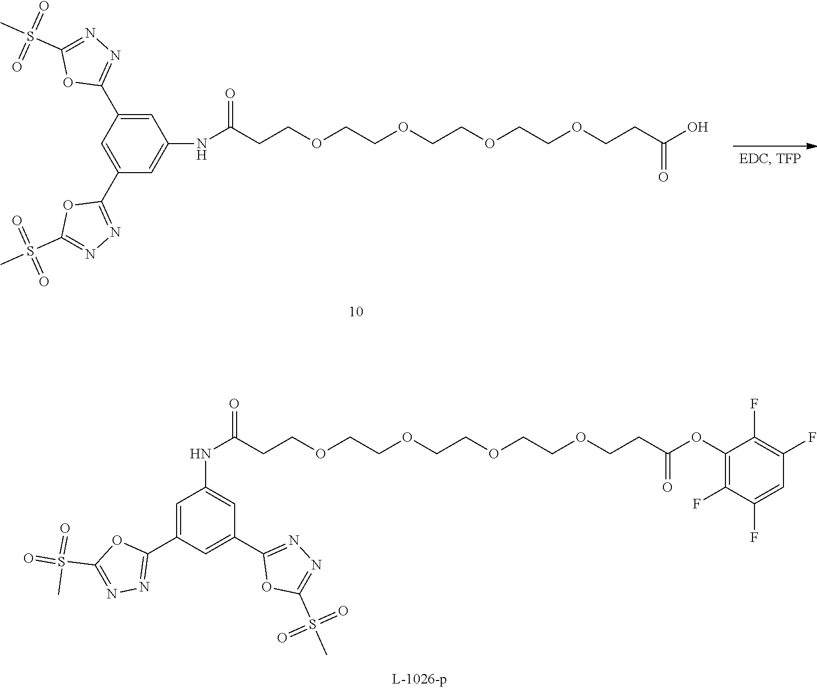 [SMILES: CS(=O)(=O)c1nnc(-c2cc(NC(=O)CCOCCOCCOCCOCCC(=O)O)cc(-c3nnc(S(C)(=O)=O)o3)c2)o1 ; CS(=O)(=O)c1nnc(-c2cc(NC(=O)CCOCCOCCOCCOCCC(=O)Oc3c(F)c(F)cc(F)c3F)cc(-c3nnc(S(C)(=O)=O)o3)c2)o1]

To a solution of compound 10 (1.03 g, 1.56 mmol) in DCM:ACN [4:1] (15 mL) at 0° C. was added EDC (0.448 g, 2.34 mmol) followed by a solution of TFP (0.310 g, 1.87 mmol) in DCM:ACN [4:1] (5 mL). After 5 m, the reaction mixture was warmed to ambient temperature. After 1.5 h, the reaction mixture was concentrated to dryness. The crude was purified by preparative reverse phase HPLC (Phenomenex Gemini C18 50 mm×250 mm, 10 um) using a gradient of water/acetonitrile containing 0.1% TFA. Product-containing fractions were concentrated under reduced pressure. Yield of L-1026-p: 1.10 g (87%). Calculated mw for compound L-1026-p: 809.71 g/mol, found m/z (ESI, positive mode): 810.62. ¹H NMR (400 MHz, [D₆]DMSO, 25° C.): δ=2.64 (t, 2H), 3.00 (t, 2H), 3.49 (m, 12H), 3.74 (m, 10H), 7.92 (m, 1H), 8.34 (t, 1H), 8.68 (d, 2H), 10.67 (s, 1H).

### Synthesis of L20-p

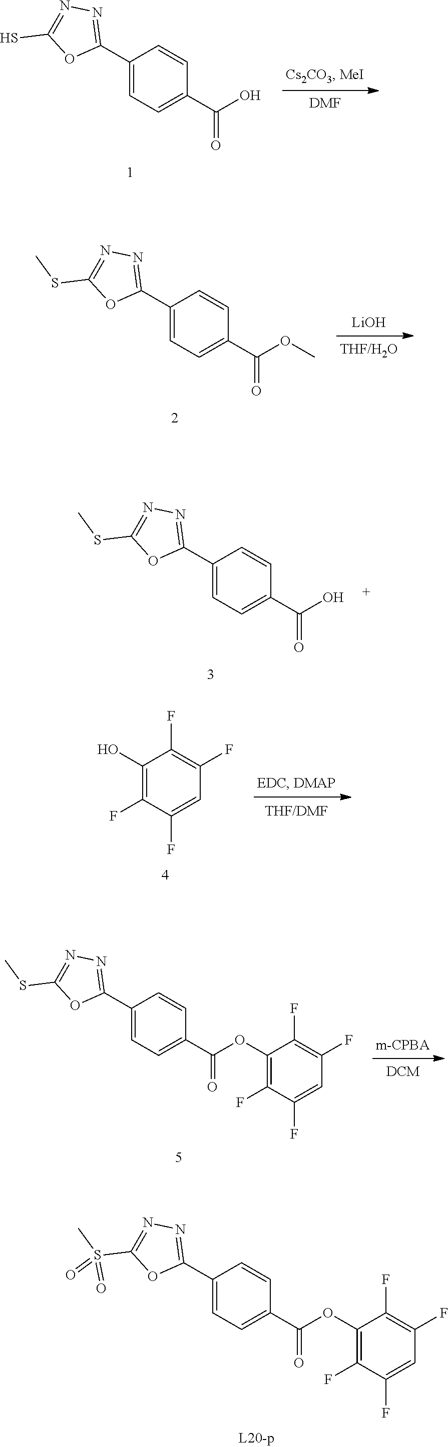

To a suspension of compound 1 (5.00 g, 22.50 mmol) and Cs₂CO₃ (25.66 g, 78.75 mmol) in anhydrous DMF (80 mL) was added methyl iodide (4.20 mL, 67.50 mmol) at room temperature. The reaction mixture was stirred at room temperature for 48 hours. The reaction mixture was quenched with water (200 mL) and the mixture was extracted with EtOAc (3×100 mL). The organic phase was combined and washed with water and brine. The organic layer was dried over anhydrous Na₂SO₄ and concentrated. Compound 2 was obtained as a light yellow solid, 5.41 g, 96%. Compound 2 was used directly without further purification. LC-MS: [M+H] calculated 251.05, found 251.18.

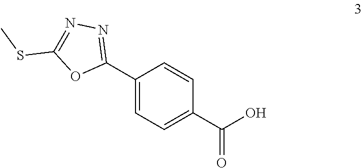 [SMILES: CSc1nnc(-c2ccc(C(=O)O)cc2)o1]

To a solution of compound 2 (5.41 g, 21.62 mmol) in THF/H2O (50 mL/50 mL) was added LiOH (2.59 g, 108.08 mmol) at room temperature. The reaction mixture was stirred at room temperature for 1 hour. After removing THE under vacuum, the pH was adjusted to ˜2 by [C] HCl. Then EtOAc (3×60 mL) was used to extract. The organic layers were combined, washed with brine, then dried over anhydrous Na₂SO₄, and concentrated. Compound 3 was obtained as an off-white solid, 5 g, 98%. Compound 3 was used directly without further purification. LC-MS: calculated [M+H] 237.03, found 237.26.

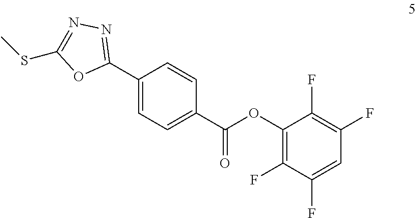 [SMILES: CSc1nnc(-c2ccc(C(=O)Oc3c(F)c(F)cc(F)c3F)cc2)o1]

To a solution of compound 3 (5.81 g, 24.60 mmol) in THF/DMF (80 mL/20 mL) was added EDC (7.07 g, 36.90 mmol), DMAP (0.30 g, 2.46 mmol) and compound 4 (6.13 g, 36.90 mmol) at room temperature. The reaction mixture was stirred at room temperature overnight. After removing solvent under vacuum, the residue was loaded on a 120 g column and compound 5 was eluted with 0-50% EtOAc in hexanes. Compound 5 was obtained as a white solid, 9.36 g, 99%. LC-MS: calculated [M+H] 385.03, found 385.46.

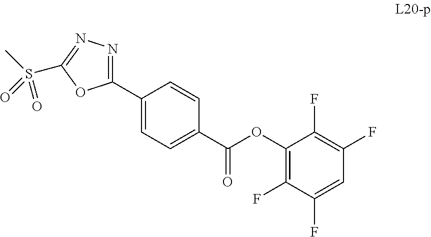 [SMILES: CS(=O)(=O)c1nnc(-c2ccc(C(=O)Oc3c(F)c(F)cc(F)c3F)cc2)o1]

To a solution of compound 5 (2.29 g, 5.96 mmol) in DCM (110 mL) was added 70% m-CPBA (5.14 g, 27.79 mmol) at 0° C. The reaction mixture was stirred at room temperature for 6 hours. Another 1.8 g m-CPBA was added at room temperature. The reaction mixture was stirred at room temperature overnight. After filtration, the solvent was removed under vacuum. The residue was recrystallized from DCM/EtOAc (50 mL/50 mL) twice. Compound L20-p was obtained as white needle crystals, 1.93 g, 78%. LC-MS: calculated [M+H] 417, found 417.

### F. Conjugation of RNAi Agents to Fabs.

RNAi agents described herein comprising a free amine were conjugated to L20-p

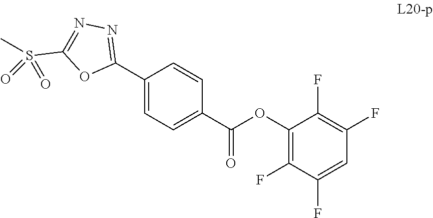 [SMILES: CS(=O)(=O)c1nnc(-c2ccc(C(=O)Oc3c(F)c(F)cc(F)c3F)cc2)o1]

using standard amide reaction chemistry following cleavage from the solid phase. To a solution of Fab in PBS (0.2 umol, 1.0-10.0 mg/mL in PBS) was added a freshly prepared solution of (tris(2-carboxyethyl)phosphine) hydrochloride (TCEP-HCl) in PBS (5-20 eq, 70 mM). The reaction was held overnight at room temperature and covered from light. The next day, TCEP was removed by loading the reaction mixture on a PD-10 desalting column equilibrated with PBS and eluted with PBS. The concentration of Fab in the eluate was determined using the theoretical absorptivity factor at 280 nm. A solution of L20-modified sense strand in sodium phosphate buffer was prepared, and the concentration was determined using the theoretical absorptivity factor at 260 nm. To the desalted Fab solution was added L20-modified sense strand (1-1.3 eq, 0.5-2.5 mM), and the reaction was mixed end-over-end. Analysis by SEC Method 1 and AIEX Method 1 show a mixture of starting Fab, DAR1, and DAR2. After 1 hour, a solution of CP-1113-p (see Table 10 for structure) in DMSO and added to the reaction mixture (3 eq, 36 mM). After 1 hour, a solution of L-cysteine in PBS was added to the reaction mixture (6-10 eq, 165 mM). Finally, the conjugate was annealed by addition of antisense strand (1.2-1.5 eq, 0.5-2.5 mM). The conjugate was purified by an AKTA Pure FPLC system equipped with 20 mM tris pH 8 (Buffer A), 20 mM tris 1500 mM NaCl (Buffer B), and a 5×200 mm column packed with Tosoh SuperQ 5PW (20 micron). The crude reaction mixture was pump loaded onto the column and eluted with a gradient of 10-40% Buffer B. DAR1 and DAR2 fractions were differentiated by SEC Method 1, AEX Method 1, and Nanodrop 260/280 readings. DAR1 fractions were pooled and buffer exchanged to PBS using a PD-10 desalting column. The purified conjugate was analyzed by SEC Method 1 and eluted as a monomeric peak with a retention time of 13.2 minutes.

### SEC Method 1

| | |
|---|---|
| Mobile phases | Phosphate Buffered Saline pH 7.4 |
| Column | Superdex 200 Increase 10/300 GL |
| | Cytiva PN 29219757 |
| Column | 25° C. |
| temperature | |
| Autosampler | ambient |
| Injection volume | 40 uL of 1 mg/mL protein (variable) |
| Flow rate | 1.0 mL/min isocratic |
| Wavelength | PDA 190-450 nm; monitor 230 nm, 260 nm, 280 nm |
| Run time | 30 minutes |

### AIEX Method 1

| Mobile phases | C: 20 mM tris pH 8.0, D: 20 mM tris 1500 | | |
|---|---|---|---|
| | mM NaCl pH 8.0 | | |
| Column | ProPac SAX-10 4 mm × 250 mm, 10 um | | |
| | Thermo Fisher Scientific PN 054997 | | |
| Column | 30° C. | | |
| temperature | | | |
| Autosampler | 5° C. | | |
| Injection volume | 20 μl of 0.2 mg/mL oligo (variable) | | |
| Flow rate | 1.0 mL/min (variable) | | |
| Wavelength | PDA 190-450 nm; monitor 230 nm, 260 nm, 280 nm | | |
| Run time | 12.5 minutes | | |
| | Time(min) | Event | Value |
| Gradient | 0 | D. Conc | 0 |
| | 0.10 | D. Conc | 0 |
| | 0.11 | D. Conc | 25 |
| | 10.11 | D. Conc | 75 |
| | 10.11 | T. Flow | 1 |
| | 10.12 | D. Conc | 0 |
| | 10.12 | T. Flow | 1.5 |
| | 12.50 | Controller | Stop |

RNAi agents described herein comprising a free amine were conjugated to L1026-p:

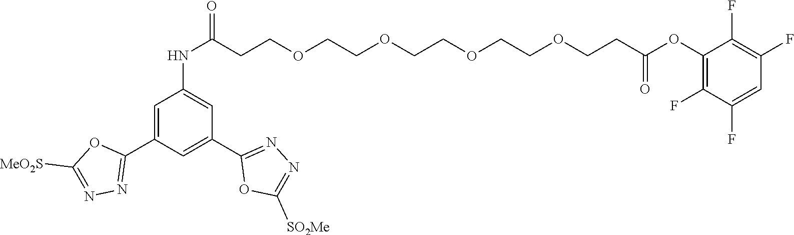 [SMILES: CS(=O)(=O)c1nnc(-c2cc(NC(=O)CCOCCOCCOCCOCCC(=O)Oc3c(F)c(F)cc(F)c3F)cc(-c3nnc(S(C)(=O)=O)o3)c2)o1]

following cleavage from the solid phase according to the following procedure:

To a solution of Fab0070 (28 mg, 0.59 umol, 5.55 mg/mL in PBS) was added a freshly prepared solution of TCEP-HCl in PBS (5 eq, 70 mM, 42 uL). The reduction was mixed end-over-end at ambient temperature for 15 minutes then held at 5° C. overnight without agitation. The next day, TCEP was removed by loading the reaction mixture on two PD-10 desalting columns (Cytiva) equilibrated with 20 mM tris 50 mM NaCl pH 7.6 (alternatively, 20 mM tris pH 8 or PBS buffer can be used) and eluted with the same buffer. The concentration of the Fab in the eluate was determined using the theoretical absorptivity factor at 280 nm. A solution of L-1026-modified sense strand (CS915332) in 10 mM sodium phosphate buffer pH 6.0-6.5 was prepared, and the concentration was determined using the theoretical absorptivity factor at 260 nm. To the desalted Fab solution was added L-1026-modified C5915332 (1.15 eq, 2.75 mM, 240 uL), and the reaction was mixed end-over-end at ambient temperature. Analysis by SEC Method 1 and ALEX Method 1 show a mixture of starting Fab0070, DAR1 product, and DAR2 product. After 30 m, a solution of L-cysteine in 20 mM tris 50 mM NaCl pH 7.6 (alternatively, some L-1026 conjugates have been prepared in 20 mM tris pH 8 or PBS buffer solutions) was added to the reaction mixture (10 eq, 165 mM, 36 uL). After 30 m, the conjugate was annealed by addition of antisense strand (CA003820) (1.3 eq, 1.45 mM in water, 529 uL). The conjugate was purified by an AKTA Pure FPLC system equipped with 20 mM tris pH 8 (Buffer A), 20 mM tris 1500 mM NaCl (Buffer B), and a 5×200 mm column packed with Tosoh SuperQ 5PW (20 micron). The crude reaction mixture was loaded onto the column and eluted with a gradient of 10-40% Buffer B. DAR1 and DAR2 fractions were differentiated by SEC Method 1, ALEX Method 1, and UV-Vis 260/280 measurements. DAR1 fractions were pooled and buffer exchanged to PBS using two PD-10 columns. The purified conjugate was analyzed by SEC Method 1 and eluted as a monomeric peak with a retention time of 7.2 minutes.

### SEC Method 1

| | |
|---|---|
| Mobile phases | 2x Phosphate Buffered Saline pH 7.4 |
| Column | ACQUITY UPLC Protein BEH SEC Column, 200 Å, |
| | 1.7 μm, 4.6 mm × 300 mm |
| | Waters PN 186005226 |
| Column | 30° C. |
| temperature | |
| Autosampler | ambient |
| Injection volume | 2-5 uL |
| Flow rate | 0.3 mL/min |
| Wavelength | PDA 190-450 nm |
| Run time | 20 minutes |

### AIEX Method 1

| Mobile phases | C: 20 mM tris pH 8.0, D: 20 mM tris 1500 | | |
|---|---|---|---|
| | mM NaCl pH 8.0 | | |
| Column | ProPac SAX-10 4 mm × 250 mm, 10 um | | |
| | Thermo Fisher Scientific PN 054997 | | |
| Column | 30° C. | | |
| temperature | | | |
| Autosampler | 5° C. | | |
| Injection volume | 5-20 μl | | |
| Flow rate | 1.0 mL/min (variable) | | |
| Wavelength | PDA 190-450 nm | | |
| Run time | 12.5 minutes | | |
| | Time(min) | Event | Value |
| Gradient | 0 | D. Conc | 10 |
| | 0.10 | D. Conc | 10 |
| | 0.11 | D. Conc | 25 |
| | 10.11 | D. Conc | 75 |
| | 10.11 | T. Flow | 1 |
| | 10.12 | D. Conc | 10 |
| | 10.12 | T. Flow | 1.5 |
| | 12.50 | Controller | Stop |

### Example 2. Synthesis of Lipids

### Synthesis of LP-183 Phosphoramidite

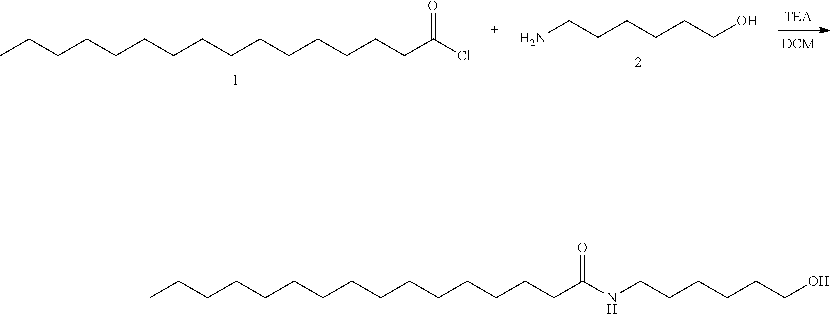 [SMILES: CCCCCCCCCCCCCCCC(=O)Cl ; NCCCCCCO ; CCCCCCCCCCCCCCCC(=O)NCCCCCCO]

To a solution of compound 2 (2.00 g) in DCM was added TEA (2.27 mL) followed by compound 1 (4.931 g) dropwise at room temperature. Then the mixture was stirred at room temperature for 2 h. The mixture was then filtered. The white solid was dried overnight. Product is as white solid, yield, 4.267 g, 74%. LC-MS: calculated [M+H] 356.35, found 356.63.

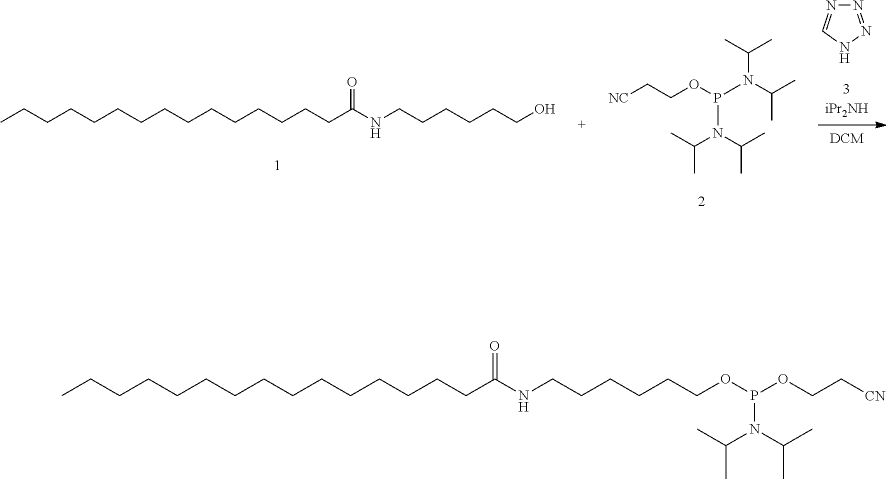 [SMILES: CCCCCCCCCCCCCCCC(=O)NCCCCCCO ; CC(C)N(C(C)C)P(OCCC#N)N(C(C)C)C(C)C ; c1nnn[nH]1 ; CCCCCCCCCCCCCCCC(=O)NCCCCCCOP(OCCC#N)N(C(C)C)C(C)C]

To a mixture of compound 1 (2.54 g) in 120 mL DCM was added compound 3 (0.61 g) followed by compound 2 (5.37 g) dropwise at room temperature. Then the mixture was stirred at room temperature overnight. 5 mL TEA was added followed by Celite. After removing solvent in vacuo, the residue was loaded on a 40 g column by dry method. Hexanes (2% TEA) to 50% EtOAc (2% TEA) in Hexanes (2% TEA) as gradient was used to purify the product. Product is a white waxy solid, yield 3.462 g, 87%. LC-MS: calculated [M+H] 556.46, found 556.64.

### Synthesis of LP-293 Phosphoramidite

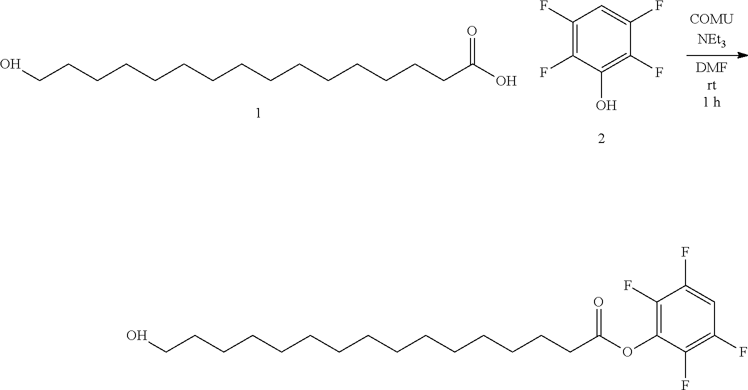

To a solution of compound 1 (73 mg), NEt₃ (0.112 mL), and COMU (126 mg) in DMF was added compound 2 (48.9 mg) under ambient conditions. The reaction was stirred until full conversion was observed by LC-MS. Conversion was not able to be clearly observed by LC-MS, and instead, reaction was allowed to stir for 30 min. until bright yellow color (before the addition of compound 2) transitioned to a honey orange color and all material was observed to be mainly dissolved. The reaction mixture was then washed with water, extracted with DCM, dried over Na₂SO₄, filtered, and concentrated under vacuum. The residue was purified by CombiFlash® via DCM liquid-load onto a 12-g column with a gradient hexanes to 100% EtOAc in which product eluted at 30% B. The product was concentrated under vacuum to provide a white solid residue and confirmed by 1H NMR in CDCl₃.

### Conjugation of Lipid PK/PD Modulator Precursors

Either prior to or after annealing, one or more lipid PK/PD modulator precursors can be linked to the RNAi agents disclosed herein. The following describes the general conjugation process used to link lipid PK/PD modulator precursors to the constructs set forth in the Examples depicted herein.

### A. Conjugation of Activated Ester PK/PD Modulators

The following procedure was used to conjugate PK/PD modulators having an activated ester moiety such as TFP (tetrafluorophenoxy) or PNP (para-nitrophenol) to an RNAi agent with an amine-functionalized sense strand, such as C6-NH2, NH2-C6, or (NH2-C6). An annealed RNAi Agent dried by lyophilization was dissolved in DMSO and 10% water (v/v %) at 25 mg/mL. Then 50-100 equivalents of TEA and 3 equivalents of activated ester PK/PD modulator were added to the solution. The solution was allowed to react for 1-2 hours, while monitored by RP-HPLC-MS (mobile phase A 100 mM HFIP, 14 mM TEA; mobile phase B: acetonitrile on an Waters™ XBridge C18 column, Waters Corp.)

The product was then precipitated by adding 12 mL acetonitrile and 0.4 mL PBS and centrifuging the solid to a pellet. The pellet was then re-dissolved in 0.4 mL of 1×PBS and 12 mL of acetonitrile. The resulting pellet was dried on high vacuum for one hour.

### B. Conjugation of Phosphoramidite PK/PD Modulators

PK/PD modulators having a phosphoramidite moiety may be attached on resin using typical oligonucleotide manufacturing conditions.

### C. Hydrolysis of PK/PD Modulators

Certain PK/PD modulators are hydrolyzed in the cleavage and deprotection conditions described in Example 1, above.

### Example 3. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 11.

FVB-Tg(YAC 128)53Hay/J mice (common name: “YAC 128” mice) express the human huntingtin protein.

TABLE 11
Dosing for mice of Example 3.

| Group ID | Dose (RNAi Agent) | # of Animals |
|---|---|---|
| 1. aCSF | Day 1: Single ICV Injection | n = 4 |
| 2. Naive | No Injection | n = 4 |
| 3. 0.1 mg AC911294 | Day 1: Single ICV Injection | n = 4 |
| 4. 0.1 mg AC911295 | Day 1: Single ICV Injection | n = 4 |
| 5. 0.1 mg AC911296 | Day 1: Single ICV Injection | n = 4 |
| 6. 0.1 mg AC911297 | Day 1: Single ICV Injection | n = 4 |
| 7. 0.1 mg AC911298 | Day 1: Single ICV Injection | n = 4 |
| 8. 0.1 mg AC911299 | Day 1: Single ICV Injection | n = 4 |
| 9. 0.1 mg AC911300 | Day 1: Single ICV Injection | n = 4 |
| 10. 0.1 mg AC911301 | Day 1: Single ICV Injection | n = 4 |
| 11. 0.1 mg AC911303 | Day 1: Single ICV Injection | n = 4 |
| 12. 0.1 mg AC911304 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis. hHTT expression was analyzed via qPCR in the cortex, thoracic spinal cord, and cerebellum, with mPPIA as endogenous gene, normalized to Group 1 mice dosed with aCSF. The hHTT expression data is shown in the following Table 12.

TABLE 12
HTT expression in mice brain tissues of Example 3.

| | Day 8 | | | | | | | | |
|---|---|---|---|---|---|---|---|---|---|
| | Cortex | | | Thoracic Spinal Cord | | | Cerebellum | | |
| | Rel Exp | Error | Error | Rel Exp | Error | Error | Rel Exp | Error | Error |
| Group ID | HTT | Low | High | HTT | Low | High | HTT | Low | High |
| 1. aCSF | 1.000 | 0.068 | 0.073 | 1.000 | 0.165 | 0.198 | 1.000 | 0.095 | 0.105 |
| 2. Naive | 1.157 | 0.105 | 0.116 | 0.833 | 0.160 | 0.198 | 1.167 | 0.141 | 0.160 |
| 3. 0.1 mg AC911294 | 1.013 | 0.132 | 0.151 | 0.989 | 0.100 | 0.111 | 1.085 | 0.090 | 0.098 |
| 4. 0.1 mg AC911295 | 0.984 | 0.073 | 0.079 | 1.000 | 0.108 | 0.122 | 1.318 | 0.153 | 0.172 |
| 5. 0.1 mg AC911296 | 0.649 | 0.125 | 0.154 | 0.668 | 0.048 | 0.052 | 0.912 | 0.041 | 0.043 |
| 6. 0.1 mg AC911297 | 0.703 | 0.071 | 0.079 | 0.869 | 0.085 | 0.095 | 1.124 | 0.191 | 0.231 |
| 7. 0.1 mg AC911298 | 0.662 | 0.062 | 0.068 | 0.757 | 0.080 | 0.089 | 1.167 | 0.116 | 0.129 |
| 8. 0.1 mg AC911299 | 0.771 | 0.111 | 0.130 | 0.708 | 0.052 | 0.056 | 1.257 | 0.221 | 0.269 |
| 9. 0.1 mg AC911300 | 0.477 | 0.078 | 0.093 | 0.642 | 0.068 | 0.076 | 1.107 | 0.149 | 0.172 |
| 10. 0.1 mg AC911301 | 0.750 | 0.078 | 0.087 | 0.884 | 0.142 | 0.169 | 1.359 | 0.175 | 0.201 |
| 11. 0.1 mg AC911303 | 0.509 | 0.051 | 0.056 | 0.544 | 0.103 | 0.127 | 1.105 | 0.159 | 0.185 |
| 12. 0.1 mg AC911304 | 0.775 | 0.150 | 0.185 | 0.549 | 0.036 | 0.038 | 1.317 | 0.204 | 0.241 |

In the cortex, Groups 5-12 showed reduction in HTT transcripts, while Groups 2-4 showed negligible to no reduction. In the thoracic spinal cord, Groups 5 and 7-12 showed reduction in HTT transcripts, while Groups 2-4 and 6 showed negligible to no reduction. In the cerebellum, all of the test Groups 2-12 showed negligible to no reduction in HTT transcripts. Most notably, a single dose 0.1 mg AC911300 achieved ˜52% HTT transcript inhibition (0.477) in the cortex.

### Example 4. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 13.

FVB-Tg(YAC 128)53Hay/J mice (common name: “YAC 128” mice) express the human huntingtin protein.

TABLE 13
Dosing for mice of Example 4.

| Group ID | Dose (RNAi Agent) | # of Animals |
|---|---|---|
| 1. aCSF | Day 1: Single ICV Injection | n = 4 |
| 2. 0.1 mg AC911227 | Day 1: Single ICV Injection | n = 4 |
| 3. 0.1 mg AC911302 | Day 1: Single ICV Injection | n = 4 |
| 4. 0.1 mg AC911305 | Day 1: Single ICV Injection | n = 4 |
| 5. 0.1 mg AC911306 | Day 1: Single ICV Injection | n = 4 |
| 6. 0.1 mg AC911307 | Day 1: Single ICV Injection | n = 4 |
| 7. 0.1 mg AC911308 | Day 1: Single ICV Injection | n = 4 |
| 8. 0.1 mg AC911309 | Day 1: Single ICV Injection | n = 4 |
| 9. 0.1 mg AC911310 | Day 1: Single ICV Injection | n = 4 |
| 10. 0.1 mg AC911311 | Day 1: Single ICV Injection | n = 4 |
| 11. 0.1 mg AC911312 | Day 1: Single ICV Injection | n = 4 |
| 12. 0.1 mg AC911313 | Day 1: Single ICV Injection | n = 4 |
| 13. 0.1 mg AC911314 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis. hHTT expression was analyzed via qPCR in the cortex, thoracic spinal cord, and cerebellum, with mPPIA as endogenous gene, normalized to Group 1 mice dosed with aCSF. The hHTT expression data is shown in the following Table 14.

TABLE 14
HTT expression in mice brain tissues of Example 4.

| | Day 8 | | | | | | | | |
|---|---|---|---|---|---|---|---|---|---|
| | Cortex | | | Thoracic Spinal Cord | | | Cerebellum | | |
| | Rel Exp | Error | Error | Rel Exp | Error | Error | Rel Exp | Error | Error |
| Group ID | HTT | Low | High | HTT | Low | High | HTT | Low | High |
| 1. aCSF | 1.000 | 0.227 | 0.293 | 1.000 | 0.151 | 0.178 | 1.000 | 0.238 | 0.313 |
| 2. 0.1 mg AC911227 | 0.805 | 0.186 | 0.242 | 0.760 | 0.139 | 0.171 | 0.562 | 0.105 | 0.129 |
| 3. 0.1 mg AC911302 | 0.879 | 0.102 | 0.115 | 0.982 | 0.129 | 0.148 | 0.722 | 0.105 | 0.123 |
| 4. 0.1 mg AC911305 | 0.834 | 0.141 | 0.170 | 0.817 | 0.123 | 0.144 | 0.760 | 0.260 | 0.395 |
| 5. 0.1 mg AC911306 | 0.805 | 0.165 | 0.208 | 0.947 | 0.093 | 0.103 | 0.557 | 0.120 | 0.152 |
| 6. 0.1 mg AC911307 | 0.918 | 0.124 | 0.143 | 0.862 | 0.091 | 0.102 | 0.506 | 0.152 | 0.216 |
| 7. 0.1 mg AC911308 | 0.522 | 0.152 | 0.214 | 0.561 | 0.078 | 0.091 | 0.595 | 0.073 | 0.083 |
| 8. 0.1 mg AC911309 | 0.815 | 0.125 | 0.147 | 0.915 | 0.131 | 0.154 | 0.662 | 0.114 | 0.138 |
| 9. 0.1 mg AC911310 | 0.515 | 0.067 | 0.078 | 0.713 | 0.081 | 0.092 | 0.789 | 0.105 | 0.121 |
| 10. 0.1 mg AC911311 | 0.492 | 0.026 | 0.028 | 0.631 | 0.093 | 0.109 | 0.547 | 0.068 | 0.077 |
| 11. 0.1 mg AC911312 | 0.554 | 0.119 | 0.152 | 0.680 | 0.074 | 0.083 | 0.559 | 0.099 | 0.121 |
| 12. 0.1 mg AC911313 | 0.566 | 0.180 | 0.263 | 0.753 | 0.162 | 0.207 | 0.847 | 0.233 | 0.321 |
| 13. 0.1 mg AC911314 | 0.685 | 0.149 | 0.190 | 0.790 | 0.118 | 0.138 | 0.775 | 0.216 | 0.300 |

In the cortex, Groups 7 and 9-13 showed reduction in HTT transcripts, while Groups 2-6 and 8 showed negligible to no reduction. In the thoracic spinal cord, Groups 2, 7, and 9-13 showed reduction in HTT transcripts, while Groups 3-6 and 8 showed negligible to no reduction. In the cerebellum, Groups 2-11 and 13 showed reduction in HTT transcripts, while Group 12 showed negligible to no reduction. Most notably, a single dose 0.1 mg AC911311 achieved ˜51% HTT transcript inhibition (0.492) in the cortex.

### Example 5. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 15.

FVB-Tg(YAC 128)53Hay/J mice (common name: “YAC 128” mice) express the human huntingtin protein.

TABLE 15
Dosing for mice of Example 5.

| Group ID | Dose (RNAi Agent) | # of Animals |
|---|---|---|
| 1. aCSF | Day 1: Single ICV Injection | n = 4 |
| 2. 0.1 mg AC911227 | Day 1: Single ICV Injection | n = 4 |
| 3. 0.1 mg AC911290 | Day 1: Single ICV Injection | n = 4 |
| 4. 0.1 mg AC911291 | Day 1: Single ICV Injection | n = 4 |
| 5. 0.1 mg AC911292 | Day 1: Single ICV Injection | n = 4 |
| 6. 0.1 mg AC911293 | Day 1: Single ICV Injection | n = 4 |
| 7. 0.1 mg AC911315 | Day 1: Single ICV Injection | n = 4 |
| 8. 0.1 mg AC911316 | Day 1: Single ICV Injection | n = 4 |
| 9. 0.1 mg AC911317 | Day 1: Single ICV Injection | n = 4 |
| 10. 0.1 mg AC911318 | Day 1: Single ICV Injection | n = 4 |
| 11. 0.1 mg AC911319 | Day 1: Single ICV Injection | n = 4 |
| 12. 0.1 mg AC911320 | Day 1: Single ICV Injection | n = 4 |
| 13. 0.1 mg AC911321 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis. hHTT expression was analyzed via qPCR in the cortex, thoracic spinal cord, and cerebellum, with mPPIA as endogenous gene, normalized to Group 1 mice dosed with aCSF. The hHTT expression data is shown in the following Table 16.

TABLE 16
HTT expression in mice brain tissues of Example 5.

| | Day 8 | | | | | | | | |
|---|---|---|---|---|---|---|---|---|---|
| | Cortex | | | Thoracic Spinal Cord | | | Cerebellum | | |
| | Rel Exp | Error | Error | Rel Exp | Error | Error | Rel Exp | Error | Error |
| Group ID | HTT | Low | High | HTT | Low | High | HTT | Low | High |
| 1. aCSF | 1.000 | 0.111 | 0.125 | 1.000 | 0.160 | 0.190 | 1.000 | 0.140 | 0.163 |
| 2. 0.1 mg AC911227 | 0.560 | 0.117 | 0.148 | 0.751 | 0.061 | 0.066 | 0.707 | 0.120 | 0.144 |
| 3. 0.1 mg AC911290 | 0.595 | 0.108 | 0.132 | 0.804 | 0.114 | 0.133 | 0.895 | 0.136 | 0.161 |
| 4. 0.1 mg AC911291 | 0.530 | 0.080 | 0.094 | 0.901 | 0.120 | 0.138 | 0.785 | 0.090 | 0.101 |
| 5. 0.1 mg AC911292 | 0.607 | 0.109 | 0.134 | 1.011 | 0.138 | 0.160 | 0.617 | 0.129 | 0.163 |
| 6. 0.1 mg AC911293 | 0.481 | 0.076 | 0.091 | 0.816 | 0.144 | 0.175 | 0.446 | 0.047 | 0.052 |
| 7. 0.1 mg AC911315 | 0.400 | 0.090 | 0.116 | 0.595 | 0.225 | 0.363 | 0.473 | 0.120 | 0.161 |
| 8. 0.1 mg AC911316 | 0.332 | 0.037 | 0.041 | 0.580 | 0.058 | 0.064 | 0.560 | 0.184 | 0.274 |
| 9. 0.1 mg AC911317 | 0.317 | 0.099 | 0.145 | 0.582 | 0.121 | 0.152 | 0.433 | 0.106 | 0.140 |
| 10. 0.1 mg AC911318 | 0.309 | 0.030 | 0.033 | 0.679 | 0.073 | 0.081 | 0.648 | 0.119 | 0.146 |
| 11. 0.1 mg AC911319 | 0.289 | 0.024 | 0.026 | 0.562 | 0.048 | 0.052 | 0.418 | 0.143 | 0.217 |
| 12. 0.1 mg AC911320 | 0.430 | 0.069 | 0.083 | 0.651 | 0.090 | 0.104 | 0.778 | 0.131 | 0.158 |
| 13. 0.1 mg AC911321 | 0.394 | 0.021 | 0.022 | 0.683 | 0.102 | 0.120 | 0.695 | 0.043 | 0.046 |

In the cortex, Groups 2-13 showed reduction in HTT transcripts. In the thoracic spinal cord, Groups 2 and 7-13 showed reduction in HTT transcripts, while Groups 3-6 showed negligible to no reduction. In the cerebellum, Groups 2 and 4-13 showed reduction in HTT transcripts, while Group 3 showed negligible to no reduction. Most notably, a single dose 0.1 mg AC911319 achieved ˜71% HTT transcript inhibition (0.289) in the cortex.

### Example 6. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 17.

FVB-Tg(YAC 128)53Hay/J mice (common name: “YAC 128” mice) express the human huntingtin protein.

TABLE 17
Dosing for mice of Example 6.

| Group ID | Dose (RNAi Agent) | # of Animals |
|---|---|---|
| 1. aCSF | Day 1: Single ICV Injection | n = 4 |
| 2. 0.1 mg AC911227 | Day 1: Single ICV Injection | n = 4 |
| 3. 0.1 mg AC911290 | Day 1: Single ICV Injection | n = 4 |
| 4. 0.1 mg AC911291 | Day 1: Single ICV Injection | n = 4 |
| 5. 0.1 mg AC911292 | Day 1: Single ICV Injection | n = 4 |
| 6. 0.1 mg AC911293 | Day 1: Single ICV Injection | n = 4 |
| 7. 0.1 mg AC911300 | Day 1: Single ICV Injection | n = 4 |
| 8. 0.1 mg AC911303 | Day 1: Single ICV Injection | n = 4 |
| 9. 0.1 mg AC911308 | Day 1: Single ICV Injection | n = 4 |
| 10. 0.1 mg AC911311 | Day 1: Single ICV Injection | n = 4 |
| 11. 0.1 mg AC911312 | Day 1: Single ICV Injection | n = 4 |
| 12. 0.1 mg AC911315 | Day 1: Single ICV Injection | n = 4 |
| 13. 0.1 mg AC911316 | Day 1: Single ICV Injection | n = 4 |
| 14. 0.1 mg AC911317 | Day 1: Single ICV Injection | n = 4 |
| 15. 0.1 mg AC911318 | Day 1: Single ICV Injection | n = 4 |
| 16. 0.1 mg AC911319 | Day 1: Single ICV Injection | n = 4 |
| 17. 0.1 mg AC911320 | Day 1: Single ICV Injection | n = 4 |
| 18. 0.1 mg AC911599 | Day 1: Single ICV Injection | n = 4 |
| 19. 0.1 mg AC911600 | Day 1: Single ICV Injection | n = 4 |
| 20. 0.1 mg AC911601 | Day 1: Single ICV Injection | n = 4 |
| 21. 0.1 mg AC911602 | Day 1: Single ICV Injection | n = 4 |
| 22. 0.1 mg AC911603 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis. hHTT expression was analyzed via qPCR in the cortex, thoracic spinal cord, cerebellum, and striatum, with mPPIA as endogenous gene, normalized to Group 1 mice dosed with aCSF. The hHTT expression data is shown in the following Table 18.

TABLE 18
HTT expression in mice brain tissues of Example 6.

| | Day 8 | | | | | |
|---|---|---|---|---|---|---|
| | Cortex | | | Thoracic Spinal Cord | | |
| | Rel Exp | Error | Error | Rel Exp | Error | Error |
| Group ID | HTT | Low | High | HTT | Low | High |
| 1. aCSF | 1.000 | 0.092 | 0.101 | 1.000 | 0.151 | 0.178 |
| 2. 0.1 mg AC911227 | 0.539 | 0.108 | 0.135 | 0.849 | 0.070 | 0.076 |
| 3. 0.1 mg AC911290 | 0.905 | 0.160 | 0.194 | 1.080 | 0.152 | 0.178 |
| 4. 0.1 mg AC911291 | 0.683 | 0.041 | 0.043 | 1.134 | 0.132 | 0.149 |
| 5. 0.1 mg AC911292 | 0.613 | 0.066 | 0.075 | 1.052 | 0.174 | 0.209 |
| 6. 0.1 mg AC911293 | 0.688 | 0.147 | 0.188 | 0.009 | 0.006 | 0.014 |
| 7. 0.1 mg AC911300 | 0.419 | 0.044 | 0.050 | 0.602 | 0.054 | 0.060 |
| 8. 0.1 mg AC911303 | 0.436 | 0.034 | 0.037 | 0.877 | 0.135 | 0.160 |
| 9. 0.1 mg AC911308 | 0.418 | 0.041 | 0.045 | 0.642 | 0.046 | 0.049 |
| 10. 0.1 mg AC911311 | 0.402 | 0.031 | 0.033 | 0.637 | 0.040 | 0.042 |
| 11. 0.1 mg AC911312 | 0.473 | 0.062 | 0.071 | 0.744 | 0.087 | 0.099 |
| 12. 0.1 mg AC911315 | 0.495 | 0.090 | 0.110 | 0.519 | 0.220 | 0.380 |
| 13. 0.1 mg AC911316 | 0.490 | 0.082 | 0.098 | 0.653 | 0.138 | 0.175 |
| 14. 0.1 mg AC911317 | 0.380 | 0.091 | 0.119 | 0.720 | 0.037 | 0.039 |
| 15. 0.1 mg AC911318 | 0.510 | 0.092 | 0.112 | 0.792 | 0.031 | 0.033 |
| 16. 0.1 mg AC911319 | 0.461 | 0.064 | 0.074 | 0.586 | 0.067 | 0.076 |
| 17. 0.1 mg AC911320 | 0.580 | 0.023 | 0.024 | 0.825 | 0.031 | 0.032 |
| 18. 0.1 mg AC911599 | 0.608 | 0.033 | 0.035 | 0.774 | 0.092 | 0.105 |
| 19. 0.1 mg AC911600 | 0.661 | 0.031 | 0.032 | 1.022 | 0.072 | 0.078 |
| 20. 0.1 mg AC911601 | 0.578 | 0.052 | 0.058 | 0.940 | 0.104 | 0.117 |
| 21. 0.1 mg AC911602 | 0.892 | 0.109 | 0.124 | 1.070 | 0.176 | 0.211 |
| 22. 0.1 mg AC911603 | 0.729 | 0.044 | 0.047 | 0.963 | 0.074 | 0.081 |
| | Cerebellum | | | Striatum | | |
| | Rel Exp | Error | Error | Rel Exp | Error | Error |
| Group ID | HTT | Low | High | HTT | Low | High |
| 1. aCSF | 1.000 | 0.148 | 0.174 | 1.000 | 0.178 | 0.216 |
| 2. 0.1 mg AC911227 | 0.674 | 0.064 | 0.070 | 0.662 | 0.094 | 0.109 |
| 3. 0.1 mg AC911290 | 0.887 | 0.154 | 0.187 | 0.787 | 0.143 | 0.174 |
| 4. 0.1 mg AC911291 | 0.859 | 0.103 | 0.118 | 0.727 | 0.124 | 0.150 |
| 5. 0.1 mg AC911292 | 0.861 | 0.162 | 0.200 | 0.721 | 0.055 | 0.060 |
| 6. 0.1 mg AC911293 | 0.882 | 0.174 | 0.217 | 0.548 | 0.163 | 0.232 |
| 7. 0.1 mg AC911300 | 0.499 | 0.109 | 0.139 | 0.534 | 0.069 | 0.080 |
| 8. 0.1 mg AC911303 | 0.449 | 0.092 | 0.116 | 0.443 | 0.068 | 0.080 |
| 9. 0.1 mg AC911308 | 0.964 | 0.105 | 0.117 | 0.507 | 0.062 | 0.070 |
| 10. 0.1 mg AC911311 | 0.510 | 0.103 | 0.130 | 0.560 | 0.113 | 0.142 |
| 11. 0.1 mg AC911312 | 0.736 | 0.173 | 0.225 | 0.548 | 0.144 | 0.195 |
| 12. 0.1 mg AC911315 | 0.686 | 0.109 | 0.129 | 0.576 | 0.134 | 0.175 |
| 13. 0.1 mg AC911316 | 1.090 | 0.178 | 0.213 | 0.672 | 0.042 | 0.044 |
| 14. 0.1 mg AC911317 | 0.617 | 0.171 | 0.236 | 0.545 | 0.071 | 0.081 |
| 15. 0.1 mg AC911318 | 0.643 | 0.154 | 0.203 | 0.629 | 0.092 | 0.108 |
| 16. 0.1 mg AC911319 | 0.590 | 0.104 | 0.126 | 0.611 | 0.047 | 0.051 |
| 17. 0.1 mg AC911320 | 0.929 | 0.227 | 0.301 | 0.654 | 0.118 | 0.144 |
| 18. 0.1 mg AC911599 | 0.665 | 0.098 | 0.115 | 0.794 | 0.105 | 0.121 |
| 19. 0.1 mg AC911600 | 0.783 | 0.154 | 0.192 | 0.652 | 0.075 | 0.085 |
| 20. 0.1 mg AC911601 | 0.774 | 0.176 | 0.228 | 0.811 | 0.070 | 0.076 |
| 21. 0.1 mg AC911602 | 0.894 | 0.197 | 0.253 | 0.881 | 0.082 | 0.091 |
| 22. 0.1 mg AC911603 | 0.698 | 0.116 | 0.138 | 0.791 | 0.071 | 0.078 |

In the cortex, Groups 2, 4-20, and 22 showed reduction in HTT transcripts, while Group 3 and 21 showed negligible to no reduction. In the thoracic spinal cord, Groups 6, 7, 9-16, and 18 showed reduction in HTT transcripts, while Groups 2-5, 8, 17, and 19-22 showed negligible to no reduction. In the cerebellum, Groups 2, 7, 8, 10-12, 14-16, 18-20, and 22 showed reduction in HTT transcripts, while Groups 3-6, 9, 13, 17, and 21 showed negligible to no reduction. In the striatum, Groups 2-19 and 22 showed reduction in HTT transcripts, while Groups 20 and 21 showed negligible to no reduction. Most notably, a single dose 0.1 mg AC911293 achieved ˜99% HTT transcript inhibition (0.009) in the thoracic spinal cord.

### Example 7. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 19.

FVB-Tg(YAC 128)53Hay/J mice (common name: “YAC 128” mice) express the human huntingtin protein.

TABLE 19
Dosing for mice of Example 7.

| Group ID | Dose (RNAi Agent) | # of Animals |
|---|---|---|
| 1. aCSF | Day 1: Single ICV Injection | n = 4 |
| 2. 0.1 mg AC911293 | Day 1: Single ICV Injection | n = 4 |
| 3. 0.1 mg AC911300 | Day 1: Single ICV Injection | n = 4 |
| 4. 0.1 mg AC911308 | Day 1: Single ICV Injection | n = 4 |
| 5. 0.1 mg AC911317 | Day 1: Single ICV Injection | n = 4 |
| 6. 0.1 mg AC911303 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis.

hHTT expression was analyzed via qPCR in the cortex, thoracic spinal cord, and striatum, with mPPIA as endogenous gene, normalized to Group 1 mice dosed with aCSF. The hHTT expression data is shown in the following Table 20.

TABLE 20
HTT expression in mice brain tissues of Example 7.

| | Day 8 | | | | | | | | |
|---|---|---|---|---|---|---|---|---|---|
| | Cortex | | | Thoracic Spinal Cord | | | Striatum | | |
| | Rel Exp | Error | Error | Rel Exp | Error | Error | Rel Exp | Error | Error |
| Group ID | HTT | Low | High | HTT | Low | High | HTT | Low | High |
| 1. aCSF | 1.000 | 0.152 | 0.179 | 1.000 | 0.234 | 0.306 | 1.000 | 0.199 | 0.248 |
| 2. 0.1 mg AC911293 | 0.883 | 0.236 | 0.323 | 1.234 | 0.244 | 0.303 | 0.742 | 0.085 | 0.097 |
| 3. 0.1 mg AC911300 | 0.576 | 0.103 | 0.125 | 0.943 | 0.094 | 0.104 | 0.789 | 0.059 | 0.064 |
| 4. 0.1 mg AC911308 | 0.670 | 0.118 | 0.144 | 0.882 | 0.076 | 0.083 | 0.669 | 0.063 | 0.069 |
| 5. 0.1 mg AC911317 | 0.432 | 0.104 | 0.137 | 0.841 | 0.072 | 0.079 | 0.590 | 0.082 | 0.096 |
| 6. 0.1 mg AC911303 | 0.763 | 0.126 | 0.151 | 0.814 | 0.055 | 0.060 | 0.715 | 0.187 | 0.254 |

In the cortex, Groups 3-6 showed reduction in HTT transcripts, while Group 2 showed negligible to no reduction. In the thoracic spinal cord, Groups 2-6 showed negligible to no reduction in HTT transcripts. In the striatum, Groups 2-6 showed reduction in HTT transcripts. Most notably, a single dose 0.1 mg AC911317 achieved ˜57% HTT transcript inhibition (0.432) in the cortex.

HTT protein expression was analyzed via Jess assay in the cortex and thoracic spinal cord, with vinculin as endogenous control protein, normalized to Group 1 mice dosed with aCSF. The hHTT expression data is shown in the following Table 21.

TABLE 21
HTT protein expression in mice brain tissues of Example 7.

| | Day 8 | | | |
|---|---|---|---|---|
| | Cortex | | Thoracic Spinal Cord | |
| | Rel Exp | Std | Rel Exp | Std |
| Group ID | HTT | Dev+/− | HTT | Dev+/− |
| 1. aCSF | 1.114 | 0.239 | 1.287 | 0.620 |
| 2. 0.1 mg AC911293 | 1.322 | 0.239 | 1.426 | 0.465 |
| 3. 0.1 mg AC911300 | 0.655 | 0.215 | 1.144 | 0.797 |
| 4. 0.1 mg AC911308 | 0.723 | 0.104 | 1.270 | 0.839 |
| 5. 0.1 mg AC911317 | 0.459 | 0.193 | 0.522 | 0.306 |
| 6. 0.1 mg AC911303 | 0.536 | 0.038 | 1.311 | 0.705 |

In the cortex, Groups 3-6 showed reduction in HTT protein, while Group 2 showed negligible to no reduction. In the thoracic spinal cord, Group 5 showed reduction in HTT protein, while Groups 2-4 and 6 showed negligible to no reduction. Most notably, a single dose 0.1 mg AC911317 achieved ˜54% HTT protein inhibition (0.459) in the cortex.

### Example 8. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 22.

FVB-Tg(YAC 128)53Hay/J mice (common name: “YAC 128” mice) express the human huntingtin protein.

TABLE 22
Dosing for mice of Example 8.

| Group ID | Dose (RNAi Agent) | # of Animals |
|---|---|---|
| 1. aCSF | Day 1: Single ICV Injection | n = 4 |
| 2. 0.1 mg AC911311 | Day 1: Single ICV Injection | n = 4 |
| 3. 0.1 mg AC911312 | Day 1: Single ICV Injection | n = 4 |
| 4. 0.1 mg AC911313 | Day 1: Single ICV Injection | n = 4 |
| 5. 0.1 mg AC911310 | Day 1: Single ICV Injection | n = 4 |
| 6. 0.1 mg AC911315 | Day 1: Single ICV Injection | n = 4 |
| 7. 0.1 mg AC911321 | Day 1: Single ICV Injection | n = 4 |
| 8. 0.1 mg AC911316 | Day 1: Single ICV Injection | n = 4 |
| 9. 0.1 mg AC911319 | Day 1: Single ICV Injection | n = 4 |
| 10. 0.1 mg AC911300 | Day 1: Single ICV Injection | n = 4 |
| 11. 0.1 mg AC911317 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 8, the mice were euthanized. From the mice, right half of the brain and thoracic spinal cord were harvested and collected for analysis.

hHTT expression was analyzed via qPCR in the cortex, thoracic spinal cord, and cerebellum, with mPPIA as endogenous gene, normalized to Group 1 mice dosed with aCSF. The hHTT expression data is shown in the following Table 23.

TABLE 23
HTT expression in mice brain tissues of Example 8.

| | Day 8 | | | | | | | | |
|---|---|---|---|---|---|---|---|---|---|
| | Cortex | | | Thoracic Spinal Cord | | | Cerebellum | | |
| | Rel Exp | Error | Error | Rel Exp | Error | Error | Rel Exp | Error | Error |
| Group ID | HTT | Low | High | HTT | Low | High | HTT | Low | High |
| 1. aCSF | 1.000 | 0.136 | 0.158 | 1.000 | 0.087 | 0.096 | 1.000 | 0.154 | 0.182 |
| 2. 0.1 mg AC911311 | 0.594 | 0.072 | 0.081 | 0.857 | 0.041 | 0.043 | 0.980 | 0.109 | 0.123 |
| 3. 0.1 mg AC911312 | 0.685 | 0.075 | 0.084 | 1.005 | 0.055 | 0.058 | 0.920 | 0.099 | 0.110 |
| 4. 0.1 mg AC911313 | 0.714 | 0.080 | 0.090 | 0.914 | 0.085 | 0.093 | 1.058 | 0.092 | 0.101 |
| 5. 0.1 mg AC911310 | 0.557 | 0.063 | 0.071 | 0.751 | 0.041 | 0.043 | 0.936 | 0.081 | 0.089 |
| 6. 0.1 mg AC911315 | 0.593 | 0.025 | 0.026 | 0.853 | 0.063 | 0.068 | 0.946 | 0.061 | 0.065 |
| 7. 0.1 mg AC911321 | 0.410 | 0.027 | 0.029 | 0.678 | 0.068 | 0.075 | 0.961 | 0.070 | 0.076 |
| 8. 0.1 mg AC911316 | 0.437 | 0.042 | 0.047 | 0.719 | 0.037 | 0.039 | 0.988 | 0.073 | 0.079 |
| 9. 0.1 mg AC911319 | 0.317 | 0.039 | 0.044 | 0.595 | 0.082 | 0.095 | 0.896 | 0.098 | 0.110 |
| 10. 0.1 mg AC911300 | 0.413 | 0.039 | 0.043 | 0.710 | 0.036 | 0.038 | 1.035 | 0.089 | 0.097 |
| 11. 0.1 mg AC911317 | 0.358 | 0.052 | 0.061 | 0.711 | 0.069 | 0.076 | 0.749 | 0.245 | 0.364 |

In the cortex, Groups 2-11 showed reduction in HTT transcripts. In the thoracic spinal cord, Groups 5 and 7-11 showed reduction in HTT transcripts, while Groups 2-4 and 6 showed negligible to no reduction. In the cerebellum, Group 11 showed reduction in HTT transcripts, while Groups 2-10 showed negligible to no reduction. Most notably, a single dose 0.1 mg AC911319 achieved ˜68% HTT transcript inhibition (0.317) in the cortex.

HTT protein expression was analyzed via bicinchoninic acid (BCA) assay in the cortex, normalized to Group 1 mice dosed with aCSF. The hHTT expression data is shown in the following Table 24.

TABLE 24
HTT protein expression in mice brain tissues of Example 8.

Day 8
Cortex

| Group ID | Rel Exp HTT | Std Dev+/− |
|---|---|---|
| 1. aCSF | 1.000 | 0.115 |
| 2. 0.1 mg AC911311 | 0.475 | 0.101 |
| 3. 0.1 mg AC911312 | 0.756 | 0.053 |
| 4. 0.1 mg AC911313 | 0.696 | 0.056 |
| 5. 0.1 mg AC911310 | 0.653 | 0.109 |
| 6. 0.1 mg AC911315 | 0.633 | 0.086 |
| 7. 0.1 mg AC911321 | 0.683 | 0.073 |
| 8. 0.1 mg AC911316 | 0.649 | 0.074 |
| 9. 0.1 mg AC911319 | 0.517 | 0.039 |
| 10. 0.1 mg AC911300 | 0.598 | 0.067 |
| 11. 0.1 mg AC911317 | 0.545 | 0.124 |

In the cortex, Groups 2-11 showed reduction in HTT protein. Most notably, a single dose 0.1 mg AC911311 achieved ˜52% HTT protein inhibition (0.475) in the cortex.

### Example 9. In Vivo Administration of HTT RNAi Agents in YAC128 Mice

HTT RNAi agents were evaluated in vivo in mice. On Days 1, 4, 7, and 10, four (n=4) FVB-Tg(YAC128)53Hay/J mice (2 male, 2 female) for each group were dosed, via intravenous (IV) injection, with HTT RNAi agents formulated in saline (at 1.5 mg/kg) or with PBS. The HTT RNAi agents were formulated at 200 μL/20 g animal body weight. The dosing was in accordance with the following Table 25.

FVB-Tg(YAC 128)53Hay/J mice (common name: “YAC 128” mice) express the human huntingtin protein.

TABLE 25
Dosing for mice of Example 9.

| Group ID | Dose (RNAi Agent) | # of Animals |
|---|---|---|
| 1. PBS | Days 1, 4, 7, 10: 4x Single | n = 4 |
| | IV Injections | |
| 2. 1.5 mg/kg AC004497 | Days 1, 4, 7, 10: 4x Single | n = 4 |
| | IV Injections | |
| 3. 1.5 mg/kg AC004498 | Days 1, 4, 7, 10: 4x Single | n = 4 |
| | IV Injections | |
| 4. 1.5 mg/kg AC004499 | Days 1, 4, 7, 10: 4x Single | n = 4 |
| | IV Injections | |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 22, the mice were euthanized. From the mice, right half of the brain and thoracic spinal cord were harvested and collected for analysis.

hHTT expression was analyzed via qPCR in the cortex, thoracic spinal cord, cerebellum, and striatum, with mPPIA as endogenous gene, normalized to Group 1 mice dosed with PBS. The hHTT expression data is shown in the following Table 26.

TABLE 26
HTT expression in mice brain tissues of Example 9.

| | Day 22 | | | | | |
|---|---|---|---|---|---|---|
| | Cortex | | | Thoracic Spinal Cord | | |
| | Rel Exp | Error | Error | Rel Exp | Error | Error |
| Group ID | HTT | Low | High | HTT | Low | High |
| 1. PBS | 1.000 | 0.165 | 0.198 | 1.000 | 0.098 | 0.109 |
| 2. 1.5 mg/kg AC004497 | 0.748 | 0.249 | 0.374 | 0.639 | 0.213 | 0.319 |
| 3. 1.5 mg/kg AC004498 | 0.905 | 0.110 | 0.125 | 0.810 | 0.109 | 0.126 |
| 4. 1.5 mg/kg AC004499 | 0.653 | 0.118 | 0.144 | 0.708 | 0.078 | 0.088 |
| | Cerebellum | | | Striatum | | |
| | Rel Exp | Error | Error | Rel Exp | Error | Error |
| Group ID | HTT | Low | High | HTT | Low | High |
| 1. PBS | 1.000 | 0.093 | 0.102 | 1.000 | 0.129 | 0.148 |
| 2. 1.5 mg/kg AC004497 | 0.785 | 0.246 | 0.359 | 0.786 | 0.233 | 0.330 |
| 3. 1.5 mg/kg AC004498 | 0.661 | 0.094 | 0.110 | 1.062 | 0.122 | 0.138 |
| 4. 1.5 mg/kg AC004499 | 0.817 | 0.139 | 0.167 | 0.898 | 0.070 | 0.076 |

In the cortex, Groups 2 and 4 showed reduction in HTT transcripts, while Group 3 showed negligible to no reduction. In the thoracic spinal cord, Groups 2 and 4 showed reduction in HTT transcripts, while Group 3 showed negligible to no reduction. In the cerebellum, Groups 2 and 3 showed reduction in HTT transcripts, while Group 4 showed negligible to no reduction. In the striatum, Group 2 showed reduction in HTT transcripts, while Groups 3 and 4 showed negligible to no reduction. Most notably, 4× doses 1.5 mg/kg AC004497 achieved ˜36% HTT transcript inhibition (0.639) in the thoracic spinal cord.

### Example 10. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.3 mg total HTT RNAi agent) or with aCSF. The HTT RNAi agents were formulated at 30 mg/mL at 10 μL total injection volume. The dosing was in accordance with the following Table 27.

FVB-Tg(YAC 128)53Hay/J mice (common name: “YAC 128” mice) express the human huntingtin protein.

TABLE 27
Dosing for mice of Example 10.

| Group ID | Dose (RNAi Agent) | # of Animals |
|---|---|---|
| 1. aCSF | Day 1: Single ICV Injection | n = 4 |
| 2. 0.3 mg AC911290 | Day 1: Single ICV Injection | n = 4 |
| 3. 0.3 mg AC911291 | Day 1: Single ICV Injection | n = 4 |
| 4. 0.3 mg AC911292 | Day 1: Single ICV Injection | n = 4 |
| 5. 0.3 mg AC911293 | Day 1: Single ICV Injection | n = 4 |
| 6. 0.3 mg AC911294 | Day 1: Single ICV Injection | n = 4 |
| 7. 0.3 mg AC911295 | Day 1: Single ICV Injection | n = 4 |
| 8. 0.3 mg AC911599 | Day 1: Single ICV Injection | n = 4 |
| 9. 0.3 mg AC911600 | Day 1: Single ICV Injection | n = 4 |
| 10. 0.3 mg AC911601 | Day 1: Single ICV Injection | n = 4 |
| 11. 0.3 mg AC911602 | Day 1: Single ICV Injection | n = 4 |
| 12. 0.3 mg AC911603 | Day 1: Single ICV Injection | n = 4 |
| 13. 0.3 mg AC911321 | Day 1: Single ICV Injection | n = 4 |
| 14. 0.3 mg AC911319 | Day 1: Single ICV Injection | n = 4 |
| 15. 0.3 mg AC911317 | Day 1: Single ICV Injection | n = 4 |
| 16. 0.3 mg AC911300 | Day 1: Single ICV Injection | n = 4 |
| 17. 0.3 mg AC004592* | Day 1: Single ICV Injection | n = 4 |
| 18. 0.3 mg AC004593** | Day 1: Single ICV Injection | n = 4 |
| 19. 0.3 mg CA005562*** | Day 1: Single ICV Injection | n = 4 |

*AC004592 is an RNAi agent having the sequences:
Antisense: vpusAfsucdAgCfuuuudCcAfgdGgucgcscsg (Seq ID No. 458)
Sense: gscsgaccC16CfuGfGfAfaaagcugasusa (Seq ID No. 576)
**AC004593 is an RNAi agent having the sequences:
Antisense: vpusAfsucdAgC_{UNA}uuuudCcAfgdGgucgcscsg (Seq ID No. 459)
Sense: gscsgaccC16CfuGfGfAfaaagcugasusa (Seq ID No. 576)
Wherein cC16 is 2′-O-hexadecyl cytidine, vp is 5′-vinyl phosphonate, dN is a deoxy nucleotide, lower case nucleotides are of 2′-O-methyl modified nucleotides, Nf are of 2′-fluoro modified nucleotides, s is a phosphorothioate linkage, C_{UNA} 2′,3′-seco-cytidine; see PCT Publication WO 2022/212231
***CA005562 is an antisense oligonucleotide having the sequence
mCMs, TM, mCM, AM, GMs, dTs, dAs, dAs, mdCs, dAs, dTs, dTs, dGs, dAs, mdCs, AM, mCM, mCM, AMs, mCM, wherein mC is a 5-methyl cytosine nucleobase, M is a 2′-MOE sugar moiety, dN is a deoxy nucleotide, and s is a phosphorothioate nucleoside linkage; see PCT Publication WO2021/168183

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with aCSF. HTT protein was quantified using Anti-Polyglutamine-Expansion Diseases Marker Antibody (Sigma-Aldrich®, Cat. MAB 1574) and Anti-Huntingtin Protein Antibody (Sigma-Aldrich®, Cat. MAB2166). The HTT expression data is shown in the following Table 28.

TABLE 28
HTT protein expression in mice brain tissues of Example 10.

| | Day 29 | | | |
|---|---|---|---|---|
| | Cortex | | | |
| | MAB1574 | | MAB2166 | |
| | Rel Exp | Std | Rel Exp | Std |
| Group ID | HTT | Dev+/− | HTT | Dev+/− |
| 1. aCSF | 1.000 | 0.039 | 1.158 | 0.150 |
| 2. 0.3 mg AC911290 | 0.603 | 0.096 | 0.843 | 0.157 |
| 3. 0.3 mg AC911291 | 0.952 | 0.070 | 0.444 | 0.076 |
| 4. 0.3 mg AC911292 | 0.883 | 0.123 | 0.455 | 0.102 |
| 5. 0.3 mg AC911293 | 0.444 | 0.069 | 0.279 | 0.093 |
| 6. 0.3 mg AC911294 | 0.858 | 0.123 | 0.852 | 0.053 |
| 7. 0.3 mg AC911295 | 0.757 | 0.072 | 0.497 | 0.127 |
| 8. 0.3 mg AC911599 | 0.663 | 0.118 | 0.487 | 0.084 |
| 9. 0.3 mg AC911600 | 0.601 | 0.129 | 0.135 | 0.025 |
| 10. 0.3 mg AC911601 | 0.822 | 0.072 | 0.698 | 0.204 |
| 11. 0.3 mg AC911602 | 0.898 | 0.076 | 1.314 | 0.075 |
| 12. 0.3 mg AC911603 | 0.589 | 0.082 | 0.741 | 0.120 |
| 13. 0.3 mg AC911321 | 0.273 | 0.032 | 0.191 | 0.039 |
| 14. 0.3 mg AC911319 | 0.155 | 0.051 | 0.479 | 0.191 |
| 15. 0.3 mg AC911317 | 0.241 | 0.083 | 0.315 | 0.087 |
| 16. 0.3 mg AC911300 | 0.137 | 0.023 | 1.091 | 0.133 |
| 17. 0.3 mg AC004592 | 0.573 | 0.079 | 0.233 | 0.034 |
| 18. 0.3 mg AC004593 | 0.635 | 0.039 | 0.934 | 0.076 |
| 19. 0.3 mg CA005562 | 0.333 | 0.057 | 1.158 | 0.150 |

When quantified using MAB1574, Groups 2, 5, 7-9, 12, 13-19 showed inhibition of HTT protein. Most notably, a single 0.3 mg AC911300 dose achieved ˜86% HTT protein inhibition (0.137) in the cortex. When quantified using MAB2166, Groups 3-5, 7-10, 12-15, and 17 showed inhibition of HTT protein. Most notably, a single 0.3 mg AC911600 dose achieved ˜86% HTT protein inhibition (0.135) in the cortex.

### Example 11. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 29.

FVB-Tg(YAC 128)53Hay/J mice (common name: “YAC 128” mice) express the human huntingtin protein.

TABLE 29
Dosing for mice of Example 11.

| Group ID | Dose (RNAi Agent) | # of Animals |
|---|---|---|
| 1. aCSF | Day 1: Single ICV Injection | n = 4 |
| 2. 0.1 mg AC911321 | Day 1: Single ICV Injection | n = 4 |
| 3. 0.1 mg AC005500 | Day 1: Single ICV Injection | n = 4 |
| 4. 0.1 mg AC004575 | Day 1: Single ICV Injection | n = 4 |
| 5. 0.1 mg AC005501 | Day 1: Single ICV Injection | n = 4 |
| 6. 0.1 mg AC005533 | Day 1: Single ICV Injection | n = 4 |
| 7. 0.1 mg AC005503 | Day 1: Single ICV Injection | n = 4 |
| 8. 0.1 mg AC005504 | Day 1: Single ICV Injection | n = 4 |
| 9. 0.1 mg AC005510 | Day 1: Single ICV Injection | n = 4 |
| 10. 0.1 mg AC005505 | Day 1: Single ICV Injection | n = 4 |
| 11. 0.1 mg AC005506 | Day 1: Single ICV Injection | n = 4 |
| 12. 0.1 mg AC005507 | Day 1: Single ICV Injection | n = 4 |
| 13. 0.1 mg AC005508 | Day 1: Single ICV Injection | n = 4 |
| 14. 0.1 mg AC005509 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with aCSF. HTT protein was quantified using Anti-Huntingtin Protein Antibody (Sigma-Aldrich®, Cat. MAB2166). The HTT expression data is shown in the following Table 30.

TABLE 30
HTT protein expression in mice brain tissues of Example 11.

Day 29
Cortex

| Group ID | Rel Exp HTT | Std Dev+/− |
|---|---|---|
| 1. aCSF | 0.998 | 0.044 |
| 2. 0.1 mg AC911321 | 0.470 | 0.116 |
| 3. 0.1 mg AC005500 | 0.223 | 0.033 |
| 4. 0.1 mg AC004575 | 0.645 | 0.032 |
| 5. 0.1 mg AC005501 | 0.669 | 0.124 |
| 6. 0.1 mg AC005533 | 0.539 | 0.198 |
| 7. 0.1 mg AC005503 | 0.795 | 0.023 |
| 8. 0.1 mg AC005504 | 0.526 | 0.084 |
| 9. 0.1 mg AC005510 | 0.462 | 0.010 |
| 10. 0.1 mg AC005505 | 0.724 | 0.100 |
| 11. 0.1 mg AC005506 | 0.519 | 0.098 |
| 12. 0.1 mg AC005507 | 0.482 | 0.063 |
| 13. 0.1 mg AC005508 | 0.786 | 0.085 |
| 14. 0.1 mg AC005509 | 0.792 | 0.104 |

In the cortex, Groups 2-14 showed reduction in HTT protein. Most notably, a single dose 0.1 mg AC005500 achieved ˜78% HTT protein inhibition (0.223) in the cortex.

### Example 12. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 31.

FVB-Tg(YAC 128)53Hay/J mice (common name: “YAC 128” mice) express the human huntingtin protein.

TABLE 31
Dosing for mice of Example 12.

| Group ID | Dose (RNAi Agent) | # of Animals |
|---|---|---|
| 1. aCSF | Day 1: Single ICV Injection | n = 4 |
| 2. 0.1 mg AC911319 | Day 1: Single ICV Injection | n = 4 |
| 3. 0.1 mg AC005522 | Day 1: Single ICV Injection | n = 4 |
| 4. 0.1 mg AC004576 | Day 1: Single ICV Injection | n = 4 |
| 5. 0.1 mg AC005523 | Day 1: Single ICV Injection | n = 4 |
| 6. 0.1 mg AC005524 | Day 1: Single ICV Injection | n = 4 |
| 7. 0.1 mg AC005525 | Day 1: Single ICV Injection | n = 4 |
| 8. 0.1 mg AC005526 | Day 1: Single ICV Injection | n = 4 |
| 9. 0.1 mg AC005527 | Day 1: Single ICV Injection | n = 4 |
| 10. 0.1 mg AC005528 | Day 1: Single ICV Injection | n = 4 |
| 11. 0.1 mg AC005529 | Day 1: Single ICV Injection | n = 4 |
| 12. 0.1 mg AC005530 | Day 1: Single ICV Injection | n = 4 |
| 13. 0.1 mg AC005531 | Day 1: Single ICV Injection | n = 4 |
| 14. 0.1 mg AC005532 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with aCSF. HTT protein was quantified using Anti-Polyglutamine-Expansion Diseases Marker Antibody (Sigma-Aldrich®, Cat. MAB1574). The HTT expression data is shown in the following Table 32.

TABLE 32
HTT protein expression in mice brain tissues of Example 12.

| | Day 29 | | | |
|---|---|---|---|---|
| | Cortex | | Striatum | |
| | Rel Exp | | Rel Exp | |
| Group ID | HTT | Std Dev+/− | HTT | Std Dev+/− |
| 1. aCSF | 1.000 | 0.046 | 0.996 | 0.089 |
| 2. 0.1 mg AC911319 | 0.300 | 0.104 | 0.440 | 0.181 |
| 3. 0.1 mg AC005522 | 0.337 | 0.117 | 0.368 | 0.036 |
| 4. 0.1 mg AC004576 | 0.390 | 0.033 | 0.419 | 0.066 |
| 5. 0.1 mg AC005523 | 0.328 | 0.114 | 0.346 | 0.107 |
| 6. 0.1 mg AC005524 | 0.236 | 0.034 | 0.401 | 0.042 |
| 7. 0.1 mg AC005525 | 0.239 | N/A* | 0.256 | N/A* |
| 8. 0.1 mg AC005526 | 0.464 | 0.166 | 0.612 | 0.090 |
| 9. 0.1 mg AC005527 | 0.486 | 0.089 | 0.516 | 0.072 |
| 10. 0.1 mg AC005528 | 0.727 | 0.118 | 0.732 | 0.060 |
| 11. 0.1 mg AC005529 | 0.284 | 0.162 | 0.383 | 0.181 |
| 12. 0.1 mg AC005530 | 0.433 | 0.129 | 0.510 | 0.042 |
| 13. 0.1 mg AC005531 | 0.276 | 0.178 | 0.387 | 0.152 |
| 14. 0.1 mg AC005532 | 0.261 | 0.106 | 0.300 | 0.102 |

*Standard deviation not available due to only 1 sample data.

In the cortex and striatum, Groups 2-14 showed reduction in HTT protein. Most notably, in the cortex, a single dose 0.1 mg AC005524 achieved ˜76% HTT protein inhibition (0.236). Also notably, in the striatum, a single dose 0.1 mg AC005525 achieved ˜74% HTT protein inhibition (0.256).

### Example 13. In Vivo Administration of HTT RNAi Agents in YAC128 Mice

HTT RNAi agents were evaluated in vivo in mice. On Day 1, five (n=5) FVB-Tg(YAC128)53Hay/J mice (of mixed gender M/F) for each group were dosed, via subcutaneous (SC) injection, with HTT RNAi agents formulated in phosphate buffered saline (PBS) (at 3.0 mg/kg, adjusted for individual animal body weight) or with PBS. The HTT RNAi agents were formulated at 250 μl/25 g dose volume. The dosing was in accordance with the following Table 33.

FVB-Tg(YAC 128)53Hay/J mice (common name: “YAC 128” mice) express the human huntingtin protein.

TABLE 33
Dosing for mice of Example 13.

| Group ID | Dose (RNAi Agent) | # of Animals |
|---|---|---|
| 1. PBS | Day 1, 2, 3, 4: SC Injection | n = 5 |
| 2. 3.0 mg/kg AC004499 | Day 1, 2, 3, 4: SC Injection | n = 5 |
| 3. 3.0 mg/kg AC006000 | Day 1, 2, 3, 4: SC Injection | n = 5 |
| 4. 3.0 mg/kg AC006001 | Day 1, 2, 3, 4: SC Injection | n = 5 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with PBS. HTT protein was quantified using Anti-Polyglutamine-Expansion Diseases Marker Antibody (Sigma-Aldrich®, Cat. MAB1574). The HTT expression data is shown in the following Table 34.

TABLE 34
HTT protein expression in mice brain tissues of Example 13.

| | MAB1574 | | | |
|---|---|---|---|---|
| | Cortex | | Striatum | |
| | Rel Exp | | Rel Exp | |
| Group ID | HTT | Std Dev+/− | HTT | Std Dev+/− |
| 1. PBS | 1.000 | 0.041 | 1.272 | 0.573 |
| 2. 3.0 mg/kg AC004499 | 0.575 | 0.019 | 0.483 | 0.099 |
| 3. 3.0 mg/kg AC006000 | 0.589 | 0.039 | 0.631 | 0.076 |
| 4. 3.0 mg/kg AC006001 | 0.791 | 0.044 | 0.732 | 0.065 |
| | Thoracic Spinal Cord | | Cerebellum | |
| | Rel Exp | | Rel Exp | |
| Group ID | HTT | Std Dev+/− | HTT | Std Dev+/− |
| 1. PBS | 1.000 | 0.059 | 1.000 | 0.060 |
| 2. 3.0 mg/kg AC004499 | 0.592 | 0.032 | 0.739 | 0.047 |
| 3. 3.0 mg/kg AC006000 | 0.704 | 0.108 | 0.744 | 0.022 |
| 4. 3.0 mg/kg AC006001 | 0.890 | 0.056 | 0.915 | 0.042 |

When quantified using MAB 1574, Groups 2-4 showed inhibition of HTT protein in the cortex. Most notably, 4× doses of 3/0 mg/kg AC004499 achieved ˜42% protein inhibition (0.575).

### Example 14. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.1 mg total HTT RNAi agent) or with aCSF. The HTT RNAi agents were formulated at 10 mg/mL at 10 μL total injection volume. The dosing was in accordance with the following Table 35.

FVB-Tg(YAC 128)53Hay/J mice (common name: “YAC 128” mice) express the human huntingtin protein.

TABLE 35
Dosing for mice of Example 14.

| Group ID | Dose (RNAi Agent) | # of Animals |
|---|---|---|
| 1. aCSF | Day 1: Single ICV Injection | n = 4 |
| 2. 0.1 mg AC911317 | Day 1: Single ICV Injection | n = 4 |
| 3. 0.1 mg AC005511 | Day 1: Single ICV Injection | n = 4 |
| 4. 0.1 mg AC004577 | Day 1: Single ICV Injection | n = 4 |
| 5. 0.1 mg AC005512 | Day 1: Single ICV Injection | n = 4 |
| 6. 0.1 mg AC005513 | Day 1: Single ICV Injection | n = 4 |
| 7. 0.1 mg AC005514 | Day 1: Single ICV Injection | n = 4 |
| 8. 0.1 mg AC005515 | Day 1: Single ICV Injection | n = 4 |
| 9. 0.1 mg AC005516 | Day 1: Single ICV Injection | n = 4 |
| 10. 0.1 mg AC005517 | Day 1: Single ICV Injection | n = 4 |
| 11. 0.1 mg AC005518 | Day 1: Single ICV Injection | n = 4 |
| 12. 0.1 mg AC005519 | Day 1: Single ICV Injection | n = 4 |
| 13. 0.1 mg AC005520 | Day 1: Single ICV Injection | n = 4 |
| 14. 0.1 mg AC005521 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with aCSF. HTT protein was quantified using Anti-Polyglutamine-Expansion Diseases Marker Antibody (Sigma-Aldrich®, Cat. MAB1574). The HTT expression data is shown in the following Table 36.

TABLE 36
HTT protein expression in mice brain tissues of Example 14.

| | Day 29 | | | |
|---|---|---|---|---|
| | Cortex | | Striatum | |
| | Rel Exp | | Rel Exp | |
| Group ID | HTT | Std Dev+/− | HTT | Std Dev+/− |
| 1. aCSF | 1.000 | 0.033 | 1.000 | 0.063 |
| 2. 0.1 mg AC911317 | 0.479 | 0.087 | 0.664 | 0.137 |
| 3. 0.1 mg AC005511 | 0.305 | 0.082 | 0.371 | 0.059 |
| 4. 0.1 mg AC004577 | 0.384 | 0.056 | 0.547 | 0.085 |
| 5. 0.1 mg AC005512 | 0.385 | 0.107 | 0.545 | 0.131 |
| 6. 0.1 mg AC005513 | 0.439 | 0.115 | 0.550 | 0.063 |
| 7. 0.1 mg AC005514 | 0.333 | 0.033 | 0.517 | 0.058 |
| 8. 0.1 mg AC005515 | 0.608 | 0.075 | 0.648 | 0.079 |
| 9. 0.1 mg AC005516 | 0.924 | 0.068 | 0.830 | 0.113 |
| 10. 0.1 mg AC005517 | 0.396 | 0.132 | 0.484 | 0.135 |
| 11. 0.1 mg AC005518 | 0.857 | 0.083 | 0.922 | 0.054 |
| 12. 0.1 mg AC005519 | 0.438 | 0.148 | 0.377 | 0.024 |
| 13. 0.1 mg AC005520 | 0.462 | 0.109 | 0.480 | 0.057 |
| 14. 0.1 mg AC005521 | 0.373 | 0.063 | 0.476 | 0.094 |

In the cortex and striatum, Groups 2-8, 10, and 12-14 showed reduction in HTT protein; Groups 9 and 11 showed little to negligible reduction. Most notably, in the cortex, a single dose 0.1 mg AC005511 achieved ˜69% HTT protein inhibition (0.305). Also notably, in the striatum, a single dose 0.1 mg AC005511 achieved ˜63% HTT protein inhibition (0.371).

### Example 15. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 30 μg, 100 μg, or 300 μg total HTT RNAi agent) or with aCSF (control). The HTT RNAi agents were formulated at 3 mg/mL, 10 mg/mL, or 30 mg/mL, at 10 μL total injection volume. The dosing was in accordance with the following Table 37.

FVB-Tg(YAC 128)53Hay/J mice (common name: “YAC 128” mice) express the human huntingtin protein.

TABLE 37
Dosing for mice of Example 15.

| Group ID | Dose (RNAi Agent) | # of Animals |
|---|---|---|
| 1. aCSF | Day 1: Single ICV Injection | n = 4 |
| 2. 30 μg AC006707 | Day 1: Single ICV Injection | n = 4 |
| 3. 100 μg AC006707 | Day 1: Single ICV Injection | n = 4 |
| 4. 300 μg AC006707 | Day 1: Single ICV Injection | n = 4 |
| 5. 30 μg AC004593 | Day 1: Single ICV Injection | n = 4 |
| 6. 100 μg AC004593 | Day 1: Single ICV Injection | n = 4 |
| 7. 300 μg AC004593 | Day 1: Single ICV Injection | n = 4 |
| 8. 30 μg CA005562 | Day 1: Single ICV Injection | n = 4 |
| 9. 100 μg CA005562 | Day 1: Single ICV Injection | n = 4 |
| 10. 300 μg CA005562 | Day 1: Single ICV Injection | n = 4 |

*CA005562 is an antisense oligonucleotide having the sequence mCMs, TM, mCM, AM, GMs, dTs, dAs, dAs, mdCs, dAs, dTs, dTs, dGs, dAs, mdCs, AM, mCM, mCM, AMs, mCM, wherein mC is a 5-methyl cytosine nucleobase, M is a 2′-MOE sugar moiety, dN is a deoxy nucleotide, and s is a phosphorothioate nucleoside linkage; see PCT Publication WO2021/168183

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex and striatum, normalized to Group 1 mice dosed with aCSF. HTT protein was quantified using Anti-Polyglutamine-Expansion Diseases Marker Antibody (Sigma-Aldrich®, Cat. MAB1574). The HTT expression data is shown in the following Table 38.

TABLE 38
HTT protein expression in mice brain tissues of Example 15.

| | Day 29 | | | |
|---|---|---|---|---|
| | MAB1574 | | | |
| | Cortex | | Striatum | |
| | Rel Exp | | Rel Exp | |
| Group ID | HTT | Std Dev+/− | HTT | Std Dev+/− |
| 1. aCSF | 0.989 | 0.074 | 1.000 | 0.043 |
| 2. 30 μg AC006707 | 0.670 | 0.206 | 0.554 | 0.134 |
| 3. 100 μg AC006707 | 0.422 | 0.083 | 0.356 | 0.085 |
| 4. 300 μg AC006707 | 0.284 | 0.156 | 0.251 | 0.094 |
| 5. 30 μg AC004593 | 0.844 | 0.127 | 0.939 | 0.086 |
| 6. 100 μg AC004593 | 0.770 | 0.100 | 0.684 | 0.120 |
| 7. 300 μg AC004593 | 0.745 | 0.205 | 0.683 | 0.142 |
| 8. 30 μg CA005562 | 0.905 | 0.195 | 0.768 | 0.148 |
| 9. 100 μg CA005562 | 0.611 | 0.101 | 0.629 | 0.063 |
| 10. 300 μg CA005562 | 0.545 | 0.000 | 0.507 | 0.109 |

In the cortex, Groups 2-4, 6, 7, 9, and 10 showed significant reduction in HTT protein; Groups 5 and 8 showed negligible HTT reduction. Most notably, in the cortex, a single dose 300 μg AC006707 achieved ˜72% HTT protein inhibition (0.284). A dose response was observed for AC006707, AC004593, and CA005562.

In the striatum, Groups 2-4 and 6-10 showed reduction in HTT protein; Group 5 showed negligible HTT reduction. Most notably, in the striatum, a single dose of 300 μg of AC006707 achieved ˜75% HTT protein inhibition (0.251). A dose response was observed for AC006707, AC004593, and CA005562.

In both the cortex and striatum, AC006707 achieved superior HTT protein reduction in comparison to AC004593 and CA005562, at each respective dose (30 μg, 100 μg, 300 μg).

### Example 16. In Vivo Administration of HTT RNAi Agents in YAC128 Mice

HTT RNAi agents were evaluated in vivo in mice. On Day 1, four (n=4) FVB-Tg(YAC128)53Hay/J mice (2 male, 2 female) for each group were dosed, via intracerebroventricular (ICV) injection, with HTT RNAi agents formulated in artificial cerebrospinal fluid (aCSF) (at 0.07 mg total HTT RNAi agent) or with aCSF. The HTT RNAi agents were formulated at 7 mg/mL at 10 μL total injection volume. The dosing was in accordance with the following Table 39.

FVB-Tg(YAC 128)53Hay/J mice (common name: “YAC 128” mice) express the human huntingtin protein.

TABLE 39
Dosing for mice of Example 16.

| Group ID | Dose (RNAi Agent) | # of Animals |
|---|---|---|
| 1. aCSF | Day 1: Single ICV Injection | n = 4 |
| 2. 0.07 mg AC005500 | Day 1: Single ICV Injection | n = 4 |
| 3. 0.07 mg AC006704 | Day 1: Single ICV Injection | n = 4 |
| 4. 0.07 mg AC006705 | Day 1: Single ICV Injection | n = 4 |
| 5. 0.07 mg AC006706 | Day 1: Single ICV Injection | n = 4 |
| 6. 0.07 mg AC005511 | Day 1: Single ICV Injection | n = 4 |
| 7. 0.07 mg AC006707 | Day 1: Single ICV Injection | n = 4 |
| 8. 0.07 mg AC006708 | Day 1: Single ICV Injection | n = 4 |
| 9. 0.07 mg AC006709 | Day 1: Single ICV Injection | n = 4 |
| 10. 0.07 mg AC005522 | Day 1: Single ICV Injection | n = 4 |
| 11. 0.07 mg AC005524 | Day 1: Single ICV Injection | n = 4 |
| 12. 0.07 mg AC006805 | Day 1: Single ICV Injection | n = 4 |
| 13. 0.07 mg AC006806 | Day 1: Single ICV Injection | n = 4 |
| 14. 0.07 mg AC006807 | Day 1: Single ICV Injection | n = 4 |

Each of the HTT RNAi agents included modified nucleotides that were conjugated at the 5′ terminal end of the sense strand to a targeting ligand that included a lipid moiety or antigen binding moiety having the modified sequences as set forth in the duplex structures herein. (See Tables 3, 4, 5, 6, 7, 8, 9, and 10 for specific modifications and structure information related to the HTT RNAi agents, including the lipid and antigen binding moieties).

On Day 29, the mice were euthanized. From the mice, right half of the brain and thoracic spinal cord were harvested and collected for analysis.

HTT protein expression was analyzed via Jess protein assay in the cortex, normalized to Group 1 mice dosed with aCSF. HTT protein was quantified using Anti-Polyglutamine-Expansion Diseases Marker Antibody (Sigma-Aldrich®, Cat. MAB1574). The HTT expression data is shown in the following Table 40.

TABLE 40
HTT protein expression in mice brain tissues of Example 16.

| | Day 29 | | | |
|---|---|---|---|---|
| | MAB1574 | | | |
| | Cortex | | Striatum | |
| | Rel Exp | | Rel Exp | |
| Group ID | HTT | Std Dev+/− | HTT | Std Dev+/− |
| 1. aCSF | 1.000 | 0.120 | 1.000 | 0.066 |
| 2. 0.07 mg AC005500 | 0.552 | 0.093 | 0.699 | 0.066 |
| 3. 0.07 mg AC006704 | 0.470 | 0.132 | 0.683 | 0.120 |
| 4. 0.07 mg AC006705 | 0.528 | 0.115 | 0.641 | 0.117 |
| 5. 0.07 mg AC006706 | 0.540 | 0.076 | 0.643 | 0.102 |
| 6. 0.07 mg AC005511 | 0.299 | 0.093 | 0.400 | 0.060 |
| 7. 0.07 mg AC006707 | 0.329 | 0.097 | 0.356 | 0.062 |
| 8. 0.07 mg AC006708 | 0.404 | 0.112 | 0.526 | 0.176 |
| 9. 0.07 mg AC006709 | 0.391 | 0.097 | 0.470 | 0.053 |
| 10. 0.07 mg AC005522 | 0.465 | 0.136 | 0.358 | 0.100 |
| 11. 0.07 mg AC005524 | 0.361 | 0.066 | 0.430 | 0.114 |
| 12. 0.07 mg AC006805 | 0.363 | 0.068 | 0.465 | 0.046 |
| 13. 0.07 mg AC006806 | 0.339 | 0.046 | 0.553 | 0.036 |
| 14. 0.07 mg AC006807 | 0.454 | 0.172 | 0.419 | 0.043 |

In the cortex and striatum, Groups 2-14 showed reduction in HTT protein. Most notably, in the cortex, a single dose 0.07 mg AC005511 achieved ˜70% HTT protein inhibition (0.299). Also notably, in the striatum, a single dose 0.07 mg AC006707 achieved ˜-64% HTT protein inhibition (0.356).

### Example 17. In Vivo Administration of HTT RNAi Agents in Cynomolgus Monkeys

HTT RNAi agents were evaluated in vivo in Cynomolgus monkeys. On Days 1, 8, and 15, four (n=4) male Cynomolgus monkeys for each test group were dosed with HTT RNAi agents formulated in PBS at 3.0 mg/kg (adjusted for individual animal body weight), 2.0 ml/kg dose volume, at 1.5 mg/ml dose concentration, or dosed with PBS. Each dose was administered via subcutaneous (SC) injection, and each dose was administered based on each respective animal's most recent body weight. The dosing was in accordance with the following Table 41.

TABLE 41
Dosing for Cynomolgus monkeys of Example 17.

| Group ID, Dose | Dose | | Dose | # of Animals |
|---|---|---|---|---|
| (RNAi Agent) | Volume | Dosing Route | Concentration | (n=) |
| 1. PBS | N/A | SC Injection | N/A | n = 4 |
| 2. 3.0 mg/kg AC007867 | 2.0 ml/kg | SC Injection | 1.5 mg/ml | n = 4 |
| 3. 3.0 mg/kg AC007952 | 2.0 ml/kg | SC Injection | 1.5 mg/ml | n = 4 |
| 4. 3.0 mg/kg AC007953 | 2.0 ml/kg | SC Injection | 1.5 mg/ml | n = 4 |
| 5. 3.0 mg/kg AC007865 | 2.0 ml/kg | SC Injection | 1.5 mg/ml | n = 4 |

The test animals were of non-human primate, Cynomolgus fascicularis monkeys, male, non-naïve, aged 3-5 years. The test animals were acclimated to laboratory housing, per facility and acclimation standard operating procedures, for at least 14 days prior to the initiation of dosing. The RNAi agent test articles were administered via subcutaneous (SC) administration with a syringe and needle in the mid-scapular region.

Dose sites were shaved prior to dosing. Day 1 dose was delivered to the animals' left scapular region, Day 8 dose was delivered to the right scapular region, and Day 15 dose was delivered to the left scapular region. Each dose was given using syringe with a 23-25 gauge needle.

The test animals' individual body weights were recorded once pre-treatment Day −7, and then weekly through the duration of the study, and once prior to necropsy.

Cerebrospinal fluid (CSF), ˜0.5 mL, was collected on Day−7 for all Groups. On Day 43 (day of necropsy), ˜2.0 mL CSF was collected for all Groups. For the CSF collection procedure, all test animals were anesthetized by intravenous (N) injection of ketamine (10 mg/kg, IV) and dexmedetomidine (0.02 mg/kg, intramuscular IM) and positioned in lateral recumbency. The skin covering the insertion point was shaved and wiped several times with individual chlorhexidine scrubs. The head of the animal was gently flexed to where the chin nearly touches the chest, but the airway was not obstructed. The professional palpated the occipital protuberance and wings of the atlas (C₁). The needle was inserted perpendicularly above the atlas through the skin to access the subarachnoid space. If the bone was encountered, the needle was redirected either anteriorly or posteriorly until the designated access point was found. The CSF would flow freely through the needle once the proper placement has been achieved and collected into a cryotube tube. The needle was removed, and direct pressure was applied to the puncture site for at least two minutes. Once the procedure was complete, the animals were administered Carprofen 2-4 mg/kg SC every 12 hours for 1 day. Atipamezole (0.225 mg/kg, IM), was administered if necessary.

At Day 43, the Cynomolgus monkeys were euthanized. From the test animals, the following tissues were collected: left and right brain hemisphere, spinal cord, dorsal root ganglion (DRG). Tissues other than the aforementioned tissues may also be collected, and should such extra tissues be collected, their biological data were similarly presented below. The collected tissues were analyzed for biological parameters.

From the collected tissues, cHTT mRNA transcript levels were quantified via gPCR, with cPPIB as endogenous control gene, normalized to Group 1 animals dosed with PBS. The cHTT expression data is shown in the following Table 42.

TABLE 42
cHTT expression in tissues of Example 17.

| | Day 43 | | | | | |
|---|---|---|---|---|---|---|
| | Temporal Cortex | | | Frontal Cortex | | |
| | Rel Exp | Error | Error | Rel Exp | Error | Error |
| Group ID | HTT | Low | High | HTT | Low | High |
| 1. PBS | 1.000 | 0.169 | 0.203 | 1.000 | 0.161 | 0.192 |
| 2. 3.0 mg/kg AC007867 | 0.331 | 0.063 | 0.078 | 0.333 | 0.056 | 0.067 |
| 3. 3.0 mg/kg AC007952 | 0.557 | 0.055 | 0.060 | 0.442 | 0.073 | 0.088 |
| 4. 3.0 mg/kg AC007953 | 0.518 | 0.077 | 0.090 | 0.503 | 0.065 | 0.074 |
| 5. 3.0 mg/kg AC007865 | 0.408 | 0.102 | 0.137 | 0.358 | 0.055 | 0.065 |
| | Motor Cortex | | | Caudate | | |
| | Rel Exp | Error | Error | Rel Exp | Error | Error |
| Group ID | HTT | Low | High | HTT | Low | High |
| 1. PBS | 1.000 | 0.235 | 0.307 | 1.000 | 0.165 | 0.198 |
| 2. 3.0 mg/kg AC007867 | 0.393 | 0.072 | 0.088 | 0.413 | 0.072 | 0.087 |
| 3. 3.0 mg/kg AC007952 | 0.557 | 0.112 | 0.141 | 0.475 | 0.079 | 0.095 |
| 4. 3.0 mg/kg AC007953 | 0.775 | 0.099 | 0.114 | 0.424 | 0.060 | 0.069 |
| 5. 3.0 mg/kg AC007865 | 0.405 | 0.061 | 0.072 | 0.340 | 0.079 | 0.102 |
| | Putamen | | | Hippocampus | | |
| | Rel Exp | Error | Error | Rel Exp | Error | Error |
| Group ID | HTT | Low | High | HTT | Low | High |
| 1. PBS | 1.000 | 0.106 | 0.119 | 1.000 | 0.164 | 0.196 |
| 2. 3.0 mg/kg AC007867 | 0.411 | 0.030 | 0.032 | 0.306 | 0.071 | 0.092 |
| 3. 3.0 mg/kg AC007952 | 0.795 | 0.100 | 0.114 | 0.515 | 0.089 | 0.108 |
| 4. 3.0 mg/kg AC007953 | 0.698 | 0.083 | 0.095 | 0.498 | 0.062 | 0.071 |
| 5. 3.0 mg/kg AC007865 | 0.456 | 0.068 | 0.080 | 0.302 | 0.044 | 0.051 |

| | Cerebellum | | |
|---|---|---|---|
| | Rel Exp | Error | Error |
| Group ID | HTT | Low | High |
| 1. PBS | 1.000 | 0.357 | 0.556 |
| 2. 3.0 mg/kg AC007867 | 0.883 | 0.307 | 0.471 |
| 3. 3.0 mg/kg AC007952 | 0.989 | 0.167 | 0.201 |
| 4. 3.0 mg/kg AC007953 | 1.168 | 0.188 | 0.224 |
| 5. 3.0 mg/kg AC007865 | 0.795 | 0.266 | 0.401 |

In the temporal cortex, frontal cortex, motor cortex, caudate, hippocampus, Groups 2-5 showed reduction in HTT transcripts out to at least Day 43 post dose. In the putamen, Groups 2, 4, and 5 showed reduction in HTT transcripts out to at least Day 43 post dose. Most notably, the most significant HTT transcript reduction was seen in the hippocampus, three doses of 3.0 mg/kg AC007867 showed ˜69% HTT transcript inhibition (0.306), and three doses of 3.0 mg/kg AC007865 showed ˜70% HTT transcript inhibition (0.302).

HTT protein expression was analyzed via Jess protein assay in the tissues, normalized to Group 1 animals dosed with PBS. HTT protein was quantified using Anti-Huntingtin Protein Antibody (Sigma-Aldrich®, Cat. MAB2166). The HTT protein expression data is shown in the following Table 43.

TABLE 43
HTT protein expression in tissues of Example 17.

| | Day 43 | | | | | |
|---|---|---|---|---|---|---|
| | Temporal Cortex | | Frontal Cortex | | Motor Cortex | |
| | Rel Exp | Std | Rel Exp | Std | Rel Exp | Std |
| Group ID | HTT | Dev+/− | HTT | Dev+/− | HTT | Dev+/− |
| 1. PBS | 1.000 | 0.103 | 1.000 | 0.019 | 1.000 | 0.130 |
| 2. 3.0 mg/kg AC007867 | 0.160 | 0.041 | 0.171 | 0.016 | 0.143 | 0.025 |
| 3. 3.0 mg/kg AC007952 | 0.486 | 0.057 | 0.390 | 0.031 | 0.336 | 0.027 |
| 4. 3.0 mg/kg AC007953 | 0.407 | 0.056 | 0.353 | 0.092 | 0.287 | 0.034 |
| 5. 3.0 mg/kg AC007865 | 0.189 | 0.030 | 0.168 | 0.058 | 0.146 | 0.033 |
| | Caudate | | Putamen | | Hippocampus | |
| | Rel Exp | Std | Rel Exp | Std | Rel Exp | Std |
| Group ID | HTT | Dev+/− | HTT | Dev+/− | HTT | Dev+/− |
| 1. PBS | 1.000 | 0.082 | 1.000 | 0.090 | 1.000 | 0.050 |
| 2. 3.0 mg/kg AC007867 | 0.176 | 0.027 | 0.178 | 0.059 | 0.179 | 0.034 |
| 3. 3.0 mg/kg AC007952 | 0.407 | 0.102 | 0.520 | 0.120 | 0.479 | 0.076 |
| 4. 3.0 mg/kg AC007953 | 0.346 | 0.079 | 0.432 | 0.125 | 0.468 | 0.031 |
| 5. 3.0 mg/kg AC007865 | 0.198 | 0.044 | 0.199 | 0.041 | 0.201 | 0.015 |

| | Cerebellum | |
|---|---|---|
| | Rel Exp | Std |
| Group ID | HTT | Dev+/− |
| 1. PBS | 1.000 | 0.191 |
| 2. 3.0 mg/kg AC007867 | 0.655 | 0.049 |
| 3. 3.0 mg/kg AC007952 | 0.704 | 0.154 |
| 4. 3.0 mg/kg AC007953 | 0.659 | 0.082 |
| 5. 3.0 mg/kg AC007865 | 0.636 | 0.192 |

In the temporal cortex, frontal cortex, motor cortex, caudate, putamen, hippocampus, and cerebellum, Groups 2-5 showed reduction in HTT protein out to at least Day 43 post dose. Most notably, the most significant HTT protein reduction is seen in the motor cortex, three doses of 3.0 mg/kg AC007867 showed ˜86% HTT protein inhibition (0.143), and three doses of 3.0 mg/kg AC007865 showed ˜85% HTT protein inhibition (0.146).

### Example 18. In Vivo Administration of HTT RNAi Agents in Cynomolgus Monkeys

HTT RNAi agents were evaluated in vivo in Cynomolgus monkeys. On Days 1, 8, and 15, four (n=4) male Cynomolgus monkeys for each test group were dosed with HTT RNAi agents formulated in PBS at 0.3 mg/kg, 1.0 mg/kg, or 3.0 mg/kg (mass of oligo per animal body weight-Fab not included in mass calculation; adjusted for individual animal body weight), 2.0 ml/kg dose volume, at 1.5 mg/ml, 0.5 mg/ml, or 0.15 mg/ml dose concentration, or dosed with PBS. Each dose was administered via subcutaneous (SC) injection, and each dose was administered based on each respective animal's most recent body weight. The dosing was in accordance with the following Table 44.

TABLE 44
Dosing for Cynomolgus monkeys of Example 18.

| Group ID, Dose | | Sacrifice | # of Animals |
|---|---|---|---|
| (RNAi Agent) | Dosing Route | Day | (n=) |
| 1. PBS | SC Injection on Day 1, 8, 15 | Day 43 | n = 4 |
| 2. 0.3 mg/kg AC007867 | SC Injection on Day 1, 8, 15 | Day 43 | n = 4 |
| 3. 1.0 mg/kg AC007867 | SC Injection on Day 1, 8, 15 | Day 43 | n = 4 |
| 4. 3.0 mg/kg AC007867 | SC Injection on Day 1, 8, 15 | Day 43 | n = 4 |
| 5. 3.0 mg/kg AC007867 | SC Injection on Day 1 | Day 29 | n = 4 |
| 6. 3.0 mg/kg AC007867 | SC Injection on Day 1, 8, 15 | Day 92* | n = 4 |

Each animal from Groups 1-4 and 6 were dosed on Day 1, 8, and 15, while Group 5 animals were dosed on only Day 1. The RNAi agent test articles were administered via subcutaneous (SC) administration with a syringe and needle in the mid-scapular region. Dose sites were shaved before dosing and remarked as necessary throughout the study. Dose one (on Day 1) was delivered to the animal's left scapular region, dose two (on Day 8) was delivered to the right scapular region, and dose three (on Day 15) was delivered to the left scapular region. Each subcutaneous dose was delivered using a syringe with 23-25-gauze needle.

The test animals' individual body weights were recorded once pre-treatment Day−7, and then weekly through the duration of the study, and once prior to necropsy.

Cerebrospinal fluid (CSF), (˜1.0 mL), was collected on Day−7 and on day of necropsy for all Groups. CSF (˜2.0 mL), was collected for all the groups from the lumbar region (lumbar puncture). For Group 1 only, another ˜2.0 ml of CSF was collected (Terminal collection). For the CSF collection procedure, animals were sedated using a combination of ketamine (5-10 mg/Kg-IM) and dexmedetomidine (0.05 mg/Kg-IM) and positioned in lateral recumbency. Under sterile surgical material and strict aseptic technique, a Gertie Marx Needle (22G×50 mm) was inserted through the dura mater/arachnoid membrane. 2.0 ml (or 0.5 mL) of CSF was collected via freely flow through the needle. The needle was removed after CSF collection. Once the procedure was complete, the animals were administered 2-4 mg/kg of Carprofen subcutaneously every 12 hours for 1 day. Atipamezole (0.225 mg/kg, IM), was administered if necessary.

At Day 29, 43, or 92, the Cynomolgus monkeys were euthanized, in accordance with Table 44. From the test animals, the following tissues were collected: left and right brain hemisphere, spinal cord, dorsal root ganglion (DRG). Tissues other than the aforementioned tissues may also be collected, and should such extra tissues be collected, their biological data were similarly presented below. The collected tissues were analyzed for biological parameters.

From the collected tissues, cHTT mRNA transcript levels were quantified via gPCR, with cPPIB as endogenous control gene, normalized to Group 1 animals dosed with PBS. The cHTT expression data is shown in the following Table 45 and Table 46.

TABLE 45
cHTT mRNA transcript expression in tissues of Example 18.

| | Frontal Cortex | | | Temporal Cortex | | |
|---|---|---|---|---|---|---|
| | Rel Exp | Error | Error | Rel Exp | Error | Error |
| Group ID | HTT | Low | High | HTT | Low | High |
| 1. PBS, D 43 | 1.000 | 0.272 | 0.374 | 1.000 | 0.204 | 0.257 |
| 2. 3 × 0.3 mg/kg AC007867, D 43 | 0.968 | 0.051 | 0.054 | 0.576 | 0.136 | 0.179 |
| 3. 3 × 1.0 mg/kg AC007867, D 43 | 0.753 | 0.139 | 0.171 | 0.310 | 0.087 | 0.121 |
| 4. 3 × 3.0 mg/kg AC007867, D 43 | 0.480 | 0.125 | 0.170 | 0.218 | 0.057 | 0.077 |
| 5. 1 × 3.0 mg/kg AC007867, D 29 | 0.821 | 0.085 | 0.095 | 0.528 | 0.061 | 0.069 |
| | Caudate | | | Putamen | | |
| | Rel Exp | Error | Error | Rel Exp | Error | Error |
| Group ID | HTT | Low | High | HTT | Low | High |
| 1. PBS, D 43 | 1.000 | 0.198 | 0.248 | 1.000 | 0.078 | 0.085 |
| 2. 3 × 0.3 mg/kg AC007867, D 43 | 0.958 | 0.082 | 0.089 | 0.732 | 0.068 | 0.075 |
| 3. 3 × 1.0 mg/kg AC007867, D 43 | 0.720 | 0.229 | 0.336 | 0.474 | 0.128 | 0.176 |
| 4. 3 × 3.0 mg/kg AC007867, D 43 | 0.447 | 0.120 | 0.164 | 0.403 | 0.041 | 0.046 |
| 5. 1 × 3.0 mg/kg AC007867, D 29 | 0.529 | 0.088 | 0.106 | 0.474 | 0.098 | 0.123 |

In the frontal cortex, Groups 3 and 4 showed reduction in HTT transcripts. In the temporal cortex and putamen, Groups 2-5 showed reduction in HTT transcripts. In the caudate, Groups 3-5 showed reduction in HTT transcripts. Most notably, the most significant HTT transcript reduction was seen in the temporal cortex, three doses of 3.0 mg/kg AC007867 achieved ˜78% HTT transcript inhibition (0.218) at Day 43. At Day 43, a dose-response was observed for AC007867 (Groups 2-4) in all of the CNS tissues.

In the temporal cortex, caudate, and putamen, a single dose of 3.0 mg/kg showed reduction in HTT transcripts out to at least Day 29. Notably, a single dose 3.0 mg/kg AC007867 achieved ˜53% HTT transcript inhibition (0.474) out to at least Day 29 in the putamen.

TABLE 46
cHTT mRNA transcript expression in tissues of Example 18.

| | Frontal Cortex | | | Temporal Cortex | | |
|---|---|---|---|---|---|---|
| | Rel Exp | Error | Error | Rel Exp | Error | Error |
| Group ID | HTT | Low | High | HTT | Low | High |
| 1. PBS, D 43 | 1.000 | 0.292 | 0.412 | 1.000 | 0.216 | 0.275 |
| 6. 3 × 3.0 mg/kg AC007867, D 92 | 0.661 | 0.088 | 0.101 | 0.539 | 0.093 | 0.112 |
| | Caudate | | | Putamen | | |
| | Rel Exp | Error | Error | Rel Exp | Error | Error |
| Group ID | HTT | Low | High | HTT | Low | High |
| 1. PBS, D 43 | 1.000 | 0.179 | 0.219 | 1.000 | 0.094 | 0.104 |
| 6. 3 × 3.0 mg/kg AC007867, D 92 | 0.605 | 0.097 | 0.116 | 0.664 | 0.099 | 0.116 |

In the frontal cortex, temporal cortex, caudate, and putamen, three doses of 3.0 mg/kg showed reduction in HTT transcripts out to at least Day 92. Notably, a single dose 3.0 mg/kg AC007867 achieved ˜46% HTT transcript inhibition (0.539) out to at least Day 92 in the temporal cortex.

HTT protein expression was analyzed via Jess protein assay in the tissues, normalized to Group 1 animals dosed with PBS. HTT protein was quantified using Anti-Huntingtin Protein Antibody (Sigma-Aldrich®, Cat. MAB2166). The HTT protein expression data is shown in the following Table 47.

TABLE 47
HTT protein expression in tissues of Example 18.

| | | MAB2166 | | | |
|---|---|---|---|---|---|
| | | Temporal Cortex | | Frontal Cortex | |
| | | Rel. Exp. | | Rel. Exp. | |
| Group ID | Sac Day | HTT | Std. Dev+/− | HTT | Std. Dev+/− |
| 1. PBS | Day 43 | 1.000 | 0.075 | 1.000 | 0.039 |
| 2. 3 × 0.3 mg/kg AC007867 | Day 43 | 0.753 | 0.135 | 0.606 | 0.169 |
| 3. 3 × 1.0 mg/kg AC007867 | Day 43 | 0.437 | 0.039 | 0.382 | 0.039 |
| 4. 3 × 3.0 mg/kg AC007867 | Day 43 | 0.174 | 0.076 | 0.164 | 0.030 |
| 5. 1 × 3.0 mg/kg AC007867 | Day 29 | 0.453 | 0.023 | 0.521 | 0.124 |
| 6. 3 × 3.0 mg/kg AC007867 | Day 92 | 0.355 | 0.077 | 0.338 | 0.097 |
| | | Caudate | | Putamen | |
| | | Rel. Exp. | | Rel. Exp. | |
| Group ID | Sac Day | HTT | Std. Dev+/− | HTT | Std. Dev+/− |
| 1. PBS | Day 43 | 1.000 | 0.039 | 1.000 | 0.084 |
| 2. 3 × 0.3 mg/kg AC007867 | Day 43 | 0.747 | 0.255 | 0.776 | 0.183 |
| 3. 3 × 1.0 mg/kg AC007867 | Day 43 | 0.425 | 0.076 | 0.467 | 0.140 |
| 4. 3 × 3.0 mg/kg AC007867 | Day 43 | 0.242 | 0.023 | 0.245 | 0.031 |
| 5. 1 × 3.0 mg/kg AC007867 | Day 29 | 0.482 | 0.038 | 0.462 | 0.056 |
| 6. 3 × 3.0 mg/kg AC007867 | Day 92 | 0.413 | 0.137 | 0.448 | 0.123 |

HTT RNAi agent AC007867 achieved HTT protein knockdown out to at least Day 92 in the temporal cortex, frontal cortex, caudate, and putamen with 3× doses. A single dose 3.0 mg/kg AC007867 achieved HTT protein knockdown out to at least Day 29. The most notable and significant knockdown was observed in the frontal cortex, with 3× 3.0 mg/kg subcutaneous (SC) doses achieving ˜84% HTT protein inhibition (0.164) on Day 43. A dose response was observed for RNAi agent AC007867 in the temporal cortex, frontal cortex, caudate, and putamen.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-78. (canceled)

79. A conjugate, or a pharmaceutically acceptable salt thereof, comprising an RNAi agent and an antibody fragment (Fab), wherein the conjugate comprises:
i. an antisense strand comprising the nucleotide sequence (5′ to 3′) cPrpusAfscaAfcgagacUfgAfaUfugccssu (SEQ ID NO: 468), and
ii. a sense strand comprising Fab0070-L1026-C6s(invAb)saggcaauuCfaGfuCfucguuguas(invAb) (5′ to 3′) (SEQ ID NO: 821);
wherein:
(a) a represents 2′-O-methyl adenosine, c represents 2′-O-methyl cytidine, g represents 2′-O-methyl guanosine, u represents 2′-O-methyl uridine, Af represents 2′-fluoro adenosine, Cf represents 2′-fluoro cytidine, Gf represents 2′-fluoro guanosine, Uf represents 2′-fluoro uridine, s represents a phosphorothioate linkage, and ss represents a phosphorodithioate linkage;
(b) cPrpus represents 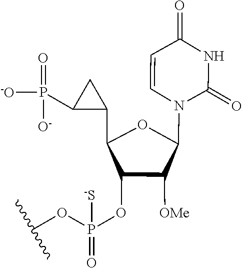
(c) (invAb) represents
linkage towards 5′ end 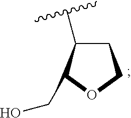
(d) (invAb)s represents
linkage towards 5′ end 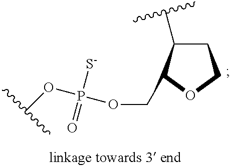
(e) C6s represents 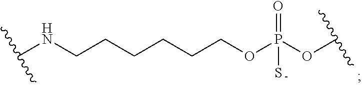 [SMILES: *NCCCCCCOP(=O)(S)O*]
(f) L1026 represents 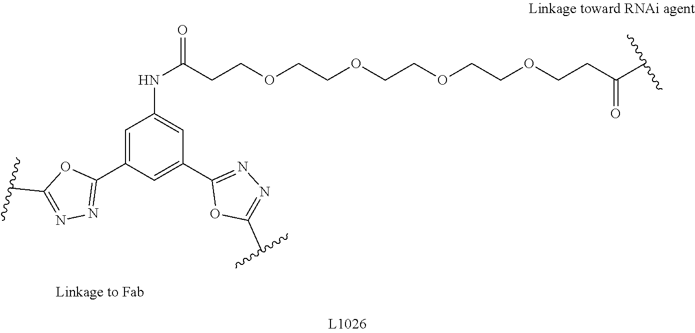 [SMILES: *C(=O)CCOCCOCCOCCOCCC(=O)Nc1cc(-c2nnc(*)o2)cc(-c2nnc(*)o2)c1]
and
(g) Fab0070 represents the antibody fragment (Fab), and wherein the antibody fragment comprises:
A. a variable light chain (VL) comprising: a VL complementary determining region (CDR) 1 having an amino acid sequence of RASDKLYSNLA (SEQ ID NO: 8), a VL CDR2 having an amino acid sequence of DATLLAS (SEQ ID NO: 9), and a VL CDR3 having an amino acid sequence of QHFWGTPLT (SEQ ID NO: 15); and
B. a variable heavy chain (VH) comprising: a VH CDR1 having an amino acid sequence of GFTFTSYWMH (SEQ ID NO: 18), a VH CDR2 having an amino acid sequence of EINPTNGRTNYIEKFKS (SEQID NO: 23), and VH CDR3 having an amino acid sequence of GTRAYHY (SEQ ID NO: 25);
wherein [symbol] the indicates a point of connection.

80. The conjugate or a pharmaceutically acceptable salt thereof of claim 79, wherein the antisense strand consists of the nucleotide sequence (5′ to 3′) cPrpusAfscaAfcgagacUfgAfaUfugccssu (SEQ ID NO: 468).

81. The conjugate or a pharmaceutically acceptable salt thereof of claim 79, wherein:
A. the variable light chain of the Fab consists of an amino acid sequence
(SEQ IDNO: 32)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIK,
and
B. the variable heavy chain of the Fab consists of an amino acid sequence
(SEQ ID NO: 40)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSS.

82. The conjugate or a pharmaceutically acceptable salt thereof of claim 79, wherein:
A. the light chain of the Fab comprises an amino acid sequence
(SEQ ID NO: 3)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,
and
B. the heavy chain of the Fab comprises an amino acid sequence
(SEQ ID NO: 5)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKRVEPKSCDKTH.

83. The conjugate of claim 79, wherein the conjugate is 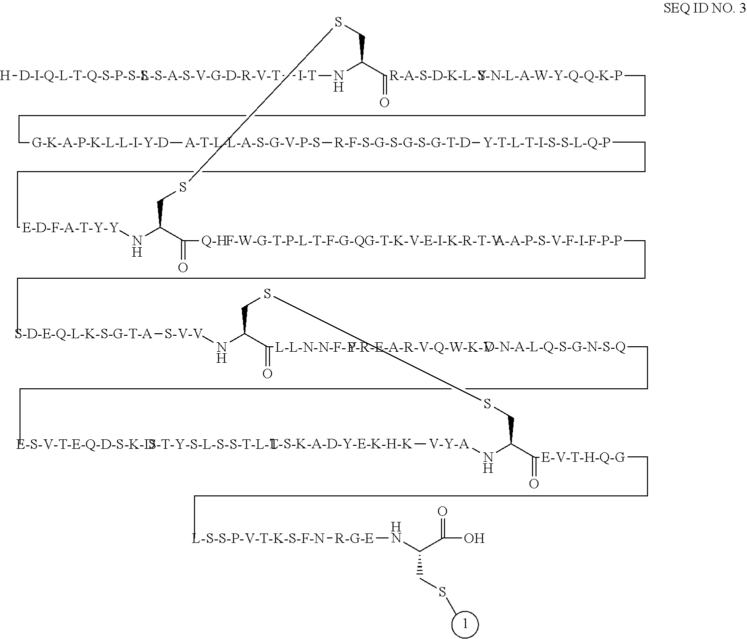 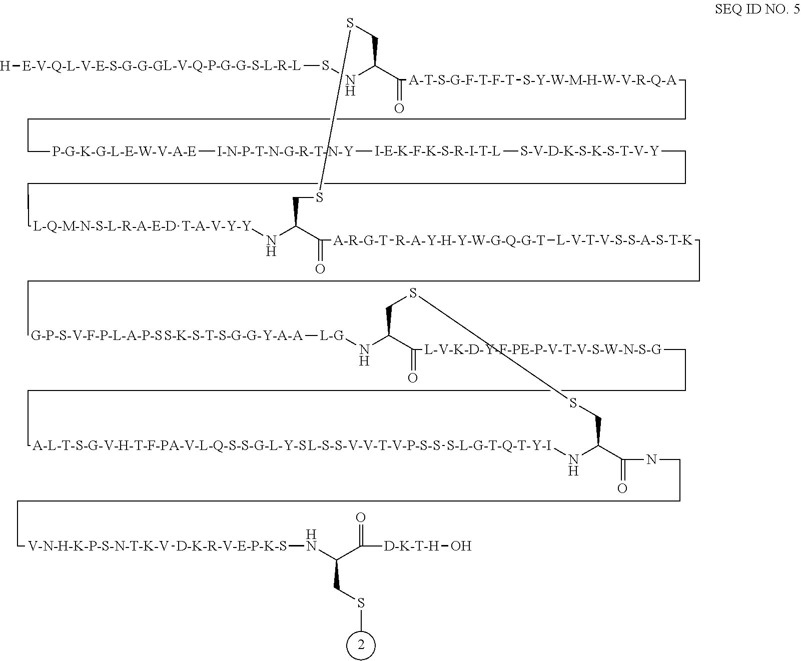 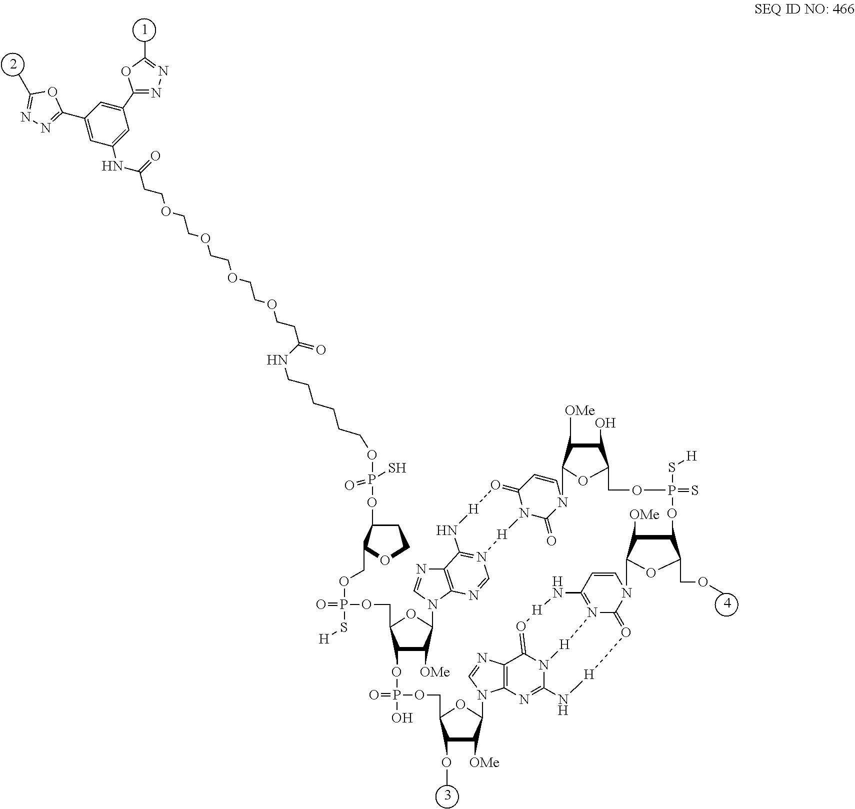 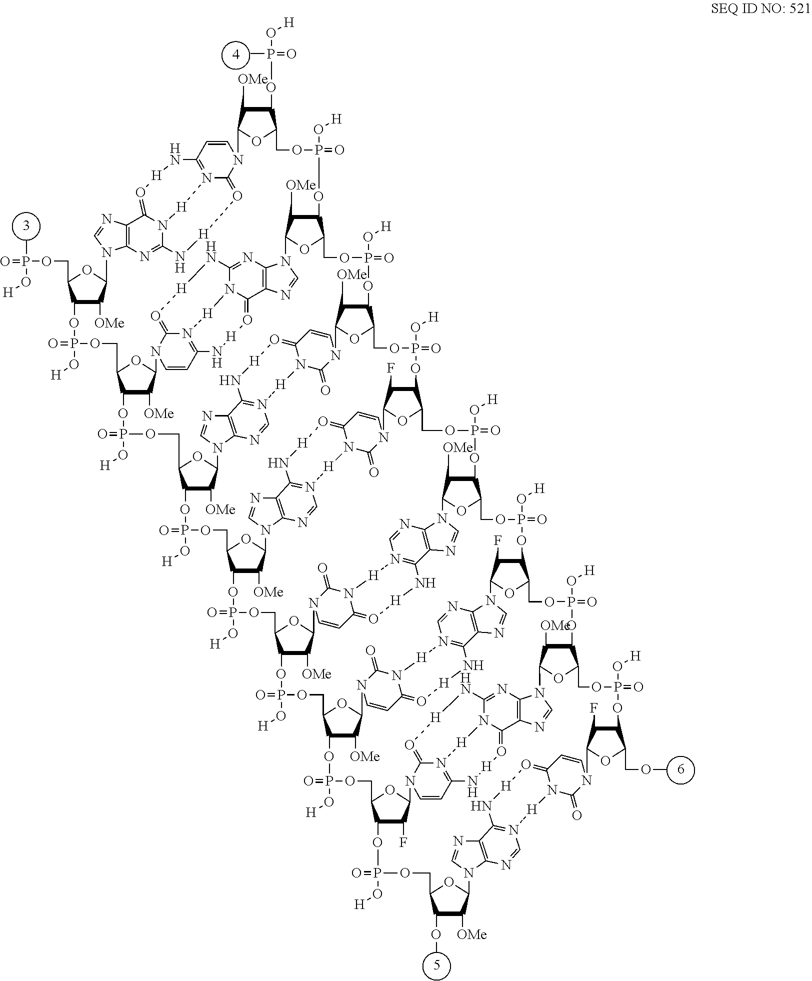 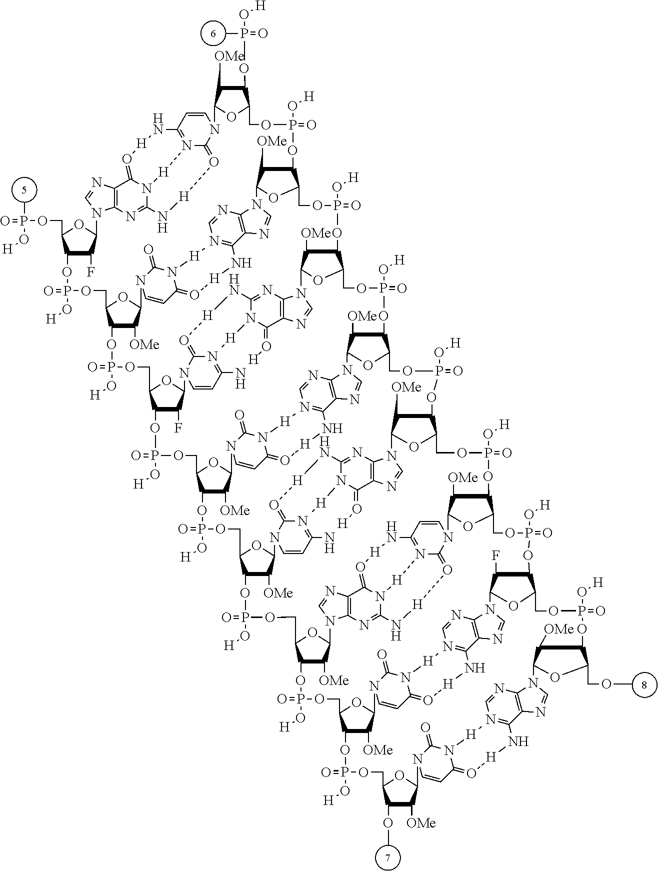 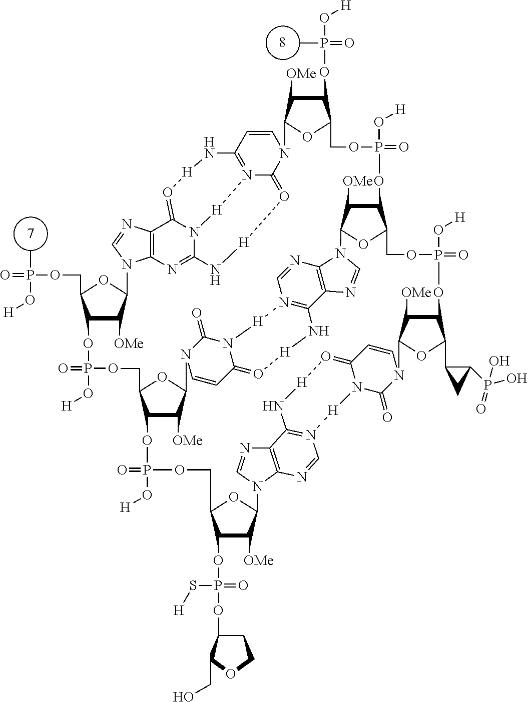
or a pharmaceutically acceptable salt thereof.

84. The conjugate of claim 79, wherein the conjugate is 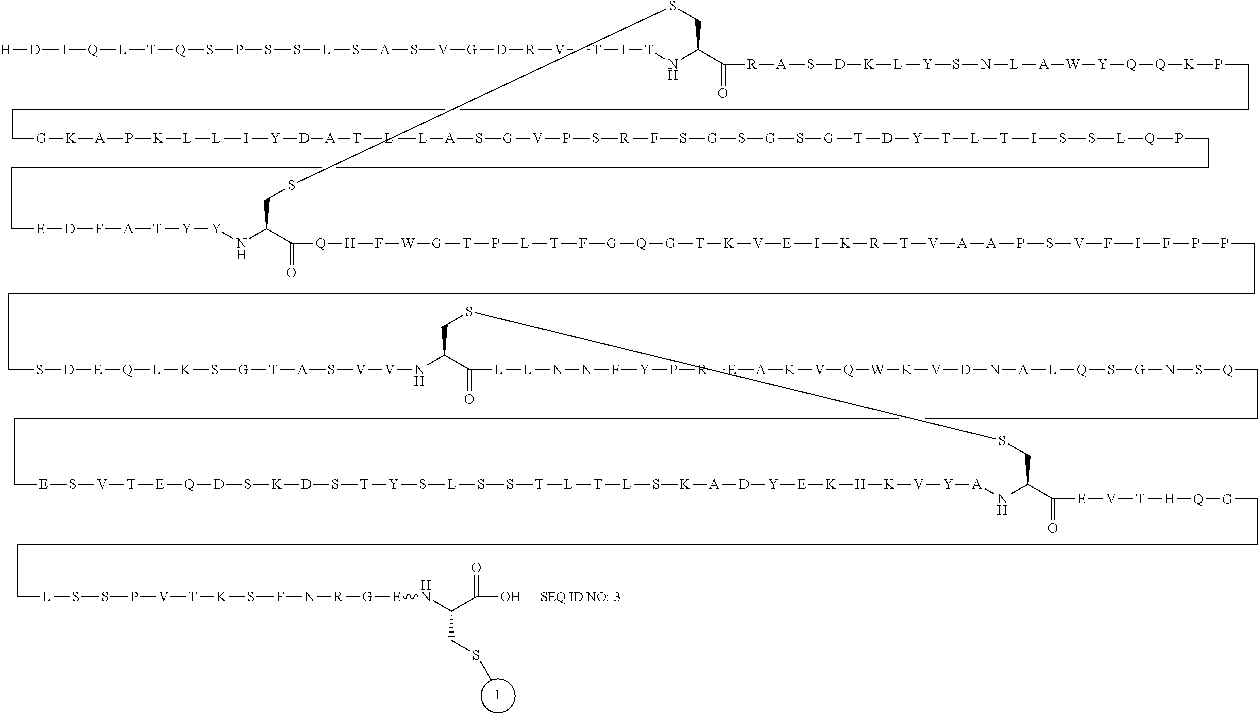 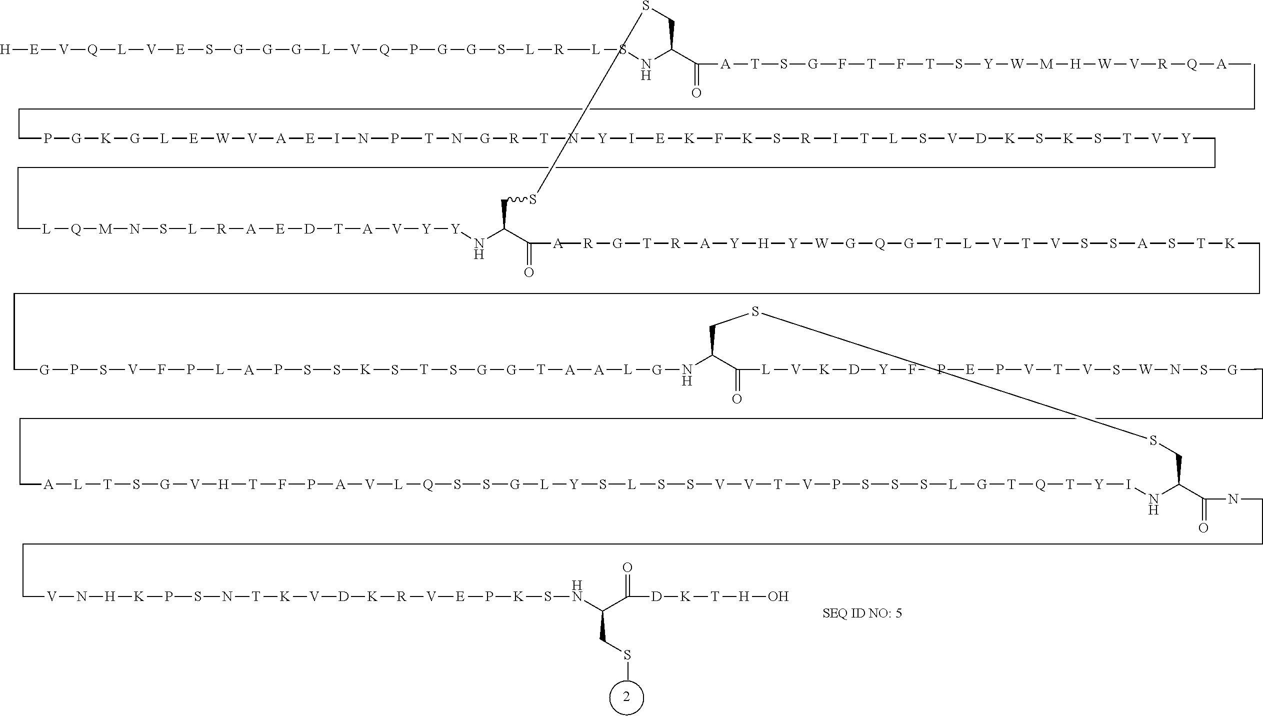 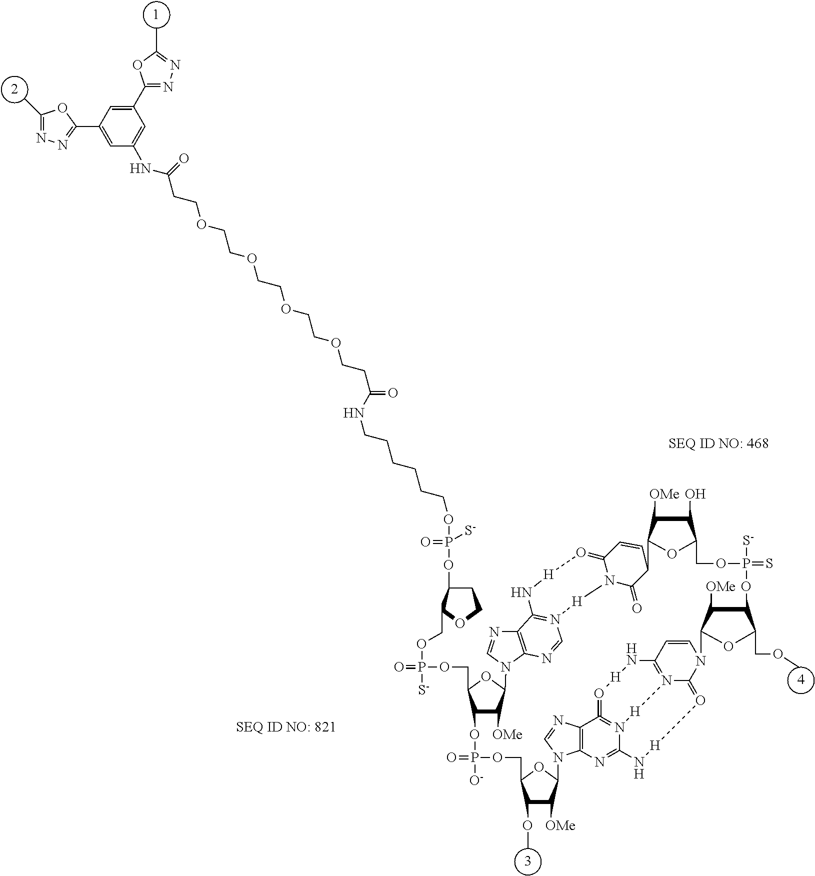 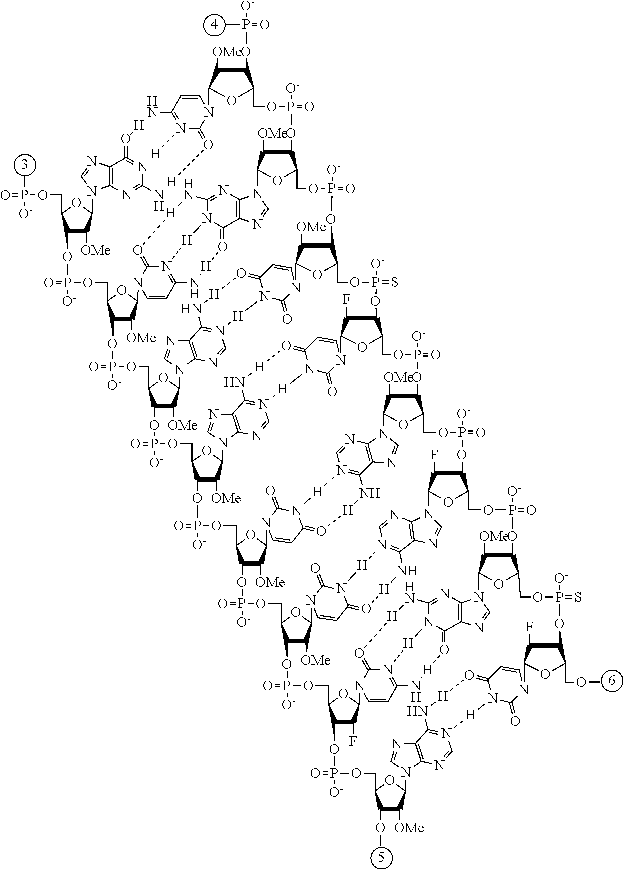 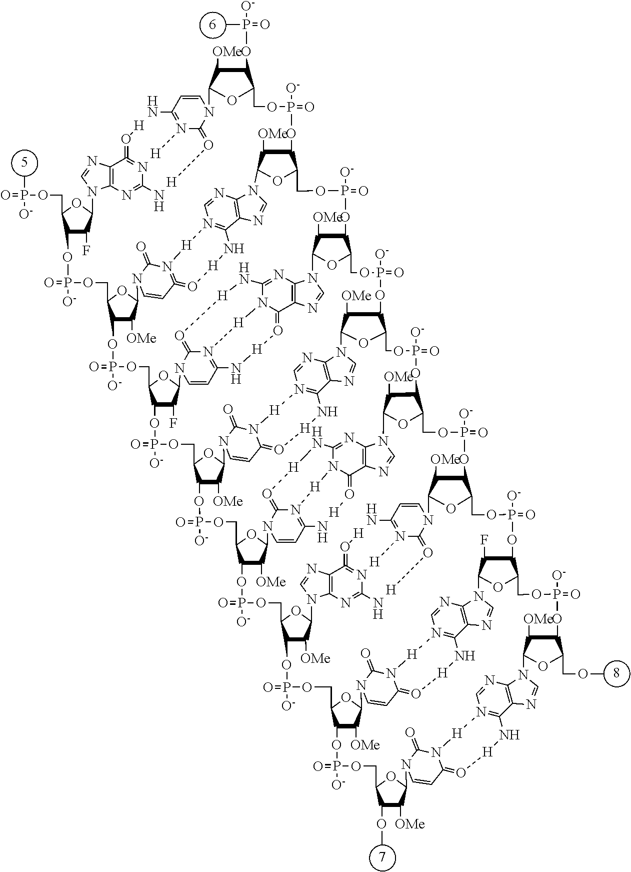 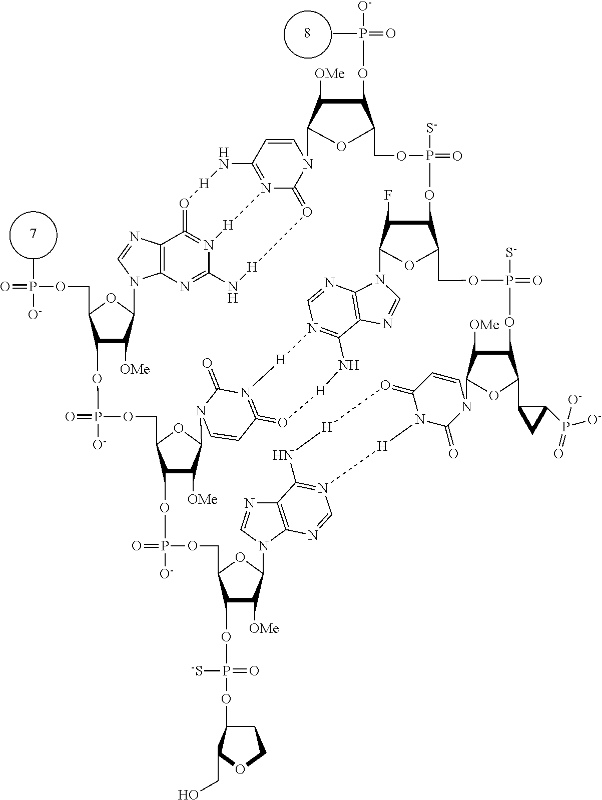
or a pharmaceutically acceptable salt thereof.

85. A composition comprising:
I. a conjugate, or a pharmaceutically acceptable salt thereof, comprising an RNAi agent and an antibody fragment (Fab), wherein the conjugate comprises:
i. an antisense strand comprising the nucleotide sequence (5′ to 3′) cPrpusAfscaAfcgagacUfgAfaUfugeessu (SEQ ID NO: 468), and
ii. a sense strand comprising Fab0070-L1026-C6s(invAb)saggcaauuCfaGfuCfucguuguas(invAb) (5′ to 3′) (SEQ ID NO: 821);
wherein:
(a) a represents 2′-O-methyl adenosine, c represents 2′-O-methyl cytidine, g represents 2′-O-methyl guanosine, u represents 2′-O-methyl uridine, Af represents 2′-fluoro adenosine, Cf represents 2′-fluoro cytidine, Gf represents 2′-fluoro guanosine, Uf represents 2′-fluoro uridine, s represents a phosphorothioate linkage, and ss represents a phosphorodithioate linkage;
(b) cPrpus represents 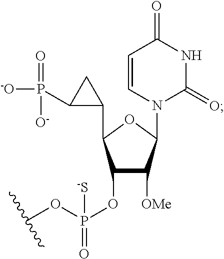
(c) (invAb) represents
linkage towards 5′ end 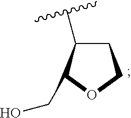
(d) (invAb)s represents
linkage towards 5′ end 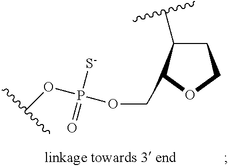
(e) C6s represents 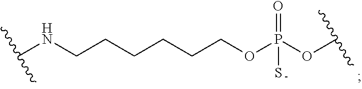 [SMILES: *NCCCCCCOP(=O)(S)O*]
(f) L1026 represents 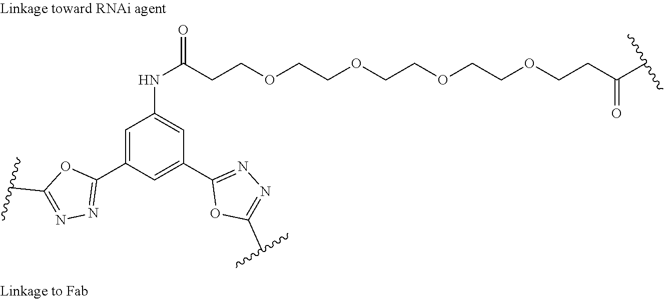 [SMILES: *C(=O)CCOCCOCCOCCOCCC(=O)Nc1cc(-c2nnc(*)o2)cc(-c2nnc(*)o2)c1]
and
(g) Fab0070 represents the antibody fragment (Fab), and wherein the antibody fragment comprises:
A. a variable light chain (VL) comprising: a VL complementary determining region (CDR) 1 having an amino acid sequence of RASDKLYSNLA (SEQ ID NO: 8), a VL CDR2 having an amino acid sequence of DATLLAS (SEQ ID NO: 9), and a VL CDR3 having an amino acid sequence of QHFWGTPLT (SEQ ID NO: 15); and
B. a variable heavy chain (VH) comprising: a VH CDR1 having an amino acid sequence of GFTFTSYWMH (SEQ ID NO: 18), a VH CDR2 having an amino acid sequence of EINPTNGRTNYIEKFKS (SEQID NO: 23), and VH CDR3 having an amino acid sequence of GTRAYHY (SEQ ID NO: 25);
wherein the [symbol] indicates a point of connection; and
II. a pharmaceutically acceptable excipient.

86. The composition of claim 85, wherein the antisense strand consists of the nucleotide sequence (5′ to 3′) cPrpusAfscaAfcgagacUfgAfaUfugccssu (SEQ ID NO: 468).

87. The composition of claim 85, wherein:
A. the variable light chain of the Fab consists of an amino acid sequence
(SEQ IDNO: 32)
DIQLTQSPSSLSASVGDRVTITCRASDKLYSNLAWYQQKPGKAPKLLIYD
ATLLASGVPSRFSGSGSGTDYTLTISSLQPEDFATYYCQHFWGTPLTFGQ
GTKVEIK,
and
B. the variable heavy chain of the Fab consists of an amino acid sequence
(SEQ ID NO: 40)
EVQLVESGGGLVQPGGSLRLSCATSGFTFTSYWMHWVRQAPGKGLEWVAE
INPTNGRTNYIEKFKSRITLSVDKSKSTVYLQMNSLRAEDTAVYYCARGT
RAYHYWGQGTLVTVSS.

88. The composition of claim 85, wherein:
A. the light chain of the Fab comprises an amino acid sequence
(SEQ ID NO: 3)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC,
and
B. the heavy chain of the Fab comprises an amino acid sequence
(SEQ ID NO: 5)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKRVEPKSCDKTH.

89. The composition of claim 85, wherein the conjugate is 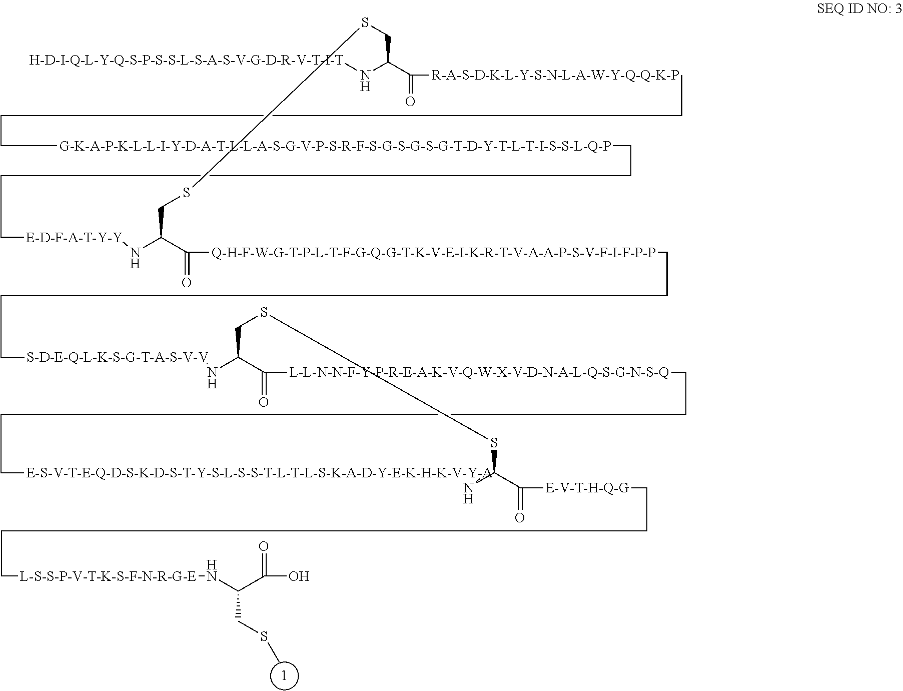 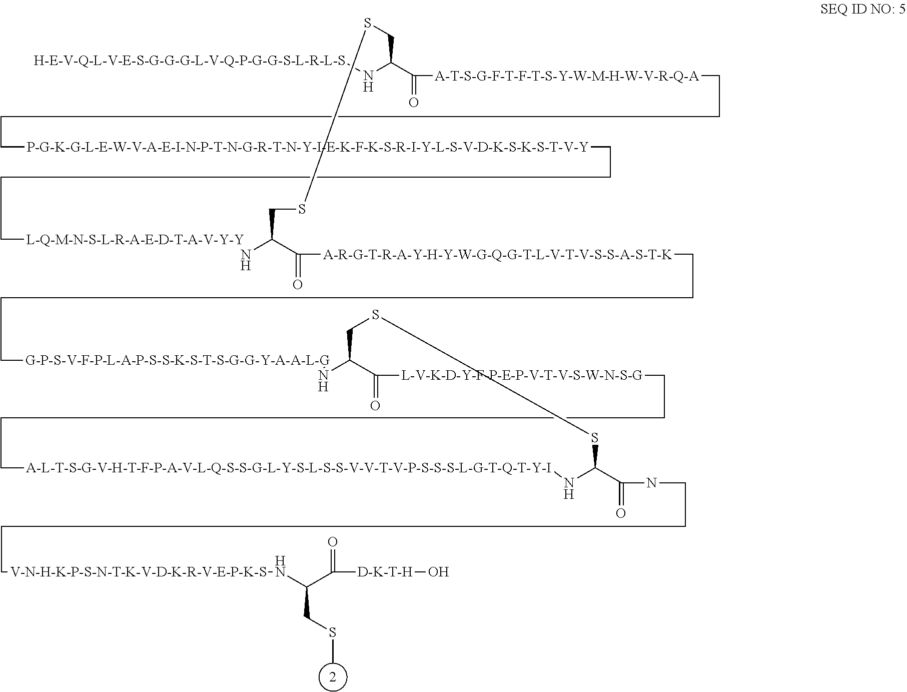 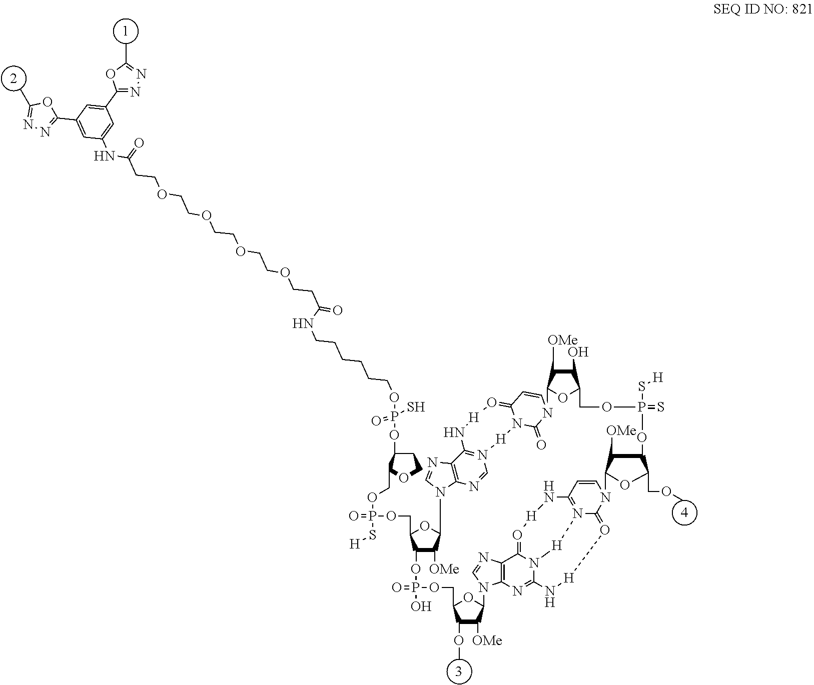 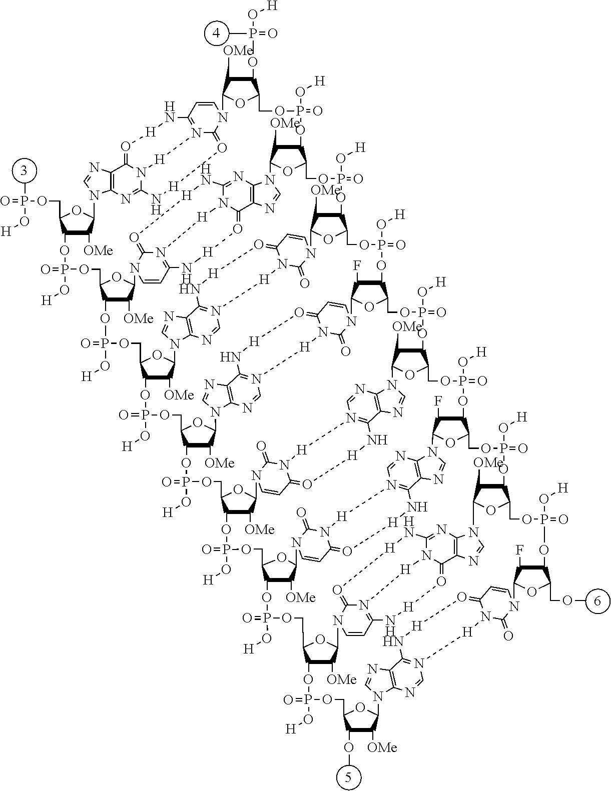 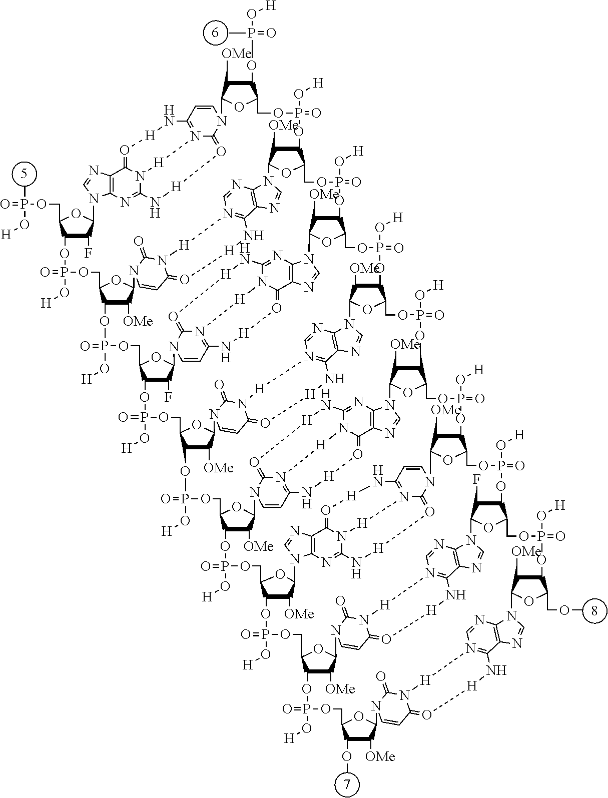 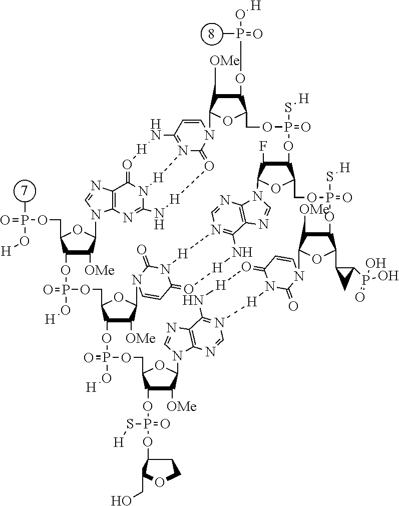
or a pharmaceutically acceptable salt thereof.

90. The composition of claim 85, wherein the conjugate is 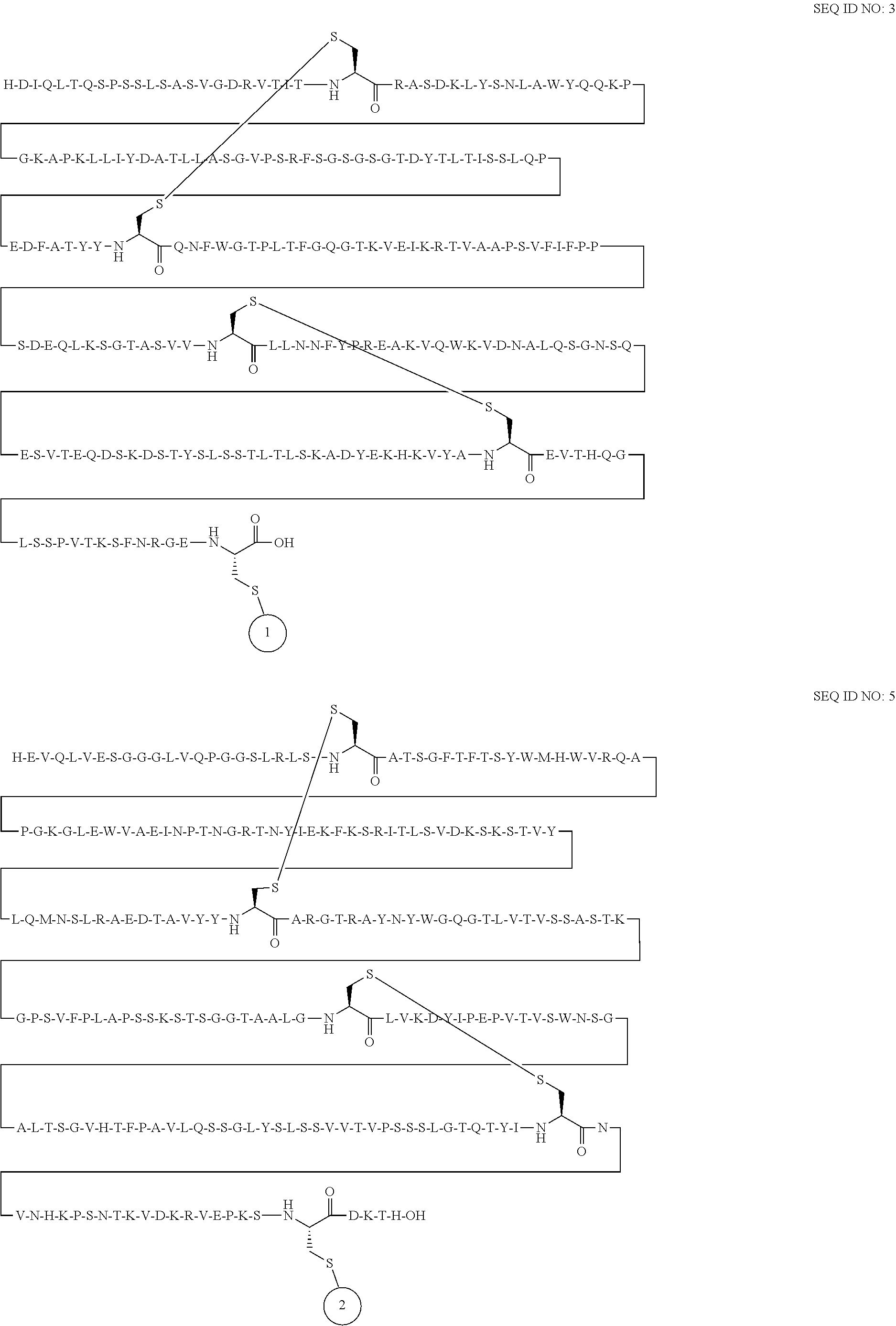 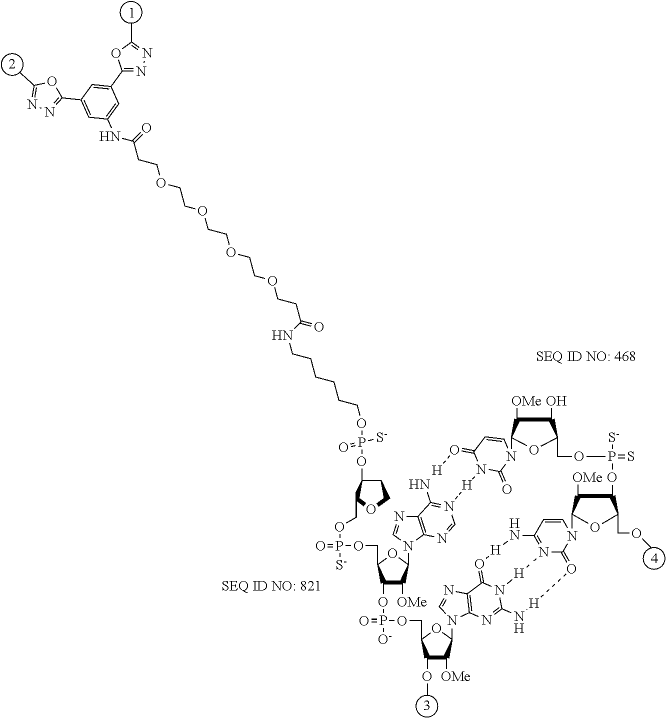 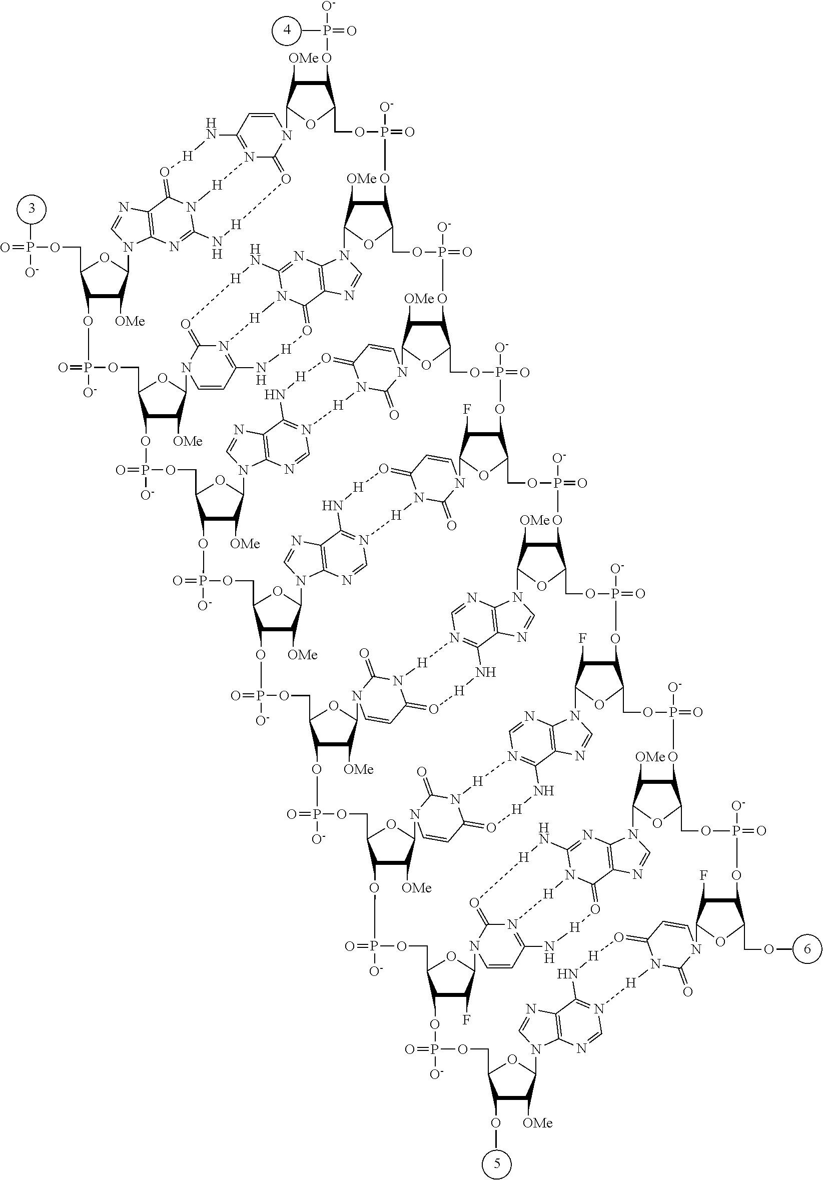 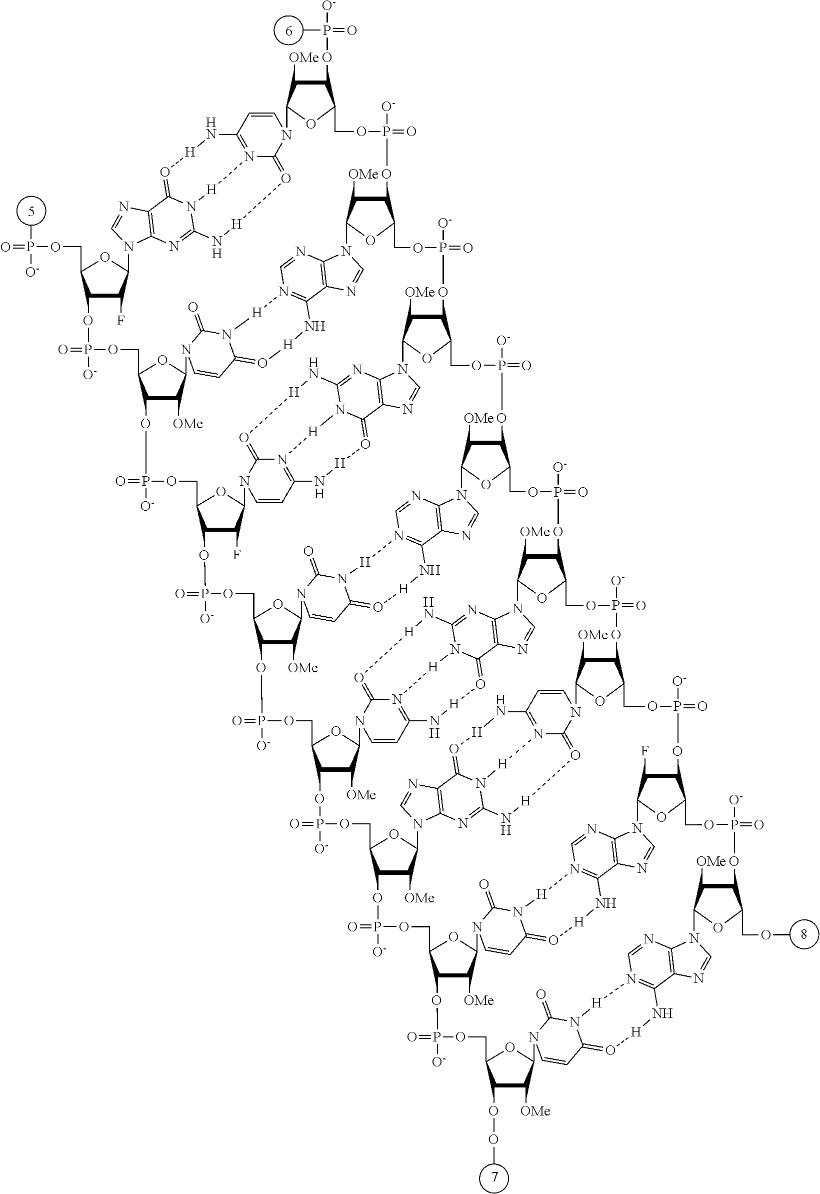 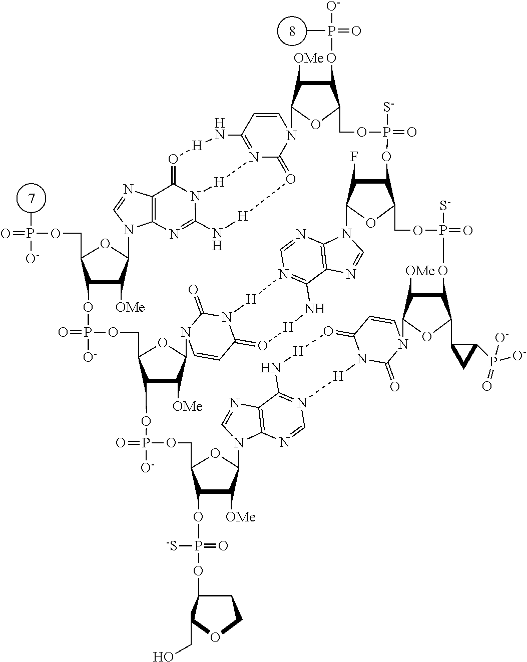
or a pharmaceutically acceptable salt thereof.

91. A method for inhibiting expression of a huntingtin (HTT) gene in a cell, the method comprising introducing into the cell an effective amount of a conjugate, or a pharmaceutically acceptable salt thereof, comprising an RNAi agent and an antibody fragment (Fab), wherein the conjugate comprises:
i. an antisense strand comprising the nucleotide sequence (5′ to 3′) cPrpusAfscaAfcgagacUfgAfaUfugccssu (SEQ ID NO: 468), and
ii. a sense strand comprising Fab0070-L1026-C6s(invAb)saggcaauuCfaGfuCfucguuguas(invAb) (5′ to 3′) (SEQ ID NO: 821);
wherein:
(a) a represents 2′-O-methyl adenosine, c represents 2′-O-methyl cytidine, g represents 2′-O-methyl guanosine, u represents 2′-O-methyl uridine, Af represents 2′-fluoro adenosine, Cf represents 2′-fluoro cytidine, Gf represents 2′-fluoro guanosine, Uf represents 2′-fluoro uridine, s represents a phosphorothioate linkage, and ss represents a phosphorodithioate linkage;
(b) cPrpus represents 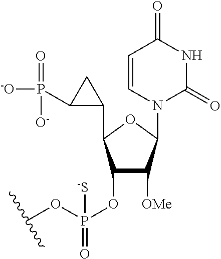
(c) (invAb) represents 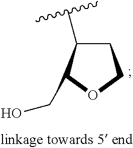
(d) (invAb)s represents 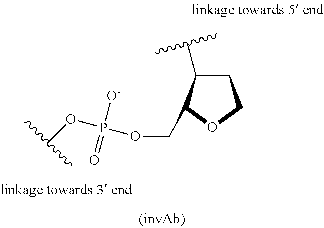
(e) C6s represents 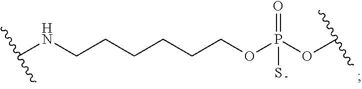 [SMILES: *NCCCCCCOP(=O)(S)O*]
(f) L1026 represents 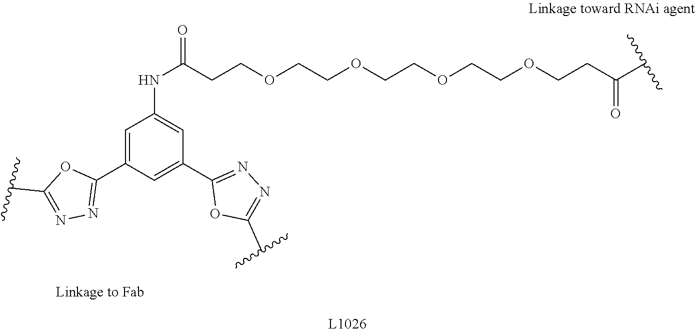 [SMILES: *C(=O)CCOCCOCCOCCOCCC(=O)Nc1cc(-c2nnc(*)o2)cc(-c2nnc(*)o2)c1]
(g) Fab0070 represents the antibody fragment (Fab), and wherein the antibody fragment comprises:
A. a variable light chain (VL) comprising: a VL complementary determining region (CDR) 1 having an amino acid sequence of RASDKLYSNLA (SEQ ID NO: 8), a VL CDR2 having an amino acid sequence of DATLLAS (SEQ ID NO: 9), and a VL CDR3 having an amino acid sequence of QHFWGTPLT (SEQ ID NO: 15); and
B. a variable heavy chain (VH) comprising: a VH CDR1 having an amino acid sequence of GFTFTSYWMH (SEQ ID NO: 18), a VH CDR2 having an amino acid sequence of EINPTNGRTNYIEKFKS (SEQID NO: 23), and VH CDR3 having an amino acid sequence of GTRAYHY (SEQ ID NO: 25);
wherein the [symbol] indicates a point of connection.

92. The method of claim 91, wherein the antisense strand consists of the nucleotide sequence (5′ to 3′) cPrpusAfscaAfcgagacUfgAfaUfugccssu (SEQ ID NO: 468).

93. The method of claim 91, wherein:
A. the variable light chain of the Fab consists of an amino acid sequence
(SEQ ID NO: 32)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIK,
and
B. the variable heavy chain of the Fab consists of an amino acid sequence
(SEQ ID NO: 40)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSS.

94. The method of claim 91, wherein:
A. the light chain of the Fab comprises an amino acid sequence
(SEQ ID NO: 3)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC,
and
B. the heavy chain of the Fab comprises an amino acid sequence
(SEQ ID NO: 5)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKRVEPKSCDKTH.

95. The method of claim 91, wherein the conjugate is 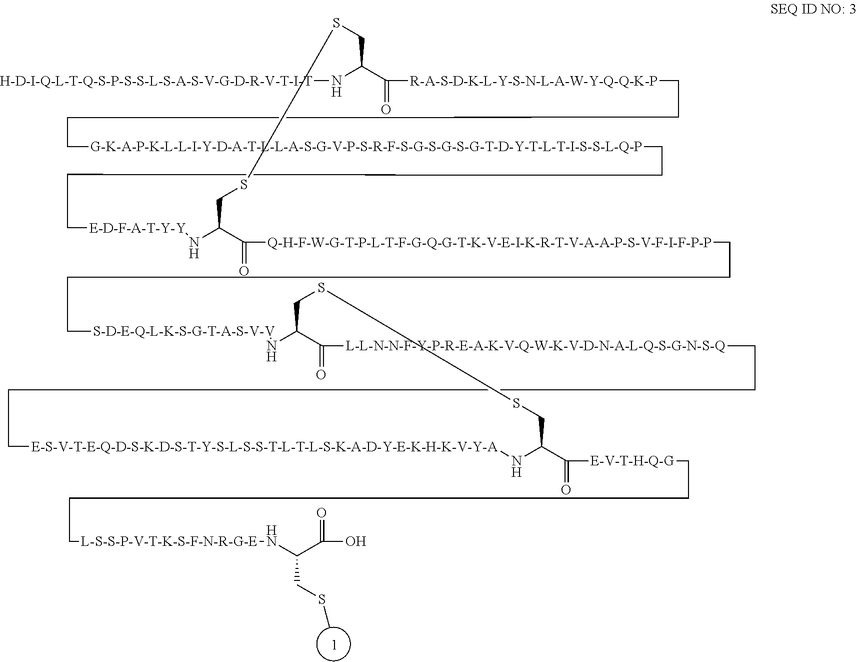 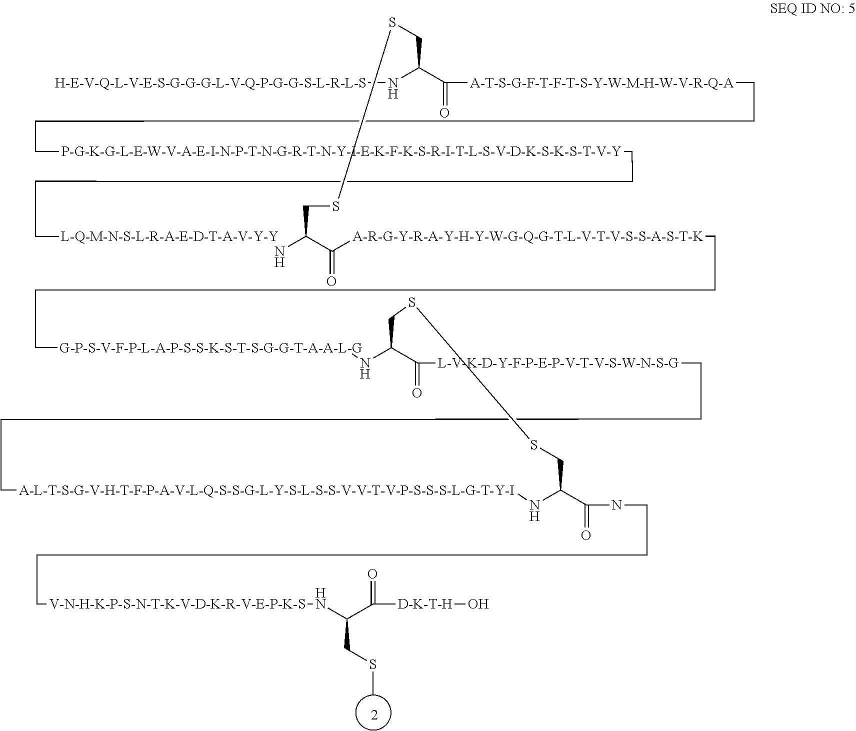 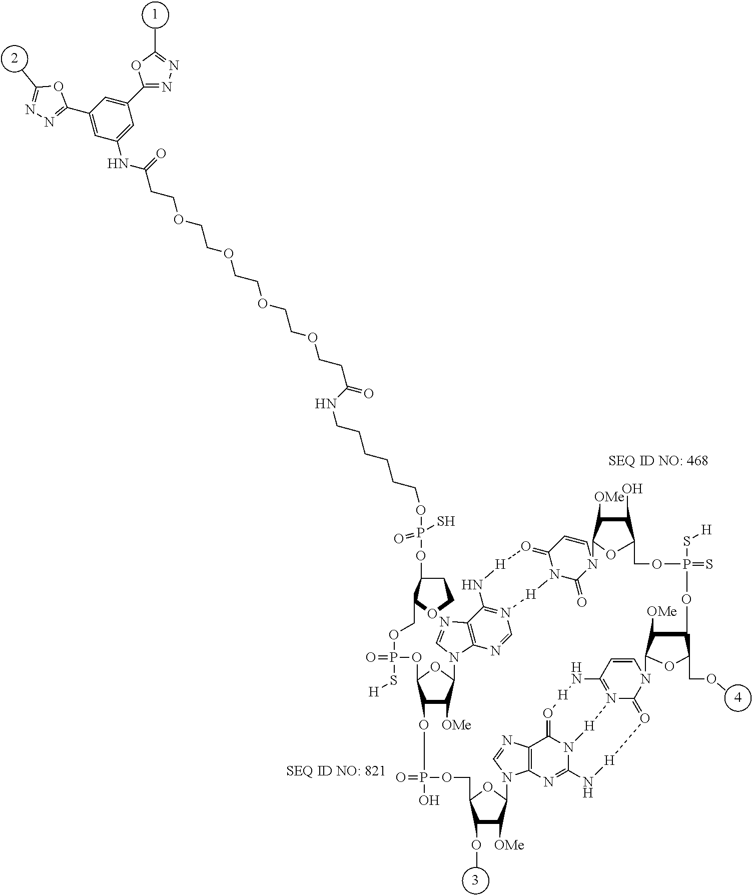 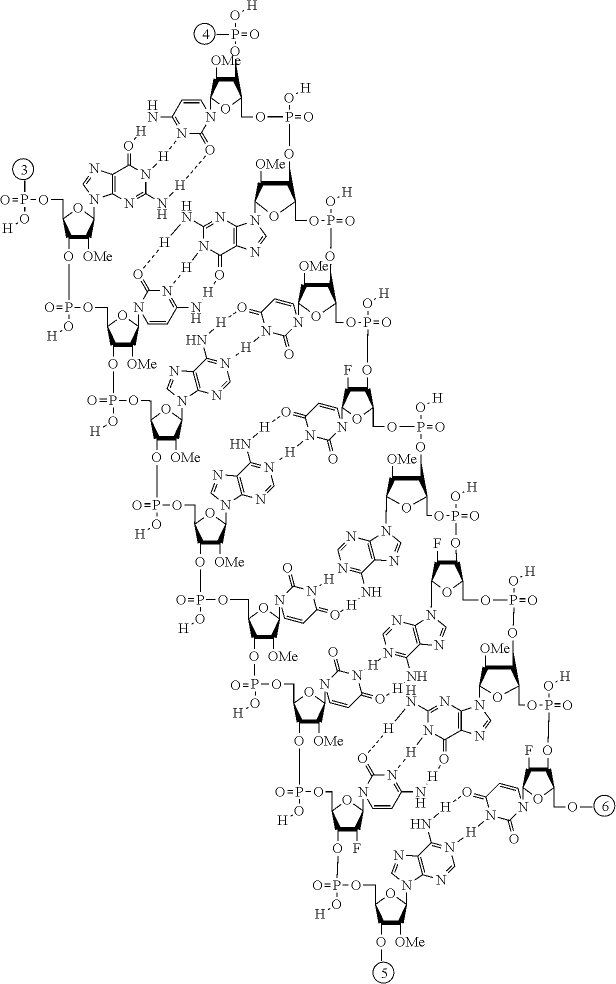 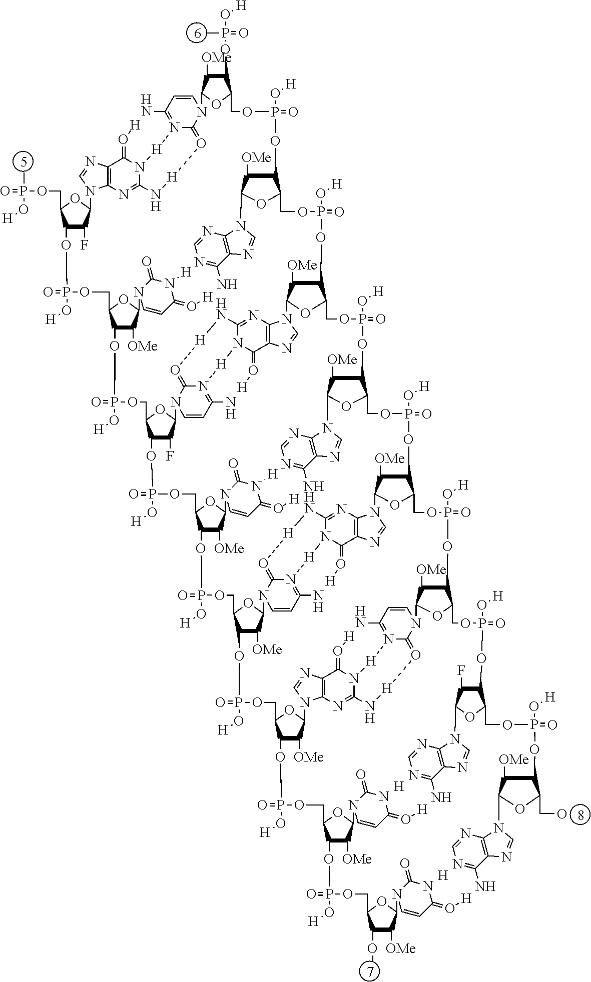 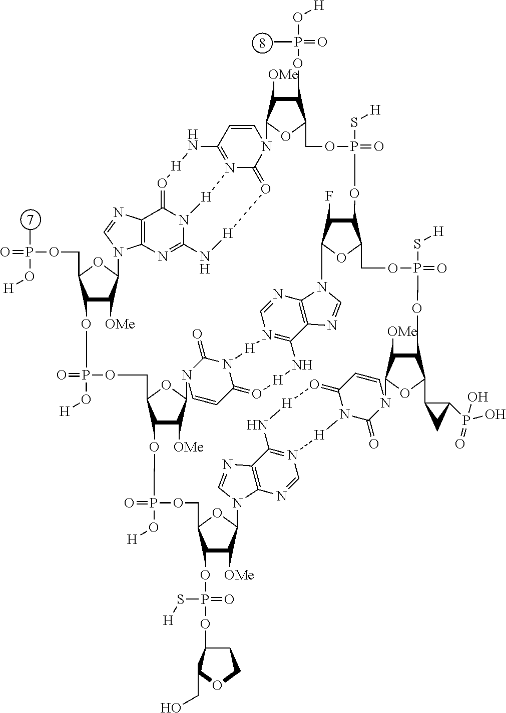
or a pharmaceutically acceptable salt thereof.

96. The method of claim 91, wherein the conjugate is 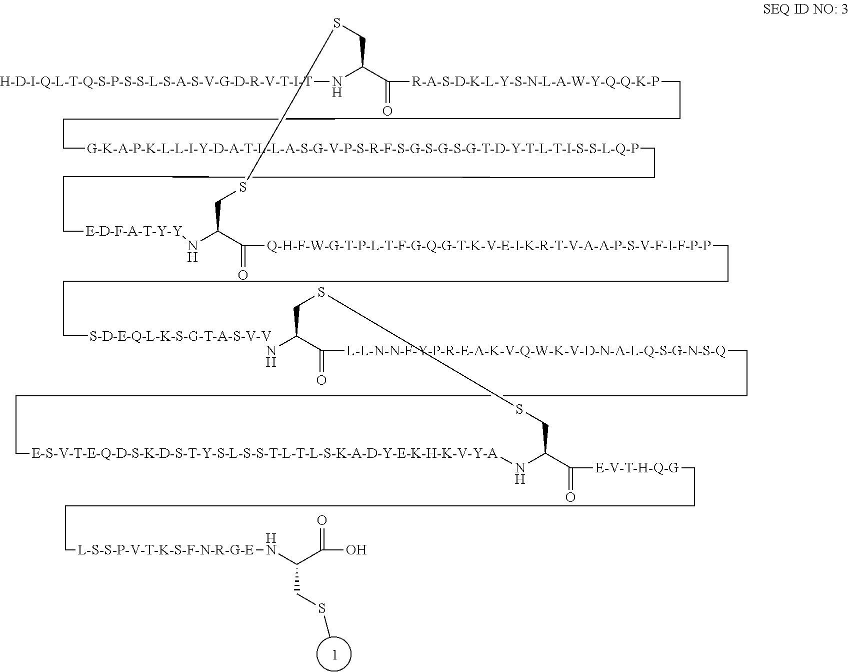 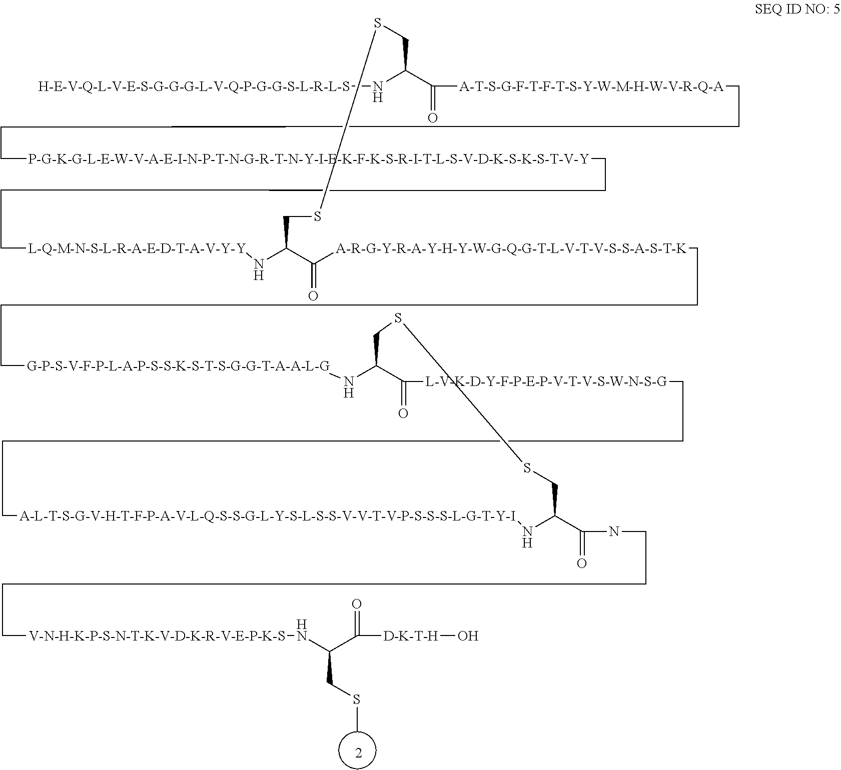 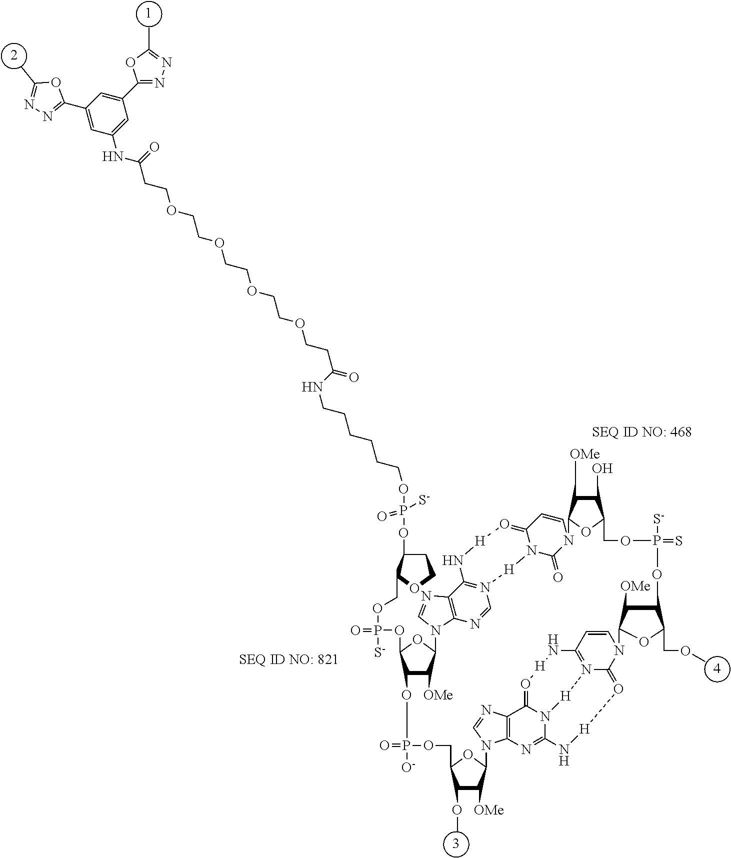 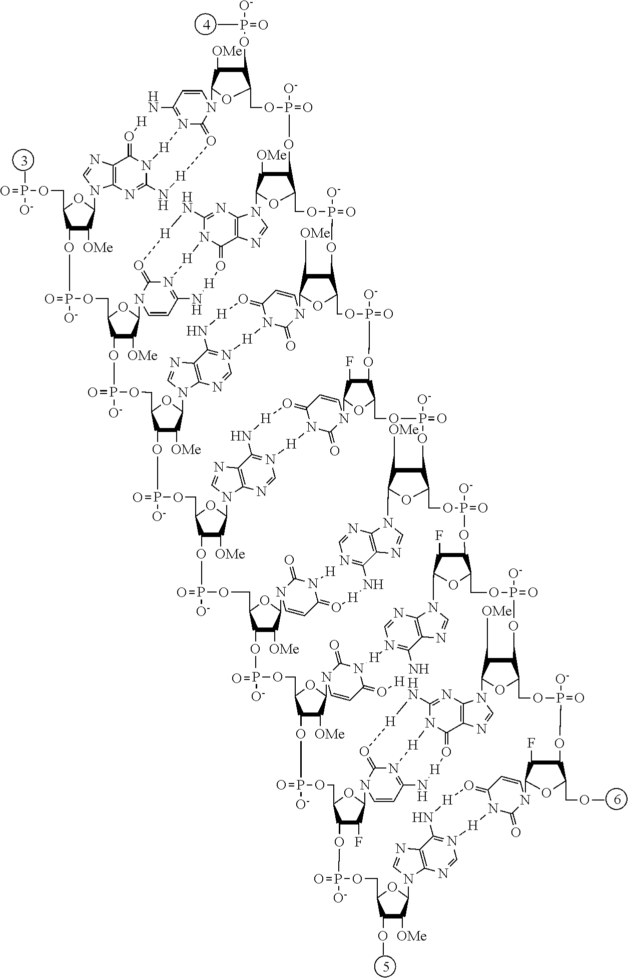 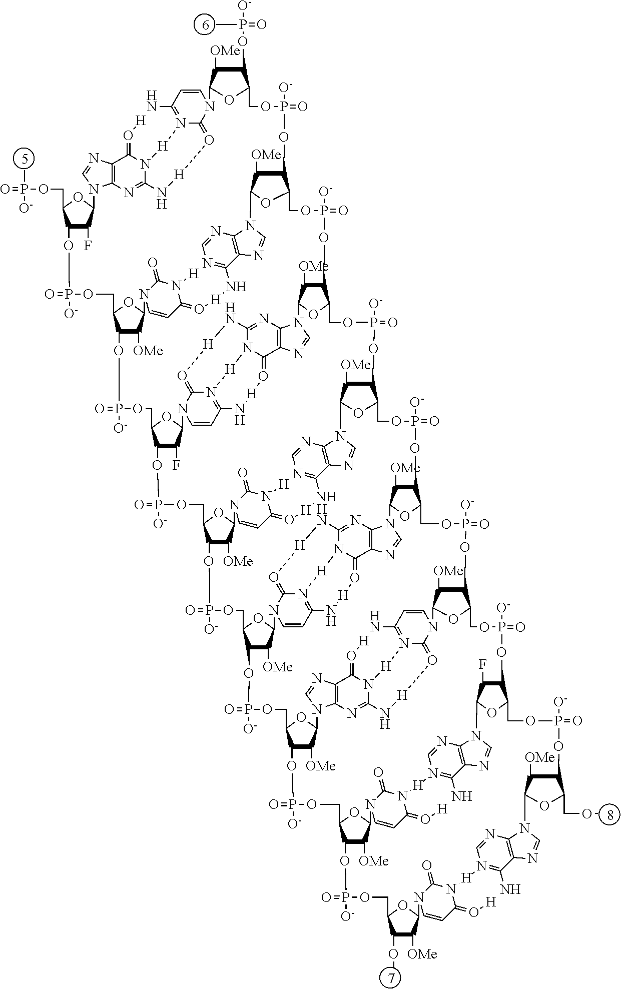 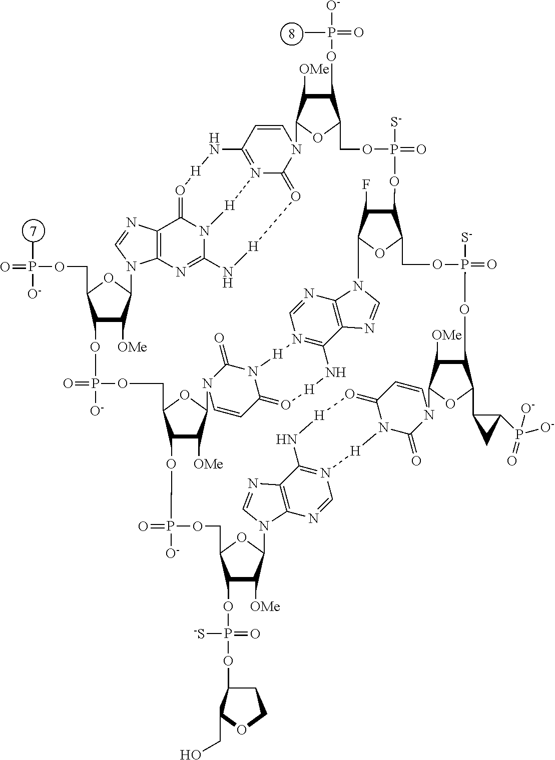
or a pharmaceutically acceptable salt thereof.

97. A method of treating one or more symptoms or diseases associated with enhanced or elevated mutant huntingtin (HTT) activity levels, the method comprising administering to a human subject in need thereof a therapeutically effective amount of a conjugate, or a pharmaceutically acceptable salt thereof, comprising an RNAi agent and an antibody fragment (Fab), wherein the conjugate comprises:
i. an antisense strand comprising the nucleotide sequence (5′ to 3′) cPrpusAfscaAfcgagacUfgAfaUfugccssu (SEQ ID NO: 468), and
ii. a sense strand comprising Fab0070-L1026-C6s(invAb)saggcaauuCfaGfuCfucguuguas(invAb) (5′ to 3′) (SEQ ID NO: 821);
wherein:
(a) a represents 2′-O-methyl adenosine, c represents 2′-O-methyl cytidine, g represents 2′-O-methyl guanosine, u represents 2′-O-methyl uridine, Af represents 2′-fluoro adenosine, Cf represents 2′-fluoro cytidine, Gf represents 2′-fluoro guanosine, Uf represents 2′-fluoro uridine, s represents a phosphorothioate linkage, and ss represents a phosphorodithioate linkage;
(b) cPrpus represents 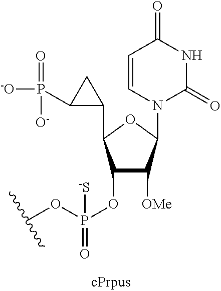
(c) (invAb) represents 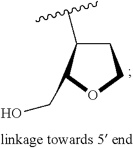
(d) (invAb)s represents 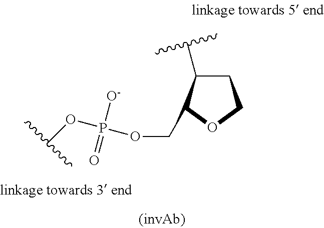
(e) C6s represents 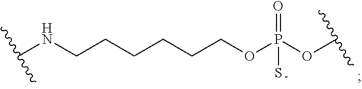 [SMILES: *NCCCCCCOP(=O)(S)O*]
(f) L1026 represents 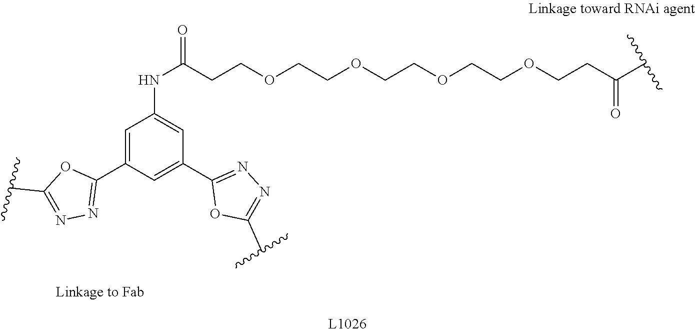 [SMILES: *C(=O)CCOCCOCCOCCOCCC(=O)Nc1cc(-c2nnc(*)o2)cc(-c2nnc(*)o2)c1]
and
(g) Fab0070 represents the antibody fragment (Fab), and wherein the antibody fragment comprises:
A. a variable light chain (VL) comprising: a VL complementary determining region (CDR) 1 having an amino acid sequence of RASDKLYSNLA (SEQ ID NO: 8), a VL CDR2 having an amino acid sequence of DATLLAS (SEQ ID NO: 9), and a VL CDR3 having an amino acid sequence of QHFWGTPLT (SEQ ID NO: 15); and
B. a variable heavy chain (VH) comprising: a VH CDR1 having an amino acid sequence of GFTFTSYWMH (SEQ ID NO: 18), a VH CDR2 having an amino acid sequence of EINPTNGRTNYIEKFKS (SEQID NO: 23), and VH CDR3 having an amino acid sequence of GTRAYHY (SEQ ID NO: 25);
wherein the [symbol] indicates a point of connection.

98. The method of claim 97, wherein the antisense strand consists of the nucleotide sequence (5′ to 3′) cPrpusAfscaAfcgagacUfgAfaUfugccssu (SEQ ID NO: 468).

99. The method of claim 97, wherein:
A. the variable light chain of the Fab consists of an amino acid sequence
(SEQ ID NO: 32)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIK,
and
B. the variable heavy chain of the Fab consists of an amino acid sequence
(SEQ ID NO: 40)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSS.

100. The method of claim 97, wherein:
A. the light chain of the Fab comprises an amino acid sequence
(SEQ ID NO: 3)
DIQLTQSPSSLSASVGDRVTITCRASDKLYSNLAWYQQKPGKAPKL
LIYDATLLASGVPSRFSGSGSGTDYTLTISSLQPEDFATYYCQHFW
GTPLTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC,
and
B. the heavy chain of the Fab comprises an amino acid sequence
(SEQ ID NO: 5)
EVQLVESGGGLVQPGGSLRLSCATSGFTFTSYWMHWVRQAPGKGLE
WVAEINPTNGRTNYIEKFKSRITLSVDKSKSTVYLQMNSLRAEDTA
VYYCARGTRAYHYWGQGTLVTVSSASTKGPSVFPLAPSSKSTSGGT
AALGCLVKDYFPEPVTVSWNSGALTSGVHTFPAVLQSSGLYSLSSV
VTVPSSSLGTQTYICNVNHKPSNTKVDKRVEPKSCDKTH.

101. The method of claim 97, wherein the conjugate is 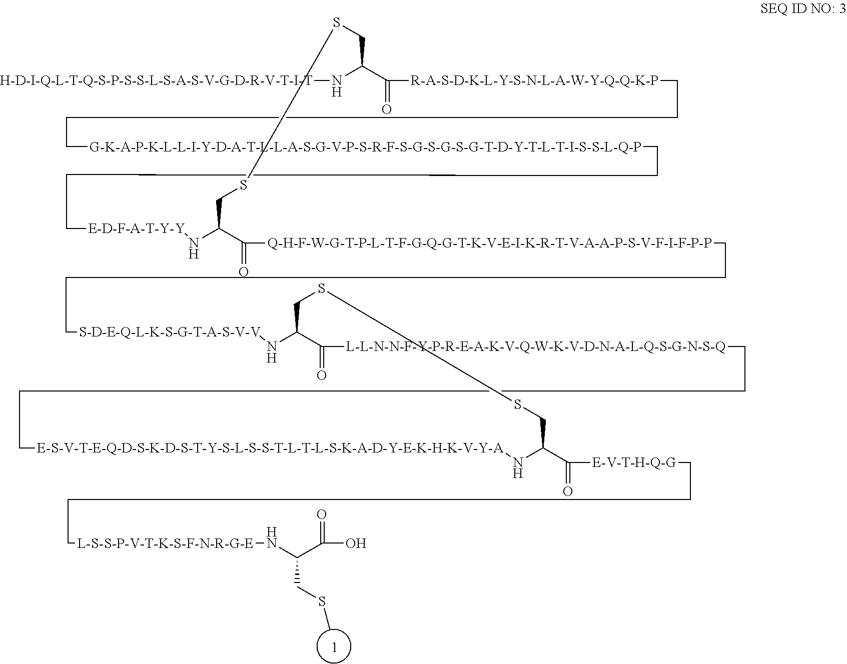 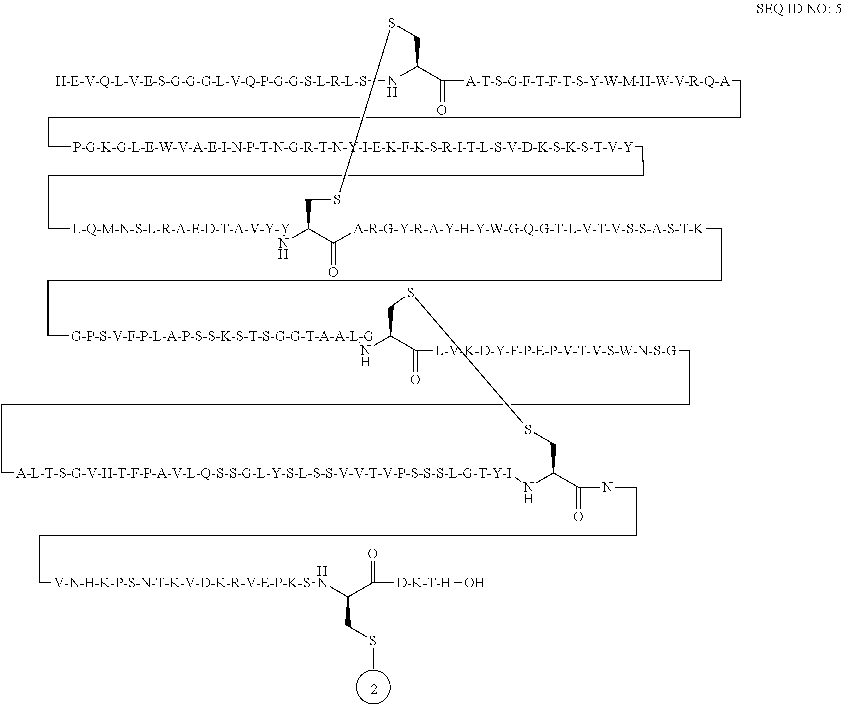 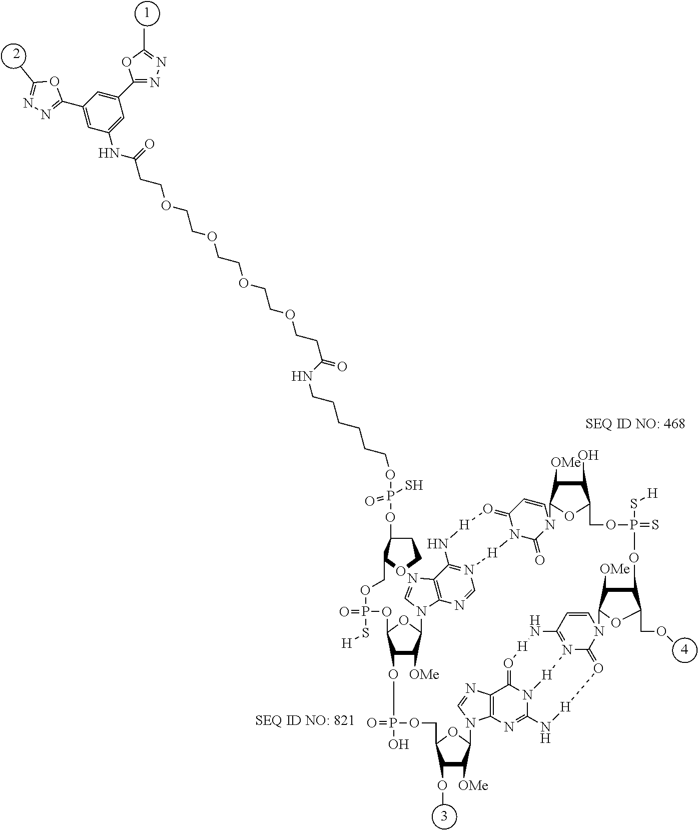 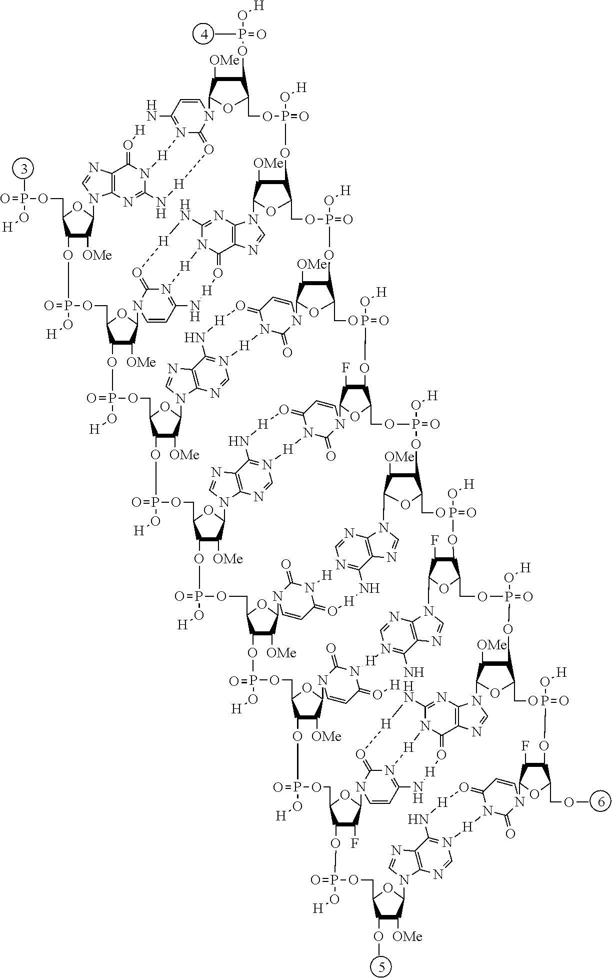 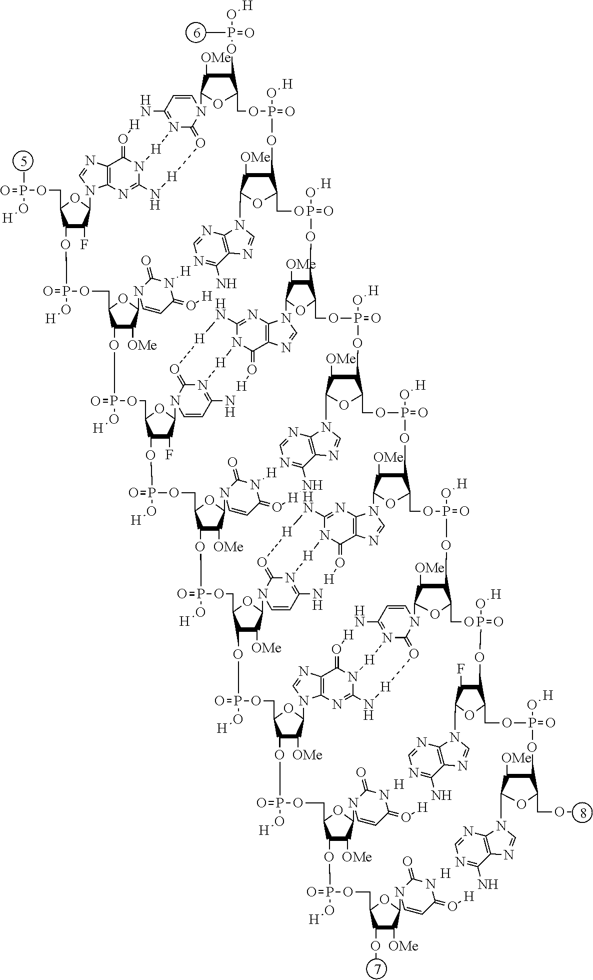 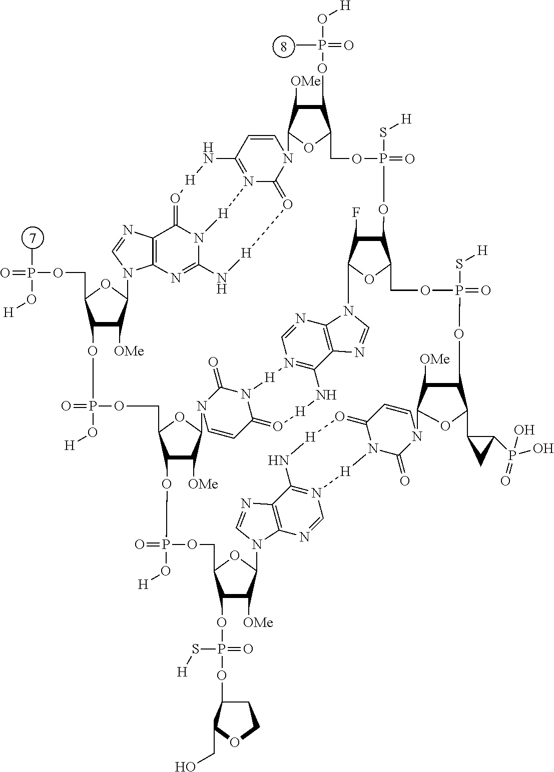
or a pharmaceutically acceptable salt thereof.

102. The method of claim 97, wherein the conjugate is 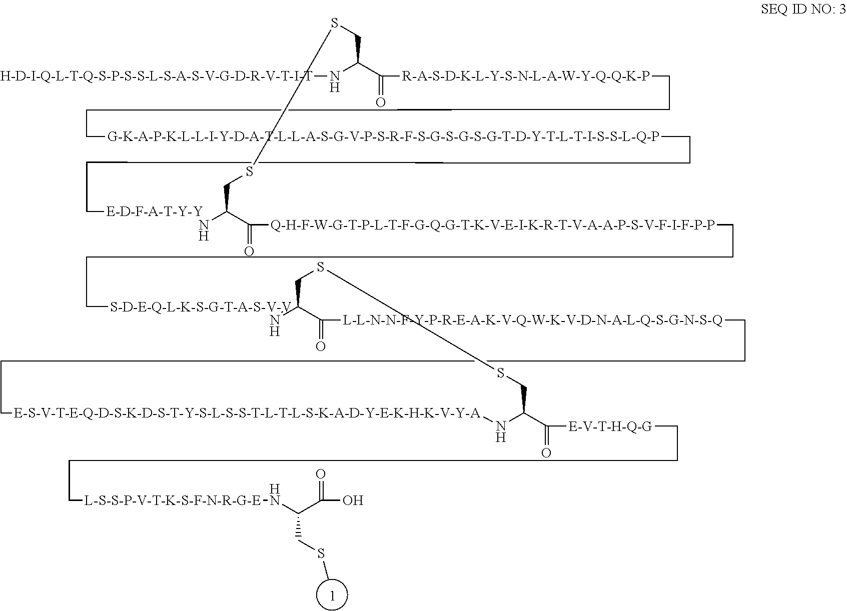 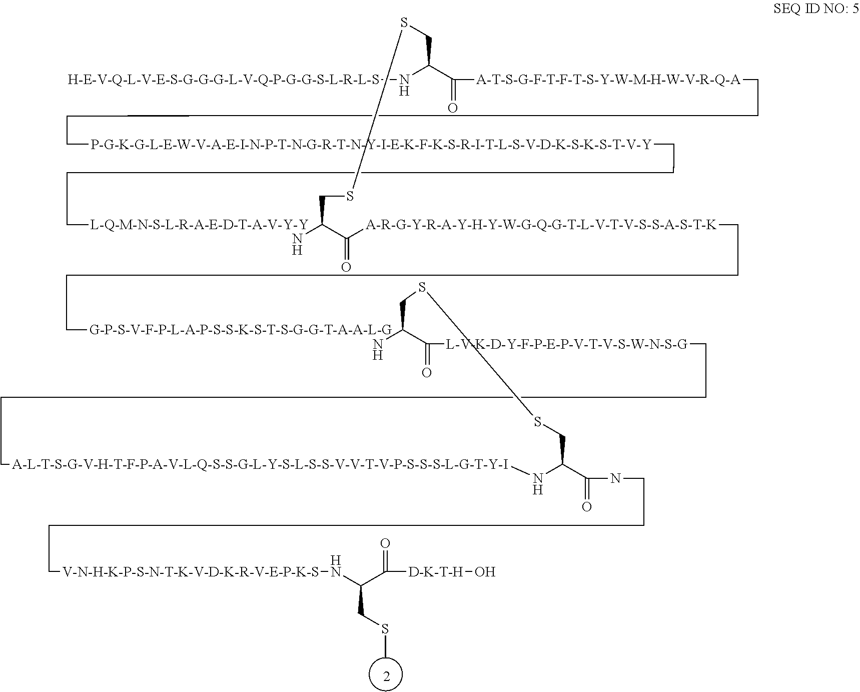 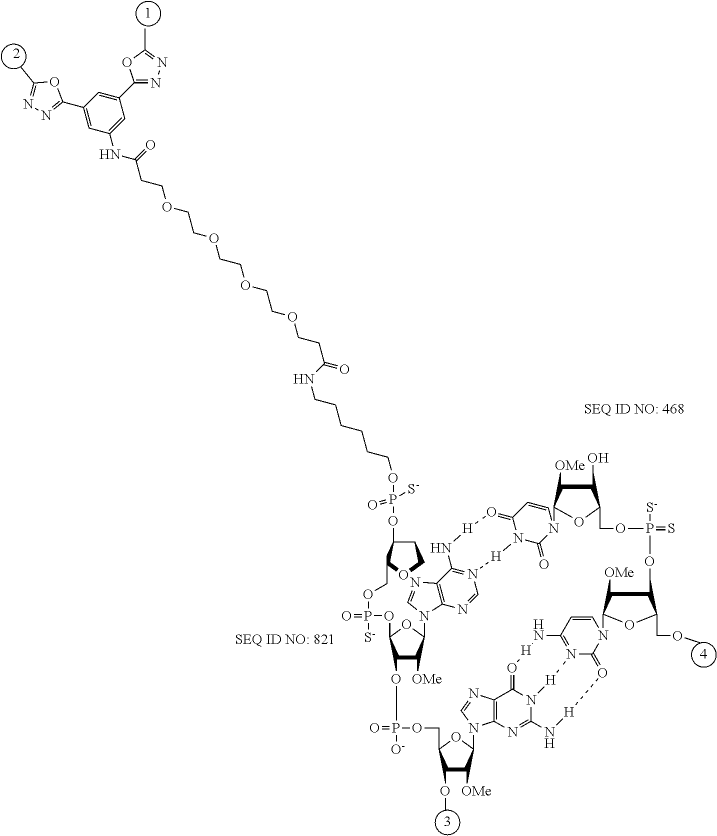 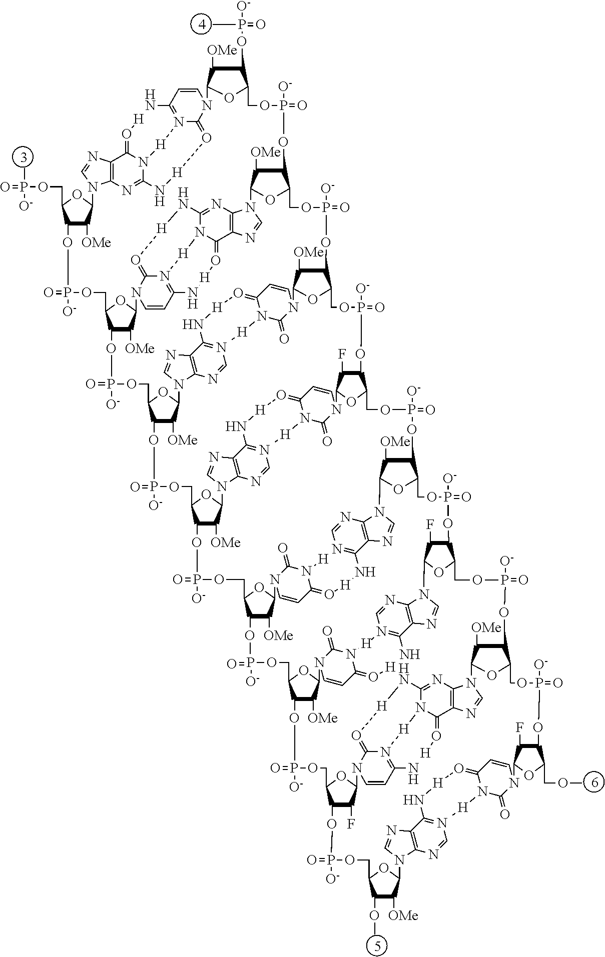 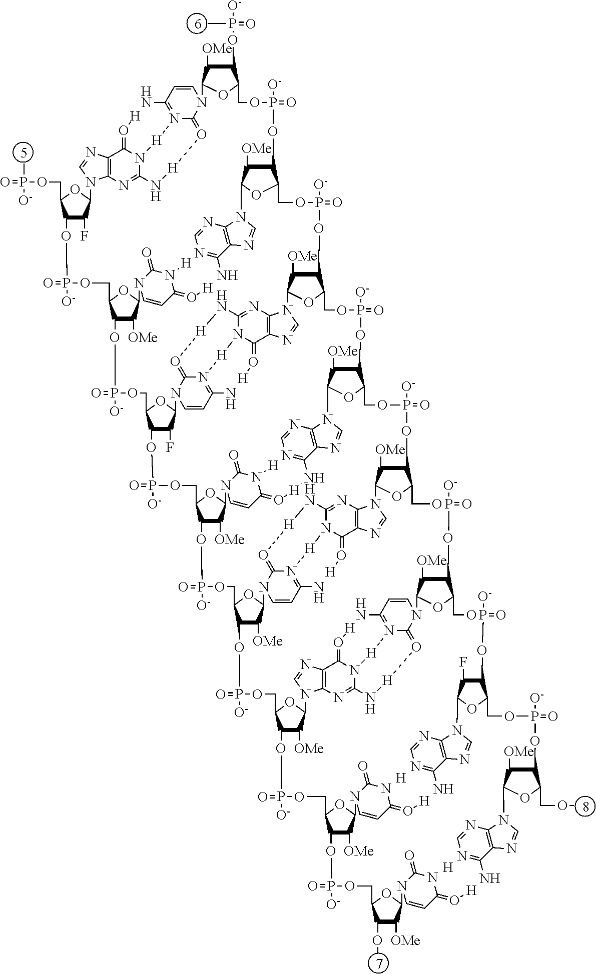 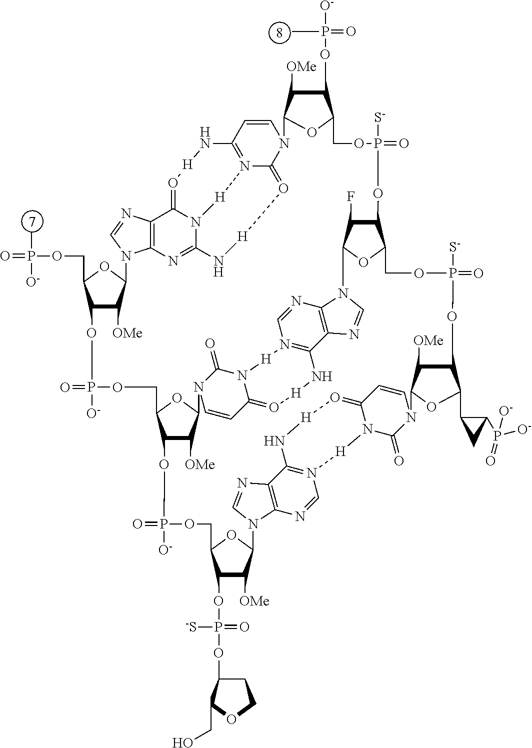
or a pharmaceutically acceptable salt thereof.
